(19) United States
(12) Patent Application Publication
(10) Pub. No.: US 2016/0122342 A1
(43) Pub. Date: May 5, 2016

(54) ENHANCER OF ZESTE HOMOLOG 2 INHIBITORS

(71) Applicant: GLAXOSMITHKLINE INTELLECTUAL PROPERTY (NO.2) LIMITED, Brentford, Middlesex (GB)

(72) Inventors: David T. FOSBENNER, Collegeville, PA (US); Bryan W. KING, Collegeville, PA (US); Steven David KNIGHT, Collegeville, PA (US); Louis Vincent LaFRANCE, III, Collegeville, PA (US); Mei LI, Collegeville, PA (US); Kenneth C. McNULTY, Collegeville, PA (US); Stuart Paul ROMERIL, Collegeville, PA (US); Mark Andrew SEEFELD, Collegeville, PA (US)

(21) Appl. No.: 14/895,947

(22) PCT Filed: Jun. 6, 2014
(86) PCT No.: PCT/IB2014/062025

§ 371 Date: Dec. 4, 2015

Related U.S. Application Data
(60) Provisional application No. 61/977,666, filed on Apr. 10, 2014.
(60) Provisional application No. 61/949,399, filed on Mar. 7, 2014.
(60) Provisional application No. 61/831,649, filed on Jun. 6, 2013.

(51) Int. Cl. C07D 471/04; C07D 471/14; C07D 498/04

(52) U.S. Cl. CPC C07D 471/04; C07D 498/04; C07D 471/14

(57) ABSTRACT

This invention relates to novel compounds according to Formula (I) which are inhibitors of Enhancer of Zeste Homolog 2 (EZH2), to pharmaceutical compositions containing them, to processes for their preparation, and to their use in therapy for the treatment of cancers.

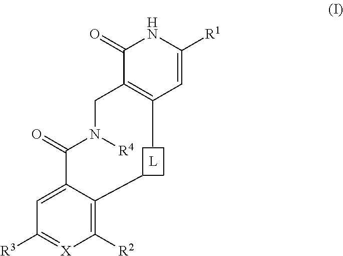

### FIELD OF THE INVENTION

This invention relates to compounds which inhibit Enhancer of Zeste Homolog 2 (EZH2) and thus are useful for inhibiting the proliferation of and/or inducing apoptosis in cancer cells.

### BACKGROUND OF THE INVENTION

Epigenetic modifications play an important role in the regulation of many cellular processes including cell proliferation, differentiation, and cell survival. Global epigenetic modifications are common in cancer, and include global changes in DNA and/or histone methylation, dysregulation of non-coding RNAs and nucleosome remodeling leading to aberrant activation or inactivation of oncogenes, tumor suppressors and signaling pathways. However, unlike genetic mutations which arise in cancer, these epigenetic changes can be reversed through selective inhibition of the enzymes involved. Several methylases involved in histone or DNA methylation are known to be dysregulated in cancer. Thus, selective inhibitors of particular methylases will be useful in the treatment of proliferative diseases such as cancer.

EZH2 (human EZH2 gene: Cardoso, C, et al; European J of Human Genetics, Vol. 8, No. 3 Pages 174-180, 2000) is the catalytic subunit of the Polycomb Repressor Complex 2 (PRC2) which functions to silence target genes by tri-methylating lysine 27 of histone H3 (H3K27me3). Histone H3 is one of the five main histone proteins involved in the structure of chromatin in eukaryotic cells. Featuring a main globular domain and a long N-terminal tail, Histones are involved with the structure of the nucleosomes, a ‘beads on a string’ structure. Histone proteins are highly post-translationally modified however Histone H3 is the most extensively modified of the five histones. The term “Histone H3” alone is purposely ambiguous in that it does not distinguish between sequence variants or modification state. Histone H3 is an important protein in the emerging field of epigenetics, where its sequence variants and variable modification states are thought to play a role in the dynamic and long term regulation of genes.

Increased EZH2 expression has been observed in numerous solid tumors including those of the prostate, breast, skin, bladder, liver, pancreas, head and neck and correlates with cancer aggressiveness, metastasis and poor outcome (Varambally et al., 2002; Kleer et al., 2003; Breuer et al., 2004; Bachmann et al., 2005; Weikert et al., 2005; Sudo et al., 2005; Bachmann et al., 2006). For instance, there is a greater risk of recurrence after prostatectomy in tumors expressing high levels of EZH2, increased metastasis, shorter disease-free survival and increased death in breast cancer patients with high EZH2 levels (Varambally et al., 2002; Kleer et al., 2003). More recently, inactivating mutations in UTX (ubiquitously transcribed tetratricopeptide repeats X), a H3K27 demethylase which functions in opposition to EZH2, have been identified in multiple solid and hematological tumor types (including renal, glioblastoma, esophageal, breast, colon, non-small cell lung, small cell lung, bladder, multiple myeloma, and chronic myeloid leukemia tumors), and low UTX levels correlate with poor survival in breast cancer suggesting that loss of UTX function leads to increased H3K27me3 and repression of target genes (Wang et al., 2010). Together, these data suggest that increased H3K27me3 levels contribute to cancer aggressiveness in many tumor types and that inhibition of EZH2 activity may provide therapeutic benefit.

Numerous studies have reported that direct knockdown of EZH2 via siRNA or shRNA or indirect loss of EZH2 via treatment with the SAH hydrolase inhibitor 3-deazaneplanocin A (DZNep) decreases cancer cell line proliferation and invasion in vitro and tumor growth in vivo (Gonzalez et al., 2008, GBM 2009). While the precise mechanism by which aberrant EZH2 activity leads to cancer progression is not known, many EZH2 target genes are tumor suppressors suggesting that loss of tumor suppressor function is a key mechanism. In addition, EZH2 overexpression in immortalized or primary epithelial cells promotes anchorage independent growth and invasion and requires EZH2 catalytic activity (Kleer et al., 2003; Cao et al., 2008).

Thus, there is strong evidence to suggest that inhibition of EZH2 activity decreases cellular proliferation and invasion. Accordingly, compounds that inhibit EZH2 activity would be useful for the treatment of cancer.

### SUMMARY OF THE INVENTION

The present invention relates to compounds according to Formula (I):

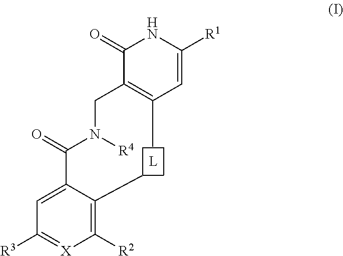

wherein:

X is CH or N;

L is (C₂-C₈)alkylenyl or (C₂-C₈)alkenylenyl, each optionally substituted by hydroxyl, wherein any one methylene unit of said (C₂-C₈)alkylenyl or (C₂-C₈)alkenylenyl is optionally replaced by —O—, —NH—, or —N(C₁-C₄)alkyl-;

R¹ is hydrogen, halogen, (C₁-C₆)alkyl, (C₂-C₆)alkenyl, (C₂-C₆)alkynyl, halo(C₁-C₄)alkyl, (C₃-C₆)cycloalkyl, (C₃-C₆)cycloalkyl(C₁-C₆)alkyl, (C₃-C₆)cycloalkyl(C₂-C₆)alkenyl, (C₅-C₆)cycloalkenyl, (C₅-C₆)cycloalkenyl(C₁-C₆)alkyl, (C₅-C₆)cycloalkenyl(C₂-C₆)alkenyl, (C₆-C₁₀)bicycloalkyl, heterocycloalkyl, heterocycloalkyl(C₁-C₆)alkyl-, heterocycloalkyl(C₂-C₆)alkenyl, phenyl, phenyl(C₁-C₆)alkyl, phenyl(C₂-C₆)alkenyl, heteroaryl, heteroaryl(C₁-C₆)alkyl, heteroaryl(C₂-C₆)alkenyl, cyano, —C(O)R^{a}, —CO₂R^{a}, —C(O)NR^{a}R^{b}, —C(O)NR^{a}NR^{a}R^{b}, —SR^{a}, —S(O)R^{a}, —SO₂R^{a}, —SO₂NR^{a}R^{b}, nitro, —NR^{a}R^{b}, R^{a}R^{b}N(C₁-C₄)alkyl-, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, or —OC(O)NR^{a}R^{b}, wherein each cycloalkyl, cycloalkenyl, bicycloalkyl, heterocycloalkyl, phenyl, or heteroaryl group is optionally substituted 1, 2, or 3 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;

R² is (C₄-C₈)alkyl, (C₁-C₈)alkoxy, (C₄-C₈)cycloalkyl, (C₃-C₈)cycloalkyloxy-, heterocycloalkyl, heterocycloalkyloxy-, aryl, heteroaryl, or —NR^{a}R^{b}, wherein said (C₄-C₈)alkyl, (C₃-C₈)alkoxy, (C₄-C₈)cycloalkyl, (C₃-C₈)cycloalkyloxy-, heterocycloalkyl, heterocycloalkyloxy-, aryl, or heteroaryl is optionally substituted 1, 2, or 3 times, independently, by halogen, —OR^{a}, —NR^{a}R^{b}, —NHCO₂R^{a}, nitro, (C₁-C₃)alkyl, R^{a}R^{b}N(C₁-C₃)alkyl-, R^{a}O(C₁-C₃)alkyl-, (C₃-C₈)cycloalkyl, cyano, —CO₂R^{a}, —C(O)NR^{a}R^{b}, —SO₂NR^{a}R^{b}, aryl, or heteroaryl;

R³ is selected from the group consisting of hydrogen, halogen, (C₁-C₆)alkyl, (C₂-C₆)alkenyl, (C₂-C₆)alkynyl, (C₁-C₄)alkoxy, —B(OH)₂, (C₃-C₆)cycloalkyl, (C₃-C₆)cycloalkyl(C₁-C₄)alkyl-, (C₆-C₁₀)bicycloalkyl, heterocycloalkyl, heterocycloalkyl(C₁-C₄)alkyl-, phenyl, phenyl(C₁-C₂)alkyl, heteroaryl, heteroaryl(C₁-C₂)alkyl, cyano, —C(O)R^{a}, —CO₂R^{a}, —C(O)NR^{a}R^{b}, —C(O)NR^{a}NR^{a}R^{b}, —SR^{a}, —S(O)R^{a}, —SO₂R^{a}, —SO₂NR^{a}R^{b}, nitro, —NR^{a}R^{b}, R^{a}R^{b}N(C₁-C₄)alkyl-, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, R^{a}O(C₁-C₄)alkyl-, R^{a}O(C₃-C₆)alkynyl-, —OC(O)R^{a}, and —OC(O)NR^{a}R^{b}, wherein each cycloalkyl, bicycloalkyl, heterocycloalkyl, phenyl, or heteroaryl group is optionally substituted 1, 2, or 3 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;

R⁴ is hydrogen, (C₁-C₄)alkyl, or hydroxy(C₂-C₄)alkyl-;

each R^{c} is independently —S(O)R^{a}, —SO₂R^{a}, —NR^{a}R^{b}, —NR^{a}C(O)OR^{a}, —NR^{a}SO₂R^{b}, or —CO₂R^{a}; and

R^{a} and R^{b} are each independently hydrogen, (C₁-C₄)alkyl, hydroxy(C₁-C₄)alkyl-, (C₁-C₄)alkoxy(C₁-C₄)alkyl-, (C₃-C₆)cycloalkyl, (C₆-C₁₀)bicycloalkyl, heterocycloalkyl, phenyl, phenyl(C₁-C₂)alkyl-, heteroaryl(C₁-C₄)alkyl-, or heteroaryl, wherein any said cycloalkyl, bicycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, —NH(halo(C₁-C₄)alkyl), —N(halo(C₁-C₄)alkyl)₂, —N((C₁-C₄)alkyl)(halo(C₁-C₄)alkyl), (C₁-C₄)alkyl, halo(C₁-C₄)alkyl, hydroxy(C₁-C₄)alkyl-, (C₁-C₄)alkoxy(C₁-C₄)alkyl-, (C₃-C₆)cycloalkyl, (C₃-C₆)cycloalkyl(C₁-C₄)alkyl-, heterocycloalkyl optionally substituted by one or two halogens, heterocycloalkyl(C₁-C₄)alkyl-, heteroaryl optionally substituted by (C₁-C₄)alkyl, heteroaryl(C₁-C₄)alkyl- optionally substituted by (C₁-C₄)alkyl, (C₁-C₄)alkoxycarbonyl(C₁-C₄)alkyl-, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂;

or R^{a} and R^{b} taken together with the nitrogen to which they are attached represent a 5- or 6-membered saturated or unsaturated ring, optionally containing an additional heteroatom selected from oxygen, nitrogen, and sulfur, wherein said ring is optionally substituted 1, 2, or 3 times, independently, by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-, wherein said ring is optionally fused to a (C₃-C₆)cycloalkyl, heterocycloalkyl, phenyl, or heteroaryl ring;

or R^{a} and R^{b} taken together with the nitrogen to which they are attached represent a 6- to 10-membered bridged bicyclic ring system optionally fused to a (C₃-C₆)cycloalkyl, heterocycloalkyl, phenyl, or heteroaryl ring;

or a pharmaceutically acceptable salt thereof.

Another aspect of this invention relates to a method of inducing apoptosis in cancer cells of solid tumors; treating solid tumor cancers.

Another aspect of the invention relates to pharmaceutical preparations comprising compounds of Formula (I) and pharmaceutically acceptable excipients.

In another aspect, there is provided the use of a compound of Formula (I) or a pharmaceutically acceptable salt or solvate thereof, in the preparation of a medicament for use in the treatment of a disorder mediated by EZH2, such as by inducing apoptosis in cancer cells.

In another aspect, this invention provides for the use of a compound of Formula (I) or a pharmaceutically acceptable salt thereof for the treatment of diseases mediated by EZH2. The invention further provides for the use of a compound of Formula (I) or a pharmaceutically acceptable salt thereof as an active therapeutic substance in the treatment of a disease mediated by EZH2.

In another aspect, the invention provides a compound of Formula (I) or a pharmaceutically acceptable salt thereof for use in therapy.

In another aspect, there is provided a compound of Formula (I) or a pharmaceutically acceptable salt thereof for use in the treatment of a disorder mediated by EZH2.

In another aspect, there is provided a compound of Formula (I) or a pharmaceutically acceptable salt thereof for use in the treatment of cellular proliferation diseases.

In another aspect, there is provided a compound of Formula (I) or a pharmaceutically acceptable salt thereof for use in the treatment of cancer, including the treatment of solid tumors, for example brain (gliomas), glioblastomas, leukemias, lymphomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, gastric, bladder, head and neck, kidney, lung, liver, melanoma, renal, ovarian, pancreatic, prostate, sarcoma, osteosarcoma, giant cell tumor of bone, and thyroid.

In another aspect there is provided methods of co-administering the presently invented compounds of Formula (I) with other active ingredients.

In another aspect there is provided a combination of a compound of Formula (I) or a pharmaceutically acceptable salt thereof and at least one anti-neoplastic agent for use in the treatment of a disorder mediated by EZH2.

In another aspect there is provided a combination of a compound of Formula (I) or a pharmaceutically acceptable salt thereof and at least one anti-neoplastic agent for use in the treatment of cellular proliferation diseases.

In another aspect there is provided a combination of a compound of Formula (I) or a pharmaceutically acceptable salt thereof and at least one anti-neoplastic agent for use in the treatment of cancer, including the treatment of solid tumors, for example brain (gliomas), glioblastomas, leukemias, lymphomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, gastric, bladder, head and neck, kidney, lung, liver, melanoma, renal, ovarian, pancreatic, prostate, sarcoma, osteosarcoma, giant cell tumor of bone, and thyroid.

### DETAILED DESCRIPTION OF THE INVENTION

This invention relates to compounds of the Formula (I) as defined above.

In one embodiment, this invention relates to compounds of Formula (I), wherein:

X is CH or N;

L is (C₂-C₈)alkylenyl or (C₂-C₈)alkenylenyl, each optionally substituted by hydroxyl, wherein any one methylene unit of said (C₂-C₈)alkylenyl or (C₂-C₈)alkenylenyl is optionally replaced by —O—, —NH—, or —N(C₁-C₄)alkyl-;

R¹ is hydrogen, halogen, (C₁-C₆)alkyl, (C₂-C₆)alkenyl, (C₂-C₆)alkynyl, halo(C₁-C₄)alkyl, (C₃-C₆)cycloalkyl, (C₃-C₆)cycloalkyl(C₁-C₆)alkyl, (C₃-C₆)cycloalkyl(C₂-C₆)alkenyl, (C₅-C₆)cycloalkenyl, (C₅-C₆)cycloalkenyl(C₁-C₆)alkyl, (C₅-C₆)cycloalkenyl(C₂-C₆)alkenyl, (C₆-C₁₀)bicycloalkyl, heterocycloalkyl, heterocycloalkyl(C₁-C₆)alkyl-, heterocycloalkyl(C₂-C₆)alkenyl, phenyl, phenyl(C₁-C₆)alkyl, phenyl(C₂-C₆)alkenyl, heteroaryl, heteroaryl(C₁-C₆)alkyl, heteroaryl(C₂-C₆)alkenyl, cyano, —C(O)R^{a}, —CO₂R^{a}, —C(O)NR^{a}R^{b}, —C(O)NR^{a}NR^{a}R^{b}, —SR^{a}, —S(O)R^{a}, —SO₂R^{a}, —SO₂NR^{a}R^{b}, nitro, —NR^{a}R^{b}, R^{a}R^{b}N(C₁-C₄)alkyl-, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, or —OC(O)NR^{a}R^{b}, wherein each cycloalkyl, cycloalkenyl, bicycloalkyl, heterocycloalkyl, phenyl, or heteroaryl group is optionally substituted 1, 2, or 3 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;

R² is (C₄-C₈)alkyl, (C₁-C₈)alkoxy, (C₄-C₈)cycloalkyl, (C₃-C₈)cycloalkyloxy-, heterocycloalkyl, heterocycloalkyloxy-, aryl, heteroaryl, or —NR^{a}R^{b}, wherein said (C₄-C₈)alkyl, (C₃-C₈)alkoxy, (C₄-C₈)cycloalkyl, (C₃-C₈)cycloalkyloxy-, heterocycloalkyl, heterocycloalkyloxy-, aryl, or heteroaryl is optionally substituted 1, 2, or 3 times, independently, by halogen, —OR^{a}, —NR^{a}R^{b}, —NHCO₂R^{a}, nitro, (C₁-C₃)alkyl, R^{a}R^{b}N(C₁-C₃)alkyl-, R^{a}O(C₁-C₃)alkyl-, (C₃-C₈)cycloalkyl, cyano, —CO₂R^{a}, —C(O)NR^{a}R^{b}, —SO₂NR^{a}R^{b}, aryl, or heteroaryl;

R³ is selected from the group consisting of hydrogen, halogen, (C₁-C₆)alkyl, (C₂-C₆)alkenyl, (C₂-C₆)alkynyl, (C₁-C₄)alkoxy, —B(OH)₂, (C₃-C₆)cycloalkyl, (C₃-C₆)cycloalkyl(C₁-C₄)alkyl-, (C₆-C₁₀)bicycloalkyl, heterocycloalkyl, heterocycloalkyl(C₁-C₄)alkyl-, phenyl, phenyl(C₁-C₂)alkyl, heteroaryl, heteroaryl(C₁-C₂)alkyl, cyano, —C(O)R^{a}, —CO₂R^{a}, —C(O)NR^{a}R^{b}, —C(O)NR^{a}NR^{a}R^{b}, —SR^{a}, —S(O)R^{a}, —SO₂R^{a}, —SO₂NR^{a}R^{b}, nitro, —NR^{a}R^{b}, R^{a}R^{b}N(C₁-C₄)alkyl-, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, R^{a}O(C₁-C₄)alkyl-, R^{a}O(C₃-C₆)alkynyl-, —OC(O)R^{a}, and —OC(O)NR^{a}R^{b}, wherein each cycloalkyl, bicycloalkyl, heterocycloalkyl, phenyl, or heteroaryl group is optionally substituted 1, 2, or 3 times, independently, by R^{c}—(C₁-C₆)alkyl-O—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;

R⁴ is hydrogen, (C₁-C₄)alkyl, or hydroxy(C₂-C₄)alkyl-;

each R^{c} is independently —S(O)R^{a}, —SO₂R^{a}, —NR^{a}R^{b}, —NR^{a}C(O)OR^{a}, —NR^{a}SO₂R^{b}, or —CO₂R^{a}; and

R^{a} and R^{b} are each independently hydrogen, (C₁-C₄)alkyl, (C₁-C₄)alkoxy(C₁-C₄)alkyl-, (C₃-C₆)cycloalkyl, heterocycloalkyl, phenyl, phenyl(C₁-C₂)alkyl-, heteroaryl(C₁-C₄)alkyl-, or heteroaryl, wherein any said cycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, (C₁-C₄)alkyl, halo(C₁-C₄)alkyl, hydroxy(C₁-C₄)alkyl-, (C₁-C₄)alkoxy(C₁-C₄)alkyl-, (C₃-C₆)cycloalkyl, (C₃-C₆)cycloalkyl(C₁-C₄)alkyl-, heterocycloalkyl, heterocycloalkyl(C₁-C₄)alkyl-, heteroaryl optionally substituted by (C₁-C₄)alkyl, heteroaryl(C₁-C₄)alkyl- optionally substituted by (C₁-C₄)alkyl, (C₁-C₄)alkoxycarbonyl(C₁-C₄)alkyl-, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂;

or R^{a} and R^{b} taken together with the nitrogen to which they are attached represent a 5- or 6-membered saturated or unsaturated ring, optionally containing an additional heteroatom selected from oxygen, nitrogen, and sulfur, wherein said ring is optionally substituted 1, 2, or 3 times, independently, by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-, wherein said ring is optionally fused to a (C₃-C₆)cycloalkyl, heterocycloalkyl, phenyl, or heteroaryl ring;

or R^{a} and R^{b} taken together with the nitrogen to which they are attached represent a 6- to 10-membered bridged bicyclic ring system optionally fused to a (C₃-C₆)cycloalkyl, heterocycloalkyl, phenyl, or heteroaryl ring;

or a pharmaceutically acceptable salt thereof.

In another embodiment, this invention relates to compounds of Formula (I), wherein:

X is CH or N;

L is (C₂-C₈)alkylenyl or (C₂-C₈)alkenylenyl;

R¹ is hydrogen, halogen, (C₁-C₆)alkyl, (C₂-C₆)alkenyl, (C₂-C₆)alkynyl, halo(C₁-C₄)alkyl, (C₃-C₆)cycloalkyl, (C₃-C₆)cycloalkyl(C₁-C₆)alkyl, (C₃-C₆)cycloalkyl(C₂-C₆)alkenyl, (C₅-C₆)cycloalkenyl, (C₅-C₆)cycloalkenyl(C₁-C₆)alkyl, (C₅-C₆)cycloalkenyl(C₂-C₆)alkenyl, (C₆-C₁₀)bicycloalkyl, heterocycloalkyl, heterocycloalkyl(C₁-C₆)alkyl-, heterocycloalkyl(C₂-C₆)alkenyl, phenyl, phenyl(C₁-C₆)alkyl, phenyl(C₂-C₆)alkenyl, heteroaryl, heteroaryl(C₁-C₆)alkyl, heteroaryl(C₂-C₆)alkenyl, cyano, —C(O)R^{a}, —CO₂R^{a}, —C(O)NR^{a}R^{b}, —C(O)NR^{a}NR^{a}R^{b}, —SR^{a}, —S(O)R^{a}, —SO₂R^{a}, —SO₂NR^{a}R^{b}, nitro, —NR^{a}R^{b}, R^{a}R^{b}N(C₁-C₄)alkyl-, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, or —OC(O)NR^{a}R^{b}, wherein each cycloalkyl, cycloalkenyl, bicycloalkyl, heterocycloalkyl, phenyl, or heteroaryl group is optionally substituted 1, 2, or 3 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;

R² is (C₄-C₈)alkyl, (C₃-C₈)alkoxy, (C₄-C₈)cycloalkyl, (C₃-C₈)cycloalkyloxy-, heterocycloalkyl, heterocycloalkyloxy-, aryl, heteroaryl, or —NR^{a}R^{b}, wherein said (C₄-C₈)alkyl, (C₃-C₈)alkoxy, (C₄-C₈)cycloalkyl, (C₃-C₈)cycloalkyloxy-, heterocycloalkyl, heterocycloalkyloxy-, aryl, or heteroaryl is optionally substituted 1, 2, or 3 times, independently, by halogen, —OR^{a}, —NR^{a}R^{b}, —NHCO₂R^{a}, nitro, (C₁-C₃)alkyl, R^{a}R^{b}N(C₁-C₃)alkyl-, R^{a}O(C₁-C₃)alkyl-, (C₃-C₈)cycloalkyl, cyano, —CO₂R^{a}, —C(O)NR^{a}R^{b}, —SO₂NR^{a}R^{b}, aryl, or heteroaryl;

R³ is selected from the group consisting of hydrogen, halogen, (C₁-C₆)alkyl, (C₂-C₆)alkenyl, (C₂-C₆)alkynyl, (C₁-C₄)alkoxy, —B(OH)₂, (C₃-C₆)cycloalkyl, (C₃-C₆)cycloalkyl(C₁-C₄)alkyl-, (C₆-C₁₀)bicycloalkyl, heterocycloalkyl, heterocycloalkyl(C₁-C₄)alkyl-, phenyl, phenyl(C₁-C₂)alkyl, heteroaryl, heteroaryl(C₁-C₂)alkyl, cyano, —C(O)R^{a}, —CO₂R^{a}, —C(O)NR^{a}R^{b}, —C(O)NR^{a}NR^{a}R^{b}, —SR^{a}, —S(O)R^{a}, —SO₂R^{a}, —SO₂NR^{a}R^{b}, nitro, —NR^{a}R^{b}, R^{a}R^{b}N(C₁-C₄)alkyl-, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, R^{a}O(C₁-C₄)alkyl-, R^{a}O(C₃-C₆)alkynyl-, —OC(O)R^{a}, and —OC(O)NR^{a}R^{b}, wherein each cycloalkyl, bicycloalkyl, heterocycloalkyl, phenyl, or heteroaryl group is optionally substituted 1, 2, or 3 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;

R⁴ is hydrogen;

each R^{c} is independently —S(O)R^{a}, —SO₂R^{a}, —NR^{a}R^{b}, —NR^{a}C(O)OR^{a}, —NR^{a}SO₂R^{b}, or —CO₂R^{a}; and

R^{a} and R^{b} are each independently hydrogen, (C₁-C₄)alkyl, (C₁-C₄)alkoxy(C₁-C₄)alkyl-, (C₃-C₆)cycloalkyl, heterocycloalkyl, phenyl, phenyl(C₁-C₂)alkyl-, heteroaryl(C₁-C₄)alkyl-, or heteroaryl, wherein any said cycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, (C₁-C₄)alkyl, halo(C₁-C₄)alkyl, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂;

or R^{a} and R^{b} taken together with the nitrogen to which they are attached represent a 5- or 6-membered saturated or unsaturated ring, optionally containing an additional heteroatom selected from oxygen, nitrogen, and sulfur, wherein said ring is optionally substituted 1, 2, or 3 times, independently, by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-, wherein said ring is optionally fused to a (C₃-C₆)cycloalkyl, heterocycloalkyl, phenyl, or heteroaryl ring;

or R^{a} and R^{b} taken together with the nitrogen to which they are attached represent a 6- to 10-membered bridged bicyclic ring system optionally fused to a (C₃-C₆)cycloalkyl, heterocycloalkyl, phenyl, or heteroaryl ring;

or a pharmaceutically acceptable salt thereof.

In another embodiment, this invention relates to compounds of Formula (I), wherein R¹ is hydrogen, halogen, (C₁-C₆)alkyl, halo(C₁-C₄)alkyl, (C₃-C₆)cycloalkyl, (C₃-C₆)cycloalkyl(C₁-C₄)alkyl, phenyl, or phenyl(C₁-C₂)alkyl. In another embodiment, this invention relates to compounds of Formula (I), wherein R¹ is (C₁-C₄)alkyl. In a specific embodiment, this invention relates to compounds of Formula (I), wherein R¹ is methyl.

In another embodiment, this invention relates to compounds of Formula (I), wherein R² is (C₄-C₈)alkyl, (C₃-C₈)alkoxy, (C₄-C₈)cycloalkyl, (C₃-C₈)cycloalkyloxy-, heterocycloalkyl, heterocycloalkyloxy-, aryl, heteroaryl, or —NR^{a}R^{b}, wherein said (C₄-C₈)alkyl, (C₃-C₈)alkoxy, (C₄-C₈)cycloalkyl, (C₃-C₈)cycloalkyloxy-, heterocycloalkyl, heterocycloalkyloxy-, aryl, or heteroaryl is optionally substituted 1, 2, or 3 times, independently, by halogen, —OR^{a}, —NHCO₂R^{a}, nitro, (C₁-C₃)alkyl, R^{a}R^{b}N(C₁-C₃)alkyl-, R^{a}O(C₁-C₃)alkyl-, (C₃-C₈)cycloalkyl, cyano, —CO₂R^{a}, —C(O)NR^{a}R^{b}, —SO₂NR^{a}R^{b}, aryl, or heteroaryl.

In another embodiment, this invention relates to compounds of Formula (I), wherein R² is (C₃-C₆)alkoxy, (C₃-C₆)cycloalkyloxy-, heterocycloalkyloxy-, heterocycloalkyl, —NH((C₃-C₆)cycloalkyl), —N((C₁-C₃)alkyl)((C₃-C₆)cycloalkyl), —NH(heterocycloalkyl), or —N((C₁-C₃)alkyl)(heterocycloalkyl), wherein any said (C₃-C₆)alkoxy, (C₃-C₆)cycloalkyloxy-, heterocycloalkyloxy-, heterocycloalkyl, or (C₃-C₆)cycloalkyl is optionally substituted 1 or 2 times, independently, by halogen, hydroxyl, (C₁-C₃)alkoxy, amino, —NH(C₁-C₃)alkyl, —N((C₁-C₃)alkyl)₂, (C₁-C₃)alkyl, (C₁-C₃)alkoxy(C₁-C₃)alkyl-, amino(C₁-C₃)alkyl-, ((C₁-C₃)alkyl)NH(C₁-C₃)alkyl-, ((C₁-C₃)alkyl)₂N(C₁-C₃)alkyl-, (C₃-C₈)cycloalkyl, cyano, —CO₂R^{a}, —C(O)NR^{a}R^{b}, —SO₂NR^{a}R^{b}, phenyl, or heteroaryl.

In another embodiment, this invention relates to compounds of Formula (I), wherein R² is (C₃-C₆)alkoxy, (C₃-C₈)cycloalkyloxy-, or heterocycloalkyloxy-, each of which is optionally substituted by hydroxyl, (C₁-C₃)alkoxy, amino, —NH(C₁-C₃)alkyl, —N((C₁-C₃)alkyl)₂, (C₁-C₃)alkyl, —CO₂R^{a}, —C(O)NR^{a}R^{b}, —SO₂NR^{a}R^{b}, phenyl, or heteroaryl.

In another embodiment, this invention relates to compounds of Formula (I), wherein R² is (C₃-C₆)cycloalkyloxy- which is optionally substituted 1, 2, or 3 times, independently, by halogen, —OR′, —NR^{a}R^{b}, nitro, (C₁-C₃)alkyl, R^{a}R^{b}N(C₁-C₃)alkyl-, R^{a}O(C₁-C₃)alkyl-, (C₃-C₈)cycloalkyl, cyano, —CO₂R^{a}, —C(O)NR^{a}R^{b}, —SO₂NR^{a}R^{b}, aryl, or heteroaryl. In another embodiment, this invention relates to compounds of Formula (I), wherein R² is (C₃-C₆)cycloalkyloxy- which is optionally substituted 1 or 2 times, independently, by halogen, hydroxyl, (C₁-C₃)alkoxy, amino, —NH(C₁-C₃)alkyl, —N((C₁-C₃)alkyl)₂, (C₁-C₃)alkyl, (C₁-C₃)alkoxy(C₁-C₃)alkyl-, amino(C₁-C₃)alkyl-, ((C₁-C₃)alkyl)NH(C₁-C₃)alkyl-, ((C₁-C₃)alkyl)₂N(C₁-C₃)alkyl-, (C₃-C₈)cycloalkyl, cyano, —CO₂R^{a}, —C(O)NR^{a}R^{b}, —SO₂NR^{a}R^{b}, phenyl, or heteroaryl. In another embodiment, this invention relates to compounds of Formula (I), wherein R² is (C₃-C₆)cycloalkyloxy- which is optionally substituted by amino, —NH(C₁-C₃)alkyl, or —N((C₁-C₃)alkyl)₂.

In another embodiment, this invention relates to compounds of Formula (I), wherein R² is heterocycloalkyloxy- which is optionally substituted 1, 2, or 3 times, independently, by halogen, —OR′, —NR^{a}R^{b}, nitro, (C₁-C₃)alkyl, R^{a}R^{b}N(C₁-C₃)alkyl-, R^{a}O(C₁-C₃)alkyl-, (C₃-C₈)cycloalkyl, cyano, —CO₂R^{a}, —C(O)NR^{a}R^{b}, —SO₂NR^{a}R^{b}, aryl, or heteroaryl. In another embodiment, this invention relates to compounds of Formula (I), wherein R² is heterocycloalkyloxy- which is optionally substituted 1 or 2 times, independently, by halogen, hydroxyl, (C₁-C₃)alkoxy, amino, —NH(C₁-C₃)alkyl, —N((C₁-C₃)alkyl)₂, (C₁-C₃)alkyl, (C₁-C₃)alkoxy(C₁-C₃)alkyl-, amino(C₁-C₃)alkyl-, ((C₁-C₃)alkyl)NH(C₁-C₃)alkyl-, ((C₁-C₃)alkyl)₂N(C₁-C₃)alkyl-, (C₃-C₈)cycloalkyl, cyano, —CO₂R^{a}, —C(O)NR^{a}R^{b}, —SO₂NR^{a}R^{b}, phenyl, or heteroaryl.

In another embodiment, this invention relates to compounds of Formula (I), wherein R² is cyclopentyloxy, cyclohexyloxy, pyrrolidinyloxy, piperidinyloxy, or tetrahydropyranyloxy, each of which is optionally substituted by hydroxyl, (C₁-C₃)alkoxy, amino, —NH(C₁-C₃)alkyl, —N((C₁-C₃)alkyl)₂, (C₁-C₃)alkyl, —CO₂R^{a}, —C(O)NR^{a}R^{b}, —SO₂NR^{a}R^{b}, phenyl, furanyl, thienyl, pyrrolyl, imidazolyl, pyrazolyl, triazolyl, tetrazolyl, oxazolyl, thiazolyl, isoxazolyl, isothiazolyl, oxadiazolyl, thiadiazolyl, pyridinyl, pyridazinyl, pyrazinyl, or pyrimidinyl, wherein R^{a} is (C₁-C₄)alkyl or phenyl(C₁-C₂)alkyl and R^{b} is hydrogen or (C₁-C₄)alkyl. In another embodiment, this invention relates to compounds of Formula (I), wherein R² is cyclopentyloxy or cyclohexyloxy, each of which is optionally substituted by amino, —NH(C₁-C₃)alkyl, or —N((C₁-C₃)alkyl)₂. In another embodiment, this invention relates to compounds of Formula (I), wherein R² is cyclohexyloxy which is optionally substituted by amino, —NH(C₁-C₃)alkyl, or —N((C₁-C₃)alkyl)₂.

In another embodiment, this invention relates to compounds of Formula (I), wherein R² is —NR^{a}R^{b}. In another embodiment, this invention relates to compounds of Formula (I), wherein R² is —NR^{a}R^{b}; R^{a} is azetidinyl, oxetanyl, pyrrolidinyl, piperidinyl, piperazinyl, morpholinyl, thiomorpholinyl, or tetrahydropyranyl, each of which is optionally substituted 1 or 2 times, independently, by (C₁-C₄)alkyl; and R^{b} is hydrogen or (C₁-C₄)alkyl. In another embodiment, this invention relates to compounds of Formula (I), wherein R² is —NR^{a}R^{b}; R^{a} is azetidinyl, oxetanyl, pyrrolidinyl, piperidinyl, piperazinyl, morpholinyl, thiomorpholinyl, or tetrahydropyranyl; and R^{b} is methyl or ethyl.

In another embodiment, this invention relates to compounds of Formula (I), wherein R² is —NR^{a}R^{b}; R^{a} is cyclopentyl or cyclohexyl, each of which is optionally substituted by amino, —NH(C₁-C₄)alkyl, or —N((C₁-C₄)alkyl)₂; and R^{b} is hydrogen or (C₁-C₄)alkyl. In another embodiment, this invention relates to compounds of Formula (I), wherein R² is —NR^{a}R^{b}; R^{a} is cyclopentyl or cyclohexyl, each of which is optionally substituted by —N((C₁-C₂)alkyl)₂; and R^{b} is methyl or ethyl.

In another embodiment, this invention relates to compounds of Formula (I), wherein R² is (C₁-C₄)alkoxy, cyclohexyloxy, or —NR^{a}R^{b}, wherein said cyclohexyloxy is optionally substituted by amino, —NH(C₁-C₃)alkyl, or —N((C₁-C₃)alkyl)₂.

In another embodiment, this invention relates to compounds of Formula (I), wherein R³ is selected from the group consisting of hydrogen, halogen, (C₁-C₆)alkyl, (C₁-C₄)alkoxy, —B(OH)₂, (C₃-C₆)cycloalkyl, (C₃-C₆)cycloalkyl(C₁-C₄)alkyl-, (C₆-C₁₀)bicycloalkyl, heterocycloalkyl, heterocycloalkyl(C₁-C₄)alkyl-, phenyl, phenyl(C₁-C₂)alkyl, heteroaryl, heteroaryl(C₁-C₂)alkyl, cyano, —C(O)R^{a}, —CO₂R^{a}, —C(O)NR^{a}R^{b}, —C(O)NR^{a}NR^{a}R^{b}, —SR^{a}, —S(O)R^{a}, —SO₂R^{a}, —SO₂NR^{a}R^{b}, nitro, —NR^{a}R^{b}, R^{a}R^{b}N(C₁-C₄)alkyl-, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, and —OC(O)NR^{a}R^{b}, wherein each cycloalkyl, bicycloalkyl, heterocycloalkyl, phenyl, or heteroaryl group is optionally substituted 1, 2, or 3 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl.

In another embodiment, this invention relates to compounds of Formula (I), wherein R³ is heteroaryl which is optionally substituted 1 or 2 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl; each R^{c} is independently —S(O)R^{a}, —SO₂R^{a}, —NR^{a}R^{b}, —NR^{a}C(O)OR^{a}, —NR^{a}SO₂R^{b}, or —CO₂R^{a}; and R^{a} and R^{b} are each independently hydrogen, (C₁-C₄)alkyl, (C₁-C₄)alkoxy(C₁-C₄)alkyl-, (C₃-C₆)cycloalkyl, heterocycloalkyl, phenyl, phenyl(C₁-C₂)alkyl-, heteroaryl(C₁-C₂)alkyl-, or heteroaryl, wherein any said cycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, (C₁-C₄)alkyl, halo(C₁-C₄)alkyl, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂; or R^{a} and R^{b} taken together with the nitrogen to which they are attached represent a 5- or 6-membered saturated or unsaturated ring, optionally containing an additional heteroatom selected from oxygen, nitrogen, and sulfur, wherein said ring is optionally substituted 1, 2, or 3 times, independently, by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-, wherein said ring is optionally fused to a (C₃-C₆)cycloalkyl, heterocycloalkyl, phenyl, or heteroaryl ring; or R^{a} and R^{b} taken together with the nitrogen to which they are attached represent a 6- to 10-membered bridged bicyclic ring system optionally fused to a (C₃-C₆)cycloalkyl, heterocycloalkyl, phenyl, or heteroaryl ring.

In another embodiment, this invention relates to compounds of Formula (I), wherein R³ is heteroaryl which is optionally substituted by (C₁-C₄)alkoxy, —NR^{a}R^{b}, R^{a}R^{b}N(C₁-C₄)alkyl-, (C₁-C₄)alkylheterocycloalkyl-, halogen, (C₁-C₄)alkyl, (C₃-C₈)cycloalkyl, or heterocycloalkyl. In another embodiment, this invention relates to compounds of Formula (I), wherein R³ is heteroaryl which is optionally substituted by heterocycloalkyl or (C₁-C₄)alkyl-heterocycloalkyl-. In another embodiment, this invention relates to compounds of Formula (I), wherein R³ is heteroaryl which is optionally substituted by —NR^{a}R^{b}. In another embodiment, this invention relates to compounds of Formula (I), wherein R³ is furanyl, thiophenyl, thienyl, pyrrolyl, imidazolyl, pyrazolyl, triazolyl, tetrazolyl, thiazolyl, isothiazolyl, 1,2,3-triazolyl, 1,2,4-triazolyl, oxazolyl, isoxazolyl, 1,2,3-oxadiazolyl, 1,2,5-oxadiazolyl, thiadiazolyl, isothiazolyl, tetrazolyl, pyridinyl, pyridazinyl, pyrazinyl, pyrimidinyl, or triazinyl, each of which is optionally substituted by —NR^{a}R^{b}. In another embodiment, this invention relates to compounds of Formula (I), wherein R³ is furanyl, thiophenyl, thienyl, pyrrolyl, imidazolyl, pyrazolyl, triazolyl, tetrazolyl, thiazolyl, isothiazolyl, 1,2,3-triazolyl, 1,2,4-triazolyl, oxazolyl, isoxazolyl, 1,2,3-oxadiazolyl, 1,2,5-oxadiazolyl, thiadiazolyl, isothiazolyl, tetrazolyl, pyridinyl, pyridazinyl, pyrazinyl, pyrimidinyl, or triazinyl, each of which is optionally substituted by pyrrolidinyl, piperidinyl, piperazinyl, 4-methylpiperazinyl, morpholinyl, or thiomorpholinyl.

In another embodiment, this invention relates to compounds of Formula (I), wherein R³ is pyridinyl which is optionally substituted by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl; each R^{c} is independently —S(O)R^{a}, —SO₂R^{a}, —NR^{a}R^{b}, —NR^{a}C(O)OR^{a}, —NR^{a}SO₂R^{b}, or —CO₂R^{a}; and R^{a} and R^{b} are each independently hydrogen, (C₁-C₄)alkyl, (C₁-C₄)alkoxy(C₁-C₄)alkyl-, (C₃-C₆)cycloalkyl, heterocycloalkyl, phenyl, phenyl(C₁-C₂)alkyl-, heteroaryl(C₁-C₂)alkyl-, or heteroaryl, wherein any said cycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, (C₁-C₄)alkyl, halo(C₁-C₄)alkyl, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂; or R^{a} and R^{b} taken together with the nitrogen to which they are attached represent a 5- or 6-membered saturated or unsaturated ring, optionally containing an additional heteroatom selected from oxygen, nitrogen, and sulfur, wherein said ring is optionally substituted 1, 2, or 3 times, independently, by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-, wherein said ring is optionally fused to a (C₃-C₆)cycloalkyl, heterocycloalkyl, phenyl, or heteroaryl ring; or R^{a} and R^{b} taken together with the nitrogen to which they are attached represent a 6- to 10-membered bridged bicyclic ring system optionally fused to a (C₃-C₆)cycloalkyl, heterocycloalkyl, phenyl, or heteroaryl ring.

In another embodiment, this invention relates to compounds of Formula (I), wherein R³ is pyridinyl which is optionally substituted by (C₁-C₄)alkoxy, —NR^{a}R^{b}, R^{a}R^{b}N(C₁-C₄)alkyl-, (C₁-C₄)alkylheterocycloalkyl-, halogen, (C₁-C₄)alkyl, (C₃-C₈)cycloalkyl, or heterocycloalkyl. In another embodiment, this invention relates to compounds of Formula (I), wherein R³ is pyridinyl which is optionally substituted by heterocycloalkyl or (C₁-C₄)alkyl-heterocycloalkyl-. In another embodiment, this invention relates to compounds of Formula (I), wherein R³ is pyridinyl which is optionally substituted by —NR^{a}R^{b}. In another embodiment, this invention relates to compounds of Formula (I), wherein R³ is pyridinyl which is optionally substituted by pyrrolidinyl, piperidinyl, piperazinyl, 4-methylpiperazinyl, morpholinyl, or thiomorpholinyl. In another embodiment, this invention relates to compounds of Formula (I), wherein R³ is pyridinyl which is substituted by piperazinyl.

In another embodiment, this invention relates to compounds of Formula (I), wherein R³ is selected from the group consisting of hydrogen, —SO₂(C₁-C₄)alkyl, halogen, (C₁-C₆)alkyl, (C₂-C₆)alkenyl, (C₂-C₆)alkynyl, hydroxy(C₁-C₄)alkyl-, hydroxy(C₃-C₆)alkynyl-, (C₁-C₄)alkoxy, phenyl, heteroaryl, and cyano, wherein said phenyl or heteroaryl group is optionally substituted 1 or 2 times, independently, by (C₁-C₄)alkoxy, —NR^{a}R^{b}, R^{a}R^{b}N(C₁-C₄)alkyl-, (C₁-C₄)alkylheterocycloalkyl-, halogen, (C₁-C₄)alkyl, (C₃-C₈)cycloalkyl, or heterocycloalkyl. In another embodiment, this invention relates to compounds of Formula (I), wherein R³ is selected from the group consisting of hydrogen, —SO₂(C₁-C₄)alkyl, halogen, (C₁-C₆)alkyl, (C₁-C₄)alkoxy, phenyl, heteroaryl, and cyano, wherein said phenyl or heteroaryl group is optionally substituted 1 or 2 times, independently, by (C₁-C₄)alkoxy, —NR^{a}R^{b}, R^{a}R^{b}N(C₁-C₄)alkyl-, (C₁-C₄)alkylheterocycloalkyl-, halogen, (C₁-C₄)alkyl, (C₃-C₈)cycloalkyl, or heterocycloalkyl. In another embodiment, this invention relates to compounds of Formula (I), wherein R³ is selected from the group consisting of hydrogen, halogen, phenyl, and heteroaryl, wherein said phenyl or heteroaryl group is optionally substituted 1 or 2 times, independently, by (C₁-C₄)alkoxy, —NR^{a}R^{b}, R^{a}R^{b}N(C₁-C₄)alkyl-, (C₁-C₄)alkylheterocycloalkyl-, halogen, (C₁-C₄)alkyl, (C₃-C₈)cycloalkyl, or heterocycloalkyl. In another embodiment, this invention relates to compounds of Formula (I), wherein R³ is selected from the group consisting of halogen, phenyl, and heteroaryl, wherein said phenyl or heteroaryl group is optionally substituted by heterocycloalkyl or (C₁-C₄)alkyl-heterocycloalkyl-.

In another embodiment, this invention relates to compounds of Formula (I), wherein R³ is selected from the group consisting of hydrogen, cyano, halogen, (C₁-C₄)alkoxy, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, phenyl, pyridinyl, pyridazinyl, pyrazinyl, pyrimidinyl, and triazinyl, wherein said furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, phenyl, pyridinyl, pyridazinyl, pyrazinyl, pyrimidinyl, or triazinyl is optionally substituted by (C₁-C₄)alkoxy, —NR^{a}R^{b}, R^{a}R^{b}N(C₁-C₄)alkyl-, (C₁-C₄)alkylheterocycloalkyl-, halogen, (C₁-C₄)alkyl, (C₃-C₈)cycloalkyl, or heterocycloalkyl.

In another embodiment, this invention relates to compounds of Formula (I), wherein R³ is phenyl which is optionally substituted by —NR^{a}R^{b} or R^{a}R^{b}N(C₁-C₄)alkyl-.

In another embodiment, this invention relates to compounds of Formula (I), wherein R³ is cyano, halogen, (C₁-C₄)alkyl, (C₂-C₆)alkenyl, (C₂-C₆)alkynyl, hydroxy(C₁-C₄)alkyl-, hydroxy(C₃-C₆)alkynyl-, or (C₁-C₄)alkoxy. In another embodiment, this invention relates to compounds of Formula (I), wherein R³ is hydroxy(C₃-C₆)alkynyl-. In a specific embodiment, this invention relates to compounds of Formula (I), wherein R³ is cyano.

In another embodiment, this invention relates to compounds of Formula (I), wherein R³ is halogen, (C₁-C₄)alkyl, (C₂-C₆)alkenyl, (C₂-C₆)alkynyl, hydroxy(C₁-C₄)alkyl-, hydroxy(C₃-C₆)alkynyl-, or (C₁-C₄)alkoxy. In another embodiment, this invention relates to compounds of Formula (I), wherein R³ is hydrogen, halogen, (C₁-C₄)alkyl, or (C₁-C₄)alkoxy. In another embodiment, this invention relates to compounds of Formula (I), wherein R³ is hydrogen or halogen. In a specific embodiment, this invention relates to compounds of Formula (I), wherein R³ is hydrogen, fluorine, chlorine, or bromine. In another specific embodiment, this invention relates to compounds of Formula (I), wherein R³ is hydrogen or chlorine. In a more specific embodiment, this invention relates to compounds of Formula (I), wherein R³ is hydrogen.

In another embodiment, this invention relates to compounds of Formula (I), wherein R³ is halogen. In a specific embodiment, this invention relates to compounds of Formula (I), wherein R³ is fluorine, chlorine, or bromine. In a more specific embodiment, this invention relates to compounds of Formula (I), wherein R³ is chlorine.

In another embodiment, this invention relates to compounds of Formula (I), wherein R⁴ is hydrogen or (C₁-C₄)alkyl. In a specific embodiment, this invention relates to compounds of Formula (I), wherein R⁴ is hydrogen.

In another embodiment, this invention relates to compounds of Formula (I), wherein R^{a} and R^{b} are each independently hydrogen, (C₁-C₄)alkyl, (C₁-C₄)alkoxy(C₁-C₄)alkyl-, (C₃-C₆)cycloalkyl, heterocycloalkyl, phenyl, phenyl(C₁-C₂)alkyl-, heteroaryl(C₁-C₄)alkyl-, or heteroaryl, wherein any said cycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, (C₁-C₄)alkyl, halo(C₁-C₄)alkyl, hydroxy(C₁-C₄)alkyl-, (C₁-C₄)alkoxy(C₁-C₄)alkyl-, (C₃-C₆)cycloalkyl, (C₃-C₆)cycloalkyl(C₁-C₄)alkyl-, heterocycloalkyl, heterocycloalkyl(C₁-C₄)alkyl-, heteroaryl optionally substituted by (C₁-C₄)alkyl, heteroaryl(C₁-C₄)alkyl- optionally substituted by (C₁-C₄)alkyl, (C₁-C₄)alkoxycarbonyl(C₁-C₄)alkyl-, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂.

In another embodiment, this invention relates to compounds of Formula (I), wherein R^{a} and R^{b} are each independently hydrogen, (C₁-C₄)alkyl, (C₁-C₄)alkoxy(C₁-C₄)alkyl-, (C₃-C₆)cycloalkyl, heterocycloalkyl, phenyl, phenyl(C₁-C₂)alkyl-, heteroaryl(C₁-C₄)alkyl-, or heteroaryl, wherein any said cycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, (C₁-C₄)alkyl, halo(C₁-C₄)alkyl, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂; or R^{a} and R^{b} taken together with the nitrogen to which they are attached represent a 5- or 6-membered saturated or unsaturated ring, optionally containing an additional heteroatom selected from oxygen, nitrogen, and sulfur, wherein said ring is optionally substituted 1, 2, or 3 times, independently, by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-, wherein said ring is optionally fused to a (C₃-C₆)cycloalkyl, heterocycloalkyl, phenyl, or heteroaryl ring; or R^{a} and R^{b} taken together with the nitrogen to which they are attached represent a 6- to 10-membered bridged bicyclic ring system optionally fused to a (C₃-C₆)cycloalkyl, heterocycloalkyl, phenyl, or heteroaryl ring.

In another embodiment, this invention relates to compounds of Formula (I), wherein R^{a} and R^{b} are each independently hydrogen, (C₁-C₄)alkyl, (C₁-C₄)alkoxy(C₁-C₄)alkyl-, (C₃-C₆)cycloalkyl, heterocycloalkyl, phenyl, phenyl(C₁-C₂)alkyl-, heteroaryl(C₁-C₄)alkyl-, or heteroaryl, wherein any said cycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, (C₁-C₄)alkyl, halo(C₁-C₄)alkyl, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂.

In another embodiment, this invention relates to compounds of Formula (I), wherein R^{a} and R^{b} are each independently hydrogen, (C₁-C₄)alkyl, cyclohexyl, tetrahydropyranyl, and piperidinyl, wherein said cyclohexyl or piperidinyl is optionally substituted 1 or 2 times, independently, by halogen, (C₁-C₄)alkyl, halo(C₁-C₄)alkyl, hydroxy(C₁-C₄)alkyl-, (C₃-C₆)cycloalkyl(C₁-C₄)alkyl-, heterocycloalkyl, heteroaryl(C₁-C₄)alkyl- optionally substituted by (C₁-C₄)alkyl, (C₁-C₄)alkoxycarbonyl(C₁-C₄)alkyl-, —SO₂(C₁-C₄)alkyl, amino, —NH(C₁-C₄)alkyl, or —N((C₁-C₄)alkyl)₂.

In another embodiment, this invention relates to compounds of Formula (I), wherein R^{a} is hydrogen, (C₁-C₄)alkyl, cyclohexyl, tetrahydropyranyl, and piperidinyl, wherein said cyclohexyl or piperidinyl is optionally substituted 1 or 2 times, independently, by halogen, (C₁-C₄)alkyl, halo(C₁-C₄)alkyl, hydroxy(C₁-C₄)alkyl-, (C₃-C₆)cycloalkyl(C₁-C₄)alkyl-, heterocycloalkyl, heteroaryl(C₁-C₄)alkyl- optionally substituted by (C₁-C₄)alkyl, (C₁-C₄)alkoxycarbonyl(C₁-C₄)alkyl-, —SO₂(C₁-C₄)alkyl, amino, —NH(C₁-C₄)alkyl, or —N((C₁-C₄)alkyl)₂; and R^{b} is hydrogen or (C₁-C₄)alkyl.

In another embodiment, this invention relates to compounds of Formula (I), wherein R^{a} is hydrogen, methyl, ethyl, cyclohexyl, tetrahydropyranyl, or piperidinyl, wherein said cyclohexyl is optionally substituted 1 or 2 times, independently, by fluorine, amino, dimethylamino, diethylamino, or morpholinyl, and wherein said piperidinyl is optionally substituted by methyl, ethyl, isopropyl, 2,2,2-trifluoroethyl, 3,3,3-trifluoropropyl, 2-hydroxyethyl, 1,3-dihydroxypropan-2-yl, cyclopropylmethyl, (1-methyl-1H-pyrazol-3-yl)methyl, (6-methylpyridin-2-yl)methyl, 1-ethoxy-2-methyl-1-oxopropan-2-yl, or methylsulfonyl; and R^{b} is hydrogen, methyl, or ethyl.

In another embodiment, this invention relates to compounds of Formula (I), wherein L is (C₂-C₈)alkylenyl or (C₂-C₈)alkenylenyl, each optionally substituted by hydroxyl. In another embodiment, this invention relates to compounds of Formula (I), wherein L is (C₅-C₇)alkylenyl or (C₅-C₇)alkenylenyl, each independently substituted by hydroxyl. In another embodiment, this invention relates to compounds of Formula (I), wherein L is selected from the group consisting of:

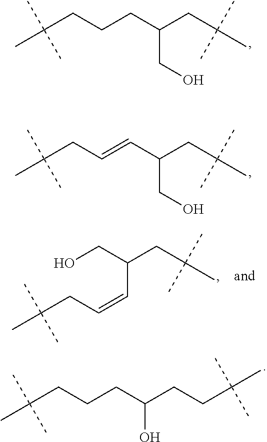

In another embodiment, this invention relates to compounds of Formula (I), wherein L is (C₂-C₈)alkylenyl or (C₂-C₈)alkenylenyl, wherein any one methylene unit of said (C₂-C₈)alkylenyl or (C₂-C₈)alkenylenyl is optionally replaced by —O—, —NH—, or —N(C₁-C₄)alkyl-. In another embodiment, this invention relates to compounds of Formula (I), wherein L is (C₅-C₇)alkylenyl or (C₅-C₇)alkenylenyl, wherein any one methylene unit of said (C₅-C₇)alkylenyl or (C₅-C₇)alkenylenyl is replaced by —O—, —NH—, or —N(C₁-C₄)alkyl-. In another embodiment, this invention relates to compounds of Formula (I), wherein L is selected from the group consisting of:

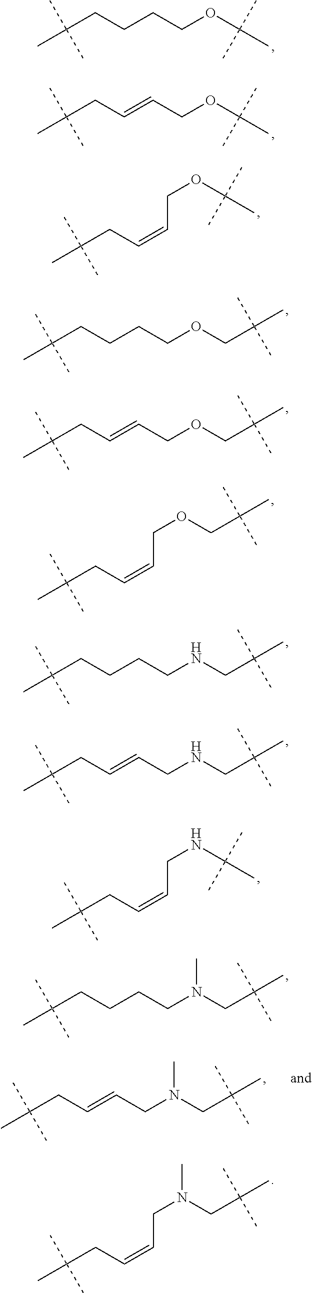

In a specific embodiment, this invention relates to compounds of Formula (I), wherein L is

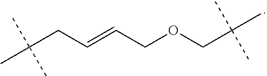

In another specific embodiment, this invention relates to compounds of Formula (I), wherein L is

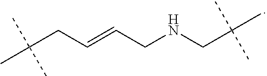

In another embodiment, this invention relates to compounds of Formula (I), wherein L is (C₂-C₈)alkylenyl or (C₂-C₈)alkenylenyl. In another embodiment, this invention relates to compounds of Formula (I), wherein L is (C₅-C₇)alkylenyl or (C₅-C₇)alkenylenyl. In another embodiment, this invention relates to compounds of Formula (I), wherein L is (C₄-C₆)alkylenyl or (C₄-C₆)alkenylenyl. In another embodiment, this invention relates to compounds of Formula (I), wherein L is selected from the group consisting of:

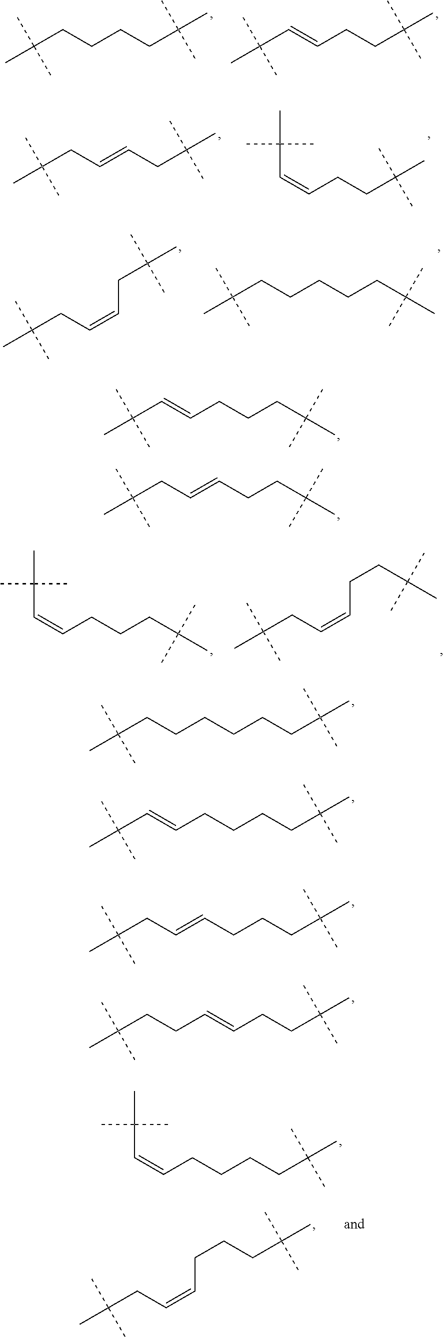

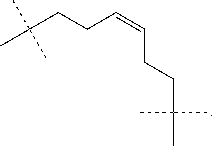

In another embodiment, this invention relates to compounds of Formula (I), wherein L is (C₅-C₆)alkylenyl or (C₅-C₆)alkenylenyl. In another embodiment, this invention relates to compounds of Formula (I), wherein L is selected from the group consisting of:

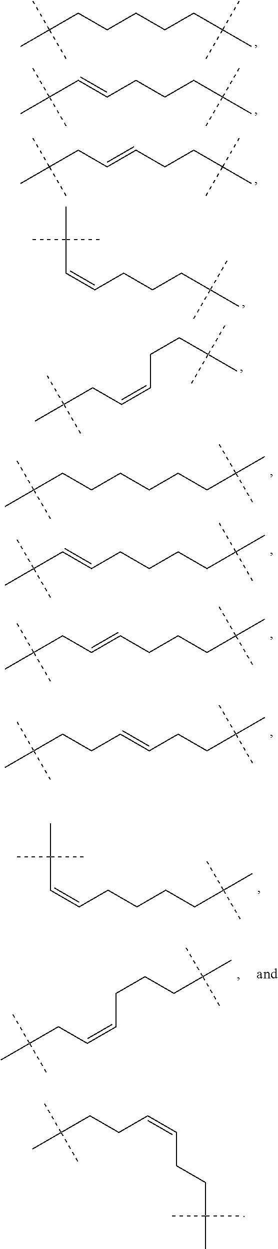

In another embodiment, this invention relates to compounds of Formula (I), wherein L is selected from the group consisting of:

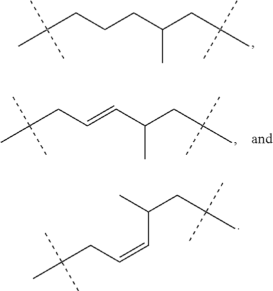

In another embodiment, this invention relates to compounds of Formula (I), wherein L is selected from the group consisting of:

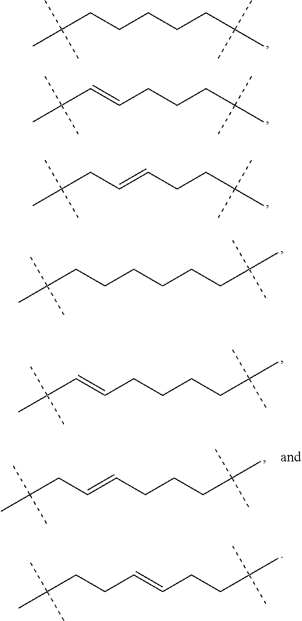

In another embodiment, this invention relates to compounds of Formula (I), wherein L is selected from the group consisting of:

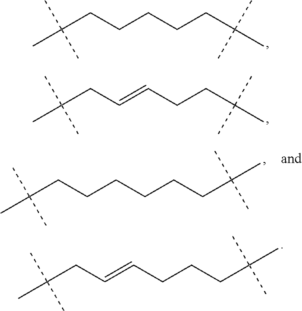

In a specific embodiment, this invention relates to compounds of Formula (I), wherein L is

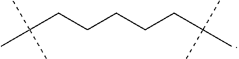

In another specific embodiment, this invention relates to compounds of Formula (I), wherein L is

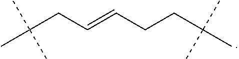

In another specific embodiment, this invention relates to compounds of Formula (I), wherein L is

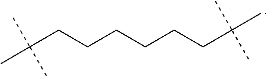

In another specific embodiment, this invention relates to compounds of Formula (I), wherein L is

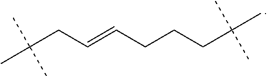

In another specific embodiment, this invention relates to compounds of Formula (I), wherein L is

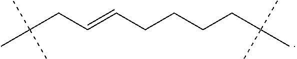

In a particular embodiment, this invention relates to compounds of Formula (I), wherein:

X is CH;

L is selected from the group consisting of:

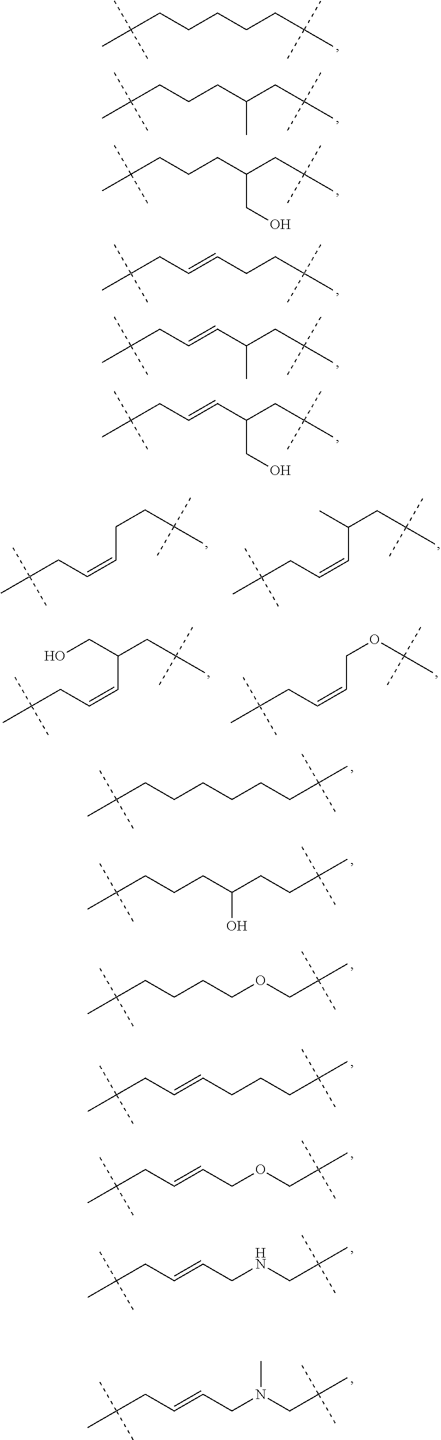

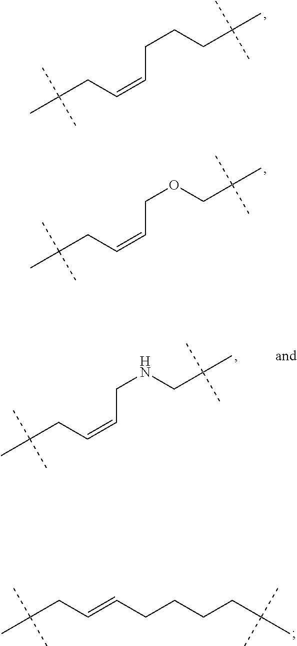

R¹ is (C₁-C₄)alkyl;

R² is (C₁-C₄)alkoxy, cyclohexyloxy, or —NR^{a}R^{b}, wherein said cyclohexyloxy is optionally substituted by amino, —NH(C₁-C₃)alkyl, or —N((C₁-C₃)alkyl)₂;

R³ is hydrogen or halogen;

R⁴ is hydrogen, (C₁-C₄)alkyl, or hydroxy(C₂-C₄)alkyl-; and

R^{a} and R^{b} are each independently hydrogen, (C₁-C₄)alkyl, cyclohexyl, tetrahydropyranyl, and piperidinyl, wherein said cyclohexyl or piperidinyl is optionally substituted 1 or 2 times, independently, by halogen, (C₁-C₄)alkyl, halo(C₁-C₄)alkyl, hydroxy(C₁-C₄)alkyl-, (C₃-C₆)cycloalkyl(C₁-C₄)alkyl-, heterocycloalkyl, heteroaryl(C₁-C₄)alkyl- optionally substituted by (C₁-C₄)alkyl, (C₁-C₄)alkoxycarbonyl(C₁-C₄)alkyl-, —SO₂(C₁-C₄)alkyl, amino, —NH(C₁-C₄)alkyl, or —N((C₁-C₄)alkyl)₂;

or a pharmaceutically acceptable salt thereof.

In another particular embodiment, this invention relates to compounds of Formula (I), wherein:

X is CH;

L is selected from the group consisting of:

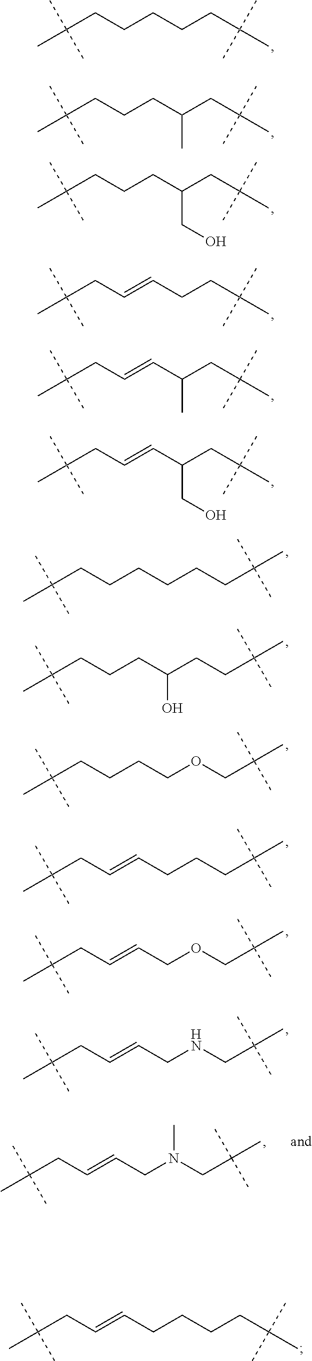

R¹ is (C₁-C₄)alkyl;

R² is (C₁-C₄)alkoxy, cyclohexyloxy, or —NR^{a}R^{b}, wherein said cyclohexyloxy is optionally substituted by amino, —NH(C₁-C₃)alkyl, or —N((C₁-C₃)alkyl)₂;

R³ is hydrogen or chlorine;

R⁴ is hydrogen;

R^{a} is hydrogen, methyl, ethyl, cyclohexyl, tetrahydropyranyl, or piperidinyl, wherein said cyclohexyl is optionally substituted 1 or 2 times, independently, by fluorine, amino, dimethylamino, diethylamino, or morpholinyl, and wherein said piperidinyl is optionally substituted by methyl, ethyl, isopropyl, 2,2,2-trifluoroethyl, 3,3,3-trifluoropropyl, 2-hydroxyethyl, 1,3-dihydroxypropan-2-yl, cyclopropylmethyl, (1-methyl-1H-pyrazol-3-yl)methyl, (6-methylpyridin-2-yl)methyl, 1-ethoxy-2-methyl-1-oxopropan-2-yl, or methylsulfonyl; and

R^{b} is hydrogen, methyl, or ethyl;

or a pharmaceutically acceptable salt thereof.

In another particular embodiment, this invention relates to compounds of Formula (I), wherein:

X is CH;

L is (C₄-C₆)alkylenyl or (C₄-C₆)alkenylenyl;

R¹ is hydrogen, halogen, (C₁-C₆)alkyl, halo(C₁-C₄)alkyl, (C₃-C₆)cycloalkyl, (C₃-C₆)cycloalkyl(C₁-C₄)alkyl, phenyl, or phenyl(C₁-C₂)alkyl;

R² is (C₃-C₆)alkoxy, (C₃-C₈)cycloalkyloxy-, or heterocycloalkyloxy-, each of which is optionally substituted by hydroxyl, (C₁-C₃)alkoxy, amino, —NH(C₁-C₃)alkyl, —N((C₁-C₃)alkyl)₂, (C₁-C₃)alkyl, —CO₂R^{a}, —C(O)NR^{a}R^{b}, —SO₂NR^{a}R^{b}, phenyl, or heteroaryl;

R³ is selected from the group consisting of hydrogen, halogen, phenyl, and heteroaryl, wherein said phenyl or heteroaryl group is optionally substituted by heterocycloalkyl or (C₁-C₄)alkyl-heterocycloalkyl-; and

R⁴ is hydrogen;

or a pharmaceutically acceptable salt thereof.

In another particular embodiment, this invention relates to compounds of Formula (I), wherein:

X is CH;

L is (C₄-C₆)alkylenyl or (C₄-C₆)alkenylenyl;

R¹ is (C₁-C₄)alkyl;

R² is —NR^{a}R^{b};

R³ is selected from the group consisting of hydrogen, halogen, phenyl, and heteroaryl, wherein said phenyl or heteroaryl group is optionally substituted by heterocycloalkyl or (C₁-C₄)alkyl-heterocycloalkyl-; and

R⁴ is hydrogen;

or a pharmaceutically acceptable salt thereof.

In another particular embodiment, this invention relates to compounds of Formula (I), wherein:

X is CH;

L is selected from the group consisting of:

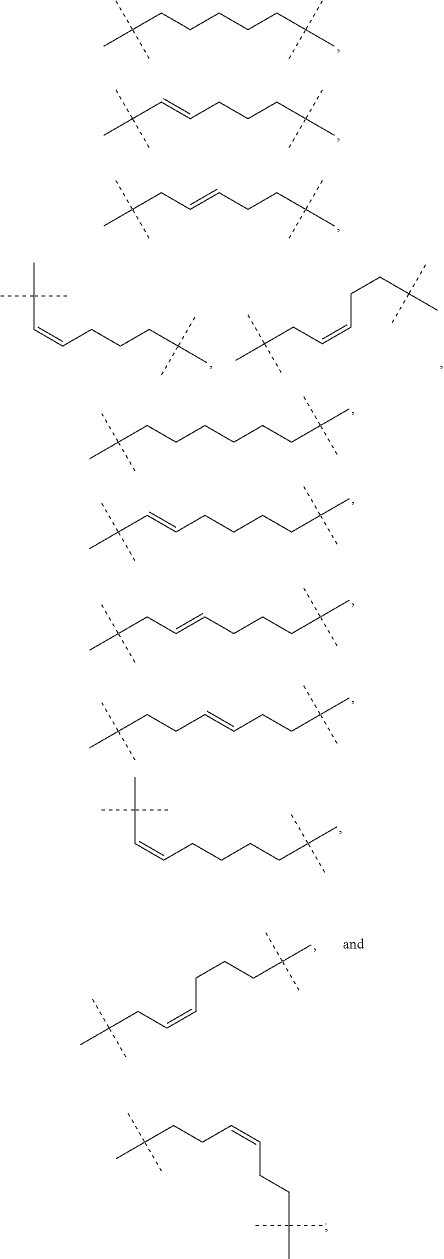

R¹ is (C₁-C₄)alkyl;

R² is cyclopentyloxy, cyclohexyloxy, pyrrolidinyloxy, piperidinyloxy, or tetrahydropyranyloxy, each of which is optionally substituted by hydroxyl, (C₁-C₃)alkoxy, amino, —NH(C₁-C₃)alkyl, —N((C₁-C₃)alkyl)₂, (C₁-C₃)alkyl, —CO₂R^{a}, —C(O)NR^{a}R^{b}, —SO₂NR^{a}R^{b}, phenyl, furanyl, thienyl, pyrrolyl, imidazolyl, pyrazolyl, triazolyl, tetrazolyl, oxazolyl, thiazolyl, isoxazolyl, isothiazolyl, oxadiazolyl, thiadiazolyl, pyridinyl, pyridazinyl, pyrazinyl, or pyrimidinyl, wherein R^{a} is (C₁-C₄)alkyl or phenyl(C₁-C₂)alkyl and R^{b} is hydrogen or (C₁-C₄)alkyl;

R³ is hydrogen or halogen; and

R⁴ is hydrogen;

or a pharmaceutically acceptable salt thereof.

In another particular embodiment, this invention relates to compounds of Formula (I), wherein:

X is CH;

L is selected from the group consisting of:

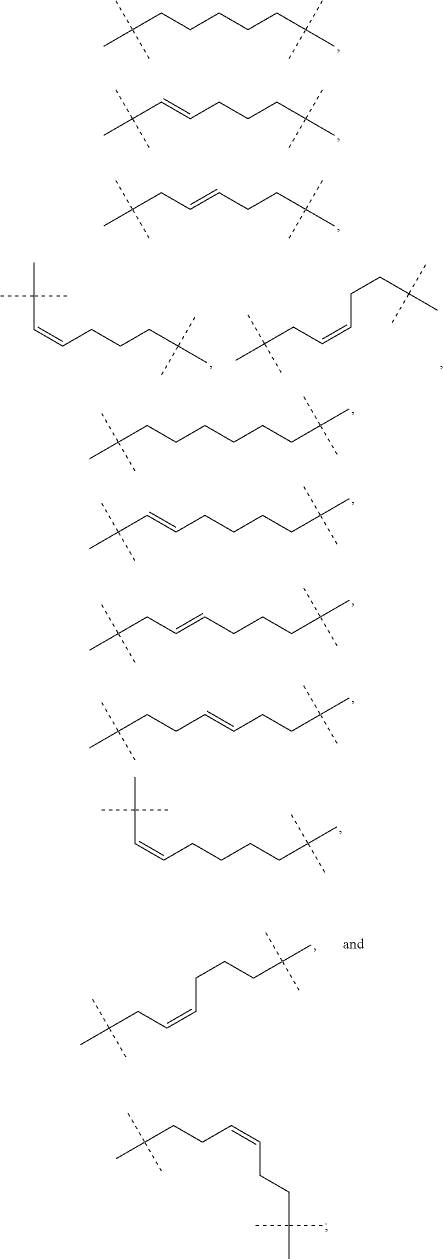

R¹ is (C₁-C₄)alkyl;

R² is —NR^{a}R^{b};

R³ is hydrogen or halogen;

R⁴ is hydrogen;

R^{a} is azetidinyl, oxetanyl, pyrrolidinyl, piperidinyl, piperazinyl, morpholinyl, thiomorpholinyl, or tetrahydropyranyl, each of which is optionally substituted 1 or 2 times, independently, by (C₁-C₄)alkyl; and

R^{b} is hydrogen or (C₁-C₄)alkyl;

or a pharmaceutically acceptable salt thereof.

Specific compounds of this invention include:
(E)-10-((trans-4-aminocyclohexyl)oxy)-12-chloro-3-methyl-5,6,15,16-tetrahydrobenzo[c]pyrido[4,3-j][1]azacyclododecine-1,14(2H,9H)-dione;
(E)-12-chloro-10-((trans-4-(dimethylamino)cyclohexyl)oxy)-3-methyl-5,6,15,16-tetrahydrobenzo[c]pyrido[4,3-j][1]azacyclododecine-1,14(2H,9H)-dione;
12-chloro-10-((trans-4-(dimethylamino)cyclohexyl)oxy)-3-methyl-6,7,8,9,15,16-hexahydrobenzo[c]pyrido[4,3-j][1]azacyclododecine-1,14(2H,5H)-dione;
(Z)-10-((trans-4-aminocyclohexyl)oxy)-12-chloro-3-methyl-5,6,15,16-tetrahydrobenzo[c]pyrido[4,3-j][1]azacyclododecine-1,14(2H,9H)-dione;
(Z)-12-chloro-10-((trans-4-(dimethylamino)cyclohexyl)oxy)-3-methyl-5,6,15,16-tetrahydrobenzo[c]pyrido[4,3-j][1]azacyclododecine-1,14(2H,9H)-dione;
(E)-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione;
(Z)-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione;
(E)-13-chloro-11-((trans-4-(di methylamino)cyclohexyl)oxy)-3-methyl-5,6,7,10,16,17-hexahydro-1H-benzo[c]pyrido[4,3-k][1]azacyclotridecine-1,15(2H)-dione;
(E)-10-(ethyl(tetrahydro-2H-pyran-4-yl)amino)-3,15-dimethyl-5,6,15,16-tetrahydrobenzo[c]pyrido[4,3-j][1]azacyclododecine-1,14(2H,9H)-dione;
(E)-11-(ethyl(tetrahydro-2H-pyran-4-yl)amino)-3,6-dimethyl-5,6,7,10,16,17-hexahydro-1H-benzo[h]pyrido[4,3-c][1,6]diazacyclotridecine-1,15(2H)-dione;
11-(ethyl(tetrahydro-2H-pyran-4-yl)amino)-3-methyl-7,8,9,10,16,17-hexahydrobenzo[h]pyrido[4,3-c][1,6]oxaazacyclotridecine-1,15(2H,5H)-dione;
(E)-11-(ethyl(tetrahydro-2H-pyran-4-yl)amino)-3-methyl-7,10,16,17-tetrahydrobenzo[h]pyrido[4,3-c][1,6]oxaazacyclotridecine-1,15(2H,5H)-dione;
(Z)-11-(ethyl(tetrahydro-2H-pyran-4-yl)amino)-3-methyl-7,10,16,17-tetrahydrobenzo[h]pyrido[4,3-c][1,6]oxaazacyclotridecine-1,15(2H,5H)-dione;
(Z)-10-(ethyl(tetrahydro-2H-pyran-4-yl)amino)-3-methyl-6,9,15,16-tetrahydro-1H-benzo[g]pyrido[4,3-b][1,5]oxaazacyclododecine-1,14(2H)-dione;
(E)-12-(ethyl(tetrahydro-2H-pyran-4-yl)amino)-3-methyl-5,6,7,8,17,18-hexahydrobenzo[c]pyrido[4,3-l][1]azacyclotetradecine-1,16(2H,11H)-dione;
(E)-12-chloro-10-methoxy-3-methyl-5,6,15,16-tetrahydrobenzo[c]pyrido[4,3-j][1]azacyclododecine-1,14(2H,9H)-dione;
(Z)-12-chloro-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione;
(E)-12-chloro-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione;
(E)-12-chloro-10-isopropoxy-3-methyl-5,6,15,16-tetrahydrobenzo[c]pyrido[4,3-j][1]azacyclododecine-1,14(2H,9H)-dione;
(E)-11-(ethyl(tetrahydro-2H-pyran-4-yl)amino)-3-methyl-5,6,7,10,16,17-hexahydro-1H-benzo[c]pyrido[4,3-k][1]azacyclotridecine-1,15(2H)-dione;
11-(ethyl(tetrahydro-2H-pyran-4-yl)amino)-3-methyl-5,6,7,8,9,10,16,17-octahydro-1H-benzo[c]pyrido[4,3-k][1]azacyclotridecine-1,15(2H)-dione;
(Z)-11-(ethyl(tetrahydro-2H-pyran-4-yl)amino)-3-methyl-5,6,7,10,16,17-hexahydro-1H-benzo[c]pyrido[4,3-k][1]azacyclotridecine-1,15(2H)-dione;
(Z)-10-(ethyl(tetrahydro-2H-pyran-4-yl)amino)-3,5-dimethyl-5,6,15,16-tetrahydrobenzo[c]pyrido[4,3-j][1]azacyclododecine-1,14(2H,9H)-dione;
(E)-10-(ethyl(tetrahydro-2H-pyran-4-yl)amino)-3,5-dimethyl-5,6,15,16-tetrahydrobenzo[c]pyrido[4,3-j][1]azacyclododecine-1,14(2H,9H)-dione;
10-(ethyl(tetrahydro-2H-pyran-4-yl)amino)-3,5-dimethyl-6, 7,8,9,15,16-hexahydrobenzo[c]pyrido[4,3-j][1]azacyclododecine-1,14(2H,5H)-dione;
(E)-10-(ethyl(tetrahydro-2H-pyran-4-yl)amino)-6-(hydroxymethyl)-3-methyl-5,6,15,16-tetrahydrobenzo[c]pyrido[4,3-j][1]azacyclododecine-1,14(2H,9H)-dione;
(Z)-10-(ethyl(tetrahydro-2H-pyran-4-yl)amino)-6-(hydroxymethyl)-3-methyl-5,6,15,16-tetrahydrobenzo[c]pyrido[4,3-j][1]azacyclododecine-1,14(2H,9H)-dione;
10-(ethyl(tetrahydro-2H-pyran-4-yl)amino)-6-(hydroxymethyl)-3-methyl-6,7,8,9,15,16-hexahydrobenzo[c]pyrido[4,3-j][1]azacyclododecine-1,14(2H,5H)-dione;
11-(ethyl(tetrahydro-2H-pyran-4-yl)amino)-7-hydroxy-3-methyl-5,6,7,8,9,10,16,17-octahydro-1H-benzo[c]pyrido[4,3-k][1]azacyclotridecine-1,15(2H)-dione;
(E)-10-(ethyl(tetrahydro-2H-pyran-4-yl)amino)-15-(2-hydroxy ethyl)-3-methyl-5,6,15,16-tetrahydrobenzo[c]pyrido[4,3-j][1]azacyclododecine-1,14(2H,9H)-dione;
(E)-10-((4,4-difluorocyclohexyl)(ethyl)amino)-3-methyl-5,6,15,16-tetrahydrobenzo[c]pyrido[4,3-j][1]azacyclododecine-1,14(2H,9H)-dione;
(E)-10-(ethyl(piperidin-4-yl)amino)-3-methyl-5,6,15,16-tetrahydrobenzo[c]pyrido[4,3-j][1]azacyclododecine-1,14(2H,9H)-dione;
(E)-10-(ethyl(1-isopropylpiperidin-4-yl)amino)-3-methyl-5,6,15,16-tetrahydrobenzo[c]pyrido[4,3-j][1]azacyclododecine-1,14(2H,9H)-dione;
(E)-10-(ethyl(1-methylpiperidin-4-yl)amino)-3-methyl-5,6,15,16-tetrahydrobenzo[c]pyrido[4,3-j][1]azacyclododecine-1,14(2H,9H)-dione;
(E)-10-(ethyl(1-(methylsulfonyl)piperidin-4-yl)amino)-3-methyl-5,6,15,16-tetrahydrobenzo[c]pyrido[4,3-j][1]azacyclododecine-1,14(2H,9H)-dione;
(E)-10-(ethyl(1-(2-hydroxyethyl)piperidin-4-yl)amino)-3-methyl-5,6,15,16-tetrahydrobenzo[c]pyrido[4,3-j][1]azacyclododecine-1,14(2H,9H)-dione;
(E)-10-((trans-4-aminocyclohexyl)(ethyl)amino)-3-methyl-5,6,15,16-tetrahydrobenzo[c]pyrido[4,3-j][1]azacyclododecine-1,14(2H,9H)-dione;
(E)-10-((trans-4-(dimethylamino)cyclohexyl)(ethyl)amino)-3-methyl-5,6,15,16-tetrahydrobenzo[c]pyrido[4,3-j][1]azacyclododecine-1,14(2H, 9H)-dione;
(E)-11-(ethyl(tetrahydro-2H-pyran-4-yl)amino)-3-methyl-5,6,7,10,16,17-hexahydro-1H-benzo[h]pyrido[4,3-c][1,6]diazacyclotridecine-1,15(2H)-dione;
(Z)-11-(ethyl(tetrahydro-2H-pyran-4-yl)amino)-3-methyl-5,6,7,10,16,17-hexahydro-1H-benzo[h]pyrido[4,3-c][1,6]diazacyclotridecine-1,15(2H)-dione;
(E)-11-(ethyl(piperidin-4-yl)amino)-3-methyl-5,6,7,10,16,17-hexahydro-1H-benzo[c]pyrido[4,3-k][1]azacyclotridecine-1,15(2H)-dione;
(E)-10-((1-(cyclopropylmethyl)piperidin-4-yl)(ethyl)amino)-3-methyl-5,6,15,16-tetrahydrobenzo[c]pyrido[4,3-j][1]azacyclododecine-1,14(2H,9H)-dione;
(E)-11-(ethyl(1-isopropylpiperidin-4-yl)amino)-3-methyl-5,6,7,10,16,17-hexahydro-1H-benzo[c]pyrido[4,3-k][1]azacyclotridecine-1,15(2H)-dione;
(E)-10-(ethyl(1-(3,3,3-trifluoropropyl)piperidin-4-yl)amino)-3-methyl-5,6,15,16-tetrahydrobenzo[c]pyrido[4,3-j][1]azacyclododecine-1,14(2H,9H)-dione;
(E)-10-(ethyl(1-ethylpiperidin-4-yl)amino)-3-methyl-5,6,15,16-tetrahydrobenzo[c]pyrido[4,3-j][1]azacyclododecine-1,14(2H,9H)-dione;
(E)-10-(ethyl(14(1-methyl-1H-pyrazol-3-yl)methyl)piperidin-4-yl)amino)-3-methyl-5,6,15,16-tetrahydrobenzo[c]pyrido[4,3-j][1]azacyclododecine-1,14(2H,9H)-dione;
(E)-ethyl 2-(4-(ethyl(3-methyl-1,14-di oxo-1,2,5,6,9,14,15,16-octahydrobenzo[c]pyrido[4,3-j][1]azacyclododecin-10-yl)amino)piperidin-1-yl)-2-methylpropanoate;
(E)-10-(ethyl(1-(2,2,2-trifluoroethyl)piperidin-4-yl)amino)-3-methyl-5,6,15,16-tetrahydrobenzo[c]pyrido[4,3-j][1]azacyclododecine-1,14(2H,9H)-dione;
(E)-10-(ethyl(14(6-methylpyridin-2-yl)methyl)piperidin-4-yl)amino)-3-methyl-5,6,15,16-tetrahydrobenzo[c]pyrido[4,3-j][1]azacyclododecine-1,14(2H,9H)-dione;
(E)-10-((trans-4-(diethylamino)cyclohexyl)(ethyl)amino)-3-methyl-5,6,15,16-tetrahydrobenzo[c]pyrido[4,3-j][1]azacyclododecine-1,14(2H, 9H)-dione;
(E)-10-(ethyl(trans-4-morpholinocyclohexyl)amino)-3-methyl-5,6,15,16-tetrahydrobenzo[c]pyrido[4,3-j][1]azacyclododecine-1,14(2H, 9H)-dione;
(E)-10-((1-(1,3-dihydroxypropan-2-yl)piperidin-4-yl)(ethyl)amino)-3-methyl-5,6,15,16-tetrahydrobenzo[c]pyrido[4,3-j][1]azacyclododecine-1,14(2H,9H)-dione;
(Z)-10-(ethyl(piperidin-4-yl)amino)-3-methyl-5,6,15,16-tetrahydrobenzo[c]pyrido[4,3-j][1]azacyclododecine-1,14(2H, 9H)-dione;
(E)-11-((trans-4-(di methylamino)cyclohexyl)(ethyl)amino)-3-methyl-5,6,7,10,16,17-hexahydro-1H-benzo[c]pyrido[4,3-k][1]azacyclotridecine-1,15(2H)-dione;
9-(ethyl(piperidin-4-yl)amino)-3-methyl-5,8,14,15-tetrahydro-1H-benzo[c]pyrido[4,3-i][1]azacycloundecine-1,13(2H)-dione;
(E)-10-((cis-4-(dimethylamino)cyclohexyl)(ethyl)amino)-3-methyl-5,6,15,16-tetrahydrobenzo[c]pyrido[4,3-j][1]azacyclododecine-1,14(2H,9H)-dione;
(E)-10-((trans-4-((2,2-difluoroethyl)amino)cyclohexyl)(ethyl)amino)-3-methyl-5,6,15,16-tetrahydrobenzo[c]pyrido[4,3-j][1]azacyclododecine-1,14(2H,9H)-dione;
(E)-10-((trans-4-((2,2-difluoroethyl)(methyl)amino)cyclohexyl)(ethyl)amino)-3-methyl-5,6,15,16-tetrahydrobenzo[c]pyrido[4,3-j][1]azacyclododecine-1,14(2H,9H)-dione;
(E)-10-(ethyl(trans-4-((2,2,2-trifluoroethyl)amino)cyclohexyl)amino)-3-methyl-5,6,15,16-tetrahydrobenzo[c]pyrido[4,3-j][1]azacyclododecine-1,14(2H,9H)-dione;
(E)-10-(ethyl(trans-4-(methyl(2,2,2-trifluoroethyl)amino)cyclohexyl)amino)-3-methyl-5,6,15,16-tetrahydrobenzo[c]pyrido[4,3-j][1]azacyclododecine-1,14(2H,9H)-dione;
(E)-10-((trans-4-(azetidin-1-yl)cyclohexyl)(ethyl)amino)-3-methyl-5,6,15,16-tetrahydrobenzo[c]pyrido[4,3-j][1]azacyclododecine-1,14(2H,9H)-dione;
(Z)-9-((trans-4-(dimethylamino)cyclohexyl)(ethyl)amino)-3-methyl-5,8,14,15-tetrahydro-1H-benzo[c]pyrido[4,3-i][1]azacycloundecine-1,13(2H)-dione;
9-((trans-4-(dimethylamino)cyclohexyl)(ethyl)amino)-3-methyl-5,6,7,8,14,15-hexahydro-1H-benzo[c]pyrido[4,3-i][1]azacycloundecine-1,13(2H)-dione;
(E)-10-(ethyl(tetrahydro-2H-pyran-4-yl)amino)-3-methyl-5,6,15,16-tetrahydrodipyrido[3,4-c:3′,4′-j][1]azacyclododecine-1,14(2H,9H)-dione;
(E)-104(2-hydroxyethyl)(tetrahydro-2H-pyran-4-yl)amino)-3-methyl-5,6,15,16-tetrahydrobenzo[c]pyrido[4,3-j][1]azacyclododecine-1,14(2H,9H)-dione;
(E)-10-((1-(dimethylamino)piperidin-4-yl)(ethyl)amino)-3-methyl-5,6,15,16-tetrahydrobenzo[c]pyrido[4,3-j][1]azacyclododecine-1,14(2H,9H)-dione;
(E)-10-(ethyl(2-azaspiro[3.5]nonan-7-yl)amino)-3-methyl-5,6,15,16-tetrahydrobenzo[c]pyrido[4,3-j][1]azacyclododecine-1,14(2H,9H)-dione;
(E)-10-(ethyl(2-methyl-2-azaspiro[3.5]nonan-7-yl)amino)-3-methyl-5,6,15,16-tetrahydrobenzo[c]pyrido[4,3-j][1]azacyclododecine-1,14(2H,9H)-dione;
(E)-10-(ethyl(7-azaspiro[3.5]nonan-2-yl)amino)-3-methyl-5,6,15,16-tetrahydrobenzo[c]pyrido[4,3-j][1]azacyclododecine-1,14(2H,9H)-dione;
(E)-10-(ethyl(7-methyl-7-azaspiro[3.5]nonan-2-yl)amino)-3-methyl-5,6,15,16-tetrahydrobenzo[c]pyrido[4,3-j][1]azacyclododecine-1,14(2H,9H)-dione;
(E)-10-((6-aminospiro[3.3]heptan-2-yl)(ethyl)amino)-3-methyl-5,6,15,16-tetrahydrobenzo[c]pyrido[4,3-j][1]azacyclododecine-1,14(2H,9H)-dione;
(E)-10-((6-(dimethylamino)spiro[3.3]heptan-2-yl)(ethyl)amino)-3-methyl-5,6,15,16-tetrahydrobenzo[c]pyrido[4,3-j][1]azacyclododecine-1,14(2H,9H)-dione;
(E)-10-(ethyl(2-methyl-2-azaspiro[3.3]heptan-6-yl)amino)-3-methyl-5,6,15,16-tetrahydrobenzo[c]pyrido[4,3-j][1]azacyclododecine-1,14(2H,9H)-dione;
(E)-10-(ethyl(trans-4-(methylamino)cyclohexyl)amino)-3-methyl-5,6,15,16-tetrahydrobenzo[c]pyrido[4,3-j][1]azacyclododecine-1,14(2H,9H)-dione;
(E)-10-(ethyl(cis-4-(methylamino)cyclohexyl)amino)-3-methyl-5,6,15,16-tetrahydrobenzo[c]pyrido[4,3-j][1]azacyclododecine-1,14(2H,9H)-dione;
(E)-10-(ethyl(cis-4-(3-fluoroazetidin-1-yl)cyclohexyl)amino)-3-methyl-5,6,15,16-tetrahydrobenzo[c]pyrido[4,3-j][1]azacyclododecine-1,14(2H,9H)-dione;
(E)-10-(ethyl(trans-4-(3-fluoroazetidin-1-yl)cyclohexyl)amino)-3-methyl-5,6,15,16-tetrahydrobenzo[c]pyrido[4,3-j][1]azacyclododecine-1,14(2H,9H)-dione;
(E)-10-(azepan-4-yl(ethyl)amino)-3-methyl-5,6,15,16-tetrahydrobenzo[c]pyrido[4,3-j][1]azacyclododecine-1,14(2H,9H)-dione;
(E)-10-(ethyl(cis-4-hydroxycyclohexyl)amino)-3-methyl-5,6,15,16-tetrahydrobenzo[c]pyrido[4,3-j][1]azacyclododecine-1,14(2H,9H)-dione;
(E)-10-(ethyl(trans-4-hydroxycyclohexyl)amino)-3-methyl-5,6,15,16-tetrahydrobenzo[c]pyrido[4,3-j][1]azacyclododecine-1,14(2H,9H)-dione;
(E)-10-((cis-4-(3,3-difluoroazetidin-1-yl)cyclohexyl)(ethyl)amino)-3-methyl-5,6,15,16-tetrahydrobenzo[c]pyrido[4,3-j][1]azacyclododecine-1,14(2H,9H)-dione;
(E)-10-((trans-4-(3,3-difluoroazetidin-1-yl)cyclohexyl)(ethyl)amino)-3-methyl-5,6,15,16-tetrahydrobenzo[c]pyrido[4,3-j][1]azacyclododecine-1,14(2H,9H)-dione;
(E)-10-(ethyl(cis-4-((3,3,3-trifluoropropyl)amino)cyclohexyl)amino)-3-methyl-5,6,15,16-tetrahydrobenzo[c]pyrido[4,3-j][1]azacyclododecine-1,14(2H,9H)-dione;
(E)-10-(ethyl(trans-4-((3,3,3-trifluoropropyl)amino)cyclohexyl)amino)-3-methyl-5,6,15,16-tetrahydrobenzo[c]pyrido[4,3-j][1]azacyclododecine-1,14(2H,9H)-dione;
(E)-10-((trans-4-(dimethylamino)cyclohexyl)oxy)-3-methyl-5,6,15,16-tetrahydrobenzo[c]pyrido[4,3-j][1]azacyclododecine-1, 14(2H, 9H)-di one;
(Z)-10-((trans-4-(dimethylamino)cyclohexyl)oxy)-3-methyl-5,6,15,16-tetrahydrobenzo[c]pyrido[4,3-j][1]azacyclododecine-1,14(2H,9H)-dione; and
(E)-10-(ethyl(cis-4-morpholinocyclohexyl)amino)-3-methyl-5,6,15,16-tetrahydrobenzo[c]pyrido[4,3-j][1]azacyclododecine-1, 14(2H, 9H)-di one;

or pharmaceutically acceptable salts thereof.

Typically, but not absolutely, the salts of the present invention are pharmaceutically acceptable salts. Salts of the disclosed compounds containing a basic amine or other basic functional group may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, γ-hydroxybutyrates, glycolates, tartrates mandelates, and sulfonates, such as xylenesulfonates, methanesulfonates, propanesulfonates, naphthalene-1-sulfonates and naphthalene-2-sulfonates.

Salts of the disclosed compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

Other salts, which are not pharmaceutically acceptable, may be useful in the preparation of compounds of this invention and these should be considered to form a further aspect of the invention. These salts, such as oxalic or trifluoroacetate, while not in themselves pharmaceutically acceptable, may be useful in the preparation of salts useful as intermediates in obtaining the compounds of the invention and their pharmaceutically acceptable salts.

The compound of Formula (I) or a salt thereof may exist in stereoisomeric forms (e.g., it contains one or more asymmetric carbon atoms). The individual stereoisomers (enantiomers and diastereomers) and mixtures of these are included within the scope of the present invention. Likewise, it is understood that a compound or salt of Formula (I) may exist in tautomeric forms other than that shown in the formula and these are also included within the scope of the present invention. It is to be understood that the present invention includes all combinations and subsets of the particular groups defined hereinabove. The scope of the present invention includes mixtures of stereoisomers as well as purified enantiomers or enantiomerically/diastereomerically enriched mixtures. It is to be understood that the present invention includes all combinations and subsets of the particular groups defined hereinabove.

The subject invention also includes isotopically-labeled compounds, which are identical to those recited in Formula (I) and following, but for the fact that one or more atoms are replaced by an atom having an atomic mass or mass number different from the atomic mass or mass number usually found in nature. Examples of isotopes that can be incorporated into compounds of the invention and pharmaceutically acceptable salts thereof include isotopes of hydrogen, carbon, nitrogen, oxygen, phosphorous, sulfur, fluorine, chlorine, and iodine, such as ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I, and ¹²⁵I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of Formula (I) and following of this invention can generally be prepared by carrying out the procedures disclosed in the Schemes and/or in the Examples below, by substituting a readily available isotopically labeled reagent for a non-isotopically labeled reagent.

The invention further provides a pharmaceutical composition (also referred to as pharmaceutical formulation) comprising a compound of Formula (I) or pharmaceutically acceptable salt thereof and one or more excipients (also referred to as carriers and/or diluents in the pharmaceutical arts). The excipients are acceptable in the sense of being compatible with the other ingredients of the formulation and not deleterious to the recipient thereof (i.e., the patient).

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of the invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

Pharmaceutical compositions may be in unit dose form containing a predetermined amount of active ingredient per unit dose. Such a unit may contain a therapeutically effective dose of the compound of Formula (I) or salt thereof or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical compositions may be prepared by any of the methods well-known in the pharmacy art.

Pharmaceutical compositions may be adapted for administration by any appropriate route, for example, by oral (including buccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the excipient(s).

When adapted for oral administration, pharmaceutical compositions may be in discrete units such as tablets or capsules; powders or granules; solutions or suspensions in aqueous or non-aqueous liquids; edible foams or whips; oil-in-water liquid emulsions or water-in-oil liquid emulsions. The compound or salt thereof of the invention or the pharmaceutical composition of the invention may also be incorporated into a candy, a wafer, and/or tongue tape formulation for administration as a “quick-dissolve” medicine.

For instance, for oral administration in the form of a tablet or capsule, the active drug component can be combined with an oral, non-toxic pharmaceutically acceptable inert carrier such as ethanol, glycerol, water, and the like. Powders or granules are prepared by comminuting the compound to a suitable fine size and mixing with a similarly comminuted pharmaceutical carrier such as an edible carbohydrate, as, for example, starch or mannitol. Flavoring, preservative, dispersing, and coloring agents can also be present.

Capsules are made by preparing a powder mixture, as described above, and filling formed gelatin or non-gelatinous sheaths. Glidants and lubricants such as colloidal silica, talc, magnesium stearate, calcium stearate, solid polyethylene glycol can be added to the powder mixture before the filling operation. A disintegrating or solubilizing agent such as agar-agar, calcium carbonate, or sodium carbonate can also be added to improve the availability of the medicine when the capsule is ingested.

Moreover, when desired or necessary, suitable binders, lubricants, disintegrating agents, and coloring agents can also be incorporated into the mixture. Suitable binders include starch, gelatin, natural sugars, such as glucose or beta-lactose, corn sweeteners, natural and synthetic gums such as acacia, tragacanth, sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methylcellulose, agar, bentonite, xanthan gum, and the like.

Tablets are formulated, for example, by preparing a powder mixture, granulating or slugging, adding a lubricant and disintegrant, and pressing into tablets. A powder mixture is prepared by mixing the compound, suitably comminuted, with a diluent or base as described above, and optionally, with a binder such as carboxymethylcellulose, and aliginate, gelatin, or polyvinyl pyrrolidone, a solution retardant such as paraffin, a resorption accelerator such as a quaternary salt, and/or an absorption agent such as bentonite, kaolin, or dicalcium phosphate. The powder mixture can be granulated by wetting a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compound or salt of the present invention can also be combined with a free-flowing inert carrier and compressed into tablets directly without going through the granulating or slugging steps. A clear opaque protective coating consisting of a sealing coat of shellac, a coating of sugar, or polymeric material, and a polish coating of wax can be provided. Dyestuffs can be added to these coatings to distinguish different dosages.

Oral fluids such as solutions, syrups, and elixirs can be prepared in dosage unit form so that a given quantity contains a predetermined amount of active ingredient. Syrups can be prepared by dissolving the compound or salt thereof of the invention in a suitably flavoured aqueous solution, while elixirs are prepared through the use of a non-toxic alcoholic vehicle. Suspensions can be formulated by dispersing the compound or salt of the invention in a non-toxic vehicle. Solubilizers and emulsifiers, such as ethoxylated isostearyl alcohols and polyoxyethylene sorbitol ethers, preservatives, flavor additives such as peppermint oil, natural sweeteners, saccharin, or other artificial sweeteners, and the like, can also be added.

Where appropriate, dosage unit formulations for oral administration can be microencapsulated. The formulation can also be prepared to prolong or sustain the release as, for example, by coating or embedding particulate material in polymers, wax, or the like.

In the present invention, tablets and capsules are preferred for delivery of the pharmaceutical composition.

In accordance with another aspect of the invention there is provided a process for the preparation of a pharmaceutical composition comprising mixing (or admixing) a compound of Formula (I) or salt thereof with at least one excipient.

The present invention also provides a method of treatment in a mammal, especially a human. The compounds and compositions of the invention are used to treat cellular proliferation diseases. Disease states which can be treated by the methods and compositions provided herein include, but are not limited to, cancer (further discussed below), autoimmune disease, fungal disorders, arthritis, graft rejection, inflammatory bowel disease, proliferation induced after medical procedures, including, but not limited to, surgery, angioplasty, and the like. It is appreciated that in some cases the cells may not be in a hyper or hypo proliferation state (abnormal state) and still requires treatment. For example, during wound healing, the cells may be proliferating “normally”, but proliferation enhancement may be desired. Thus, in one embodiment, the invention herein includes application to cells or individuals afflicted or impending affliction with any one of these disorders or states.

The compositions and methods provided herein are particularly deemed useful for the treatment of cancer including tumors such as prostate, breast, brain, skin, cervical carcinomas, testicular carcinomas, etc. They are particularly useful in treating metastatic or malignant tumors. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one or related of the above identified conditions.

The instant compounds can be combined with or co-administered with other therapeutic agents, particularly agents that may enhance the activity or time of disposition of the compounds. Combination therapies according to the invention comprise the administration of at least one compound of the invention and the use of at least one other treatment method. In one embodiment, combination therapies according to the invention comprise the administration of at least one compound of the invention and surgical therapy. In one embodiment, combination therapies according to the invention comprise the administration of at least one compound of the invention and radiotherapy. In one embodiment, combination therapies according to the invention comprise the administration of at least one compound of the invention and at least one supportive care agent (e.g., at least one anti-emetic agent). In one embodiment, combination therapies according to the present invention comprise the administration of at least one compound of the invention and at least one other chemotherapeutic agent. In one particular embodiment, the invention comprises the administration of at least one compound of the invention and at least one anti-neoplastic agent. In yet another embodiment, the invention comprises a therapeutic regimen where the EZH2 inhibitors of this disclosure are not in and of themselves active or significantly active, but when combined with another therapy, which may or may not be active as a standalone therapy, the combination provides a useful therapeutic outcome.

By the term “co-administering” and derivatives thereof as used herein refers to either simultaneous administration or any manner of separate sequential administration of an EZH2 inhibiting compound, as described herein, and a further active ingredient or ingredients, known to be useful in the treatment of cancer, including chemotherapy and radiation treatment. The term further active ingredient or ingredients, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any anti-neoplastic agent that has activity versus a susceptible tumor being treated may be co-administered in the treatment of specified cancers in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; DNA methyltransferase inhibitors such as azacitidine and decitabine; signal transduction pathway inhibitors; non-receptor tyrosine kinase angiogenesis inhibitors; immunotherapeutic agents; proapoptotic agents; and cell cycle signaling inhibitors.

Typically, any chemotherapeutic agent that has activity against a susceptible neoplasm being treated may be utilized in combination with the compounds the invention, provided that the particular agent is clinically compatible with therapy employing a compound of the invention. Typical anti-neoplastic agents useful in the present invention include, but are not limited to: alkylating agents, anti-metabolites, antitumor antibiotics, antimitotic agents, nucleoside analogues, topoisomerase I and II inhibitors, hormones and hormonal analogues; retinoids, histone deacetylase inhibitors; signal transduction pathway inhibitors including inhibitors of cell growth or growth factor function, angiogenesis inhibitors, and serine/threonine or other kinase inhibitors; cyclin dependent kinase inhibitors; antisense therapies and immunotherapeutic agents, including monoclonals, vaccines or other biological agents.

Nucleoside analogues are those compounds which are converted to deoxynucleotide triphosphates and incorporated into replicating DNA in place of cytosine. DNA methyltransferases become covalently bound to the modified bases resulting in an inactive enzyme and reduced DNA methylation. Examples of nucleoside analogues include azacitidine and decitabine which are used for the treatment of myelodysplastic disorder. Histone deacetylase (HDAC) inhibitors include vorinostat, for the treatment of cutaneous T-cell lymphoma. HDACs modify chromatin through the deacetylation of histones. In addition, they have a variety of substrates including numerous transcription factors and signaling molecules. Other HDAC inhibitors are in development.

Signal transduction pathway inhibitors are those inhibitors which block or inhibit a chemical process which evokes an intracellular change. As used herein this change is cell proliferation or differentiation or survival. Signal transduction pathway inhibitors useful in the present invention include, but are not limited to, inhibitors of receptor tyrosine kinases, non-receptor tyrosine kinases, SH2/SH3 domain blockers, serine/threonine kinases, phosphatidyl inositol-3-OH kinases, myoinositol signaling, and Ras oncogenes. Signal transduction pathway inhibitors may be employed in combination with the compounds of the invention in the compositions and methods described above.

Receptor kinase angiogenesis inhibitors may also find use in the present invention. Inhibitors of angiogenesis related to VEGFR and TIE-2 are discussed above in regard to signal transduction inhibitors (both are receptor tyrosine kinases). Other inhibitors may be used in combination with the compounds of the invention. For example, anti-VEGF antibodies, which do not recognize VEGFR (the receptor tyrosine kinase), but bind to the ligand; small molecule inhibitors of integrin (alphaᵥ beta₃) that inhibit angiogenesis; endostatin and angiostatin (non-RTK) may also prove useful in combination with the compounds of the invention. One example of a VEGFR antibody is bevacizumab) (AVASTIN®).

Several inhibitors of growth factor receptors are under development and include ligand antagonists, antibodies, tyrosine kinase inhibitors, anti-sense oligonucleotides and aptamers. Any of these growth factor receptor inhibitors may be employed in combination with the compounds of the invention in any of the compositions and methods/uses described herein. Trastuzumab (Herceptin®) is an example of an anti-erbB2 antibody inhibitor of growth factor function. One example of an anti-erbB1 antibody inhibitor of growth factor function is cetuximab (Erbitux™, C225). Bevacizumab (Avastin®) is an example of a monoclonal antibody directed against VEGFR. Examples of small molecule inhibitors of epidermal growth factor receptors include but are not limited to lapatinib (Tykerb®) and erlotinib (TARCEVA®). Imatinib mesylate (GLEEVEC®) is one example of a PDGFR inhibitor. Examples of VEGFR inhibitors include pazopanib (Votrient®), ZD6474, AZD2171, PTK787, sunitinib and sorafenib.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem, Soc., 93:2325 (1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Int. Med., 111:273, 1989) and for the treatment of breast cancer (Holmes et al., J. Nat. Cancer Inst., 83:1797, 1991). It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine[R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine[1,1-cyclobutane-dicarboxylate(2-)—O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also known as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-O—(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leukopenialeukopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leukopenialeukopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H)pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leukopenialeukopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leukopenialeukopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leukopenialeukopenia, thrombocytopenia, and anemia, is the most common dose limiting side effect of gemcitabine administration.

Methotrexate, N-[4 [[(2,4-diamino-6-pteridinyl)methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leukopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Pharmaceutical compositions may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the Formula (I), depending on the condition being treated, the route of administration and the age, weight and condition of the patient, or pharmaceutical compositions may be presented in unit dose forms containing a predetermined amount of active ingredient per unit dose. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association a compound of formal (I) with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of a compound of Formula (I). Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit pharmaceutical compositions for oral administration can be microencapsulated. The formulation can also be prepared to prolong or sustain the release as for example by coating or embedding particulate material in polymers, wax or the like.

Pharmaceutical compositions adapted for rectal administration may be presented as suppositories or as enemas.

Pharmaceutical compositions adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The pharmaceutical compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the pharmaceutical compositions may include other agents conventional in the art having regard to the type of formulation in question, for example those suitable for oral administration may include flavoring agents.

A therapeutically effective amount of a compound of the present invention will depend upon a number of factors including, for example, the age and weight of the intended recipient, the precise condition requiring treatment and its severity, the nature of the formulation, and the route of administration, and will ultimately be at the discretion of the attendant prescribing the medication. However, an effective amount of a compound of Formula (I) for the treatment of anemia will generally be in the range of 0.001 to 100 mg/kg body weight of recipient per day, suitably in the range of 0.01 to 10 mg/kg body weight per day. For a 70 kg adult mammal, the actual amount per day would suitably be from 7 to 700 mg and this amount may be given in a single dose per day or in a number (such as two, three, four, five or six) of sub-doses per day such that the total daily dose is the same. An effective amount of a salt or solvate, etc., may be determined as a proportion of the effective amount of the compound of Formula (I) per se. It is envisaged that similar dosages would be appropriate for treatment of the other conditions referred to above.

### DEFINITIONS

Terms are used within their accepted meanings. The following definitions are meant to clarify, but not limit, the terms defined.

As used herein, the term “alkyl” represents a saturated, straight or branched hydrocarbon moiety having the specified number of carbon atoms. The term “(C₁-C₆)alkyl” refers to an alkyl moiety containing from 1 to 6 carbon atoms. Exemplary alkyls include, but are not limited to methyl, ethyl, n-propyl, isopropyl, n-butyl, isobutyl, s-butyl, t-butyl, pentyl, and hexyl.

As used herein, the term “alkylenyl” represents a saturated, straight or branched divalent hydrocarbon radical having the specified number of carbon atoms. The term “(C₂-C₈)alkylenyl” refers to an alkylenyl moiety containing from 2 to 8 carbon atoms.

“Methylene unit” refers to a divalent single carbon hydrocarbon radical, i.e. —CH₂—.

When the term “alkyl” is used in combination with other substituent groups, such as “halo(C₁-C₄)alkyl”, “hydroxy(C₁-C₄)alkyl” or “phenyl(C₁-C₂)alkyl-”, the term “alkyl” is intended to encompass a divalent straight or branched-chain hydrocarbon radical, wherein the point of attachment is through the alkyl moiety. The term “halo(C₁-C₄)alkyl” is intended to mean a radical having one or more halogen atoms, which may be the same or different, at one or more carbon atoms of an alkyl moiety containing from 1 to 4 carbon atoms, which is a straight or branched-chain carbon radical. Examples of “halo(C₁-C₄)alkyl” groups useful in the present invention include, but are not limited to, —CF₃ (trifluoromethyl), —CCl₃ (trichloromethyl), 1,1-difluoroethyl, 2,2,2-trifluoroethyl, and hexafluoroisopropyl. Examples of “phenyl(C₁-C₂)alkyl-” groups useful in the present invention include, but are not limited to, benzyl(phenylmethyl), 1-methylbenzyl(1-phenylethyl), and phenethyl(2-phenylethyl). Examples of “hydroxy(C₁-C₄)alkyl” groups useful in the present invention include, but are not limited to, hydroxymethyl, hydroxyethyl, and hydroxyisopropyl.

“Alkoxy” refers to a group containing an alkyl radical, defined hereinabove, attached through an oxygen linking atom. The term “(C₁-C₄)alkoxy” refers to a straight- or branched-chain hydrocarbon radical having at least 1 and up to 4 carbon atoms attached through an oxygen linking atom. Exemplary “(C₁-C₄)alkoxy” groups useful in the present invention include, but are not limited to, methoxy, ethoxy, n-propoxy, isopropoxy, n-butoxy, s-butoxy, isobutoxy, and t-butoxy.

When the term “alkenyl” is used it refers to straight or branched hydrocarbon chains containing the specified number of carbon atoms and at least 1 and up to 4 carbon-carbon double bonds. Examples include ethenyl (or ethenylene) and propenyl (or propenylene).

When the term “alkenylenyl” is used it refers to a straight or branched divalent hydrocarbon radical containing the specified number of carbon atoms and at least 1 and up to 4 carbon-carbon double bonds.

When the term “alkynyl” (or “alkynylene”) is used it refers to straight or branched hydrocarbon chains containing the specified number of carbon atoms and at least 1 and up to 4 carbon-carbon triple bonds. Examples include ethynyl (or ethynylene) and propynyl (or propynylene).

When “cycloalkyl” is used it refers to a non-aromatic, saturated, cyclic hydrocarbon ring containing the specified number of carbon atoms. So, for example, the term “(C₃-C₈)cycloalkyl” refers to a non-aromatic cyclic hydrocarbon ring having from three to eight carbon atoms. Exemplary “(C₃-C₈)cycloalkyl” groups useful in the present invention include, but are not limited to, cyclopropyl, cyclobutyl, cyclopentyl, cyclohexyl, cycloheptyl, and cyclooctyl.

As used herein, the term “cycloalkenyl” refers to a non-aromatic, cyclic hydrocarbon ring containing the specified number of carbon atoms and at least one carbon-carbon double bond. The term “(C₅-C₈)cycloalkenyl” refers to a non-aromatic cyclic hydrocarbon ring having from five to eight ring carbon atoms. Exemplary “(C₅-C₈)cycloalkenyl” groups useful in the present invention include cyclopentenyl, cyclohexenyl, cycloheptenyl, and cyclooctenyl.

As used herein, the term “cycloalkyloxy-” refers to a group containing a cycloalkyl radical, defined hereinabove, attached through an oxygen linking atom. Exemplary “(C₃-C₈)cycloalkyloxy-” groups useful in the present invention include cyclopropyloxy, cyclobutyloxy, cyclopentyloxy, cyclohexyloxy, cycloheptyloxy, and cyclooctyloxy.

As used herein, the term “bicycloalkyl” refers to a saturated, bridged, fused, or spiro, bicyclic hydrocarbon ring system containing the specified number of carbon atoms. Exemplary “(C₆-C₁₀)bicycloalkyl” groups include, but are not limited to bicyclo[2.1.1]hexyl, bicyclo[2.1.1]heptyl, bicyclo[3.2.1]octyl, bicyclo[2.2.2]octyl, bicyclo[3.2.2]nonyl, bicyclo[3.3.1]nonyl, bicyclo[3.3.2]decyl, bicyclo[4.3.1]decyl, bicyclo[2.2.0]hexyl, bicyclo[3.1.0]hexyl, bicyclo[3.2.0]heptyl, bicyclo[4.1.0]heptyl, octahydropentalenyl, bicyclo[4.2.0]octyl, decahydronaphthalenyl, spiro[3.3]heptyl, spiro[2.4]heptyl, spiro[3.4]octyl, spiro[2.5]octyl, spiro[4.4]nonyl, spiro[3.5]nonyl, and spiro[4.5]decyl.

The terms “halogen” and “halo” represent chloro, fluoro, bromo, or iodo substituents. “Hydroxy” or “hydroxyl” is intended to mean the radical —OH.

“Heterocycloalkyl” represents a group or moiety comprising a non-aromatic, monovalent monocyclic or bicyclic radical, which is saturated or partially unsaturated, containing 3 to 10 ring atoms, which includes 1 to 3 heteroatoms independently selected from nitrogen, oxygen and sulfur, including N-oxides, sulfur oxides, and dioxides. Illustrative examples of heterocycloalkyls useful in the present invention include, but are not limited to, aziridinyl, azetidinyl, pyrrolidinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, morpholinyl, thiomorpholinyl, tetrahydropyranyl, dihydropyranyl, 1,3-dioxanyl, 1,4-dioxanyl, 1,3-oxathiolanyl, 1,3-oxathianyl, 1,3-dithianyl, 1,4-dithianyl, hexahydro-1H-1,4-diazepinyl, azaspiro[3.3]heptanyl, azaspiro[3.4]octanyl, azaspiro[3.5]nonanyl, azaspiro[4.4]nonanyl, azaspiro[4.5]decanyl, azabicyclo[3.2.1]octyl, azabicyclo[3.3.1]nonyl, azabicyclo[4.3.0]nonyl, oxabicyclo[2.2.1]heptyl, 1,1-dioxidotetrahydro-2H-thiopyranyl, and 1,5,9-triazacyclododecyl.

As used herein, the term “heteroaryl” refers to an aromatic ring system containing carbon(s) and at least one heteroatom selected from nitrogen, oxygen and sulfur, including N-oxides. Heteroaryl may be monocyclic or polycyclic, substituted or unsubstituted. A monocyclic heteroaryl group may have 1 to 4 heteroatoms in the ring, while a polycyclic heteroaryl may contain 1 to 8 heteroatoms. Bicyclic heteroaryl rings may contain from 8 to 10 member atoms. Monocyclic heteroaryl rings may contain from 5 to 6 member atoms (carbons and heteroatoms). Exemplary 5- to 6-membered heteroaryls include, but are not limited to, furanyl, thiophenyl, thienyl, pyrrolyl, imidazolyl, pyrazolyl, triazolyl, tetrazolyl, thiazolyl, isothiazolyl, 1,2,3-triazolyl, 1,2,4-triazolyl, oxazolyl, isoxazolyl, 1,2,3-oxadiazolyl, 1,2,5-oxadiazolyl, thiadiazolyl, isothiazolyl, tetrazolyl, pyridinyl, pyridazinyl, pyrazinyl, pyrimidinyl, and triazinyl. Other exemplary heteroaryl groups include, but are not limited to benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

As used herein, the term “cyano” refers to the group —CN.

As used herein, the term “optionally” means that the subsequently described event(s) may or may not occur, and includes both event(s) that occur and event(s) that do not occur.

As used herein, unless otherwise defined, the phrase “optionally substituted” or variations thereof denote an optional substitution, including multiple degrees of substitution, with one or more substituent group. The phrase should not be interpreted as duplicative of the substitutions herein described and depicted.

As used herein, the term “treatment” refers to alleviating the specified condition, eliminating or reducing one or more symptoms of the condition, slowing or eliminating the progression of the condition, and delaying the reoccurrence of the condition in a previously afflicted or diagnosed patient or subject.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula (I), as well as salts thereof, may be administered as the raw chemical. Additionally, the active ingredient may be presented as a pharmaceutical composition.

### Compound Preparation

### Abbreviations

AcOH acetic acid
BBr₃ boron tribromide
CHCl₃ chloroform
CH₂Cl₂ dichloromethane
CH₃CN acetonitrile
Cs₂CO₃ cesium carbonate
CsF cesium fluoride
DCE 1,2-dichloroethane
DCM dichloromethane
DIPEA diisopropylethylamine
DMF N,N-dimethylformamide
DMSO dimethylsulfoxide
EtOAc ethyl acetate
EDC N-(3-dimethylaminopropyl)-N′-ethylcarbodiimide hydrochloride
ES electrospray
Et₃N triethylamine
Et₂O diethyl ether
EtOH ethanol
h hour(s)
HCl hydrochloric acid
H₂O water
HOAt 1-hydroxy-7-azabenzotriazole
HOBt 1-hydroxybenzotriazole
HPLC high-performance liquid chromatography
H₂SO₄ sulfuric acid
K₂CO₃ potassium carbonate
LCMS liquid chromatography mass spectrometry
LiAlH₄ lithium aluminum hydride
LiHMDS lithium bis(trimethylsilyl)amide
MeOH methanol
MgCl₂ magnesium chloride
MgSO₄ magnesium sulfate
min minute(s)
MS mass spectrometry
NaBH₃CN sodium cyanoborohydride
Na₂CO₃ sodium carbonate
NaHCO₃ sodium bicarbonate
Na(OAc)₃BH sodium triacetoxyborohydride
NaOH sodium hydroxide
Na₂SO₄ sodium sulfate
NCS N-chlorosuccinimide
NH₄OH ammonium hydroxide
Pd/C palladium on carbon
PdCl₂(dppf).CH₂Cl₂ [1,1′-bis(diphenylphosphino)ferrocene]dichloropalladium(II), complex with dichloromethane
Pd(PPh₃)₄ tetrakis(triphenylphosphine)palladium(O)
RB round-bottom
TBME tert-butyl methyl ether
TFA trifluoroacetic acid
THF tetrahydrofuran

### Generic Synthesis Schemes

The compounds of this invention may be made by a variety of methods, including well-known standard synthetic methods. Illustrative general synthetic methods are set out below and then specific compounds of the invention are prepared in the working examples. The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. In all of the schemes described below, protecting groups for sensitive or reactive groups are employed where necessary in accordance with general principles of synthetic chemistry. Protecting groups are manipulated according to standard methods of organic synthesis (T. W. Green and P. G. M. Wuts, (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention. Starting materials are commercially available or are made from commercially available starting materials using methods known to those skilled in the art.

The compounds of Formula (I) can be prepared according to Scheme 1 or by analogous methods. An appropriately functionalized 2-alkenyl-substituted benzoic acid A is coupled to an appropriately functionalized alkenyl-substituted 3-aminomethylpyridine B using an appropriate reagent, such as EDC and/or HOAt or HOBt, with an appropriate base, such as N-methylmorpholine, in an appropriate solvent, such as dichloromethane. Ring closing metathesis of amide C using an appropriate reagent, such as the Grubbs second generation RCM catalyst, in an appropriate solvent, such as dichloromethane, provides macrocycle D. Hydrolysis of the 2-methoxypyridine moiety using an appropriate reagent, such as hydrochloric acid, in an appropriate solvent, such as 1,4-dioxane and/or methanol, affords compounds of Formula (I) wherein L is alkenylenyl. Such compounds may be further functionalized to provide other compounds of Formula (I) and/or they may be hydrogenated under appropriate conditions, such as in the presence of catalytic platinum on carbon in a hydrogen gas atmosphere, in an appropriate solvent, such as ethyl acetate and/or methanol, to provide the saturated macrocycle, representing compounds of Formula (I) wherein L is alkylenyl. Alternatively, the transformations depicted in Scheme 1 may be carried out with intermediates where any one methylene unit of the alkenyl substituent on either of the 2-alkenyl-substituted benzoic acid A or the alkenyl-substituted 3-aminomethylpyridine B are replaced by —O—, —NH—, or —N(C₁-C₄)alkyl.

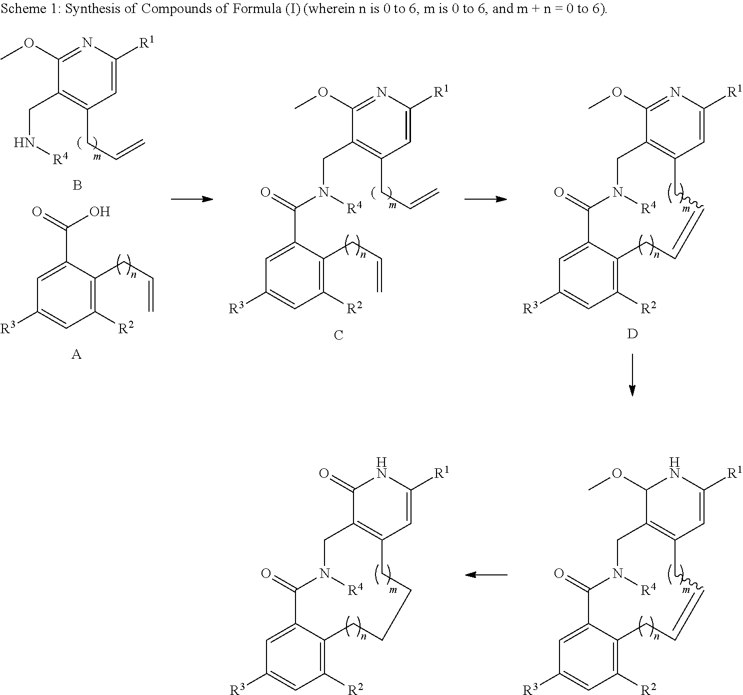

Intermediate B, wherein m is 2 or 3, can be prepared according to Scheme 2 or by analogous methods. An appropriately functionalized 3-cyano-4-methylpyridine is deprotonated with an appropriate base, such as lithium bis(trimethylsilyl)amide, in an appropriate solvent, such as tetrahydrofuran, and alkylated with an appropriate electrophile, such as 3-bromoprop-1-ene or 4-bromobut-1-ene. Reduction of the nitrile with an appropriate reagent, such as lithium aluminum hydride, in an appropriate solvent, such as diethyl ether and/or tetrahydrofuran, affords intermediate B.

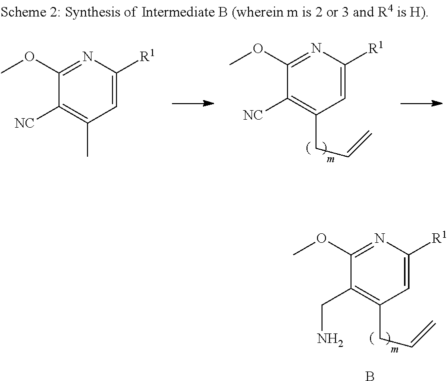

The compounds of Formula (I) can also be prepared according to Scheme 3 or by analogous methods. Macrocycle D-1 wherein R² is methoxy, prepared according to Scheme 1 or by analogous methods, is demethylated using an appropriate reagent, such as BBr₃, in an appropriate solvent, such as dichloromethane, to afford phenol E. Further functionalization of phenol E, for example by alkylation with an appropriate electrophile, such as an appropriately functionalized cycloalkyl methanesulfonate, using an appropriate base, such as Cs₂CO₃, in an appropriate solvent, such as DMF, provides intermediate F. Hydrolysis of the 2-methoxypyridine moiety using an appropriate reagent, such as hydrochloric acid, in an appropriate solvent, such as 1,4-dioxane and/or methanol, affords compounds of Formula (I). Such compounds may be further functionalized to provide other compounds of Formula (I) and/or they may be hydrogenated under appropriate conditions, such as in the presence of catalytic platinum on carbon in a hydrogen gas atmosphere, in an appropriate solvent, such as ethyl acetate and/or methanol, to provide the saturated macrocycle, representing compounds of Formula (I) wherein L is alkylenyl.

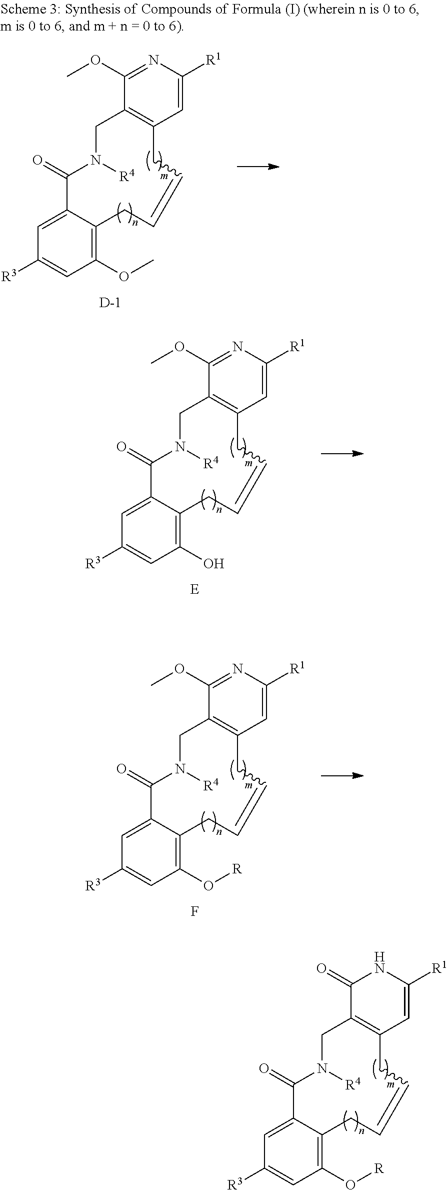

The compounds of Formula (I) can also be prepared according to Scheme 4 or by analogous methods. Macrocycle D-2 wherein R² is nitro, prepared according to Scheme 1 or by analogous methods, is reduced using an appropriate reagent, such as zinc, in an appropriate solvent, such as AcOH, to afford aniline G. Further functionalization of aniline G, for example by reductive amination with an appropriate aldehyde or ketone, using an appropriate reducing agent, such as Na(OAc)₃BH, in an appropriate solvent, such as DCM, DCE, and/or AcOH, provides intermediate H. A second reductive amination with an appropriate aldehyde or ketone under similar conditions, or an alkylation with an appropriate alkylhalide, with an appropriate base, such as DIPEA, in an appropriate solvent, such as CH₃CN, provides intermediate I. Hydrolysis of the 2-methoxypyridine moiety using an appropriate reagent, such as hydrochloric acid, in an appropriate solvent, such as 1,4-dioxane and/or methanol, affords compounds of Formula (I). Such compounds may be further functionalized to provide other compounds of Formula (I) and/or they may be hydrogenated under appropriate conditions, such as in the presence of catalytic platinum on carbon in a hydrogen gas atmosphere, in an appropriate solvent, such as ethyl acetate and/or methanol, to provide the saturated macrocycle, representing compounds of Formula (I) wherein L is alkylenyl.

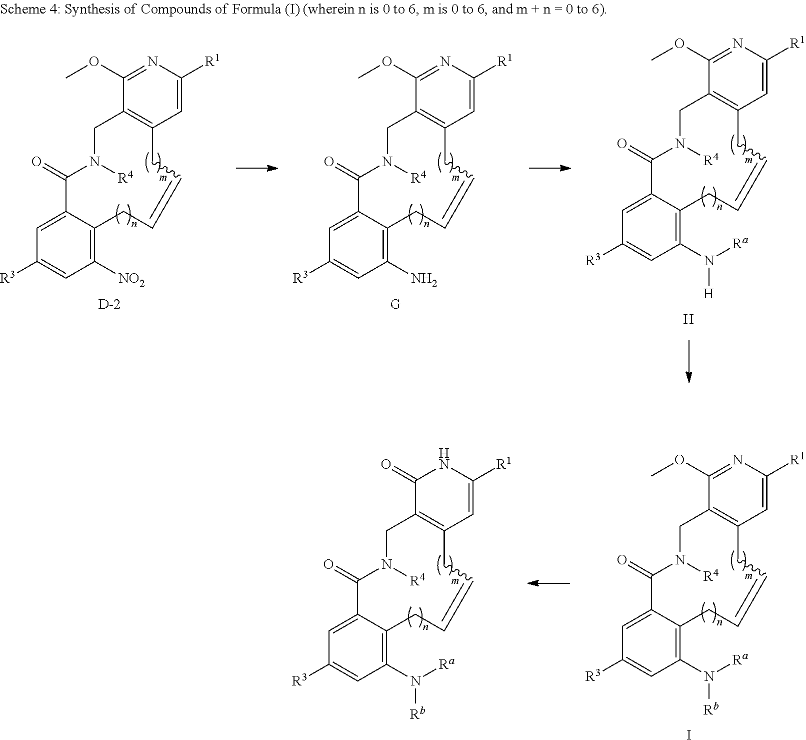

### EXPERIMENTALS

The following guidelines apply to all experimental procedures described herein. All reactions were conducted under a positive pressure of nitrogen using oven-dried glassware, unless otherwise indicated. Temperatures designated are external (i.e. bath temperatures), and are approximate. Air and moisture-sensitive liquids were transferred via syringe. Reagents were used as received. Solvents utilized were those listed as “anhydrous” by vendors. Molarities listed for reagents in solutions are approximate, and were used without prior titration against a corresponding standard. All reactions were agitated by stir bar, unless otherwise indicated. Heating was conducted using heating baths containing silicon oil, unless otherwise indicated. Reactions conducted by microwave irradiation (0-400 W at 2.45 GHz) were done so using a Biotage Initiator™ 2.0 instrument with Biotage® microwave EXP vials (0.2-20 mL) and septa and caps. Irradiation levels utilized (i.e. high, normal, low) based on solvent and ionic charge were based on vendor specifications. Cooling to temperatures below −70° C. was conducted using dry ice/acetone or dry ice/2-propanol. Magnesium sulfate and sodium sulfate used as drying agents were of anhydrous grade, and were used interchangeably. Solvents described as being removed “in vacuo” or “under reduced pressure” were done so by rotary evaporation.

Preparative normal phase silica gel chromatography was carried out using either a Teledyne ISCO CombiFlash® Companion instrument with RediSep® or ISCO® Gold silica gel cartridges (4 g-330 g), or an Analogix® IF280 instrument with SF25 silica gel cartridges (4 g-300 g), or a Biotage® SP1 instrument with HP silica gel cartridges (10 g-100 g). Purification by reverse phase HPLC was conducted using a YMC-pack column (ODS-A 75×30 mm) as solid phase, unless otherwise noted. A mobile phase of 25 mL/min A (CH₃CN-0.1% TFA): B (water-0.1% TFA), 10-80% gradient A (10 min) was utilized with UV detection at 214 nM, unless otherwise noted.

A PE Sciex® API 150 single quadrupole mass spectrometer (PE Sciex, Thornhill, Ontario, Canada) was operated using electrospray ionization in the positive ion detection mode. The nebulizing gas was generated from a zero air generator (Balston Inc., Haverhill, Mass., USA) and delivered at 65 psi and the curtain gas was high purity nitrogen delivered from a Dewar liquid nitrogen vessel at 50 psi. The voltage applied to the electrospray needle was 4.8 kV. The orifice was set at 25 V and mass spectrometer was scanned at a rate of 0.5 scan/sec using a step mass of 0.2 amu and collecting profile data.

Method A LCMS. Samples were introduced into the mass spectrometer using a CTC PAL® autosampler (LEAP Technologies, Carrboro, N.C.) equipped with a hamilton 10 uL syringe which performed the injection into a Valco 10-port injection valve. The HPLC pump was a Shimadzu® LC-10ADvp (Shimadzu Scientific Instruments, Columbia, Md.) operated at 0.3 mL/min and a linear gradient 4.5% A to 90% B in 3.2 min. with a 0.4 min. hold. The mobile phase was composed of 100% (H₂O 0.02% TFA) in vessel A and 100% (CH₃CN 0.018% TFA) in vessel B. The stationary phase is Aquasil (C18) and the column dimensions were 1 mm×40 mm. Detection was by UV at 214 nm, evaporative light-scattering (ELSD) and MS.

Method B, LCMS. Alternatively, an Agilent® 1100 analytical HPLC system with an LC/MS was used and operated at 1 mL/min and a linear gradient 5% A to 100% B in 2.2 min with a 0.4 min hold. The mobile phase was composed of 100% (H₂O 0.02% TFA) in vessel A and 100% (CH₃CN 0.018% TFA) in vessel B. The stationary phase was Zobax (C8) with a 3.5 um partical size and the column dimensions were 2.1 mm×50 mm. Detection was by UV at 214 nm, evaporative light-scattering (ELSD) and MS.

Method C, LCMS. Alternatively, an MDSSCIEX® API 2000 equipped with a capillary column of (50×4.6 mm, 5 μm) was used. HPLC was done on Agilent-1200 series UPLC system equipped with column Zorbax SB-C18 (50×4.6 mm, 1.8 μm) eluting with CH₃CN: ammonium acetate buffer. The reactions were performed in the microwave (CEM, Discover).

¹H-NMR spectra were recorded at 400 MHz using a Bruker® AVANCE 400 MHz instrument, with ACD Spect manager v. 10 used for reprocessing. Multiplicities indicated are: s=singlet, d=doublet, t=triplet, q=quartet, quint=quintet, sxt=sextet, m=multiplet, dd=doublet of doublets, dt=doublet of triplets etc. and br indicates a broad signal. All NMRs in DMSO-d₆ unless otherwise noted.

Analytical HPLC: Products were analyzed by Agilent® 1100 Analytical Chromatography system, with 4.5×75 mm Zorbax XDB-C18 column (3.5 um) at 2 mL/min with a 4 min gradient from 5% CH₃CN (0.1% formic acid) to 95% CH₃CN (0.1% formic acid) in H₂O (0.1% formic acid) and a 1 min hold.

### Preparation of Examples

### Example 1

### (E)-10-((trans-4-aminocyclohexyl)oxy)-12-chloro-3-methyl-5,6,15,16-tetrahydrobenzo[c]pyrido[4,3-j][1]azacyclododecine-1,14(2H,9H)-dione hydrochloride

### (a) 4-(but-3-en-1-yl)-2-methoxy-6-methylnicotinonitrile

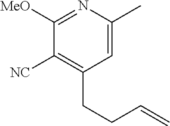

To a solution of 2-methoxy-4,6-dimethylnicotinonitrile (1.5 g, 9.25 mmol) in THF (40 mL) was added LiHMDS (10.17 mL, 10.17 mmol) at −78° C., and the mixture was stirred at −78° C. for 1 h. 3-Bromoprop-1-ene (0.880 mL, 10.17 mmol) was added and the mixture was stirred at −78° C. for 1 h and warmed to 0° C. over 1 h. The mixture was then stirred at 0° C. for 3 h. The reaction was quenched with saturated aqueous NH₄Cl solution and extracted with EtOAc (3×). The combined organics were dried over Na₂SO₄ and concentrated. The residue was purified using reverse phase HPLC using Trilution software, with a phenomenex Gemini 5 u C18(2) 100 A, AXIA 30×100 mm 5 micron, 10-minute run (30 mL/min, 40% CH₃CN/H₂O, 0.1% formic acid to 80% CH₃CN/H₂O, 0.1% formic acid) with UV detection at 254 nm to afford 4-(but-3-en-1-yl)-2-methoxy-6-methylnicotinonitrile (1.01 g, 54%) as a pale yellow oil. LC-MS (ES) m/z=203 [M+H]⁺.

### (b) (4-(but-3-en-1-yl)-2-methoxy-6-methylpyridin-3-yl)methanamine

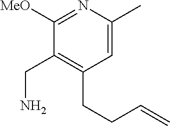

To a solution of 4-(but-3-en-1-yl)-2-methoxy-6-methylnicotinonitrile (700 mg, 3.46 mmol) in Et₂O (15 mL) at 0° C. was added LiAlH₄ (2 M in THF, 3.46 mL, 6.92 mmol), and the mixture was slowly warmed to room temperature and stirred at room temperature for 3 h. The mixture was cooled with ice-bath and quenched with minimum amount of water (until no more hydrogen was generated). The mixture was treated with DCM and filtered, and the residue was washed with DCM:MeOH (10:1). The combined organic phases were concentrated, and the residue was purified using flash chromatography (0 to 13% MeOH in DCM) to afford (4-(but-3-en-1-yl)-2-methoxy-6-methylpyridin-3-yl)methanamine (604 mg, 85%) as a pale yellow oil. LC-MS (ES) m/z=190 [M+H—NH₃]⁺ (major) 207 [M+H]⁺ (minor).

### (c) methyl 2-amino-3-methoxybenzoate

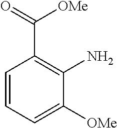 [SMILES: COC(=O)c1cccc(OC)c1N]

To a solution of 2-amino-3-methoxybenzoic acid (34.5 g, 206 mmol) in MeOH (250 mL) was added concentrated H₂SO₄ (50 mL) slowly. The reaction mixture was heated at 95° C. overnight. The reaction mixture was cooled to room temperature, and concentrated in vacuo. The residue was a beige slurry which was mixed with Et₂O and poured slowly into a mixture of Et₂O and cold aqueous saturated Na₂CO₃ containing excess Na₂CO₃. The Et₂O layer was separated and aqueous layer was extracted with Et₂O two more times. The combined Et₂O extracts were washed with diluted NaOH (2×), brine and dried over Na₂SO₄, filtered through a short silica plug and concentrated in vacuo to afford methyl 2-amino-3-methoxybenzoate (34.0 g, 91%) as a tan solid. LC-MS (ES) m/z=182 [M+H]⁺.

### (d) methyl 2-amino-5-chloro-3-methoxybenzoate

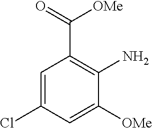 [SMILES: COC(=O)c1cc(Cl)cc(OC)c1N]

To a solution of methyl 2-amino-3-methoxybenzoate (34 g, 188 mmol) in DMF (200 mL) was added NCS (26.8 g, 197 mmol). The resulting mixture was heated at 50° C. for 3 h. The reaction mixture was cooled to room temperature and poured into cold water (300 mL). The solid was filtered and washed with water to afford methyl 2-amino-5-chloro-3-methoxybenzoate (36.2 g, 89%) as a brown solid. LC-MS (ES) m/z=216 [M+H]⁺.

### (e) methyl 2-bromo-5-chloro-3-methoxybenzoate

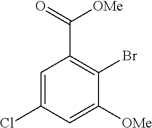 [SMILES: COC(=O)c1cc(Cl)cc(OC)c1Br]

To a solution of methyl 2-amino-5-chloro-3-methoxybenzoate (22 g, 102 mmol) in CH₃CN (300 mL) was added copper(II) bromide (68.4 g, 306 mmol). The mixture turned dark and was further stirred for 15 min at room temperature, tert-butyl nitrite (90% pure, 21.04 g, 184 mmol) was added dropwise over 10 min. The reaction mixture was stirred for additional 30 min, then heated at 60° C. overnight. The reaction mixture was concentrated in vacuo, and water and EtOAc were added. The resulting mixture was stirred until the dark green color disappeared. The organic phase became brown, and the aqueous was green with insoluble materials. The whole mixture was filtered through Celite® and washed with EtOAc. The EtOAc layer was separated, washed with brine, dried over Na₂SO₄, concentrated in vacuo and purified by flash chromatography (80-g column, dry load, 0-10% EtOAc in hexanes) to afford methyl 2-bromo-5-chloro-3-methoxybenzoate (14.4 g, 50%) as an off-white solid. LC-MS (ES) m/z=279, 281 [M+H]⁺.

### (f) methyl 2-allyl-5-chloro-3-methoxybenzoate

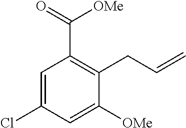 [SMILES: C=CCc1c(OC)cc(Cl)cc1C(=O)OC]

Three microwave vials were each charged with a mixture of methyl 2-bromo-5-chloro-3-methoxybenzoate (1 g, 3.58 mmol), allyltributylstannane (1.227 mL, 3.94 mmol), K₂CO₃ (0.989 g, 7.16 mmol), copper(I) iodide (0.136 g, 0.716 mmol), PdCl₂(dppf).CH₂Cl₂ (0.292 g, 0.358 mmol), and DMF (15 mL) then were sealed and heated in a microwave reactor at 100° C. for 90 min. The three reaction mixtures were combined and quenched with aqueous CsF solution. The solid was filtered, and the filtrate was extracted with Et₂O (4×). The combined organic extracts were washed with water (3×), brine, dried over Na₂SO₄, concentrated in vacuo and purified by flash chromatography (45-g column, 0-5% EtOAc in hexanes). The resulting oily residue was further purified by reverse phase HPLC (Gilson® instrument, Trilution software, Waters SunFire Prep C18 OBD 5 uM, 19×50 mm column, using 50-90% CH₃CN in water with 0.1% TFA) to afford methyl 2-allyl-5-chloro-3-methoxybenzoate (1.35 g, 52%) as a colorless oil. LC-MS (ES) m/z=241 [M+H]⁺.

### (g) 2-allyl-5-chloro-3-methoxybenzoic acid

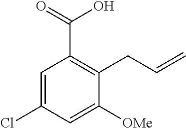

To a solution of methyl 2-allyl-5-chloro-3-methoxybenzoate (1.05 g, 4.36 mmol) in MeOH (15 mL) was added 6 N NaOH (5 mL, 30.0 mmol), and the reaction mixture was stirred at room temperature for 90 min. The volatiles were removed in vacuo. The residue was diluted with water and extracted with hexanes. The aqueous layer was acidified with 6 N HCl. The resulting suspension was filtered to afford 2-allyl-5-chloro-3-methoxybenzoic acid (920 mg, 93%) as white solid. LC-MS (ES) m/z=227 [M+H]⁺.

### (h) 2-allyl-N-((4-(but-3-en-1-yl)-2-methoxy-6-methylpyridin-3-yl)methyl)-5-chloro-3-methoxybenzamide

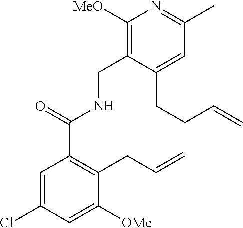 [SMILES: C=CCCc1cc(C)nc(OC)c1CNC(=O)c1cc(Cl)cc(OC)c1CC=C]

The reaction mixture of 2-allyl-5-chloro-3-methoxybenzoic acid (650 mg, 2.87 mmol), (4-(but-3-en-1-yl)-2-methoxy-6-methylpyridin-3-yl)methanamine (648 mg, 2.76 mmol), EDC (795 mg, 4.15 mmol), HOAt (564 mg, 4.15 mmol) and N-methylmorpholine (0.912 mL, 8.29 mmol) in DCM (18 mL) was stirred for 3 h at room temperature. The reaction mixture was quenched with saturated aqueous Na₂CO₃, and the layers were separated. The aqueous layer was extracted with DCM (2×). The combined organics were washed with water and brine, dried over Na₂SO₄, concentrated in vacuo and the residue was purified by flash chromatography (40-g column, 0-20% EtOAc in hexanes) to afford 2-allyl-N-((4-(but-3-en-1-yl)-2-methoxy-6-methylpyridin-3-yl)methyl)-5-chloro-3-methoxybenzamide (805 mg, 70%) as a white solid. LC-MS (ES) m/z=415 [M+H]⁺.

### (i) 12-chloro-1,10-dimethoxy-3-methyl-5,6,15,16-tetrahydrobenzo[c]pyrido[4,3-j][1]azacyclododecin-14(9H)-one

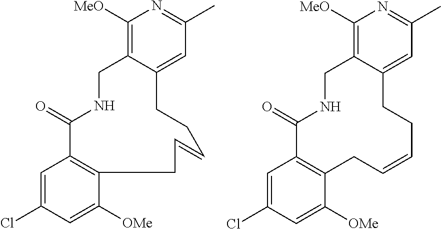 [SMILES: COc1cc(Cl)cc2c1C/C=C/CCc1cc(C)nc(OC)c1CNC2=O ; COc1cc(Cl)cc2c1C/C=C\CCc1cc(C)nc(OC)c1CNC2=O]

To a degassed solution of 2-allyl-N-((4-(but-3-en-1-yl)-2-methoxy-6-methylpyridin-3-yl)methyl)-5-chloro-3-methoxybenzamide (805 mg, 1.940 mmol) in DCM (100 mL) was added Grubbs II catalyst (165 mg, 0.194 mmol). The reaction mixture was stirred at room temperature overnight under nitrogen. The reaction mixture was concentrated in vacuo, and purified by flash chromatography (30-g column, 0-20% EtOAc in hexanes) to afford a mixture of E and Z isomers of 12-chloro-1,10-dimethoxy-3-methyl-5,6,15,16-tetrahydrobenzo[c]pyrido[4,3-j][1]azacyclododecin-14(9H)-one (517 mg, 69%) as an off-white solid. LC-MS (ES) m/z=387 [M+H]⁺.

### (j) 12-chloro-10-hydroxy-1-methoxy-3-methyl-5,6,15,16-tetrahydrobenzo[c]pyrido[4,3-j][1]azacyclododecin-14(9H)-one

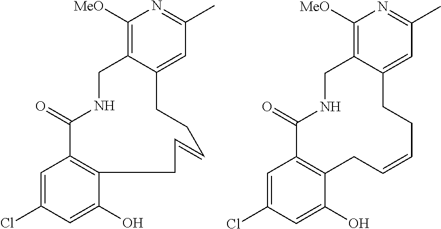 [SMILES: COc1nc(C)cc2c1CNC(=O)c1cc(Cl)cc(O)c1C/C=C/CC2 ; COc1nc(C)cc2c1CNC(=O)c1cc(Cl)cc(O)c1C/C=C\CC2]

To a solution of 12-chloro-1,10-dimethoxy-3-methyl-5,6,15,16-tetrahydrobenzo[c]pyrido[4,3-j][1]azacyclododecin-14(9H)-one (510 mg, 1.318 mmol) in DCM (40 mL) at −78° C. was added BBr₃ (1 M in DCM, 4.61 mL, 4.61 mmol) dropwise. The resulting mixture was slowly brought to room temperature and stirred overnight. The reaction mixture was diluted with DCM, and then quenched with saturated aqueous NaHCO₃ dropwise at 0° C. The resulting suspension was filtered to afford a beige solid. The layers of the filtrate were separated, and aqueous layer was extracted with DCM (2×). The combined organics were washed with brine and dried over Na₂SO₄, then concentrated to a solid. The solids were combined to afford a mixture of E and Z isomers of 12-chloro-10-hydroxy-1-methoxy-3-methyl-5,6,15,16-tetrahydrobenzo[c]pyrido[4,3-j][1]azacyclododecin-14(9H)-one (512 mg, 94%) as a beige solid. LC-MS (ES) m/z=373 [M+H]⁺.

### (k) cis-4-((tert-butoxycarbonyl)amino)cyclohexyl methanesulfonate

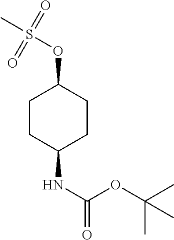 [SMILES: CC(C)(C)OC(=O)N[C@H]1CC[C@@H](OS(C)(=O)=O)CC1]

To a solution of tert-butyl(cis-4-hydroxycyclohexyl)carbamate (880 mg, 4.09 mmol) and Et₃N (1.424 mL, 10.22 mmol) in THF (20 mL) was added methanesulfonyl chloride (0.478 mL, 6.13 mmol) dropwise. The resulting mixture was stirred at room temperature for 2 h. The reaction mixture was concentrated in vacuo, diluted with EtOAc and washed with saturated aqueous NaHCO₃ (2×), brine (1×), dried over Na₂SO₄, filtered, then concentrated in vacuo to afford cis-4-((tert-butoxycarbonyl)amino)cyclohexyl methanesulfonate (1.16 g, 97%) as an off-white solid. LC-MS (ES) m/z=238 (major), 294 [M+H]⁺ (minor), 316 [M+Na]⁺ (minor).

### (l) tert-butyl(trans-4-(((E)-12-chloro-1-methoxy-3-methyl-14-oxo-5,6,9,14,15,16-hexahydrobenzo[c]pyrido[4,3-j][1]azacyclododecin-10-yl)oxy)cyclohexyl)carbamate and tert-butyl(trans-4-4(Z)-12-chloro-1-methoxy-3-methyl-14-oxo-5,6,9,14,15,16-hexahydrobenzo[c]pyrido[4,3-j][1]azacyclododecin-10-yl)oxy)cyclohexyl)carbamate

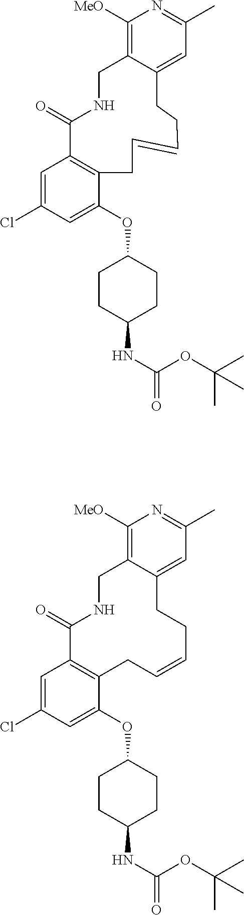 [SMILES: COc1nc(C)cc2c1CNC(=O)c1cc(Cl)cc(O[C@H]3CC[C@H](NC(=O)OC(C)(C)C)CC3)c1C/C=C/CC2 ; COc1nc(C)cc2c1CNC(=O)c1cc(Cl)cc(O[C@H]3CC[C@H](NC(=O)OC(C)(C)C)CC3)c1C/C=C\CC2]

To a solution of 12-chloro-10-hydroxy-1-methoxy-3-methyl-5,6,15,16-tetrahydrobenzo[c]pyrido[4,3-j][1]azacyclododecin-14(9H)-one (500 mg, 1.207 mmol) and cis-4-((tert-butoxycarbonyl)amino)cyclohexyl methanesulfonate (1062 mg, 3.62 mmol) in DMF (15 mL) was added Cs₂CO₃ (1966 mg, 6.03 mmol). The resulting mixture was heated at 60° C. for two days. The reaction mixture was diluted with water and extracted with EtOAc (3×). The combined organic extracts were washed with water (2×), brine (1×), dried over Na₂SO₄, filtered and concentrated in vacuo, then purified by flash chromatography (30-g column, 0-40% EtOAc in hexanes) to afford a white solid. The solid was further purified by reverse phase HPLC (Gilson® instrument, Trilution software, Waters SunFire Prep C18 OBD 5 uM, 19×50 mm column, using 50-80% CH₃CN in water with 0.1% TFA) to afford tert-butyl(trans-4-(((E)-12-chloro-1-methoxy-3-methyl-14-oxo-5,6,9,14,15,16-hexahydrobenzo[c]pyrido[4,3-j][1]azacyclododecin-10-yl)oxy)cyclohexyl)carbamate (347 mg, 50%) as a white solid. LC-MS (ES) m/z=571 [M+H]⁺.

Also isolated was tert-butyl(trans-4-(((Z)-12-chloro-1-methoxy-3-methyl-14-oxo-5,6,9,14,15,16-hexahydrobenzo[c]pyrido[4,3-j][1]azacyclododecin-10-yl)oxy)cyclohexyl)carbamate (252 mg, 37%) as a white solid. LC-MS (ES) m/z=571 [M+H]⁺.

### (m) (E)-10-((trans-4-aminocyclohexyl)oxy)-12-chloro-3-methyl-5,6,15,16-tetrahydrobenzo[c]pyrido[4,3-j][1]azacyclododecine-1,14(2H,9H)-dione hydrochloride

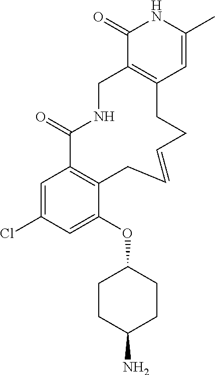 [SMILES: Cc1cc2c(c(=O)[nH]1)CNC(=O)c1cc(Cl)cc(O[C@H]3CC[C@H](N)CC3)c1C/C=C/CC2]

To a solution of tert-butyl(trans-4-(((E)-12-chloro-1-methoxy-3-methyl-14-oxo-5,6,9,14,15,16-hexahydrobenzo[c]pyrido[4,3-j][1]azacyclododecin-10-yl)oxy)cyclohexyl)carbamate (320 mg, 0.561 mmol) in 1,4-dioxane (10 mL) was added HCl (4 M in 1,4-dioxane, 3 mL, 12 mmol). The resulting mixture was heated at 70° C. for 3 h. The reaction mixture was concentrated in vacuo and the residue was triturated with EtOAc to afford (E)-10-((trans-4-aminocyclohexyl)oxy)-12-chloro-3-methyl-5,6,15,16-tetrahydrobenzo[c]pyrido[4,3-j][1]azacyclododecine-1,14(2H,9H)-dione hydrochloride (247 mg, 89%) as a white solid. LC-MS (ES) m/z=456 [M+H]⁺. ¹H NMR (DMSO-d₆): δ 11.34 (br. s., 1H), 8.20 (t, J=4.9 Hz, 1H), 8.02 (d, J=4.3 Hz, 3H), 7.23 (d, J=2.0 Hz, 1H), 6.85 (d, J=2.0 Hz, 1H), 5.85 (s, 1H), 4.99-5.17 (m, 2H), 4.31 (br. s., 1H), 4.17 (br. s., 1H), 3.31 (br. s., 2H), 3.05 (br. s., 1H), 2.16-2.26 (m, 2H), 2.12 (s, 3H), 2.05 (br. s., 2H), 1.90-2.00 (m, 2H), 1.31-1.55 (m, 4H). Note: 2H's not observed.

### Example 2

### (E)-12-chloro-10-((trans-4-(dimethylamino)cyclohexyl)oxy)-3-methyl-5,6,15,16-tetrahydrobenzo[c]pyrido[4,3-j][1]azacyclododecine-1,14(2H,9H)-dione

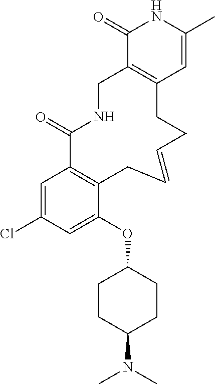 [SMILES: Cc1cc2c(c(=O)[nH]1)CNC(=O)c1cc(Cl)cc(O[C@H]3CC[C@H](N(C)C)CC3)c1C/C=C/CC2]

To a slurry of (E)-10-((trans-4-aminocyclohexyl)oxy)-12-chloro-3-methyl-5,6,15,16-tetrahydrobenzo[c]pyrido[4,3-j][1]azacyclododecine-1,14(2H,9H)-dione hydrochloride (230 mg, 0.467 mmol) in MeOH (8 mL) was added formaldehyde (0.278 mL, 3.74 mmol), NaBH₃CN (147 mg, 2.335 mmol) portionwise, then AcOH (0.027 mL, 0.467 mmol). The resulting mixture was stirred at room temperature overnight. The reaction mixture was concentrated and MeOH was added. The resulting suspension was filtered affording a residue and filtrate both containing product. The residue was purified by reverse phase HPLC (Gilson® instrument, Trilution software, Waters SunFire Prep C18 OBD 5 uM, 19×50 mm column, using 10-50% CH₃CN in water with 0.1% TFA). The resulting fractions were concentrated in vacuo and the residue was passed through a Silicycle (carbonate) cartridge (1 g) eluting with MeOH (30 mL) to afford a white solid (68 mg). The filtrate was concentrated in vacuo with silica and purified by flash chromatography (4-g column, 0-100% (1% NH₄OH+9% MeOH+90% CHCl₃) in CHCl₃) to afford a white solid (44 mg). The two solids were combined to afford (E)-12-chloro-10-((trans-4-(dimethylamino)cyclohexyl)oxy)-3-methyl-5,6,15,16-tetrahydrobenzo[c]pyrido[4,3-j][1]azacyclododecine-1,14(2H,9H)-dione (112 mg, 50%) as a white solid. LC-MS (ES) m/z=243 (major), 484 [M+H]⁺ (minor). ¹H NMR (400 MHz, METHANOL-d₄) δ: 7.11 (d, J=2.0 Hz, 1H), 6.94 (d, J=2.0 Hz, 1H), 6.12 (s, 1H), 5.25-5.36 (m, 1H), 5.12-5.23 (m, 1H), 4.37 (s, 2H), 4.23-4.34 (m, 1H), 3.42 (d, J=5.8 Hz, 2H), 3.11-3.23 (m, 1H), 2.81 (s, 6H), 2.67-2.74 (m, 2H), 2.34 (br. s., 2H), 2.20-2.30 (m, 5H), 2.12 (d, J=12.4 Hz, 2H), 1.61-1.74 (m, 2H), 1.46-1.61 (m, 2H). Note: 2 exchangeable H's not observed.

### Example 3

### 12-chloro-10-((trans-4-(dimethylamino)cyclohexyl)oxy)-3-methyl-6,7,8,9,15,16-hexahydrobenzo[c]pyrido[4,3-j][1]azacyclododecine-1,14(2H,5H)-dione

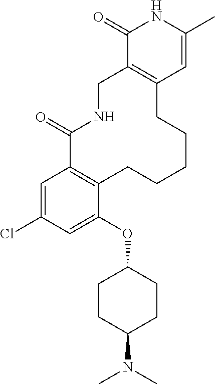 [SMILES: Cc1cc2c(c(=O)[nH]1)CNC(=O)c1cc(Cl)cc(O[C@H]3CC[C@H](N(C)C)CC3)c1CCCCC2]

A solution of (E)-12-chloro-10-((trans-4-(dimethylamino)cyclohexyl)oxy)-3-methyl-5,6,15,16-tetrahydrobenzo[c]pyrido[4,3-j][1]azacyclododecine-1,14(2H,9H)-dione (26 mg, 0.054 mmol) in EtOAc (2 mL) and MeOH (10 mL) was degassed for 5 min with nitrogen, then platinum (10 wt % on activated carbon, 10 mg) was added, and the solution was purged with nitrogen for another 5 min. The reaction mixture was stirred for 8 h under a hydrogen atmosphere (balloon). The reaction mixture was filtered and the filtrate was concentrated in vacuo to afford 12-chloro-10-((trans-4-(dimethylamino)cyclohexyl)oxy)-3-methyl-6,7,8,9,15,16-hexahydrobenzo[c]pyrido[4,3-j][1]azacyclododecine-1,14(2H,5H)-dione (20 mg, 77%) as a white solid. LC-MS (ES) m/z=244 (major), 486 [M+H]⁺ (minor). ¹H NMR (DMSO-d₆) δ: 11.41 (br. s., 1H), 8.55 (t, J=5.1 Hz, 1H), 7.04-7.16 (m, 1H), 6.79-6.89 (m, 1H), 5.87 (s, 1H), 4.39 (d, J=5.3 Hz, 2H), 4.32 (d, J=4.0 Hz, 1H), 2.55-2.65 (m, 2H), 2.43 (t, J=7.5 Hz, 2H), 2.17 (s, 7H), 2.11 (s, 3H), 1.96-2.07 (m, 2H), 1.74-1.86 (m, 2H), 1.57-1.71 (m, 2H), 1.28-1.51 (m, 8H).

### Example 4

### (Z)-10-((trans-4-aminocyclohexyl)oxy)-12-chloro-3-methyl-5,6,15,16-tetrahydrobenzo[c]pyrido[4,3-j][1]azacyclododecine-1,14(2H,9H)-dione hydrochloride

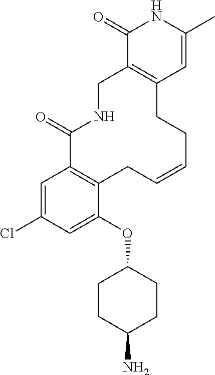 [SMILES: Cc1cc2c(c(=O)[nH]1)CNC(=O)c1cc(Cl)cc(O[C@H]3CC[C@H](N)CC3)c1C/C=C\CC2]

To a solution of tert-butyl(trans-4-(((Z)-12-chloro-1-methoxy-3-methyl-14-oxo-5,6,9,14,15,16-hexahydrobenzo[c]pyrido[4,3-j][1]azacyclododecin-10-yl)oxy)cyclohexyl)carbamate (188 mg, 0.330 mmol) in 1,4-dioxane (10 mL) was added HCl (4 M in 1,4-dioxane, 7 mL, 28 mmol). The resulting mixture was heated at 70° C. overnight. HCl (4 M in 1,4-dioxane, 4 mL) was added and the reaction mixture was heated at 70° C. for 3 days. The reaction mixture was concentrated in vacuo and the residue was triturated with EtOAc to afford (Z)-10-((trans-4-aminocyclohexyl)oxy)-12-chloro-3-methyl-5,6,15,16-tetrahydrobenzo[c]pyrido[4,3-j][1]azacyclododecine-1,14(2H,9H)-dione hydrochloride (118 mg, 73%) as an off-white solid. LC-MS (ES) m/z=456 [M+H]⁺. ¹H NMR (DMSO-d₆) δ: 11.46 (br. s., 1H), 8.29 (t, J=5.3 Hz, 1H), 8.16 (d, J=3.8 Hz, 3H), 7.22 (d, J=1.8 Hz, 1H), 6.85 (d, J=1.8 Hz, 1H), 5.95 (s, 1H), 5.07-5.27 (m, 2H), 4.39 (br. s., 2H), 3.63-3.76 (m, 1H), 3.54-3.64 (m, 1H), 3.06 (br. s., 1H), 2.55-2.66 (m, 2H), 2.33 (br. s., 2H), 2.12 (s, 3H), 2.02-2.10 (m, 2H), 1.91-2.03 (m, 2H), 1.36-1.60 (m, 4H).

### Example 5

### (Z)-12-chloro-10-((trans-4-(dimethylamino)cyclohexyl)oxy)-3-methyl-5,6,15,16-tetrahydrobenzo[c]pyrido[4,3-j][1]azacyclododecine-1,14(2H,9H)-dione

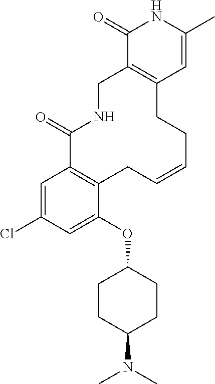 [SMILES: Cc1cc2c(c(=O)[nH]1)CNC(=O)c1cc(Cl)cc(O[C@H]3CC[C@H](N(C)C)CC3)c1C/C=C\CC2]

To a slurry of (Z)-10-((trans-4-aminocyclohexyl)oxy)-12-chloro-3-methyl-5,6,15,16-tetrahydrobenzo[c]pyrido[4,3-j][1]azacyclododecine-1,14(2H,9H)-dione hydrochloride (115 mg, 0.234 mmol) in MeOH (6 mL) was added formaldehyde (0.139 mL, 1.868 mmol), NaBH₃CN (73.4 mg, 1.168 mmol) portionwise, then AcOH (0.013 mL, 0.234 mmol). The resulting mixture was stirred at room temperature overnight. The reaction mixture was concentrated in vacuo and purified by reverse phase HPLC (Gilson® instrument, Trilution software, Waters SunFire Prep C18 OBD 5 uM, 19×50 mm column, using 10-50% CH₃CN in water with 0.1% TFA). The resulting fractions were concentrated in vacuo and the residue was passed through a Silicycle (carbonate) cartridge (1 g) eluting with MeOH to afford (Z)-12-chloro-10-((trans-4-(dimethylamino)cyclohexyl)oxy)-3-methyl-5,6,15,16-tetrahydrobenzo[c]pyrido[4,3-j][1]azacyclododecine-1,14(2H,9H)-dione (20 mg, 18%) as a white solid. LC-MS (ES) m/z=484 [M+H]⁺. ¹H NMR (400 MHz, METHANOL-d₄) δ: 7.08 (d, J=2.0 Hz, 1H), 6.88 (d, J=2.0 Hz, 1H), 6.23 (s, 1H), 5.15-5.28 (m, 2H), 4.54 (br. s., 2H), 4.25-4.35 (m, 1H), 3.47 (d, J=5.6 Hz, 2H), 2.83 (br. s., 2H), 2.53 (br. s., 2H), 2.31 (s, 7H), 2.26 (s, 3H), 2.19 (d, J=3.0 Hz, 2H), 1.96-2.04 (m, 2H), 1.40-1.54 (m, 4H). Note: 2 exchangeable H's not observed.

### Example 6

### (E)-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione hydrochloride, 0.10 1,4-dioxane solvate

### (a) methyl 3-amino-2-bromobenzoate

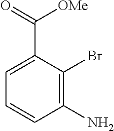 [SMILES: COC(=O)c1cccc(N)c1Br]

To a 100 mL RB flask was added 3-amino-2-bromobenzoic acid (1.75 g, 8.10 mmol), concentrated H₂SO₄ (2.159 mL, 40.5 mmol), and MeOH (80 mL). The reaction solution was heated to 65° C. for 18 h with stirring. The solvent was removed under vacuum and the residue slowly poured into iced saturated aqueous Na₂CO₃ solution. The product was extracted from the aqueous solution with EtOAc, dried over Na₂SO₄ and concentrated under vacuum to afford methyl 3-amino-2-bromobenzoate (1.8 g, 95%) as a brown oil. LC-MS (ES) m/z=230, 232 [M+H]⁺.

### (b) methyl 2-bromo-3-((tetrahydro-2H-pyran-4-yl)amino)benzoate

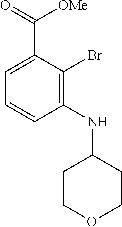 [SMILES: COC(=O)c1cccc(NC2CCOCC2)c1Br]

To a 100 mL RB flask was added dihydro-2H-pyran-4(3H)-one (1.11 g, 11.1 mmol), methyl 3-amino-2-bromobenzoate (1.7 g, 7.39 mmol), Na(OAc)₃BH (4.70 g, 22.17 mmol), AcOH (2.54 mL, 44.3 mmol) and DCE (50 mL). The reaction solution was stirred at room temperature for 60 h. The reaction solution was diluted with saturated aqueous NaHCO₃ (30 mL). The product was extracted with DCM, dried over Na₂SO₄ and concentrated under vacuum to give an orange oil. The residue was purified by flash chromatography (hexanes:EtOAc, 1:1) to afford methyl 2-bromo-3-((tetrahydro-2H-pyran-4-yl)amino)benzoate (1.9 g, 81%) as a golden colored oil which solidified upon standing. LC-MS (ES) m/z=314, 316 [M+H]⁺.

### (c) methyl 2-bromo-3-(ethyl(tetrahydro-2H-pyran-4-yl)amino)benzoate

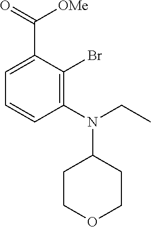 [SMILES: CCN(c1cccc(C(=O)OC)c1Br)C1CCOCC1]

To a 250 mL RB flask was added acetaldehyde (0.76 g, 17.2 mmol), methyl 2-bromo-3-((tetrahydro-2H-pyran-4-yl)amino)benzoate (1.8 g, 5.73 mmol), Na(OAc)₃BH (6.07 g, 28.6 mmol), AcOH (1.6 mL, 28.6 mmol) and DCE (50 mL). The reaction solution was stirred at room temperature for 48 h. Additional acetaldehyde (0.76 g, 17.2 mmol) and Na(OAc)₃BH (6.07 g, 28.6 mmol) were added to the reaction. After an additional 24 h, the reaction solution was diluted with saturated aqueous NaHCO₃ (50 mL). The product was extracted with DCM, dried over Na₂SO₄ and concentrated under vacuum to give an orange oil. The residue was purified by flash chromatography (hexanes:EtOAc, 2:1) to afford methyl 2-bromo-3-(ethyl(tetrahydro-2H-pyran-4-yl)amino)benzoate (1.1 g, 56%) as a yellow oil. LC-MS (ES) m/z=342, 344 [M+H]⁺.

### (d) methyl 2-allyl-3-(ethyl(tetrahydro-2H-pyran-4-yl)amino)benzoate

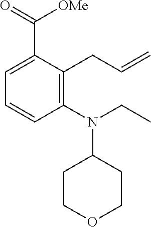 [SMILES: C=CCc1c(C(=O)OC)cccc1N(CC)C1CCOCC1]

To a 20 mL microwave vial was added methyl 2-bromo-3-(ethyl(tetrahydro-2H-pyran-4-yl)amino)benzoate (0.2 g, 0.58 mmol), allyltributylstannane (0.23 g, 0.70 mmol), copper(I) iodide (0.02 g, 0.12 mmol), K₂CO₃ (0.16 g, 1.17 mmol) and DMF (10 mL). The reaction solution was heated to 110° C. for 2 h, then 120° C. for 8 h in a microwave reactor. The reaction solution was diluted with saturated aqueous NaHCO₃ (50 mL). The product was extracted with DCM, dried over Na₂SO₄ and concentrated under vacuum to give a black oil. The residue was purified by flash chromatography (hexanes:EtOAc, 2:1) to afford methyl 2-allyl-3-(ethyl(tetrahydro-2H-pyran-4-yl)amino)benzoate (250 mg) as a yellow oil. LC-MS (ES) m/z=304 [M+H]⁺.

### (e) 2-allyl-3-(ethyl(tetrahydro-2H-pyran-4-yl)amino)benzoic acid

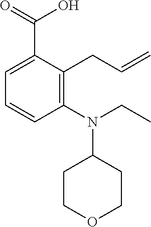 [SMILES: C=CCc1c(C(=O)O)cccc1N(CC)C1CCOCC1]

To a 50 mL RB flask was added methyl 2-allyl-3-(ethyl(tetrahydro-2H-pyran-4-yl)amino)benzoate (250 mg, 0.82 mmol) and aqueous NaOH (5 M, 1.6 mL, 8.2 mmol) in MeOH (10 mL). The reaction solution was stirred at 50° C. for 16 h. The reaction solution was concentrated under vacuum and the aqueous residue adjusted to pH=5 with aqueous HCl (3 M). The product was extracted with DCM, dried over Na₂SO₄ and concentrated under vacuum to afford 2-allyl-3-(ethyl(tetrahydro-2H-pyran-4-yl)amino)benzoic acid (200 mg, 80%) as a tan oil. LC-MS (ES) m/z=290 [M+H]⁺.

### (f) 2-allyl-N-((4-(but-3-en-1-yl)-2-methoxy-6-methylpyridin-3-yl)methyl)-3-(ethyl(tetrahydro-2H-pyran-4-yl)amino)benzamide

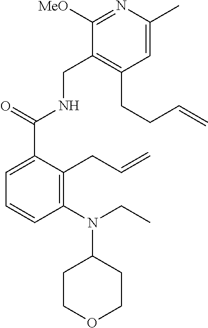 [SMILES: C=CCCc1cc(C)nc(OC)c1CNC(=O)c1cccc(N(CC)C2CCOCC2)c1CC=C]

To a 100 mL RB flask was added EDC (138 mg, 0.72 mmol), 2-allyl-3-(ethyl(tetrahydro-2H-pyran-4-yl)amino)benzoic acid (160 mg, 0.55 mmol), (4-(but-3-en-1-yl)-2-methoxy-6-methylpyridin-3-yl)methanamine (125 mg, 0.61 mmol), HOAt (98 mg, 0.72 mmol) and N-methylmorpholine (0.24 mL, 2.2 mmol) in DCM (30 mL). The reaction solution was stirred at room temperature for 20 h. The reaction contents were concentrated under vacuum and the residue was purified by flash chromatography (hexanes:EtOAc, 2:1) to afford 2-allyl-N-((4-(but-3-en-1-yl)-2-methoxy-6-methylpyridin-3-yl)methyl)-3-(ethyl(tetrahydro-2H-pyran-4-yl)amino)benzamide (210 mg, 76%) as a white foam. LC-MS (ES) m/z=478 [M+H]⁺.

### (g) (E)-10-(ethyl(tetrahydro-2H-pyran-4-yl)amino)-1-methoxy-3-methyl-5,6,15,16-tetrahydrobenzo[c]pyrido[4,3-j][1]azacyclododecin-14(9H)-one and (Z)-10-(ethyl(tetrahydro-2H-pyran-4-yl)amino)-1-methoxy-3-methyl-5,6,15,16-tetrahydrobenzo[c]pyrido[4,3-j][1]azacyclododecin-14(9H)-one

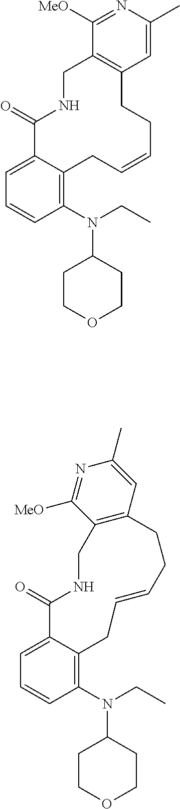 [SMILES: CCN(c1cccc2c1C/C=C\CCc1cc(C)nc(OC)c1CNC2=O)C1CCOCC1 ; CCN(c1cccc2c1C/C=C/CCc1cc(C)nc(OC)c1CNC2=O)C1CCOCC1]

To a 50 mL RB flask was added 2-allyl-N-((4-(but-3-en-1-yl)-2-methoxy-6-methylpyridin-3-yl)methyl)-3-(ethyl(tetrahydro-2H-pyran-4-yl)amino)benzamide (180 mg, 0.38 mmol), and Grubbs II catalyst (64.0 mg, 0.075 mmol) in dry DCM (15 mL). The reaction solution was stirred at room temperature for 16 h under a nitrogen atmosphere. Additional Grubbs II catalyst (64.0 mg, 0.075 mmol) was added and the reaction solution was stirred at room temperature for 20 h under a nitrogen atmosphere. The reaction contents were concentrated under vacuum and purified by flash chromatography (hexanes:EtOAc, 1:1) to afford an off-white foam (150 mg). The foam was purified by reverse phase HPLC (Gilson® instrument, Trilution software, Waters SunFire Prep C18 OBD 5 uM, 19×50 mm column, using 5-95% CH₃CN in water with 0.1% TFA) to afford (E)-10-(ethyl(tetrahydro-2H-pyran-4-yl)amino)-1-methoxy-3-methyl-5,6,15,16-tetrahydrobenzo[c]pyrido[4,3-j][1]azacyclododecin-14(9H)-one (100 mg, 58%) as a glassy solid. LC-MS (ES) m/z=450 [M+H]⁺, 366 is also prominent.

Also isolated was (Z)-10-(ethyl(tetrahydro-2H-pyran-4-yl)amino)-1-methoxy-3-methyl-5,6,15,16-tetrahydrobenzo[c]pyrido[4,3-j][1]azacyclododecin-14(9H)-one (40 mg, 23%) as a glassy solid. LC-MS (ES) m/z=450 [M+H]⁺, 366 is also prominent.

### (h) (E)-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione hydrochloride, 0.10 1,4-dioxane solvate

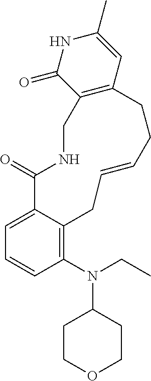 [SMILES: CCN(c1cccc2c1C/C=C/CCc1cc(C)[nH]c(=O)c1CNC2=O)C1CCOCC1]

To a 50 mL RB flask containing (E)-10-(ethyl(tetrahydro-2H-pyran-4-yl)amino)-1-methoxy-3-methyl-5,6,15,16-tetrahydrobenzo[c]pyrido[4,3-j][1]azacyclododecin-14(9H)-one (160 mg, 0.36 mmol) was added 1,4-dioxane (5 mL) and HCl (4 M in 1,4-dioxane, 6 mL, 25 mmol). The reaction solution was stirred at 60° C. for 1 h. The reaction material came out of solution as a viscous oil. Dry MeOH (1 mL) was added to the reaction (oil dissolved) and the solution was stirred at 60° C. for 2 h. Additional HCl (4 M in 1,4-dioxane, 1 mL) was added and the solution was stirred at 60° C. for an additional 13 h. The reaction solution was concentrated under vacuum to afford an off-white foam. The solid was triturated with hexanes and dried under high vacuum to afford (E)-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione hydrochloride, 0.10 1,4-dioxane solvate (110 mg, 63%) as an off-white solid. LC-MS (ES) m/z=450 [M+H]⁺. ¹H NMR (400 MHz, DMSO-d₆, 85° C.) δ: 7.63 (br. s, 1H), 7.37 (s, 1H), 7.20-7.30 (m, 1H), 7.11 (d, J=6.4 Hz, 1H), 5.89 (s, 1H), 5.20-5.32 (m, 1H), 5.09-5.20 (m, 1H), 4.24 (d, J=4.0 Hz, 2H), 3.81-3.93 (m, 2H), 3.71 (s, 2H), 3.12-3.32 (m, 5H), 2.60 (s, 2H), 2.24 (s, 2H), 2.16 (s, 3H), 1.76-1.55 (m, 4H), 0.86 (t, J=6.8 Hz, 3H).

### Example 7

### (Z)-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione hydrochloride, 0.10 1,4-dioxane solvate

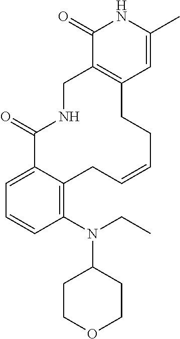 [SMILES: CCN(c1cccc2c1C/C=C\CCc1cc(C)[nH]c(=O)c1CNC2=O)C1CCOCC1]

To a 50 mL RB flask was added (Z)-10-(ethyl(tetrahydro-2H-pyran-4-yl)amino)-1-methoxy-3-methyl-5,6,15,16-tetrahydrobenzo[c]pyrido[4,3-j][1]azacyclododecin-14(9H)-one (80 mg, 0.18 mmol), 1,4-dioxane (5 mL) and HCl (6 mL, 25 mmol) 4 M in 1,4-dioxane. The reaction solution was stirred at 60° C. for 1 h. The reaction material came out of solution as a viscous oil. Dry MeOH (1 mL) was added to the reaction (oil dissolved) and the solution was stirred at 60° C. for 2 h. Additional HCl (4 M in 1,4-dioxane, 1 mL) was added and the solution was stirred at 60° C. for an additional 13 h. The reaction solution was concentrated under vacuum to afford an off-white foam. The solid was triturated with hexanes and dried under high vacuum to afford (Z)-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione hydrochloride, 0.10 1,4-dioxane solvate (40 mg, 23%) as an off-white solid. LC-MS (ES) m/z=450 [M+H]⁺. ¹H NMR (400 MHz, DMSO-d₆, 85° C.) δ: 7.79 (br. s, 1H), 7.37 (s, 1H), 7.22-7.32 (m, 1H), 7.05-7.17 (m, 1H), 5.96 (s, 1H), 5.15-5.30 (m, 1H), 5.05-5.15 (m, 1H), 4.37-4.43 (m, 2H), 3.80-3.95 (m, 2H), 3.65-3.80 (m, 2H), 3.15-3.35 (m, 5H), 2.63-2.77 (m, 2H), 2.35-2.45 (m, 2H), 2.16 (s, 3H), 1.50-1.80 (m, 4H), 0.88 (t, J=6.8 Hz, 3H).

### Example 8

### (E)-13-chloro-11-((trans-4-(dimethylamino)cyclohexyl)oxy)-3-methyl-5,6,7,10,16,17-hexahydro-1H-benzo[c]pyrido[4,3-k][1]azacyclotridecine-1,15(2H)-dione

### (a) 2-methoxy-6-methyl-4-(pent-4-en-1-yl)nicotinonitrile

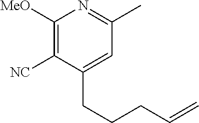 [SMILES: C=CCCCc1cc(C)nc(OC)c1C#N]

To a solution of 2-methoxy-4,6-dimethylnicotinonitrile (1.115 g, 6.87 mmol) in THF (20 mL) was added LiHMDS (1 M in toluene, 7.22 mL, 7.22 mmol) at 0° C. dropwise via syringe over 10 min, and the reaction was stirred at this temperature for 1 h. 4-Bromobut-1-ene (0.733 mL, 7.22 mmol) was added dropwise via syringe and the mixture was stirred from 0° C. to room temperature overnight. The reaction was poured into saturated aqueous ammonium chloride (50 mL) and extracted with EtOAc (3×75 mL). The combined organics were dried over Na₂SO₄, filtered, concentrated, and the residue purified by flash chromatography (0-20% EtOAc in hexanes, 40-g column, product fractions pooled and recolumned 0-10% EtOAc in hexanes, 40-g column) to afford 2-methoxy-6-methyl-4-(pent-4-en-1-yl)nicotinonitrile (619 mg, 42%) as a colorless oil. LC-MS (ES) m/z=217 [M+H]⁺.

### (b) (2-methoxy-6-methyl-4-(pent-4-en-1-yl)pyridin-3-yl)methanamine

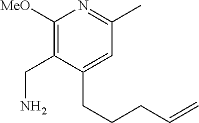 [SMILES: C=CCCCc1cc(C)nc(OC)c1CN]

To a solution of 2-methoxy-6-methyl-4-(pent-4-en-1-yl)nicotinonitrile (415 mg, 1.919 mmol) in Et₂O (10 mL) at 0° C. was added a solution of LiAlH₄ (1 M in THF) (3.84 mL, 3.84 mmol). The reaction was stirred at 0° C. for 1 h, and then allowed to warm to room temperature overnight. 150 μL of water was added and the reaction mixture stirred for 15 min at room temperature. 150 μL of 2 N NaOH was added and the reaction mixture stirred for 15 min at room temperature. 450 μL of water was added and the reaction mixture stirred for 30 min at room temperature. The reaction mixture was filtered and the residue washed with EtOAc (50 mL). The filtrate was concentrated, and the residue was purified by flash chromatography (100% EtOAc to 30% EtOH in EtOAc) to afford (2-methoxy-6-methyl-4-(pent-4-en-1-yl)pyridin-3-yl)methanamine (350 mg, 83%) as a yellow oil. LC-MS (ES) m/z=204 [M+H—NH₃]⁺ (major), 221 [M+H]⁺ (minor).

### (c) 2-allyl-5-chloro-3-methoxy-N-((2-methoxy-6-methyl-4-(pent-4-en-1-yl)pyridin-3-yl)methyl)benzamide

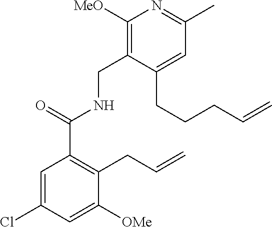 [SMILES: C=CCCCc1cc(C)nc(OC)c1CNC(=O)c1cc(Cl)cc(OC)c1CC=C]

A mixture of 2-allyl-5-chloro-3-methoxybenzoic acid (617 mg, 2.72 mmol), 2-methoxy-6-methyl-4-(pent-4-en-1-yl)pyridin-3-yl)methanamine (500 mg, 2.270 mmol), EDC (653 mg, 3.40 mmol), HOAt (463 mg, 3.40 mmol) and N-methylmorpholine (0.749 mL, 6.81 mmol) in DCM (20 mL) was stirred for 3 h at room temperature. The reaction mixture was quenched with saturated aqueous Na₂CO₃, and the layers were separated. The aqueous layer was extracted with DCM (2×). The combined organics were washed with water and brine, dried over Na₂SO₄, concentrated in vacuo and purified by flash chromatography (30-g column, 0-20% EtOAc in hexanes) to afford 2-allyl-5-chloro-3-methoxy-N-((2-methoxy-6-methyl-4-(pent-4-en-1-yl)pyridin-3-yl)methyl)benzamide (848 mg, 87%) as a white solid. LC-MS (ES) m/z=429 [M+H]⁺.

### (d) (E)-13-chloro-1,11-dimethoxy-3-methyl-6,7,16,17-tetrahydro-5H-benzo[c]pyrido[4,3-k][1]azacyclotridecin-15(10H)-one

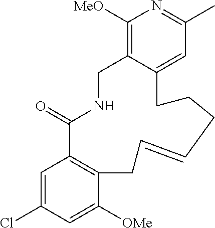 [SMILES: COc1cc(Cl)cc2c1C/C=C/CCCc1cc(C)nc(OC)c1CNC2=O]

To a degassed solution of 2-allyl-5-chloro-3-methoxy-N-((2-methoxy-6-methyl-4-(pent-4-en-1-yl)pyridin-3-yl)methyl)benzamide (840 mg, 1.958 mmol) in DCM (100 mL) was added Grubbs II catalyst (166 mg, 0.196 mmol), the reaction mixture was stirred at room temperature overnight. The reaction mixture was concentrated in vacuo, and purified by flash chromatography (30-g column, 0-30% EtOAc in hexanes, then 100% EtOAc). The resulting fractions were concentrated in vacuo and the residue was triturated with MeOH to afford (E)-13-chloro-1,11-dimethoxy-3-methyl-6,7,16,17-tetrahydro-5H-benzo[c]pyrido[4,3-k][1]azacyclotridecin-15(10H)-one (600 mg, 76%) as an off-white solid. LC-MS (ES) m/z=401 [M+H]⁺.

### (e) (E)-13-chloro-11-hydroxy-1-methoxy-3-methyl-6,7,16,17-tetrahydro-5H-benzo[c]pyrido[4,3-k][1]azacyclotridecin-15(10H)-one

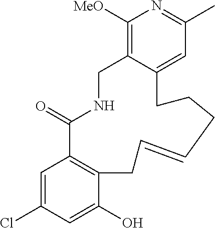 [SMILES: COc1nc(C)cc2c1CNC(=O)c1cc(Cl)cc(O)c1C/C=C/CCC2]

To a solution of (E)-13-chloro-1,11-dimethoxy-3-methyl-6,7,16,17-tetrahydro-5H-benzo[c]pyrido[4,3-k][1]azacyclotridecin-15(10H)-one (600 mg, 1.497 mmol) in DCM (70 mL) at −78° C. was added BBr₃ (1 M in DCM, 5.24 mL, 5.24 mmol) dropwise. The resulting mixture was slowly brought to room temperature and stirred overnight. The reaction mixture was diluted with DCM, then quenched with saturated aqueous NaHCO₃ dropwise at 0° C. The solid was filtered and washed with water to afford (E)-13-chloro-11-hydroxy-1-methoxy-3-methyl-6,7,16,17-tetrahydro-5H-benzo[c]pyrido[4,3-k][1]azacyclotridecin-15(10H)-one (398 mg) as a beige solid. LC-MS (ES) m/z=387 [M+H]⁺.

### (f) tert-butyl(trans-4-(((E)-13-chloro-1-methoxy-3-methyl-15-oxo-6,7,10,15,16,17-hexahydro-5H-benzo[c]pyrido[4,3-k][1]azacyclotridecin-11-yl)oxy)cyclohexyl)carbamate

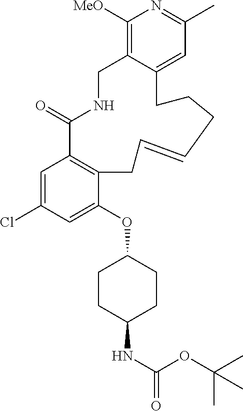 [SMILES: COc1nc(C)cc2c1CNC(=O)c1cc(Cl)cc(O[C@H]3CC[C@H](NC(=O)OC(C)(C)C)CC3)c1C/C=C/CCC2]

To a solution of E/Z-13-chloro-11-hydroxy-1-methoxy-3-methyl-6,7,16,17-tetrahydro-5H-benzo[c]pyrido[4,3-k][1]azacyclotridecin-15(10H)-one (390 mg, 1.008 mmol) and cis-4-((tert-butoxycarbonyl)amino)cyclohexyl methanesulfonate (887 mg, 3.02 mmol) in DMF (10 mL) was added Cs₂CO₃ (1642 mg, 5.04 mmol). The resulting mixture was heated at 60° C. overnight. The reaction mixture was diluted with water and extracted with EtOAc (3×). The combined organic extracts were washed with water (2×). There was waxy solid floating in the solution, so the suspension was filtered. The residue was purified by reverse phase HPLC (Gilson® instrument, Trilution software, Waters SunFire Prep C18 OBD 5 uM, 19×50 mm column, using 55-80% CH₃CN in water with 0.1% TFA) to afford a white solid (45 mg). The filtrate was concentrated in vacuo with silica, and purified by flash chromatography (12-g column, 0-40% EtOAc in hexanes followed by 100% EtOAc) and the resulting solid was purified by reverse phase HPLC (Gilson® instrument, Trilution software, Waters SunFire Prep C18 OBD 5 uM, 19×50 mm column, using 55-80% CH₃CN in water with 0.1% TFA) to afford an additional 40 mg of product. The two solids were combined to afford tert-butyl(trans-4-(((E)-13-chloro-1-methoxy-3-methyl-15-oxo-6,7,10,15,16,17-hexahydro-5H-benzo[c]pyrido[4,3-k][1]azacyclotridecin-11-yl)oxy)cyclohexyl)carbamate (85 mg, 14%) as a white solid. LC-MS (ES) m/z=584 [M+H]⁺.

### (g) (E)-11-((trans-4-aminocyclohexyl)oxy)-13-chloro-3-methyl-6,7,10,16,17,17a-hexahydro-1H-benzo[c]pyrido[4,3-k][1]azacyclotridecine-1,15(5H)-dione hydrochloride

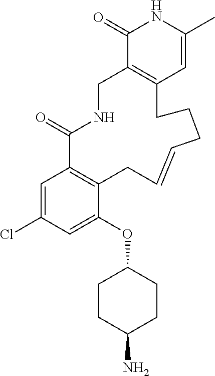 [SMILES: Cc1cc2c(c(=O)[nH]1)CNC(=O)c1cc(Cl)cc(O[C@H]3CC[C@H](N)CC3)c1C/C=C/CCC2]

To a slurry of tert-butyl(trans-4-(((E)-13-chloro-1-methoxy-3-methyl-15-oxo-6,7,10,15,16,17-hexahydro-5H-benzo[c]pyrido[4,3-k][1]azacyclotridecin-11-yl)oxy)cyclohexyl)carbamate (47 mg, 0.080 mmol) in 1,4-dioxane (3 mL) and MeOH (1 mL) was added HCl (4 M, 1,4-dioxane, 2 mL, 65.8 mmol). The resulting mixture was heated at 70° C. overnight. The reaction mixture was concentrated in vacuo and the residue was triturated with EtOAc to afford (E)-11-((trans-4-aminocyclohexyl)oxy)-13-chloro-3-methyl-6,7,10,16,17,17a-hexahydro-1H-benzo[c]pyrido[4,3-k][1]azacyclotridecine-1,15(5H)-dione hydrochloride (40 mg, 98%) as an off-white solid. LC-MS (ES) m/z=470 [M+H]⁺.

### (h) (E)-13-chloro-11-((trans-4-(dimethylamino)cyclohexyl)oxy)-3-methyl-5,6,7,10,16,17-hexahydro-1H-benzo[c]pyrido[4,3-k][1]azacyclotridecine-1,15(2H)-dione

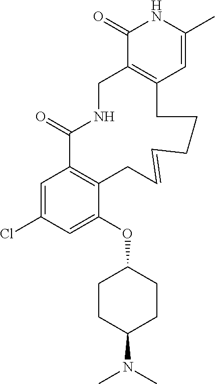 [SMILES: Cc1cc2c(c(=O)[nH]1)CNC(=O)c1cc(Cl)cc(O[C@H]3CC[C@H](N(C)C)CC3)c1C/C=C/CCC2]

To a solution of (E)-11-((trans-4-aminocyclohexyl)oxy)-13-chloro-3-methyl-5,6,7,10,16,17-hexahydro-1H-benzo[c]pyrido[4,3-k][1]azacyclotridecine-1,15(2H)-dione hydrochloride (40 mg, 0.079 mmol) in MeOH (3 mL) was added formaldehyde (0.059 mL, 0.790 mmol), NaBH₃CN (49.6 mg, 0.790 mmol), and then AcOH (4.52 μL, 0.079 mmol). The resulting mixture was stirred at room temperature 30 min. The reaction mixture was concentrated in vacuo and triturated with water. The suspension was filtered and the solid residue was purified by reverse phase HPLC (Gilson® instrument, Trilution software, Waters SunFire Prep C18 OBD 5 uM, 19×50 mm column, using 15-50% CH₃CN in water with 0.1% TFA). The resulting fractions were concentrated in vacuo and the residue was passed through a Silicycle (carbonate) cartridge (1 g) eluting with MeOH to afford (E)-13-chloro-11-((trans-4-(dimethylamino)cyclohexyl)oxy)-3-methyl-5,6,7,10,16,17-hexahydro-1H-benzo[c]pyrido[4,3-k][1]azacyclotridecine-1,15(2H)-dione (27 mg, 69%) as a white solid. LC-MS (ES) m/z=498 [M+H]⁺. ¹H NMR (400 MHz, DMSO-d₆) δ: 8.11 (t, J=4.0 Hz, 1H), 7.19 (d, J=2.0 Hz, 1H), 6.90 (d, J=2.0 Hz, 1H), 5.86 (s, 1H), 5.27-5.37 (m, 1H), 5.05 (dt, J=15.2, 7.3 Hz, 1H), 4.27-4.36 (m, 3H), 3.57 (d, J=6.1 Hz, 2H), 2.22-2.30 (m, 2H), 2.14-2.20 (s, 8H), 2.10 (s, 3H), 2.02 (t, J=5.9 Hz, 2H), 1.86-1.96 (m, 2H), 1.71-1.82 (m, 2H), 1.47 (br. s., 2H), 1.36 (t, J=9.5 Hz, 4H).

### Example 9

### (E)-10-(ethyl(tetrahydro-2H-pyran-4-yl)amino)-3,15-dimethyl-5,6,15,16-tetrahydrobenzo[c]pyrido[4,3-j][1]azacyclododecine-1,14(2H,9H)-dione

### (a) (E)-10-(ethyl(tetrahydro-2H-pyran-4-yl)amino)-1-methoxy-3,15-dimethyl-5,6,15,16-tetrahydrobenzo[c]pyrido[4,3-j][1]azacyclododecin-14(9H)-one

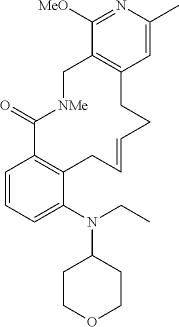 [SMILES: CCN(c1cccc2c1C/C=C/CCc1cc(C)nc(OC)c1CN(C)C2=O)C1CCOCC1]

To a stirred solution of (E)-10-(ethyl(tetrahydro-2H-pyran-4-yl)amino)-1-methoxy-3-methyl-5,6,15,16-tetrahydrobenzo[c]pyrido[4,3-j][1]azacyclododecin-14(9H)-one (100 mg, 0.222 mmol) in DMF (2 mL) at 0° C. was added sodium hydride (8.01 mg, 0.334 mmol) in one portion. The reaction was stirred for 15 min at 0° C. Methyl iodide (0.021 mL, 0.334 mmol) was added, and the reaction was stirred for 2 h at room temperature. The reaction mixture was quenched with saturated aqueous NaHCO₃ solution forming a white precipitate and stirred overnight. The precipitate was collected by filtration and the residue dried at the pump overnight to afford (E)-10-(ethyl(tetrahydro-2H-pyran-4-yl)amino)-1-methoxy-3,15-dimethyl-5,6,15,16-tetrahydrobenzo[c]pyrido[4,3-j][1]azacyclododecin-14(9H)-one (115 mg, ca. 90% pure) as a white solid. LC-MS (ES) m/z=464.4 [M+H]⁺.

### (b) (E)-10-(ethyl(tetrahydro-2H-pyran-4-yl)amino)-3,15-dimethyl-5,6,15,16-tetrahydrobenzo[c]pyrido[4,3-j][1]azacyclododecine-1,14(2H,9H)-dione

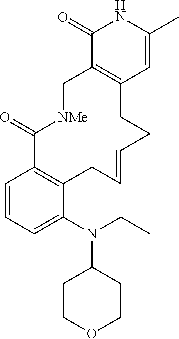 [SMILES: CCN(c1cccc2c1C/C=C/CCc1cc(C)[nH]c(=O)c1CN(C)C2=O)C1CCOCC1]

To a solution of (E)-10-(ethyl(tetrahydro-2H-pyran-4-yl)amino)-1-methoxy-3,15-dimethyl-5,6,15,16-tetrahydrobenzo[c]pyrido[4,3-j][1]azacyclododecin-14(9H)-one (115 mg, ca. 90% pure) in 1,4-dioxane (2 mL) was added HCl (4 M in dioxane, 4 mL, 132 mmol). The reaction was stirred at 80° C. over the weekend, then allowed to cool to room temperature. The reaction mixture was concentrated to afford a brown solid, then dissolved in MeOH (1 mL) and then EtOAc (50 mL). The organic solution was washed with saturated aqueous NaHCO₃ solution (20 mL), then dried over Na₂SO₄, concentrated and purified by flash chromatography (0-10% MeOH in EtOAc, then re-columned with 0-100% EtOAc in hexanes to 0-10% MeOH in EtOAc) to afford a colorless glass that was triturated with Et₂O to afford (E)-10-(ethyl(tetrahydro-2H-pyran-4-yl)amino)-3,15-dimethyl-5,6,15,16-tetrahydrobenzo[c]pyrido[4,3-j][1]azacyclododecine-1,14(2H,9H)-dione (52 mg, 0.116 mmol) as a white solid. LC-MS (ES) m/z=450.4 [M+H]⁺. ¹H NMR (400 MHz, DMSO-d₆) δ: 0.70-0.80 (m, 2H) 1.30-1.52 (m, 2H) 2.17 (s, 3H) 2.18-2.26 (m, 1H) 2.26-2.35 (m, 1H) 2.40-2.48 (m, 1H) 2.66 (s, 3H) 2.76-2.90 (m, 1H) 2.91-3.06 (m, 3H) 3.15-3.24 (m, 1H) 3.68-3.88 (m, 2H) 3.89-3.98 (m, 1H) 4.20-4.35 (m, 1H) 5.06-5.18 (m, 1H) 5.18-5.26 (m, 1H) 5.26-5.37 (m, 1H) 5.95-6.06 (m, 1H) 6.85-6.96 (m, 1H) 7.18-7.30 (m, 2H) 11.47-11.57 (m, 1H).

### Example 10

### (E)-11-(ethyl(tetrahydro-2H-pyran-4-yl)amino)-3,6-dimethyl-5,6,7,10,16,17-hexahydro-1H-benzo[h]pyrido[4,3-c][1,6]diazacyclotridecine-1,15(2H)-dione

### (a) ethyl 3-cyano-6-methyl-2-oxo-1,2-dihydropyridine-4-carboxylate

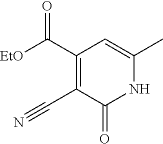

A solution of ethyl 2,4-dioxopentanoate (25 g, 158 mmol) and 2-cyanoacetamide (13.29 g, 158 mmol) in EtOH (200 mL) was stirred for 10 min and piperidine (3.91 mL, 39.5 mmol) was then added dropwise to the reaction over 4 min. The reaction was heated to 65° C. and stirred for 5 h. The reaction was allowed to cool to room temperature and stirred overnight. The reaction was poured into a mixture of aqueous HCl (1 N, 50 mL) in ice and stirred for 15 min, then placed into a freezer for 15 min. The suspension was filtered, washed with a small volume of water, and dried under a vacuum to afford ethyl 3-cyano-6-methyl-2-oxo-1,2-dihydropyridine-4-carboxylate (17.04 g, 81 mmol, 51.2% yield) as a yellow solid. LC-MS (ES) m/z=206.9 [M+H]⁺.

### (b) ethyl 3-cyano-2-methoxy-6-methylisonicotinate

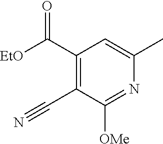 [SMILES: CCOC(=O)c1cc(C)nc(OC)c1C#N]

To a stirred suspension of ethyl 3-cyano-6-methyl-2-oxo-1,2-dihydropyridine-4-carboxylate (11.2 g, 54.3 mmol) in DCM (200 mL) was added trimethyloxonium tetrafluoroborate (10.04 g, 67.9 mmol). The reaction was stirred at 40° C. for 18 h. To the reaction was added aqueous NaOH (1 N, 100 mL)/ice-water. After stirring for 10 min the mixture was poured into a separatory funnel. The DCM phase was removed, washed with brine, dried over MgSO₄, filtered and concentrated under vacuum for 1 h to give a crude solid. This was purified via flash chromatography (220 gram silica column and a gradient of B: 3-15%; A: 1:1 heptane: DCM, B: Et₂O; collected all fractions on UV 330 nm) to afford ethyl 3-cyano-2-methoxy-6-methylisonicotinate (9.7 g, 43.2 mmol, 79% yield) as a white solid. LC-MS (ES) m/z=221.0 [M+H]⁺.

### (c) 4-(hydroxymethyl)-2-methoxy-6-methylnicotinonitrile

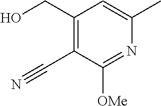

A suspension of ethyl 3-cyano-2-methoxy-6-methylisonicotinate (9.7 g, 44.0 mmol) and calcium chloride (19.55 g, 176 mmol) in THF (100 mL) and EtOH (100 mL) was stirred at 0° C. in an ice bath for 15 min, then sodium borohydride (5.00 g, 132 mmol) was added. The reaction was then allowed to warm to room temperature and stirred for 20 h, then additional sodium borohydride (1 g) was added and the reaction was stirred overnight. An equal volume of EtOAc was added and the reaction stirred for 1 h. The suspension was filtered through a pad of Celite® and washed with EtOAc (100 mL). The filtrate was transferred to a separatory funnel, washed with saturated aqueous ammonium chloride solution, dried over Na₂SO₄, filtered and concentrated under vacuum. The material was purified by flash chromatography (200 gram silica column and a gradient of B: 4-20%; A: DCM, B: EtOAc, collected all fractions on UV 290 nm) to afford 4-(hydroxymethyl)-2-methoxy-6-methylnicotinonitrile (3.31 g, 18.20 mmol, 41.3% yield) as a white solid. LC-MS (ES) m/z=179.0 [M+H]⁺.

### (d) (3-cyano-2-methoxy-6-methylpyridin-4-yl)methyl methanesulfonate

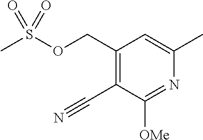

To a stirred solution of 4-(hydroxymethyl)-2-methoxy-6-methylnicotinonitrile (1000 mg, 5.61 mmol) in DCM (100 mL) at 0° C. was added Et₃N (2.347 mL, 16.84 mmol) and the reaction was stirred for 10 min followed by the addition of methanesulfonyl chloride (0.481 mL, 6.17 mmol) and the reaction was stirred for an additional 1 h. Then added in ice-water (50 mL) and stirred well for 15 min. The organic layer was separated and washed with brine, dried over MgSO₄, filtered and concentrated in vacuo to a residue. Heptane was added and then concentrated in vacuo to a residue that was dried under vacuum to afford (3-cyano-2-methoxy-6-methylpyridin-4-yl)methyl methanesulfonate (1.52 g, 4.74 mmol, 85% yield) as a solid. LC-MS (ES) m/z=257.0 [M+H]⁺.

### (e) 4-((allyl(methyl)amino)methyl)-2-methoxy-6-methylnicotinonitrile

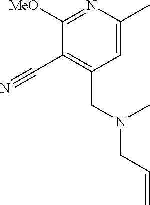 [SMILES: C=CCN(C)Cc1cc(C)nc(OC)c1C#N]

A solution of (3-cyano-2-methoxy-6-methylpyridin-4-yl)methyl methanesulfonate (1.52 g, 5.04 mmol) in DMF (30 mL) was stirred at room temperature for 5 min, then N-methylprop-2-en-1-amine (0.968 mL, 10.08 mmol) was added dropwise and the reaction was stirred for 5 min, then K₂CO₃ (0.836 g, 6.05 mmol) was added and the reaction was stirred for 2 h. A mixture of ice and saturated aqueous ammonium chloride solution was added and the reaction was stirred for 10 min then extracted with DCM (2×). The combined organics were dried over MgSO₄, filtered and concentrated in vacuo to a residue which was purified by flash chromatography (40 gram silica column and a gradient of B: 4-20%; A: DCM, B: 90/10/1 of CH₂Cl₂/MeOH/NH₄OH, collected all fractions on UV 290 nm) to afford 4-((allyl(methyl)amino)methyl)-2-methoxy-6-methylnicotinonitrile (1.05 g, 4.54 mmol, 90% yield) as a yellow oil. LC-MS (ES) m/z=232.0 [M+H]⁺.

### (f) N-((3-(aminomethyl)-2-methoxy-6-methylpyridin-4-yl)methyl)-N-methylprop-2-en-1-amine

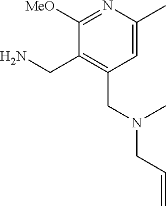 [SMILES: C=CCN(C)Cc1cc(C)nc(OC)c1CN]

To a solution of 4-((allyl(methyl)amino)methyl)-2-methoxy-6-methylnicotinonitrile (1.05 g, 4.54 mmol) in Et₂O (60 mL) was stirred for 10 min then placed into a ice bath and stirred for 15 min at 0° C. To this was added LiAlH₄ (1.0 M in Et₂O, 9.08 mL, 9.08 mmol) dropwise over about 10 min. The reaction mixture was stirred in an ice bath for 1 h then removed and allowed to warm to room temperature overnight. The reaction was placed back into the ice bath for 15 min then quenched with the following: 0.35 mL of water added slowly, 0.35 mL of 15% NaOH, then 1.03 mL of water, then removed from the bath and allowed to warm to room temperature and stirred for 30 min. The reaction was diluted with THF (60 mL), then filtered through Celite®, rinsed with THF, and the filtrate was concentrated in vacuo to afford N-((3-(aminomethyl)-2-methoxy-6-methylpyridin-4-yl)methyl)-N-methylprop-2-en-1-amine (1.01 g, 3.43 mmol, 76% yield) as a yellow liquid. LC-MS (ES) m/z=236.1 [M+H]⁺.

### (g) 2-allyl-N-((4-((allyl(methyl)amino)methyl)-2-methoxy-6-methylpyridin-3-yl)methyl)-3-(ethyl(tetrahydro-2H-pyran-4-yl)amino)benzamide

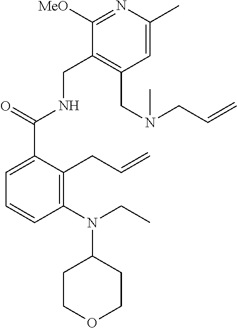 [SMILES: C=CCc1c(C(=O)NCc2c(CN(C)CC=C)cc(C)nc2OC)cccc1N(CC)C1CCOCC1]

To a solution of 2-allyl-3-(ethyl(tetrahydro-2H-pyran-4-yl)amino)benzoic acid (260 mg, 0.899 mmol), HOAt (147 mg, 1.078 mmol), EDC (207 mg, 1.078 mmol), and N-((3-(aminomethyl)-2-methoxy-6-methylpyridin-4-yl)methyl)-N-methylprop-2-en-1-amine (254 mg, 1.078 mmol) in DMF (6 mL) was added N-methylmorpholine (0.395 mL, 3.59 mmol) and the reaction was stirred at room temperature for about 3 h. The reaction was slowly diluted into ice-water (about 70 mL) with stirring and a solid precipitated. The mixture was extracted with DCM (50 mL, 2×), then EtOAc (50 mL). The combined organics were dried over MgSO₄, filtered and concentrated in vacuo. The residue was dissolved in DCM and adsorbed onto silica gel, then purified by flash chromatography (with a 12 gram silica column and a Gradient of B: 10-85%. A: Heptane. B: 3 to 1 EtOAc to EtOH+1% NH₄OH, collected all fractions on UV 254 nm) to afford 2-allyl-N-((4-((allyl(methyl)amino)methyl)-2-methoxy-6-methylpyridin-3-yl)methyl)-3-(ethyl(tetrahydro-2H-pyran-4-yl)amino)benzamide (259 mg, 0.511 mmol, 56.9% yield). LC-MS (ES) m/z=507.4 [M+H]⁺ (minor), 423.3 (minor), 254.3 (major).

### (h) 11-(ethyl(tetrahydro-2H-pyran-4-yl)amino)-1-methoxy-3,6-dimethyl-6,7,16,17-tetrahydro-5H-benzo[h]pyrido[4,3-c][1,6]diazacyclotridecin-15(10H)-one

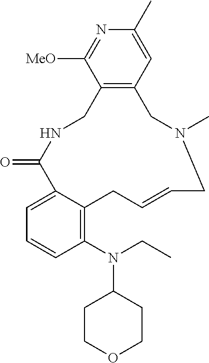 [SMILES: CCN(c1cccc2c1C/C=C/CN(C)Cc1cc(C)nc(OC)c1CNC2=O)C1CCOCC1]

2-Allyl-N-((4-((allyl(methyl)amino)methyl)-2-methoxy-6-methylpyridin-3-yl)methyl)-3-(ethyl(tetrahydro-2H-pyran-4-yl)amino)benzamide (35 mg, 0.069 mmol) was dissolved in DCM (3 mL) then stirred and degassed with a slow steam of nitrogen for 10 min, then Grubbs II (ca. 8.80 mg) was added, and the reaction was stirred overnight (24 h). Another portion of Grubbs II (ca. 5 mg) was added and the reaction was stirred for an additional 24 h. A second reaction was conducted: 2-allyl-N-((4-((allyl(methyl)amino)methyl)-2-methoxy-6-methylpyridin-3-yl)methyl)-3-(ethyl(tetrahydro-2H-pyran-4-yl)amino)benzamide (205 mg, 0.405 mmol) was dissolved in DCM (13 mL) then stirred and degassed with a slow steam of nitrogen for 10 min, then Grubbs II (51.5 mg, 0.061 mmol) was added. The reaction was stirred well at room temperature overnight (30 h). The two reaction mixtures were combined, diluted with DCM and adsorbed onto silica gel, then purified by flash chromatography (12 gram silica column with a gradient of B: 10-95%; A: heptane. B: 3:1 EtOAc:EtOH with 1% NH₄OH; collected all fractions on UV 290 nm) to afford 11-(ethyl(tetrahydro-2H-pyran-4-yl)amino)-1-methoxy-3,6-dimethyl-6,7,16,17-tetrahydro-5H-benzo[h]pyrido[4,3-c][1,6]diazacyclotridecin-15(10H)-one (55 mg, 0.098 mmol) as a solid. LC-MS (ES) m/z=479.4 [M+H]⁺.

### (i) (E)-11-(ethyl(tetrahydro-2H-pyran-4-yl)amino)-3,6-dimethyl-5,6,7,10,16,17-hexahydro-1H-benzo[h]pyrido[4,3-c][1,6]diazacyclotridecine-1,15(2H)-dione

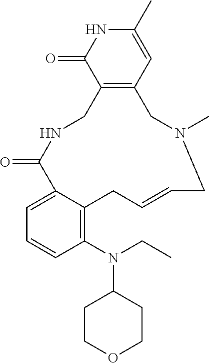 [SMILES: CCN(c1cccc2c1C/C=C/CN(C)Cc1cc(C)[nH]c(=O)c1CNC2=O)C1CCOCC1]

11-(Ethyl(tetrahydro-2H-pyran-4-yl)amino)-1-methoxy-3,6-dimethyl-6,7,16,17-tetrahydro-5H-benzo[h]pyrido[4,3-c][1,6]diazacyclotridecin-15(10H)-one (50 mg, 0.104 mmol) was treated with HCl (4 M in dioxane, 1.828 mL, 7.31 mmol) and MeOH (0.30 mL), and heated at 60° C. and stirred well until dissolved, then the reaction mixture was stirred for 3 h at 60° C. The reaction mixture was placed in a freezer overnight, then heated at 60° C. for one h, then concentrated to a residue. The residue was purified by preparative HPLC (using Sunfire 30×75 mm and a Gradient of B: 10-50%; A: water+0.1% TFA, B: CH₃CN+0.1% TFA, collected on UV 214 nm) and collected all product peaks and concentrated in vacuo to a residue. The residue was dissolved in small amount of DCM/MeOH and Et₃N (0.05 mL) was added. The solution was adsorbed onto silica and purified by flash chromatography (with a 4 gram silica column and a Gradient of B: 10-100%; A: DCM, B: 90/10/1 of CH₂Cl₂/MeOH/NH₄OH, collected all fractions on UV 330 nm). Product fractions were combined and volatiles were removed in vacuo to afford a residue which was triturated with CH₃CN to afford (E)-11-(ethyl(tetrahydro-2H-pyran-4-yl)amino)-3,6-dimethyl-5,6,7,10,16,17-hexahydro-1H-benzo[h]pyrido[4,3-c][1,6]diazacyclotridecine-1,15(2H)-dione (25 mg, 0.052 mmol, 50.0% yield) as a white solid. LC-MS (ES) 465.3 [M+H]⁺. ¹H NMR (400 MHz, DMSO-d₆) δ: 11.60 (br. s., 1H), 7.89 (br. s., 1H), 7.12-7.35 (m, 2H), 6.97 (d, J=7.1 Hz, 1H), 5.91 (s, 1H), 5.67 (dt, J=14.5, 7.1 Hz, 1H), 5.19-5.35 (m, 1H), 4.30 (d, J=3.0 Hz, 2H), 3.72-3.92 (m, 4H), 3.18-3.29 (m, 3H), 3.07-3.17 (m, 2H), 2.88-3.06 (m, 4H), 2.75-2.83 (m, 2H), 2.18 (s, 3H), 2.13 (s, 3H), 1.36-1.50 (m, 2H), 1.25 (br. s., 1H), 1.18 (t, J=7.2 Hz, 1H), 0.78 (d, J=13.9 Hz, 1H).

### Example 11

### 11-(ethyl(tetrahydro-2H-pyran-4-yl)amino)-3-methyl-7,8,9,10,16,17-hexahydrobenzo[h]pyrido[4,3-c][1,6]oxaazacyclotridecine-1,15(2H,5H)-dione

### (a) 4-((allyloxy)methyl)-2-methoxy-6-methylnicotinonitrile

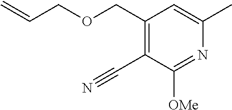 [SMILES: C=CCOCc1cc(C)nc(OC)c1C#N]

A stirred solution of 4-(hydroxymethyl)-2-methoxy-6-methylnicotinonitrile (0.40 g, 2.245 mmol) and 3-bromoprop-1-ene (0.233 mL, 2.69 mmol) in DMF (12 mL) was placed into a brine/ice bath and stirred for 15 min. NaH (60% dispersion in mineral oil, 0.103 g, 2.58 mmol) was added, and the reaction was stirred for 120 min. Additional NaH (60% dispersion in mineral oil, ca. 10 mg) was added and the reaction was stirred for an additional 1 h. The reaction was poured into ice and saturated aqueous ammonium chloride solution and the mixture was stirred for 10 min then extracted with 10% EtOAc in Et₂O, then extracted with Et₂O. The combined organics were dried over MgSO₄, filtered and concentrated in vacuo to a residue which was dissolved in DCM and purified by flash chromatography (24 gram silica column and a gradient of B: 5-40%; A: heptanes, B: EtOAc, collected all fractions on UV 290 nm) to afford 4-((allyloxy)methyl)-2-methoxy-6-methylnicotinonitrile (246 mg, 1.127 mmol, 50.2% yield) as a white solid. LC-MS (ES) m/z=219.0 [M+H]⁺.

### (b) (4-((allyloxy)methyl)-2-methoxy-6-methylpyridin-3-yl)methanamine

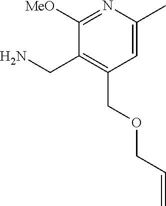 [SMILES: C=CCOCc1cc(C)nc(OC)c1CN]

A solution of 4-((allyloxy)methyl)-2-methoxy-6-methylnicotinonitrile (235 mg, 1.077 mmol) in Et₂O (20 mL) was placed into a ice bath and stirred for 15 min at 0° C. To this was added LiAlH₄ (1.0 M in Et₂O, 2.153 mL, 2.153 mmol) over about 3 min. The reaction mixture was kept in the ice-bath for 1 h then removed and allowed to warm to room temperature and was stirred for 2 h. The reaction mixture was placed back into the ice-bath for 5 min, and then quenched with the following: 0.082 mL of water added slowly, 0.082 mL of 15% NaOH, then 0.25 mL of water. The ice-bath was removed and the reaction mixture was allowed to warm to room temperature and stirred for 60 min and THF (20 mL) was added. The suspension was filtered through Celite®, and then rinsed with THF, EtOAc, and Et₂O. The filtrate was concentrated in vacuo to afford the title compound (4-((allyloxy)methyl)-2-methoxy-6-methylpyridin-3-yl)methanamine (214 mg, 0.963 mmol, 89% yield) as an oil. LC-MS (ES) m/z=223.0 [M+H]⁺.

### (c) 2-allyl-N-((4-((allyloxy)methyl)-2-methoxy-6-methylpyridin-3-yl)methyl)-3-(ethyl(tetrahydro-2H-pyran-4-yl)amino)benzamide

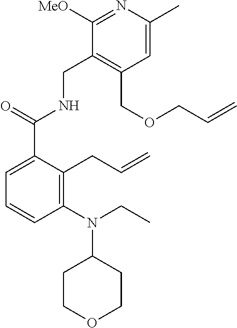 [SMILES: C=CCOCc1cc(C)nc(OC)c1CNC(=O)c1cccc(N(CC)C2CCOCC2)c1CC=C]

To a reaction vessel containing 2-allyl-3-(ethyl(tetrahydro-2H-pyran-4-yl)amino)benzoic acid (210 mg, 0.726 mmol), HOAt (119 mg, 0.871 mmol), and EDC (167 mg, 0.871 mmol) was added a solution of (4-((allyloxy)methyl)-2-methoxy-6-methylpyridin-3-yl)methanamine (215 mg) in DMF (10 mL) followed by N-methylmorpholine (0.319 mL, 2.90 mmol). The reaction was stirred at room temperature for about 18 h. The reaction mixture was poured into water (100 mL) and stirred for 10 min then extracted with Et₂O (2×80 mL). The organics were combined and dried over MgSO₄, then filtered and concentrated in vacuo to a residue which was purified via flash chromatography (40 gram silica column and a gradient of B: 5-65%; A: heptanes, B: EtOAc, collected all fractions on UV 290 nm) to afford 2-allyl-N-((4-((allyloxy)methyl)-2-methoxy-6-methylpyridin-3-yl)methyl)-3-(ethyl(tetrahydro-2H-pyran-4-yl)amino)benzamide (217 mg, 0.440 mmol, 60.6% yield) as a residue. LC-MS (ES) m/z=494.3 [M+H]⁺ (minor), 410.2 (minor), 247.7 (major).

### (d) 11-(ethyl(tetrahydro-2H-pyran-4-yl)amino)-1-methoxy-3-methyl-7,10,16,17-tetrahydrobenzo[h]pyrido[4,3-c][1,6]oxaazacyclotridecin-15(5H)-one

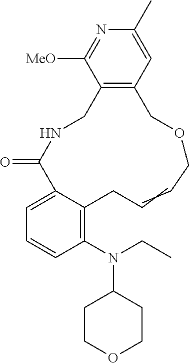 [SMILES: CCN(c1cccc2c1CC=CCOCc1cc(C)nc(OC)c1CNC2=O)C1CCOCC1]

2-Allyl-N-((4-((allyloxy)methyl)-2-methoxy-6-methylpyridin-3-yl)methyl)-3-(ethyl(tetrahydro-2H-pyran-4-yl)amino)benzamide (217 mg, 0.440 mmol) was dissolved in DCM (22 mL) and begin stirring and degassing with nitrogen for 10 min, then added Grubbs II (37.3 mg, 0.044 mmol). Capped and covered from light and stirred well at room temperature overnight (22 h). The reaction mixture was adsorbed onto silica and purified via flash chromatography (12 gram silica column and a gradient of B: 5-65%, A: heptane. B: 3 to 1 EtOAc to EtOH, collected all fractions on UV 290 nm) to afford 11-(ethyl(tetrahydro-2H-pyran-4-yl)amino)-1-methoxy-3-methyl-7,10,16,17-tetrahydrobenzo[h]pyrido[4,3-c][1,6]oxaazacyclotridecin-15(5H)-one (112 mg, 0.241 mmol, 54.7% yield) as a solid. LC-MS (ES) m/z=466.3 [M+H]⁺.

### (e) 11-(ethyl(tetrahydro-2H-pyran-4-yl)amino)-1-methoxy-3-methyl-7,8,9,10,16,17-hexahydrobenzo[h]pyrido[4,3-c][1,6]oxaazacyclotridecin-15(5H)-one

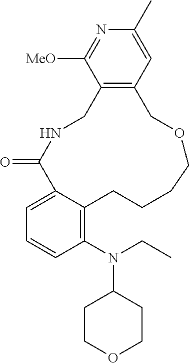 [SMILES: CCN(c1cccc2c1CCCCOCc1cc(C)nc(OC)c1CNC2=O)C1CCOCC1]

To 11-(Ethyl(tetrahydro-2H-pyran-4-yl)amino)-1-methoxy-3-methyl-7,10,16,17-tetrahydrobenzo[h]pyrido[4,3-c][1,6]oxaazacyclotridecin-15(5H)-one (52 mg, 0.112 mmol) was added EtOH (3 mL) and EtOAc (2 mL) and THF (1 mL) and heated slightly to obtain a solution. The reaction was allowed to cool to room temperature, then was purged with nitrogen for 2 min then Pd/C (10 wt % on activated carbon, 5.94 mg, 5.58 μmol) was added and the reaction placed under a hydrogen atmosphere (balloon) and the reaction was stirred overnight. Celite® and DCM (5 mL) were added to the reaction, and the mixture was filtered through Celite® and washed with 10% MeOH in DCM. The filtrate was concentrated and the residue was treated with methyl t-butyl ether and concentrated, then treated with heptane and concentrated to afford 11-(ethyl(tetrahydro-2H-pyran-4-yl)amino)-1-methoxy-3-methyl-7,8,9,10,16,17-hexahydrobenzo[h]pyrido[4,3-c][1,6]oxaazacyclotridecin-15(5H)-one (53 mg, 0.111 mmol, 99% yield) as a solid. LC-MS (ES) m/z=468.3 [M+H]⁺ (minor), 234.7 (major)

### (f) 11-(ethyl(tetrahydro-2H-pyran-4-yl)amino)-3-methyl-7,8,9,10,16,17-hexahydrobenzo[h]pyrido[4,3-c][1,6]oxaazacyclotridecine-1,15(2H,5H)-dione

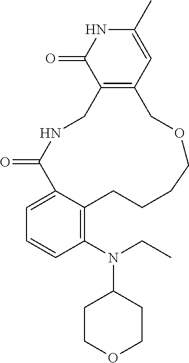 [SMILES: CCN(c1cccc2c1CCCCOCc1cc(C)[nH]c(=O)c1CNC2=O)C1CCOCC1]

11-(Ethyl(tetrahydro-2H-pyran-4-yl)amino)-1-methoxy-3-methyl-7,8,9,10,16,17-hexahydrobenzo[h]pyrido[4,3-c][1,6]oxaazacyclotridecin-15(5H)-one (49 mg, 0.105 mmol) was dissolved in HCl (4 M in dioxane, 1.572 mL, 6.29 mmol) and MeOH (0.40 mL) and the resulting solution was placed into a heat block and heated at 65° C. for 18 h. The reaction was diluted with EtOAc and concentrated in vacuo to a residue which was dissolved in DCM and MeOH with 2 drops of concentrated NH₄OH then adsorbed onto silica gel and purified by flash chromatography (4 gram silica column and a gradient of B: 10-100%. A: DCM B: 90/10/1 of CH₂Cl₂/MeOH/NH₄OH, collected all fractions on UV 254 nm) to afford a residue which was treated with methyl t-butyl ether and concentrated (twice) to afford 11-(ethyl(tetrahydro-2H-pyran-4-yl)amino)-3-methyl-7,8,9,10,16,17-hexahydrobenzo[h]pyrido[4,3-c][1,6]oxaazacyclotridecine-1,15(2H,5H)-dione (30 mg, 0.063 mmol, 60.6% yield) as a white solid. LC-MS (ES) m/z=454.3 [M+H]⁺. ¹H NMR (400 MHz, DMSO-d₆) δ: 11.65 (br. s., 1H), 8.10 (t, J=4.3 Hz, 1H), 7.09-7.21 (m, 2H), 7.00 (dd, J=7.1, 1.5 Hz, 1H), 5.97 (s, 1H), 4.22-4.39 (m, 4H), 3.77-3.87 (m, 2H), 3.49 (t, J=4.9 Hz, 2H), 3.20 (t, J=10.9 Hz, 2H), 2.87-3.05 (m, 3H), 2.79-2.87 (m, 2H), 2.14 (s, 3H), 1.60 (br. s., 2H), 1.41-1.56 (m, 6H), 0.77 (t, J=6.9 Hz, 3H).

### Example 12

### (E)- and (Z)-11-(ethyl(tetrahydro-2H-pyran-4-yl)amino)-3-methyl-7,10,16,17-tetrahydrobenzo[h]pyrido[4,3-c][1,6]oxaazacyclotridecine-1,15(2H,5H)-dione

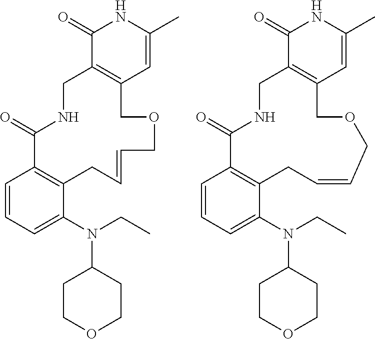 [SMILES: CCN(c1cccc2c1C/C=C/COCc1cc(C)[nH]c(=O)c1CNC2=O)C1CCOCC1 ; CCN(c1cccc2c1C/C=C\COCc1cc(C)[nH]c(=O)c1CNC2=O)C1CCOCC1]

A 20 mL screw-cap vial was charged with 11-(ethyl(tetrahydro-2H-pyran-4-yl)amino)-1-methoxy-3-methyl-7,10,16,17-tetrahydrobenzo[h]pyrido[4,3-c][1,6]oxaazacyclotridecin-15(5H)-one (52 mg, 0.112 mmol), HCl (4 M, dioxane, 1.675 mL, 6.70 mmol) and then MeOH (0.40 mL), capped and placed into heat block at 65° C. for 18 h. The reaction was diluted slightly with EtOAc and concentrated in vacuo. The residue was dissolved in DCM+MeOH and then 2 drops of NH₄OH (conc) were added. The solution was adsorbed onto silica and purified by flash chromatography (4 gram column, gradient B: 10-100%. A: DCM. B: 90/10/1 CH₂Cl₂/MeOH/NH₄OH). The product fractions were combined and concentrated in vacuo to a residue that was transferred to a submission vial via DCM, concentrated under N₂ stream, then treated with methyl t-butyl ether and concentrated (2×) to afford a mixture of (E)- and (Z)-11-(ethyl(tetrahydro-2H-pyran-4-yl)amino)-3-methyl-7,10,16,17-tetrahydrobenzo[h]pyrido[4,3-c][1,6]oxaazacyclotridecine-1,15(2H,5H)-dione (10 mg) as a white solid. LC-MS (ES) and 1H NMR analysis suggest a ca. 1:1 ratio of (E) and (Z) isomers. LC-MS (ES) m/z=452.3 [M+H]⁺. ¹H NMR (DMSO-d₆) δ: 11.58 (br. s., 1H), 8.00-8.18 (m, 1H), 7.17-7.32 (m, 2H), 7.01-7.12 (m, 1H), 5.98-6.11 (m, 1H), 5.19-5.64 (m, 2H), 4.46 (s, 1H), 4.23-4.36 (m, 3H), 4.00 (d, J=5.8 Hz, 1H), 3.75-3.91 (m, 4H), 3.67 (d, J=5.3 Hz, 1H), 3.14-3.27 (m, 2H), 2.87-3.05 (m, 3H), 2.10-2.23 (m, 3H), 1.61 (br. s., 2H), 1.30-1.46 (m, 2H), 0.69-0.81 (m, 3H).

### Example 13

### (Z)-10-(ethyl(tetrahydro-2H-pyran-4-yl)amino)-3-methyl-6,9,15,16-tetrahydro-1H-benzo[g]pyrido[4,3-b][1,5]oxaazacyclododecine-1,14(2H)-dione

### (a) 4-chloro-2-methoxy-6-methylnicotinonitrile

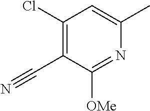

To a stirred suspension of 4-chloro-6-methyl-2-oxo-1,2-dihydropyridine-3-carbonitrile (2.96 g, 17.56 mmol) in DCM (100 mL) was added trimethyloxonium tetrafluoroborate (3.25 g, 21.95 mmol). The reaction mixture was placed under a nitrogen atmosphere and heated in an oil bath at 45° C. for 18 h. To the reaction was added 1 N NaOH/ice-water (150 mL). After stirring for 20 min the mixture was separated. The organics were removed and washed with brine, dried over MgSO₄ and filtered and concentrated in vacuo. This solid was dissolved in DCM and toluene and purified via flash chromatography (40 g column, 5-40% heptane in EtOAc; mixed fractions recolumned with 80 g column, 5-40% heptane in EtOAc) to afford 4-chloro-2-methoxy-6-methylnicotinonitrile (2.06 g, 62% yield) as a white solid. LC-MS (ES) m/z=182.9 [M+H]⁺.

### (b) 4-chloro-6-methyl-2-oxo-1,2-dihydropyridine-3-carbonitrile

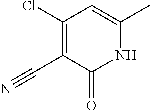

A solution of phosphorus pentachloride (18.03 g, 87 mmol) in CHCl₃ (100 mL) was stirred for 10 min, then phosphoryl chloride (8.07 mL, 87 mmol) and 4-hydroxy-6-methyl-2-oxo-1,2-dihydropyridine-3-carbonitrile (10 g, 66.6 mmol) were added. The reaction was placed into an oil bath, heated to 72° C., and stirred overnight. A portion of the volatiles was removed in vacuo then the remaining liquid was poured into ice/water with vigorous stirring The reaction was sparged with nitrogen for 1 h to remove volatiles and achieve a suspension. This was then filtered and washed with a small amount of water and dried under vacuum overnight. The isolated solid (9.7 g) was dissolved in EtOH (97 mL) and stirred and heated for 30 min and then allowed to cool for 1 h. The solid was filtered and washed with EtOH and then dried under vacuum to afford 4-chloro-6-methyl-2-oxo-1,2-dihydropyridine-3-carbonitrile (4.37 g, ca. 70% purity) as a tan solid. LC-MS (ES) m/z=168.9 [M+H]⁺.

### (c) 4-(allyloxy)-2-methoxy-6-methylnicotinonitrile

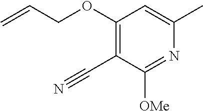

A stirred solution of allyl alcohol (0.223 mL, 3.29 mmol) in DMF (10 mL) was placed into an ice bath and stirred for 15 min. To this was added sodium hydride (60% dispersion in mineral oil, 0.142 g, 3.56 mmol) and the reaction mixture was stirred for 10 min. The ice-bath was removed and the reaction was allowed to warm to room temperature and stirred for about 45 min. The reaction was placed back into the ice-bath and stirred for 10 min, then a solution of 4-chloro-2-methoxy-6-methylnicotinonitrile (0.50 g, 2.74 mmol) in DMF (10 mL) was added to the reaction flask over 2 min and the reaction was allowed to stir for 2 h in the ice-water bath. The reaction was allowed to warm to room temperature and was stirred for 4 days. The reaction was placed back into an ice bath and allyl alcohol (0.25 mL) was added, then the reaction stirred for 15 min, then NaH (60% dispersion in mineral oil, ca. 0.15 g) was added and the reaction was stirred for 60 min in the ice-bath. The reaction mixture was added to a mixture of ice and saturated aqueous ammonium chloride solution and aqueous HCl (1 M, 2-3 mL) and the mixture was stirred for 30 min. The mixture was extracted with 75% Et₂O/EtOAc (2×80 mL) and the combined organics were dried over MgSO₄ and filtered and concentrated in vacuo to a dark residue which was purified via flash chromatography (24 gram silica column and a gradient of B: 5-65%; A: heptanes, B: EtOAc, collected all fractions on UV 254 nm) to afford 4-(allyloxy)-2-methoxy-6-methylnicotinonitrile (160 mg, 0.768 mmol, 28.0% yield) as a white solid. LC-MS (ES) m/z=204.9 [M+H]⁺.

### (d) (4-(allyloxy)-2-methoxy-6-methylpyridin-3-yl)methanamine

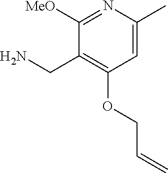

A solution of 4-(allyloxy)-2-methoxy-6-methylnicotinonitrile (150 mg, 0.734 mmol) in Et₂O (15 mL) was stirred for 10 min at room temperature then placed into an ice-bath and stirred for 15 min. LiAlH₄ (1.0 M in Et₂O, 1.50 mL, 1.50 mmol) was added dropwise, and the reaction was stirred in the ice-bath for 1 h then allowed to warm to room temperature and stirred for 2 h. The reaction was placed into the ice-bath for 5 min then quenched with the following: 0.057 mL of water added slowly, 0.057 mL of 15% NaOH, then 0.17 mL of water. The ice-bath was removed and the reaction was allowed to warm to room temperature and was stirred for 60 min, then THF (20 mL) was added. The suspension was filtered through a small Celite® pad, then rinsed with THF, EtOAc, and Et₂O. The filtrate was concentrated in vacuo to afford (4-(allyloxy)-2-methoxy-6-methylpyridin-3-yl)methanamine (140 mg, 0.672 mmol, 92% yield) as a green residue. LC-MS (ES) m/z=209.0 [M+H]⁺ (minor), 191.9 (major).

### (e) 2-allyl-N-((4-(allyloxy)-2-methoxy-6-methylpyridin-3-yl)methyl)-3-(ethyl(tetrahydro-2H-pyran-4-yl)amino)benzamide

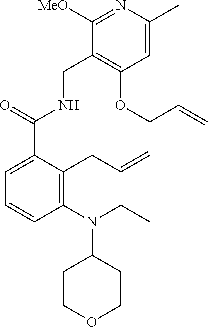 [SMILES: C=CCOc1cc(C)nc(OC)c1CNC(=O)c1cccc(N(CC)C2CCOCC2)c1CC=C]

A reaction vessel was charged with 2-allyl-3-(ethyl(tetrahydro-2H-pyran-4-yl)amino)benzoic acid (250 mg, 0.389 mmol), HOAt (63.5 mg, 0.467 mmol), and EDC (89 mg, 0.467 mmol), then a solution of (4-(allyloxy)-2-methoxy-6-methylpyridin-3-yl)methanamine (108 mg, 0.467 mmol) in DMF (4 mL) was added followed by N-methylmorpholine (0.171 mL, 1.555 mmol). The resulting mixture was stirred at room temperature for about 3 h. The reaction mixture was diluted in water (50 mL) and stirred for 10 min then extracted with DCM (2×50 mL). The organics were combined and dried over MgSO₄, then filtered and concentrated in vacuo to a residue. The residue was dissolved in DCM and adsorbed onto silica gel then purified via flash chromatography (4 gram silica column and a gradient of B: 10-85%; A: heptanes, B: 3:1 EtOAc:EtOH+1% NH₄OH, collected all fractions on UV 254 nm) to afford 2-allyl-N-((4-(allyloxy)-2-methoxy-6-methylpyridin-3-yl)methyl)-3-(ethyl(tetrahydro-2H-pyran-4-yl)amino)benzamide (142 mg, 0.296 mmol, 76% yield) as a residue. LC-MS (ES) m/z=480.3 [M+H]⁺ (minor), 396.2 (minor), 240.7 (major).

### (f) (Z)-10-(ethyl(tetrahydro-2H-pyran-4-yl)amino)-1-methoxy-3-methyl-15,16-dihydro-6H-benzo[g]pyrido[4,3-b][1,5]oxaazacyclododecin-14(9H)-one

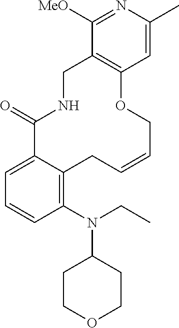 [SMILES: CCN(c1cccc2c1C/C=C\COc1cc(C)nc(OC)c1CNC2=O)C1CCOCC1]

A solution of 2-allyl-N-((4-(allyloxy)-2-methoxy-6-methylpyridin-3-yl)methyl)-3-(ethyl(tetrahydro-2H-pyran-4-yl)amino)benzamide (140 mg, 0.292 mmol) in DCM (15 mL) was degassed with a slow steam of nitrogen for 15 min, then Grubbs II (24.78 mg, 0.029 mmol) was added. The reaction vessel was capped tightly with a septum and covered away from light and the reaction mixture was stirred well at room temperature overnight (20 h). Nitrogen was bubbled through the reaction mixture for 10 min, then additional Grubbs II (24.78 mg, 0.029 mmol) was added and the reaction mixture was stirred for 48 h. Nitrogen was bubbled through the reaction mixture for 10 min, then additional Grubbs II (10 mg) was added and the reaction mixture was stirred for over the weekend. The reaction mixture was diluted with more DCM and adsorbed onto silica, then purified via flash chromatography (12 gram silica column and a gradient of B: 5-65% A: heptane. B: 3 to 1 EtOAc to EtOH, collected all fractions on UV 254 nm) to afford 10-(ethyl(tetrahydro-2H-pyran-4-yl)amino)-1-methoxy-3-methyl-15,16-dihydro-6H-benzo[g]pyrido[4,3-b][1,5]oxaazacyclododecin-14(9H)-one (25 mg, 0.055 mmol, 19% yield) as a solid. LC-MS (ES) m/z=452.3 [M+H]⁺.

### (g) (Z)-10-(ethyl(tetrahydro-2H-pyran-4-yl)amino)-3-methyl-6,9,15,16-tetrahydro-1H-benzo[g]pyrido[4,3-b][1,5]oxaazacyclododecine-1,14(2H)-dione

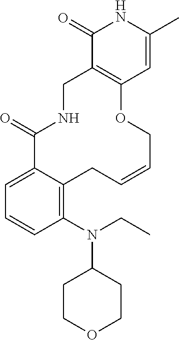 [SMILES: CCN(c1cccc2c1C/C=C\COc1cc(C)[nH]c(=O)c1CNC2=O)C1CCOCC1]

(Z)-10-(Ethyl(tetrahydro-2H-pyran-4-yl)amino)-1-methoxy-3-methyl-15,16-dihydro-6H-benzo[g]pyrido[4,3-b][1,5]oxaazacyclododecin-14(9H)-one was dissolved into HCl (4 M in dioxane, 0.997 mL, 3.99 mmol) and MeOH (0.20 mL) and heated to 60° C. for 22 h. The reaction was then diluted with EtOAc and concentrated via nitrogen stream to a residue, which was dissolved in DCM/MeOH with one drop of concentrated NH₄OH, adsorbed onto silica gel and purified by flash chromatography (4 gram silica column and a gradient of B: 8-100%; A: DCM, B: 90/10/1 of CH₂Cl₂/MeOH/NH₄OH) to afford a residue which was treated with methyl t-butyl ether and then concentrated to afford (Z)-10-(ethyl(tetrahydro-2H-pyran-4-yl)amino)-3-methyl-6,9,15,16-tetrahydro-1H-benzo[g]pyrido[4,3-b][1,5]oxaazacyclododecine-1,14(2H)-dione (9 mg, 0.020 mmol, 45.5% yield) as a solid. LC-MS (ES) m/z=438.3 [M+H]⁺. ¹H NMR (400 MHz, DMSO-d₆) δ: 11.31 (s, 1H), 7.87 (t, J=5.3 Hz, 1H), 7.12-7.27 (m, 2H), 6.94 (dd, J=7.2, 1.4 Hz, 1H), 6.15 (s, 1H), 5.54-5.72 (m, 2H), 4.60 (d, J=6.6 Hz, 2H), 4.25 (d, J=5.1 Hz, 2H), 3.83 (d, J=11.1 Hz, 2H), 3.74 (d, J=5.3 Hz, 2H), 3.23 (t, J=11.0 Hz, 2H), 2.90-3.10 (m, 3H), 2.15 (s, 3H), 1.61 (br. s., 2H), 1.37-1.55 (m, 2H), 0.82 (t, J=6.9 Hz, 3H).

### Example 14

### (E)-12-(ethyl(tetrahydro-2H-pyran-4-yl)amino)-3-methyl-5,6,7,8,17,18-hexahydrobenzo[c]pyrido[4,3-l][1]azacyclotetradecine-1,16(2H,11H)-dione hydrochloride

### (a) 2-allyl-3-(ethyl(tetrahydro-2H-pyran-4-yl)amino)-N-((4-(hex-5-en-1-yl)-2-methoxy-6-methylpyridin-3-yl)methyl)benzamide

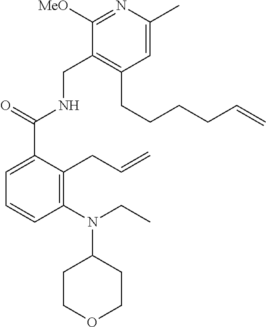 [SMILES: C=CCCCCc1cc(C)nc(OC)c1CNC(=O)c1cccc(N(CC)C2CCOCC2)c1CC=C]

To a mixture of 2-allyl-3-(ethyl(tetrahydro-2H-pyran-4-yl)amino)benzoic acid (235 mg, 0.812 mmol), HOAt (133 mg, 0.975 mmol), EDC (187 mg, 0.975 mmol), and (4-(hex-5-en-1-yl)-2-methoxy-6-methylpyridin-3-yl)methanamine (219 mg, 0.934 mmol) in DMF (6 mL) was added N-methylmorpholine (0.357 mL, 3.25 mmol), and the reaction mixture was stirred at room temperature for about 20 h. The reaction was slowly diluted into ice-water (about 50 mL) with stirring forming a thin slurry. The mixture was extracted with EtOAc and Et₂O (1:1, 50 mL, 2×), and the combined organics were washed with brine and dried over MgSO₄, filtered and the filtrate was adsorbed onto silica and purified by flash chromatography (12 gram column, gradient B; 8-100% A: heptanes, B: 3:1 EtOAc:EtOH+1% NH₄OH, collected all fractions on UV290 nm) to afford 2-allyl-3-(ethyl(tetrahydro-2H-pyran-4-yl)amino)-N-((4-(hex-5-en-1-yl)-2-methoxy-6-methylpyridin-3-yl)methyl)benzamide (205 mg, 0.385 mmol, 47.4% yield) as a residue. LC-MS (ES) m/z=506.4 [M+H]⁺ (minor), 422.3 (minor), 253.8 (major).

### (b) 12-(ethyl(tetrahydro-2H-pyran-4-yl)amino)-1-methoxy-3-methyl-5,6,7,8,17,18-hexahydrobenzo[c]pyrido[4,3-l][1]azacyclotetradecin-16(11H)-one

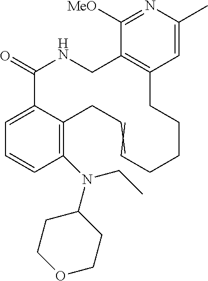 [SMILES: CCN(c1cccc2c1CC=CCCCCc1cc(C)nc(OC)c1CNC2=O)C1CCOCC1]

A solution of 2-allyl-3-(ethyl(tetrahydro-2H-pyran-4-yl)amino)-N-((4-(hex-5-en-1-yl)-2-methoxy-6-methylpyridin-3-yl)methyl)benzamide (200 mg, 0.395 mmol) in DCM (25 mL) was stirred then degassed with a steam of argon for 10 min then Grubbs II (33.6 mg, 0.040 mmol) was added and the reaction was stirred at room temperature overnight. Additional Grubbs II (spatula tip, ca. 10 mg) was added and the reaction was allowed to stir for 6 more h. The reaction mixture was adsorbed onto silica and purified by flash chromatography (12 gram silica column, gradient B 8-80%; A: heptanes, B: EtOAc, collected all fractions on UV254 nm) to afford 12-(ethyl(tetrahydro-2H-pyran-4-yl)amino)-1-methoxy-3-methyl-5,6,7,8,17,18-hexahydrobenzo[c]pyrido[4,3-l][1]azacyclotetradecin-16(11H)-one (138 mg, 0.289 mmol, 73.1% yield) as a residue. LC-MS (ES) m/z=478 [M+H]⁺ (minor), 394.3 (minor), 239.7 (major).

### (c) (E)-12-(ethyl(tetrahydro-2H-pyran-4-yl)amino)-3-methyl-5,6,7,8,17,18-hexahydrobenzo[c]pyrido[4,3-l][1]azacyclotetradecine-1,16(2H,11H)-dione hydrochloride

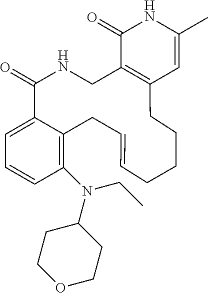 [SMILES: CCN(c1cccc2c1C/C=C/CCCCc1cc(C)[nH]c(=O)c1CNC2=O)C1CCOCC1]

To a solution of (E)-12-(ethyl(tetrahydro-2H-pyran-4-yl)amino)-1-methoxy-3-methyl-5,6,7,8,17,18-hexahydrobenzo[c]pyrido[4,3-l][1]azacyclotetradecin-16(11H)-one (138 mg, 0.3 mmol) in dioxane (4 mL) and MeOH (1 mL) was added HCl (4 M in dioxane, 2 mL, 8 mmol). The reaction solution was stirred at 70° C. for 18 h. The reaction solution was concentrated under vacuum to afford a light brown oil. The oil was purified by flash chromatography (0-100% EtOAc in DCM to 0-100% MeOH in DCM) and the product fractions were concentrated to afford a residue was dissolved in DMF, heated and a few drops of water were added. The mixture was stirred at room temperature for 1 h, then filtered and dried under high vacuum to afford (E)-12-(ethyl(tetrahydro-2H-pyran-4-yl)amino)-3-methyl-5,6,7,8,17,18-hexahydrobenzo[c]pyrido[4,3-l][1]azacyclotetradecine-1,16(2H,11H)-dione hydrochloride (66 mg, 48%) as a light beige solid. LC-MS (ES) m/z=464.3 [M+H]⁺. ¹H NMR (600 MHz, DMSO-d₆) δ: 11.43 (br. s., 1H), 7.77 (br. s., 1H), 7.26 (d, J=7.9 Hz, 1H), 7.20 (t, J=7.6 Hz, 1H), 7.05 (d, J=7.18 Hz, 1H), 5.81 (br. s., 1H), 5.21-5.33 (m, 1H), 5.03-5.18 (m, 1H), 4.11 (br. s., 2H), 3.82 (br. s., 4H), 3.18 (t, J=11.3 Hz, 2H), 2.99 (d, J=6.4 Hz, 2H), 2.94 (t, J=10.6 Hz, 1H), 2.38 (t, J=7.2 Hz, 2H), 2.10 (s, 3H), 1.93 (br. s., 2H), 1.59 (br. s., 2H), 1.43 (d, J=11.3 Hz, 4H), 1.34 (br. s., 2H), 0.76 (t, J=6.8 Hz, 3H).

### Example 15

### (E)-12-chloro-10-methoxy-3-methyl-5,6,15,16-tetrahydrobenzo[c]pyrido[4,3-j][1]azacyclododecine-1,14(2H,9H)-dione

### (a) (E)-12-chloro-1,10-dimethoxy-3-methyl-5,6,15,16-tetrahydrobenzo[c]pyrido[4,3-j][1]azacyclododecin-14(9H)-one

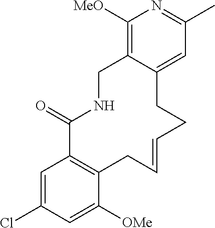 [SMILES: COc1cc(Cl)cc2c1C/C=C/CCc1cc(C)nc(OC)c1CNC2=O]

To a degassed solution of 2-allyl-N-((4-(but-3-en-1-yl)-2-methoxy-6-methylpyridin-3-yl)methyl)-5-chloro-3-methoxybenzamide (845 mg, 2.037 mmol) in DCM (100 mL) was added Grubbs II (173 mg, 0.204 mmol), the reaction mixture was stirred at room temperature overnight under nitrogen. Additional Grubbs II (60 mg) catalyst was added and the reaction was stirred overnight. The reaction mixture was concentrated, and purified by flash chromatography (CombiFlash®, 2×12 g column, 0-20% EtOAc in hexane) to afford a mixture of the E and Z isomers. The mixture was separated by HPLC (0.1% TFA in mobile phase; 35-70% CH₃CN in water) to afford (E)-12-chloro-1,10-dimethoxy-3-methyl-5,6,15,16-tetrahydrobenzo[c]pyrido[4,3-j][1]azacyclododecin-14(9H)-one (256 mg). LC-MS (ES) m/z=387.2 [M+H]⁺.

### (b) (E)-12-chloro-10-methoxy-3-methyl-5,6,15,16-tetrahydrobenzo[c]pyrido[4,3-j][1]azacyclododecine-1,14(2H,9H)-dione

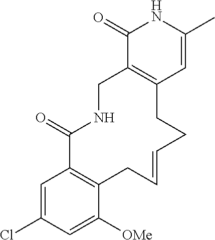 [SMILES: COc1cc(Cl)cc2c1C/C=C/CCc1cc(C)[nH]c(=O)c1CNC2=O]

To a slurry solution of (E)-12-chloro-1,10-dimethoxy-3-methyl-5,6,15,16-tetrahydrobenzo[c]pyrido[4,3-j][1]azacyclododecin-14(9H)-one (90 mg, 0.233 mmol) in dioxane (5 mL) and EtOH (2 mL) was added HCl (4 M in dioxane, 2 mL, 8.00 mmol). The resulting mixture was heated at 70° C. overnight. The reaction mixture was concentrated and the residue was triturated with EtOAc to afford (E)-12-chloro-10-methoxy-3-methyl-5,6,15,16-tetrahydrobenzo[c]pyrido[4,3-j][1]azacyclododecine-1,14(2H,9H)-dione (85 mg, 0.228 mmol, 98% yield) as an off-white solid. ¹H NMR (400 MHz, DMSO-d₆) δ: 11.28 (s, 1H), 8.18 (br. s., 1H), 7.07 (d, J=2.27 Hz, 1H), 6.84 (d, J=2.02 Hz, 1H), 5.84 (s, 1H), 5.03-5.18 (m, 2H), 4.18 (br. s., 2H), 3.79 (s, 3H), 3.33 (br. s., 2H), 2.53 (br. s., 2H), 2.21 (br. s., 2H), 2.12 (s, 3H). LC-MS (ES) m/z=373.2 [M+H]⁺.

### Example 16

### (Z)-12-chloro-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione hydrochloride

### (a) methyl 2-bromo-5-chloro-3-nitrobenzoate

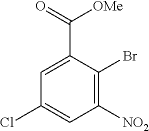 [SMILES: COC(=O)c1cc(Cl)cc([N+](=O)[O-])c1Br]

A solution of methyl 2-bromo-5-chlorobenzoate (30 g, 120 mmol) and H₂SO₄ (96 ml, 1804 mmol) in a 500 mL Erlenmeyer flask was stirred in an ice-bath for 15 min. A solution of nitric acid and H₂SO₄ (20 mL: 20 mL) cooled to 5° C. was added dropwise over 10 min to the well-stirred reaction solution. After 45 min at 5° C., the reaction slurry was slowly poured on to stirred ice-water. After 15 min, the precipitate was filtered and the solids were washed with water and dried under high vacuum to give the product (35 g) as a light yellow solid with 40% of the 6-nitro regioisomer present. LC-MS (ES) m/z=264.0, 266.0 [M+H]⁺.

### (b) methyl 3-amino-2-bromo-5-chlorobenzoate

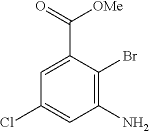 [SMILES: COC(=O)c1cc(Cl)cc(N)c1Br]

Into a 500 mL round bottom flask was added methyl 2-bromo-5-chloro-3-nitrobenzoate (4 g, 13.58 mmol) and ammonium chloride (7.05 g, 132 mmol) in MeOH (130 mL). Water (65 mL) was added and the reaction solution was heated to 70° C., then iron (4.40 g, 79 mmol) was added to the stirred reaction solution. After 3 h at 70° C., the reaction slurry was cooled to room temperature and filtered through a pad of silica gel washing with MeOH. The solution was concentrated under vacuum and the aqueous residue partitioned between EtOAc (100 mL) and saturated aqueous NaHCO₃ solution (50 mL). The phases were separated and the organic phase washed with saturated aqueous NaHCO₃ solution. The organics were dried over MgSO₄, concentrated under vacuum and the residue purified by flash chromatography (hexanes:EtOAc, 4:1) to afford methyl 3-amino-2-bromo-5-chlorobenzoate (1.6 g, contains 12% of a regioisomer) as an orange oil. LC-MS (ES) m/z=263.9, 265.9 [M+H]⁺.

### (c) methyl 2-bromo-5-chloro-3-((tetrahydro-2H-pyran-4-yl)amino)benzoate

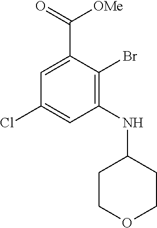 [SMILES: COC(=O)c1cc(Cl)cc(NC2CCOCC2)c1Br]

Into a 500 mL round bottom flask was added dihydro-2H-pyran-4(3H)-one (4.54 g, 45.4 mmol), methyl 3-amino-2-bromo-5-chlorobenzoate (6 g, 22.68 mmol), sodium triacetoxyhydroborate (14.42 g, 68.1 mmol), AcOH (7.79 mL, 136 mmol) and DCE (100 mL). The reaction solution was stirred at room temperature for 20 h. Additional dihydro-2H-pyran-4(3H)-one (2.25 g) and sodium triacetoxyhydroborate (7 g) were added and the reaction was stirred for an additional 8 h at room temperature. The reaction solution was diluted with saturated aqueous NaHCO₃ solution (50 mL). The product was extracted with DCM, dried over Na₂SO₄ and concentrated under vacuum to give an orange oil. The residue was purified by flash chromatography (hexanes:EtOAc, 4:1) to afford methyl 2-bromo-5-chloro-3-((tetrahydro-2H-pyran-4-yl)amino)benzoate (5.3 g, 67%) as a golden colored oil which solidified upon standing. LC-MS (ES) m/z=348.1, 350.1 [M+H]⁺.

### (d) methyl 2-bromo-5-chloro-3-(ethyl(tetrahydro-2H-pyran-4-yl)amino)benzoate

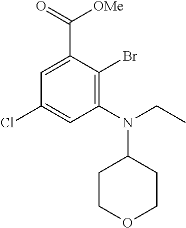 [SMILES: CCN(c1cc(Cl)cc(C(=O)OC)c1Br)C1CCOCC1]

Into a 100 mL round bottom flask was added acetaldehyde (1.744 g, 39.6 mmol), methyl 2-bromo-5-chloro-3-((tetrahydro-2H-pyran-4-yl)amino)benzoate (4.6 g, 13.19 mmol), sodium triacetoxyhydroborate (11.19 g, 52.8 mmol), AcOH (4.53 mL, 79 mmol) and DCE (50 mL). The reaction solution was stirred at room temperature for 24 h. Additional acetaldehyde (1.744 g, 39.6 mmol) and sodium triacetoxyhydroborate (11.19 g, 52.8 mmol) were added to the reaction slurry each day for 6 additional days. After 8 days total reaction time, the reaction solution was diluted with water (100 mL) and then saturated aqueous NaHCO₃ solution (50 mL). The product was extracted with DCM, dried over Na₂SO₄ and concentrated under vacuum to give a brown oil. The oil was purified by flash chromatography (hexanes:EtOAc, 4:1) to afford methyl 2-bromo-5-chloro-3-(ethyl(tetrahydro-2H-pyran-4-yl)amino)benzoate (2.5 g, 50%) as a yellow oil. LC-MS (ES) m/z=376.1, 378.1 [M+H]⁺.

### (e) methyl 2-allyl-5-chloro-3-(ethyl(tetrahydro-2H-pyran-4-yl)amino)benzoate

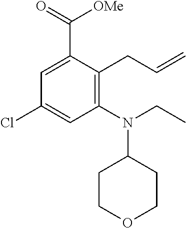 [SMILES: C=CCc1c(C(=O)OC)cc(Cl)cc1N(CC)C1CCOCC1]

Into three 20 mL microwave vials were divided equally methyl 2-bromo-5-chloro-3-(ethyl(tetrahydro-2H-pyran-4-yl)amino)benzoate (1.5 g, 3.98 mmol), allyltributylstannane (1.582 g, 4.78 mmol), copper(I) iodide (0.152 g, 0.796 mmol), K₂CO₃ (1.101 g, 7.96 mmol), Pd(dppf)Cl₂.DCM (0.325 g, 0.398 mmol) and DMF (10 mL). The reaction mixtures were heated separately for 10 h in a microwave reactor. The contents of the three vials were combined and the reaction solution was diluted with saturated aqueous NaHCO₃ solution (50 mL). The mixture was extracted with DCM, dried over Na₂SO₄ and concentrated under vacuum to give a black oil. The residue was purified by flash chromatography (hexanes/EtOAc, 4:1) to afford methyl 2-allyl-5-chloro-3-(ethyl(tetrahydro-2H-pyran-4-yl)amino)benzoate (200 mg, 15%) as a yellow oil. LC-MS (ES) m/z=338.1 [M+H]⁺.

### (f) 2-allyl-5-chloro-3-(ethyl(tetrahydro-2H-pyran-4-yl)amino)benzoic acid

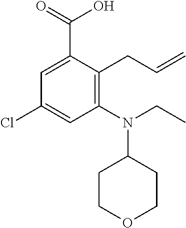 [SMILES: C=CCc1c(C(=O)O)cc(Cl)cc1N(CC)C1CCOCC1]

Into a 50 mL RB flask was added methyl 2-allyl-5-chloro-3-(ethyl(tetrahydro-2H-pyran-4-yl)amino)benzoate (200 mg, 0.592 mmol), and aqueous NaOH (5 M, 1.184 mL, 5.92 mmol) in MeOH (10 mL). The reaction solution was stirred at 50° C. for 20 h. The reaction solution was concentrated under vacuum and the aqueous residue brought to pH=5 with 3 M HCl. The product was extracted with DCM, dried over Na₂SO₄ and concentrated under vacuum to afford 2-allyl-5-chloro-3-(ethyl(tetrahydro-2H-pyran-4-yl)amino)benzoic acid (140 mg, 73%) as a tan solid. LC-MS (ES) m/z=324.2 [M+H]⁺.

### (g) 2-allyl-N-((4-(but-3-en-1-yl)-2-methoxy-6-methylpyridin-3-yl)methyl)-5-chloro-3-(ethyl(tetrahydro-2H-pyran-4-yl)amino)benzamide

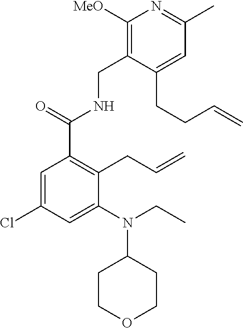 [SMILES: C=CCCc1cc(C)nc(OC)c1CNC(=O)c1cc(Cl)cc(N(CC)C2CCOCC2)c1CC=C]

A mixture of 2-allyl-5-chloro-3-(ethyl(tetrahydro-2H-pyran-4-yl)amino)benzoic acid (130 mg, 0.401 mmol) and (4-(but-3-en-1-yl)-2-methoxy-6-methylpyridin-3-yl)methanamine (99 mg, 0.482 mmol), EDC (115 mg, 0.602 mmol), HOAt (82 mg, 0.602 mmol) and N-methylmorpholine (0.132 mL, 1.204 mmol) in DCM (7 mL) was stirred overnight at room temperature. The reaction mixture was quenched with saturated aqueous Na₂CO₃ solution, and the layers were separated. The aqueous layer was extracted with DCM (2×). The combined organics were washed with water and brine, dried over Na₂SO₄, concentrated and purified by flash chromatography (CombiFlash®, 12 g column, 0-30% EtOAc in hexanes) to afford 2-allyl-N-((4-(but-3-en-1-yl)-2-methoxy-6-methylpyridin-3-yl)methyl)-5-chloro-3-(ethyl(tetrahydro-2H-pyran-4-yl)amino)benzamide (140 mg, 0.273 mmol, 68.1% yield) as a glassy solid. LC (MS) ES m/z=512.5 [M+H]⁺.

### (h) (Z)-12-chloro-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione hydrochloride

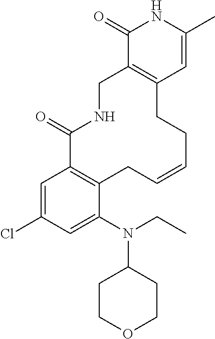 [SMILES: CCN(c1cc(Cl)cc2c1C/C=C\CCc1cc(C)[nH]c(=O)c1CNC2=O)C1CCOCC1]

To a degassed solution of 2-allyl-N-((4-(but-3-en-1-yl)-2-methoxy-6-methylpyridin-3-yl)methyl)-5-chloro-3-(ethyl(tetrahydro-2H-pyran-4-yl)amino)benzamide (140 mg, 0.273 mmol) in DCM (20 mL) was added Grubbs II (34.8 mg, 0.041 mmol), the reaction mixture was stirred at room temperature overnight under nitrogen. The reaction mixture was concentrated, and purified by flash chromatography (CombiFlash®, 2×12 g column, 0-30% EtOAc in hexane) to afford a mixture of E and Z isomers which was further purified by HPLC (0.1% TFA in mobile phases: 25-70% CH₃CN in water, 2^{nd} eluting (minor) peak was collected) to afford 29 mg of a residue. The residue was dissolved in dioxane (1 mL) and MeOH (0.5 mL) was added HCl (4 M in dioxane, 1 mL), stirred at 70° C. overnight. The reaction mixture was concentrated and the residue was triturated with EtOAc to afford (Z)-12-chloro-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione hydrochloride (10 mg, 0.020 mmol, 7.2% yield) as a light brown solid. LC-MS (ES) m/z=470.4 [M+H]⁺. ¹H NMR (400 MHz, DMSO-d₆) δ: 11.46 (br. s., 1H), 8.32 (br. s., 1H), 7.31 (s, 1H), 7.06 (br. s., 1H), 5.96 (s, 1H), 5.17 (br. s., 1H), 5.05 (br. s., 1H), 4.35 (br. s., 2H), 3.55-3.84 (m, 3H), 3.45-3.52 (m, 2H), 3.39 (s, 1H), 3.23 (t, J=11.4 Hz, 2H), 3.00 (br. s., 2H), 2.67 (br. s., 1H), 2.33 (br. s., 2H), 2.13 (s, 3H), 1.60 (br. s., 2H), 1.45 (br. s., 2H), 0.72-0.87 (m, 3H).

### Example 17

### (E)-12-chloro-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione

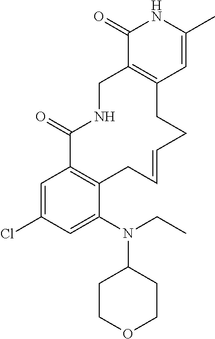 [SMILES: CCN(c1cc(Cl)cc2c1C/C=C/CCc1cc(C)[nH]c(=O)c1CNC2=O)C1CCOCC1]

To a degassed solution of 2-allyl-N-((4-(but-3-en-1-yl)-2-methoxy-6-methylpyridin-3-yl)methyl)-5-chloro-3-(ethyl(tetrahydro-2H-pyran-4-yl)amino)benzamide (140 mg, 0.273 mmol) in DCM (20 mL) was added Grubbs II (34.8 mg, 0.041 mmol), the reaction mixture was stirred at room temperature overnight under nitrogen. The reaction mixture was concentrated, and purified by flash chromatography (CombiFlash®, 2×12 g column, 0-30% EtOAc in hexanes) to afford a mixture of E and Z isomers which was further purified by HPLC (0.1% TFA in mobile phases; 25-70% CH₃CN in water, 1^{st} eluting (major) peak was collected) to afford 102 mg of a residue. The residue was dissolved in dioxane (2 mL) and MeOH (1.5 mL) was added HCl (4 M in dioxane, 1 mL), stirred at 70° C. overnight. The reaction mixture was concentrated and the residue was triturated with EtOAc to afford a light yellow solid. The solid was passed through a 1 g of Silicycle (carbonate) cartridge eluting with MeOH to afford (E)-12-chloro-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione (38 mg, 0.081 mmol, 29.6% yield) as an off-white solid. LC-MS (ES) m/z=470.4 [M+H]⁺. ¹H NMR (600 MHz, DMSO-d₆) δ: 11.30 (br. s., 1H), 8.20 (br. s., 1H), 7.25 (br. s., 1H), 6.99 (s, 1H), 5.83 (s, 1H), 5.06-5.18 (m, 2H), 4.11 (br. s., 2H), 3.80 (d, J=10.2 Hz, 2H), 3.49 (br. s., 2H), 3.18-3.23 (m, 2H), 2.90-3.02 (m, 3H), 2.50 (br. s., 2H), 2.21 (br. s., 2H), 2.11 (s, 3H), 1.57 (br. s., 2H), 1.40 (d, J=10.6 Hz, 2H), 0.75 (t, J=6.4 Hz, 3H).

### Example 18

### (E)-12-chloro-10-isopropoxy-3-methyl-5,6,15,16-tetrahydrobenzo[c]pyrido[4,3-j][1]azacyclododecine-1,14(2H,9H)-dione

### (a) (E)-12-chloro-10-isopropoxy-1-methoxy-3-methyl-5,6,15,16-tetrahydrobenzo[c]pyrido[4,3-j][1]azacyclododecin-14(9H)-one

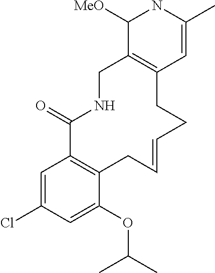 [SMILES: COC1NC(C)=CC2=C1CNC(=O)c1cc(Cl)cc(OC(C)C)c1C/C=C/CC2]

To a solution of (E)-12-chloro-10-hydroxy-1-methoxy-3-methyl-5,6,15,16-tetrahydrobenzo[c]pyrido[4,3-j][1]azacyclododecin-14(9H)-one (100 mg, 0.268 mmol) and 2-iodopropane (0.107 mL, 1.073 mmol, stabilized with copper) in DMF (3 mL) was added Cs₂CO₃ (437 mg, 1.341 mmol). The resulting mixture was stirred at room temperature for 3 h. The reaction mixture was diluted with water, and stirred for 5 min, then the solid was filtered, washed with water and hexane to afford (E)-12-chloro-10-isopropoxy-1-methoxy-3-methyl-5,6,15,16-tetrahydrobenzo[c]pyrido[4,3-j][1]azacyclododecin-14(9H)-one (100 mg, 0.241 mmol, 90% yield) as a white solid. LC-MS (ES) m/z=415.3 [M+H]⁺.

### (b) (E)-12-chloro-10-isopropoxy-3-methyl-5,6,15,16-tetrahydrobenzo[c]pyrido[4,3-j][1]azacyclododecine-1,14(2H,9H)-dione

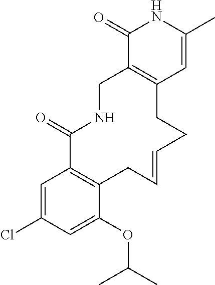 [SMILES: Cc1cc2c(c(=O)[nH]1)CNC(=O)c1cc(Cl)cc(OC(C)C)c1C/C=C/CC2]

To a solution of (E)-12-chloro-10-isopropoxy-1-methoxy-3-methyl-5,6,15,16-tetrahydrobenzo[c]pyrido[4,3-j][1]azacyclododecin-14(9H)-one (100 mg, 0.241 mmol) in dioxane (4 mL) and MeOH (1 mL) was added HCl (4 M in dioxane, 2 mL, 8.00 mmol). The resulting mixture was heated at 70° C. overnight. The reaction mixture was concentrated, and the residue was triturated with EtOAc to afford (E)-12-chloro-10-isopropoxy-3-methyl-5,6,15,16-tetrahydrobenzo[c]pyrido[4,3-j][1]azacyclododecine-1,14(2H,9H)-dione (85 mg, 0.212 mmol, 88% yield) as a white solid. LC-MS (ES) m/z=401.3 [M+H]⁺. ¹H NMR (400 MHz, DMSO-d₆) δ: 11.43 (br. s., 1H), 8.22 (t, J=4.9 Hz, 1H), 7.12 (d, J=2.0 Hz, 1H), 6.83 (d, J=2.0 Hz, 1H), 5.90 (s, 1H), 5.04-5.17 (m, 2H), 4.59-4.65 (m, 1H), 4.07-4.25 (m, 2H), 3.31 (br. s., 2H), 2.52-2.58 (m, 2H), 2.27 (s, 2H), 2.13 (s, 3H), 1.23 (s, 3H), 1.22 (s, 3H).

### Example 19

### (E)-11-(ethyl(tetrahydro-2H-pyran-4-yl)amino)-3-methyl-5,6,7,10,16,17-hexahydro-1H-benzo[c]pyrido[4,3-k][1]azacyclotridecine-1,15(2H)-dione

### (a) 2-methoxy-6-methyl-4-(pent-4-en-1-yl)nicotinonitrile

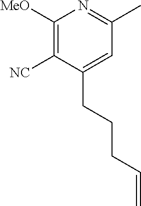 [SMILES: C=CCCCc1cc(C)nc(OC)c1C#N]

To a solution of 2-methoxy-4,6-dimethylnicotinonitrile (2.5 g, 15.41 mmol) in THF (80 mL) was added LHMDS (1 M in THF, 16.18 mL, 16.18 mmol) at 0° C. dropwise via dropping funnel over 10 min, and the reaction mixture turned an orange color. The mixture was stirred at 0° C. for 50 min, then 4-bromobut-1-ene (1.878 mL, 18.50 mmol) was added dropwise via syringe and the mixture was stirred from 0° C. to 10° C. for 2.5 h. The reaction mixture was quenched with saturated aqueous ammonium chloride solution (40 mL) and the layers were separated and the aqueous layer was extracted with EtOAc (3×). The combined organics were concentrated and the residue adsorbed onto silica and purified by flash chromatography (CombiFlash®, 0-5% EtOAc in hexane, 80 g column) to afford 2-methoxy-6-methyl-4-(pent-4-en-1-yl)nicotinonitrile (1.3 g, 6.01 mmol, 39.0% yield) as a colorless oil. LC-MS (ES) m/z=217.0 [M+H]⁺.

### (b) (2-methoxy-6-methyl-4-(pent-4-en-1-yl)pyridin-3-yl)methanamine

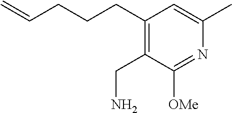 [SMILES: C=CCCCc1cc(C)nc(OC)c1CN]

To a solution of 2-methoxy-6-methyl-4-(pent-4-en-1-yl)nicotinonitrile (1.25 g, 5.78 mmol) in Et₂O (30 mL) at 0° C. was added LiAlH₄ (2 M in THF, 5.78 mL, 11.56 mmol) dropwise. The reaction was allowed to warm to room temperature overnight under nitrogen. The reaction was slowly quenched with water (˜0.7 mL) until all hydrogen production ceased. The solution was then diluted with DCM (30 mL) and stirred for 15 min. The precipitate was filtered through a Celite® pad, and the filtrate was concentrated in-vacuo. The residue was purified by flash chromatography (CombiFlash®, 40 g column, 0-50% 90:9:1 CHCl₃:MeOH:NH₄OH in CHCl₃) to afford (2-methoxy-6-methyl-4-(pent-4-en-1-yl)pyridin-3-yl)methanamine (1.1 g, 4.99 mmol, 86% yield) as a light yellow oil. LC-MS (ES) m/z=204.0 [M+H—NH₃]⁺ (major), 221.1 [M+H]⁺ (minor).

### (c) 2-allyl-3-(ethyl(tetrahydro-2H-pyran-4-yl)amino)-N-((2-methoxy-6-methyl-4-(pent-4-en-1-yl)pyridin-3-yl)methyl)benzamide

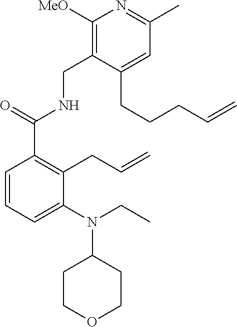 [SMILES: C=CCCCc1cc(C)nc(OC)c1CNC(=O)c1cccc(N(CC)C2CCOCC2)c1CC=C]

A mixture of 2-allyl-3-(ethyl(tetrahydro-2H-pyran-4-yl)amino)benzoic acid (240 mg, 0.829 mmol) and (2-methoxy-6-methyl-4-(pent-4-en-1-yl)pyridin-3-yl)methanamine (183 mg, 0.829 mmol), EDC (238 mg, 1.244 mmol), HOAt (169 mg, 1.244 mmol) and N-methylmorpholine (0.274 mL, 2.488 mmol) in DCM (7 mL) was stirred at room temp overnight. The reaction mixture was quenched with water and the layers were separated. The aqueous layer was extracted with DCM (2×). The combined organics were washed with water and brine, dried over Na₂SO₄, concentrated and purified by flash chromatography (CombiFlash®, 12 g column, 0-20% EtOAc in hexane) to afford 2-allyl-3-(ethyl(tetrahydro-2H-pyran-4-yl)amino)-N-((2-methoxy-6-methyl-4-(pent-4-en-1-yl)pyridin-3-yl)methyl)benzamide (350 mg, 0.712 mmol, 86% yield) as a thick colorless oil. LC-MS (ES) m/z=492.6 [M+H]⁺.

### (d) (E)- and (Z)-11-(ethyl(tetrahydro-2H-pyran-4-yl)amino)-1-methoxy-3-methyl-6,7,16,17-tetrahydro-5H-benzo[c]pyrido[4,3-k][1]azacyclotridecin-15(10H)-one

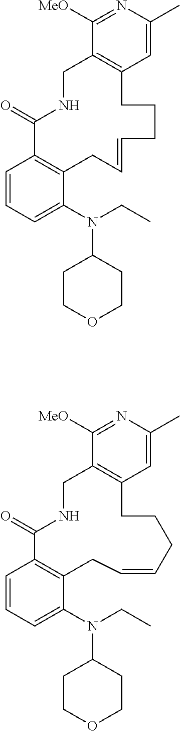 [SMILES: CCN(c1cccc2c1C/C=C/CCCc1cc(C)nc(OC)c1CNC2=O)C1CCOCC1 ; CCN(c1cccc2c1C/C=C\CCCc1cc(C)nc(OC)c1CNC2=O)C1CCOCC1]

To a degassed solution of 2-allyl-3-(ethyl(tetrahydro-2H-pyran-4-yl)amino)-N-((2-methoxy-6-methyl-4-(pent-4-en-1-yl)pyridin-3-yl)methyl)benzamide (350 mg, 0.712 mmol) in DCM (40 mL) was added Grubbs II (91 mg, 0.107 mmol), the reaction mixture was stirred at room temperature overnight under nitrogen. The reaction mixture was concentrated, and purified by flash chromatography (CombiFlash®, 12 g column, 0-30% EtOAc in hexane) to afford a mixture of E and Z isomers (LCMS:93:7) which was triturated with MeOH and filtered to afford (E)-11-(ethyl(tetrahydro-2H-pyran-4-yl)amino)-1-methoxy-3-methyl-6,7,16,17-tetrahydro-5H-benzo[c]pyrido[4,3-k][1]azacyclotridecin-15(10H)-one (190 mg, 0.410 mmol, 57.6% yield) as a white solid. LC-MS (ES) m/z=464.5 [M+H]⁺.

The filtrate was concentrated to afford a mixture of E and Z isomers, weight: 60 mg. The mixture was purified by HPLC (0.1% TFA in mobile phase; 10-60% CH₃CN in water) to afford (Z)-11-(ethyl(tetrahydro-2H-pyran-4-yl)amino)-1-methoxy-3-methyl-6,7,16,17-tetrahydro-5H-benzo[c]pyrido[4,3-k][1]azacyclotridecin-15(10H)-one, trifluoroacetate (37 mg, 0.064 mmol, 9.0% yield) as white solid. LC-MS (ES) m/z=464.5 [M+H]⁺.

### (e) (E)-11-(ethyl(tetrahydro-2H-pyran-4-yl)amino)-3-methyl-5,6,7,10,16,17-hexahydro-1H-benzo[c]pyrido[4,3-k][1]azacyclotridecine-1,15(2H)-dione

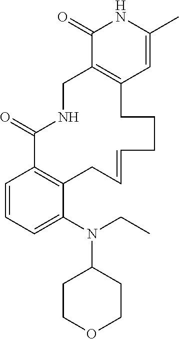 [SMILES: CCN(c1cccc2c1C/C=C/CCCc1cc(C)[nH]c(=O)c1CNC2=O)C1CCOCC1]

To a slurry of (E)-11-(ethyl(tetrahydro-2H-pyran-4-yl)amino)-1-methoxy-3-methyl-6,7,16,17-tetrahydro-5H-benzo[c]pyrido[4,3-k][1]azacyclotridecin-15(10H)-one (110 mg, 0.237 mmol) in dioxane (4 mL) and MeOH (1 mL) was added HCl (4 M in dioxane, 2 mL, 8.00 mmol). The resulting mixture was heated at 70° C. overnight. The reaction mixture was concentrated to dryness and the residue was passed through a 1 g Silicycle (carbonate) cartridge eluting with MeOH (35 mL) to afford (E)-11-(ethyl(tetrahydro-2H-pyran-4-yl)amino)-3-methyl-5,6,7,10,16,17-hexahydro-1H-benzo[c]pyrido[4,3-k][1]azacyclotridecine-1,15(2H)-dione (102 mg, 0.227 mmol, 96% yield) as an off-white solid. LC-MS (ES) m/z=450.5 [M+H]⁺. ¹H NMR (600 MHz, DMSO-d₆) δ: 11.41 (br. s., 1H), 7.95 (br. s., 1H), 7.23-7.29 (m, 1H), 7.20 (t, J=7.7 Hz, 1H), 7.06 (d, J=7.2 Hz, 1H), 5.86 (s, 1H), 5.38 (dt, J=9.91, 5.1 Hz, 1H), 4.94-5.1 (m, 1H), 4.32 (d, J=3.8 Hz, 2H), 3.81 (br. s., 4H), 3.19 (t, J=11.3 Hz, 2H), 2.91-3.04 (m, 3H), 2.25-2.34 (m, 2H), 2.04-2.13 (m, 3H), 1.91 (br. s., 2H), 1.36-1.70 (m, 6H), 0.77 (t, J=6.8 Hz, 3H).

### Example 20

### 11-(ethyl(tetrahydro-2H-pyran-4-yl)amino)-3-methyl-5,6,7,8,9,10,16,17-octahydro-1H-benzo[c]pyrido[4,3-k][1]azacyclotridecine-1,15(2H)-dione

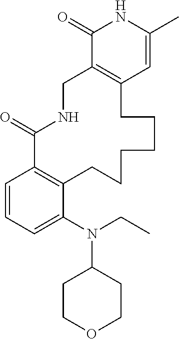 [SMILES: CCN(c1cccc2c1CCCCCCc1cc(C)[nH]c(=O)c1CNC2=O)C1CCOCC1]

A solution of (E)-11-(ethyl(tetrahydro-2H-pyran-4-yl)amino)-3-methyl-5,6,7,10,16,17-hexahydro-1H-benzo[c]pyrido[4,3-k][1]azacyclotridecine-1,15(2H)-dione (31 mg, 0.069 mmol) in EtOAc (2 mL) and MeOH (7 mL) was degassed for 5 min with nitrogen, then Pd/C (10 wt % on active carbon, 10 mg) was added, and the solution was purged with nitrogen for another 5 min then placed under a hydrogen atmosphere (balloon) and stirred for 2 h. The mixture was filtered and the filtrate was concentrated to afford 11-(ethyl(tetrahydro-2H-pyran-4-yl)amino)-3-methyl-5,6,7,8,9,10,16,17-octahydro-1H-benzo[c]pyrido[4,3-k][1]azacyclotridecine-1,15(2H)-dione (31 mg, 0.069 mmol, 100% yield) as a white solid. LC-MS (ES) m/z=452.5 [M+H]⁺. ¹H NMR (400 MHz, DMSO-d₆) δ: 11.42 (br. s., 1H), 8.17 (t, J=4.4 Hz, 1H), 7.10-7.21 (m, 2H), 6.99 (dd, J=1.5, 7.3 Hz, 1H), 5.87 (s, 1H), 4.36 (d, J=4.6 Hz, 2H), 3.77-3.87 (m, 2H), 3.22 (t, J=11.0 Hz, 2H), 2.88-3.05 (m, 3H), 2.78 (d, J=7.8 Hz, 2H), 2.38-2.46 (m, 2H), 2.11 (s, 3H), 1.61 (br. s., 2H), 1.44-1.55 (m, 4H), 1.34-1.43 (m, 6H), 0.78 (t, J=7.0 Hz, 3H).

### Example 21

### (Z)-11-(ethyl(tetrahydro-2H-pyran-4-yl)amino)-3-methyl-5,6,7,10,16,17-hexahydro-1H-benzo[c]pyrido[4,3-k][1]azacyclotridecine-1,15(2H)-dione

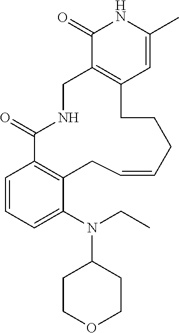 [SMILES: CCN(c1cccc2c1C/C=C\CCCc1cc(C)[nH]c(=O)c1CNC2=O)C1CCOCC1]

To a solution of (Z)-11-(ethyl(tetrahydro-2H-pyran-4-yl)amino)-1-methoxy-3-methyl-6,7,16,17-tetrahydro-5H-benzo[c]pyrido[4,3-k][1]azacyclotridecin-15(10H)-one, trifluoroacetate (37 mg, 0.064 mmol) in dioxane (3 mL) and MeOH (1 mL) was added HCl (4 M in dioxane, 1 mL, 4.00 mmol). The resulting mixture was heated at 70° C. overnight. The reaction mixture was concentrated and the residue was passed through a 1 g of Silicycle (carbonate) cartridge eluting with MeOH (35 mL) to afford (Z)-11-(ethyl(tetrahydro-2H-pyran-4-yl)amino)-3-methyl-5,6,7,10,16,17-hexahydro-1H-benzo[c]pyrido[4,3-k][1]azacyclotridecine-1,15(2H)-dione (23 mg, 0.051 mmol, 80% yield) as an off-white solid. LC-MS (ES) m/z=450.5 [M+H]⁺. ¹H NMR (600 MHz, DMSO-d₆) δ: 11.33 (br. s., 1H), 7.85 (br. s., 1H), 7.23 (d, J=7.2 Hz, 1H), 7.20 (t, J=7.6 Hz, 1H), 7.06 (d, J=7.2 Hz, 1H), 5.92 (s, 1H), 5.21 (br. s., 1H), 5.08-5.18 (m, 1H), 4.13-4.26 (m, 2H), 3.80 (d, J=9.44 Hz, 2H), 3.65-3.69 (m, 2H), 3.22 (t, J=11.3 Hz, 2H), 2.99 (br. s., 3H), 2.53 (br. s., 2H), 2.13 (s, 3H), 2.02 (br. s., 2H), 1.74 (br. s., 2H), 1.60 (br. s., 2H), 1.42 (d, J=9.1 Hz, 2H), 0.77 (t, J=6.8 Hz, 3H).

### Example 22

### (Z)-10-(ethyl(tetrahydro-2H-pyran-4-yl)amino)-3,5-dimethyl-5,6,15,16-tetrahydrobenzo[c]pyrido[4,3-j][1]azacyclododecine-1,14(2H,9H)-dione

### (a) 4-ethyl-2-methoxy-6-methylnicotinonitrile

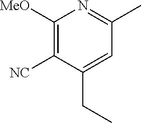

To a solution of 2-methoxy-4,6-dimethylnicotinonitrile (3.5 g, 21.58 mmol) in THF (100 mL) was added LHMDS (1M in THF) (22.66 mL, 22.66 mmol) at 0° C. dropwise via dropping funnel over 10 min, and the reaction was stirred at this temperature for 1 h. Iodomethane (2.418 mL, 22.66 mmol) was added dropwise via syringe and the mixture was stirred from 0° C. to room temperature overnight. The reaction was quenched with saturated aqueous ammonium chloride (60 mL) and the layers were separated. The aqueous layer was extracted with EtOAc (2×). The combined organics were dried over Na₂SO₄, filtered, concentrated, and purified by flash chromatography (0-5% EtOAc in hexanes, 80 g column; re-columned using 0-5% EtOAc in hexanes, 80 g column) to afford 4-ethyl-2-methoxy-6-methylnicotinonitrile (2.01 g, 90% purity) as a white crystalline solid. LC-MS (ES) m/z=176.9 [M+H]⁺.

### (b) 2-methoxy-6-methyl-4-(pent-4-en-2-yl)nicotinonitrile

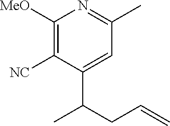 [SMILES: C=CCC(C)c1cc(C)nc(OC)c1C#N]

To a solution of 4-ethyl-2-methoxy-6-methylnicotinonitrile (2.01 g) in THF (50 mL) was added LHMDS (1 M in THF, 11.98 mL, 11.98 mmol) at 0° C. dropwise via syringe over 10 min, and the reaction mixture turned an orange color. The mixture was stirred at 0° C. for 45 min. 3-Bromoprop-1-ene (1.36 mL, 15.72 mmol) was added dropwise via syringe and the mixture was stirred from 0° C. to room temperature overnight. The reaction mixture was quenched with saturated aqueous ammonium chloride (40 mL) and separated the layers; the aqueous layer was extracted with EtOAc (2×). The combined organics were dried over Na₂SO₄, filtered, concentrated, and the residue was purified by flash chromatography (first column: 0-5% EtOAc in hexanes, 80 g column; second column: 0-5% EtOAc in hexane, 40 g column) to afford 2-methoxy-6-methyl-4-(pent-4-en-2-yl)nicotinonitrile (771 mg, 3.56 mmol) as a white solid. LC-MS (ES) m/z=217.0 [M+H]⁺.

### (c) (2-methoxy-6-methyl-4-(pent-4-en-2-yl)pyridin-3-yl)methanamine

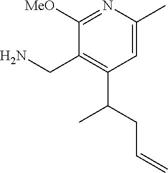 [SMILES: C=CCC(C)c1cc(C)nc(OC)c1CN]

To a solution of 2-methoxy-6-methyl-4-(pent-4-en-2-yl)nicotinonitrile (770 mg, 3.56 mmol) in Et₂O (20 mL) cooled to 0° C. was added LiAlH₄ (2 M in THF, 3.56 mL, 7.12 mmol) dropwise. The reaction allowed to warm to room temperature overnight. The reaction was slowly quenched with water (˜0.4 mL) until all hydrogen production ceased. The solution was then diluted with DCM (20 mL) and stirred for 15 min. The precipitate was filtered through a Celite® pad, and the filtrate was concentrated in-vacuo. The residue was dissolved in DCM and purified by flash chromatography (CombiFlash®, 40 g column, 0-50% 90:9:1 CHCl₃:MeOH:NH₄OH in CHCl₃) to afford (2-methoxy-6-methyl-4-(pent-4-en-2-yl)pyridin-3-yl)methanamine (613 mg, 2.78 mmol, 78% yield) as a light yellow oil. LC-MS (ES) m/z=204 [M+H—NH₃]⁺ (major), 220 [M+H]⁺ (minor).

### (d) 2-allyl-3-(ethyl(tetrahydro-2H-pyran-4-yl)amino)-N-((2-methoxy-6-methyl-4-(pent-4-en-2-yl)pyridin-3-yl)methyl)benzamide

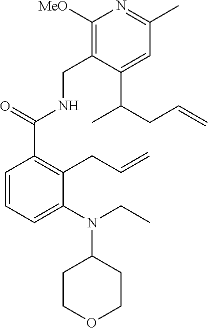 [SMILES: C=CCc1c(C(=O)NCc2c(C(C)CC=C)cc(C)nc2OC)cccc1N(CC)C1CCOCC1]

The reaction mixture of 2-allyl-3-(ethyl(tetrahydro-2H-pyran-4-yl)amino)benzoic acid (200 mg, 0.691 mmol) and (2-methoxy-6-methyl-4-(pent-4-en-2-yl)pyridin-3-yl)methanamine (152 mg, 0.691 mmol), EDC (199 mg, 1.037 mmol), HOAt (141 mg, 1.037 mmol) and N-methylmorpholine (0.228 mL, 2.073 mmol) in DCM (7 mL) was stirred at room temperature for 3 h. The reaction mixture was quenched with water and the layers were separated. The aqueous layer was extracted with DCM. The combined organics were washed with water and brine, dried over Na₂SO₄, concentrated and purified by flash chromatography (CombiFlash®, 12 g column, 0-20% EtOAc in hexane) to afford 2-allyl-3-(ethyl(tetrahydro-2H-pyran-4-yl)amino)-N-((2-methoxy-6-methyl-4-(pent-4-en-2-yl)pyridin-3-yl)methyl)benzamide (246 mg, 0.500 mmol, 72.4% yield) as a colorless thick wax. LC-MS (ES) m/z=492.6 [M+H]⁺.

### (e) (E) and (Z)-10-(ethyl(tetrahydro-2H-pyran-4-yl)amino)-1-methoxy-3,5-dimethyl-5,6,15,16-tetrahydrobenzo[c]pyrido[4,3-j][1]azacyclododecin-14(9H)-one

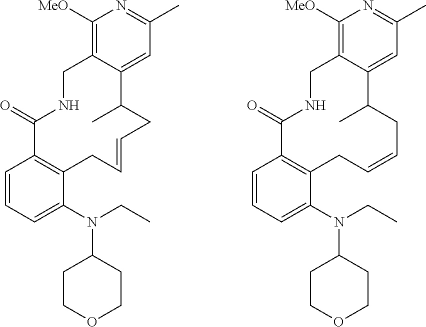 [SMILES: CCN(c1cccc2c1C/C=C/CC(C)c1cc(C)nc(OC)c1CNC2=O)C1CCOCC1 ; CCN(c1cccc2c1C/C=C\CC(C)c1cc(C)nc(OC)c1CNC2=O)C1CCOCC1]

To a degassed solution of 2-allyl-3-(ethyl(tetrahydro-2H-pyran-4-yl)amino)-N-((2-methoxy-6-methyl-4-(pent-4-en-2-yl)pyridin-3-yl)methyl)benzamide (246 mg, 0.500 mmol) in DCM (40 mL) was added Grubbs II (85 mg, 0.100 mmol), the reaction mixture was stirred at room temperature overnight under nitrogen. The reaction mixture was concentrated, and purified by flash chromatography (CombiFlash®, 40 g column, 10-30% EtOAc in hexane) to afford (Z)-10-(ethyl(tetrahydro-2H-pyran-4-yl)amino)-1-methoxy-3,5-dimethyl-5,6,15,16-tetrahydrobenzo[c]pyrido[4,3-j][1]azacyclododecin-14(9H)-one (67 mg) as a white solid. LC-MS (ES) m/z=464.4 [M+H]⁺ (minor), 380.4 (major).

The mixed fractions were concentrated and the mixture was purified by HPLC (0.1% TFA in mobile phase; 10-55% CH₃CN in water) to afford (E)-10-(ethyl(tetrahydro-2H-pyran-4-yl)amino)-1-m ethoxy-3,5-dimethyl-5,6,15,16-tetrahydrobenzo[c]pyrido[4,3-j][1]azacyclododecin-14(9H)-one, trifluoroacetate (135 mg) as a white solid. LC-MS (ES) m/z=464.4 [M+H]⁺.

### (f) (Z)-10-(ethyl(tetrahydro-2H-pyran-4-yl)amino)-3,5-dimethyl-5,6,15,16-tetrahydrobenzo[c]pyrido[4,3-j][1]azacyclododecine-1,14(2H,9H)-dione

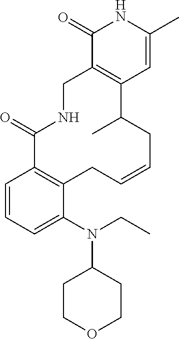 [SMILES: CCN(c1cccc2c1C/C=C\CC(C)c1cc(C)[nH]c(=O)c1CNC2=O)C1CCOCC1]

To a solution of (Z)-10-(ethyl(tetrahydro-2H-pyran-4-yl)amino)-1-methoxy-3,5-dimethyl-5,6,15,16-tetrahydrobenzo[c]pyrido[4,3-j][1]azacyclododecin-14(9H)-one (96 mg, 0.207 mmol) in dioxane (3 mL) and MeOH (1 mL) was added HCl (4 M in dioxane, 1 mL, 4.00 mmol). The resulting mixture was heated at 70° C. overnight. The reaction mixture was concentrated and the residue was passed through a 1 g of Silicycle (carbonate) cartridge eluting with MeOH (30 mL) to afford (Z)-10-(ethyl(tetrahydro-2H-pyran-4-yl)amino)-3,5-dimethyl-5,6,15,16-tetrahydrobenzo[c]pyrido[4,3-j][1]azacyclododecine-1,14(2H,9H)-dione (88 mg, 0.196 mmol, 95% yield) as a light beige solid. LC-MS (ES) m/z=450.5 [M+H]⁺. ¹H NMR (600 MHz, CHLOROFORM-d) δ: 11.41 (br. s., 1H), 7.14-7.22 (m, 2H), 7.09 (d, J=4.5 Hz, 1H), 6.04 (s, 1H), 5.83 (d, J=9.1 Hz, 1H), 5.07 (br. s., 1H), 4.96 (br. s., 1H), 4.75-4.90 (m, 1H), 4.43 (d, J=13.6 Hz, 1H), 3.89-4.01 (m, 2H), 3.85 (br. s., 1H), 3.57 (br. s., 1H), 3.51 (br. s., 1H), 3.16-3.35 (m, 2H), 2.99-3.09 (m, 2H), 2.95 (t, J=10.8 Hz, 1H), 2.73 (br. s., 1H), 2.31 (s, 3H), 1.96 (br. s., 1H), 1.66 (br. s., 2H), 1.50-1.61 (m, 2H), 1.19-1.34 (m, 3H), 0.84 (t, J=7.0 Hz, 3H).

### Example 23

### (E)-10-(ethyl(tetrahydro-2H-pyran-4-yl)amino)-3,5-dimethyl-5,6,15,16-tetrahydrobenzo[c]pyrido[4,3-j][1]azacyclododecine-1,14(2H,9H)-dione

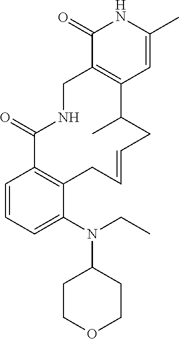 [SMILES: CCN(c1cccc2c1C/C=C/CC(C)c1cc(C)[nH]c(=O)c1CNC2=O)C1CCOCC1]

To a solution of (E)-10-(ethyl(tetrahydro-2H-pyran-4-yl)amino)-1-methoxy-3,5-dimethyl-5,6,15,16-tetrahydrobenzo[c]pyrido[4,3-j][1]azacyclododecin-14(9H)-one, trifluoroacetate (135 mg, 0.234 mmol) in dioxane (3 mL) and MeOH (1 mL) was added HCl (4 M in dioxane, 1 mL, 4.00 mmol). The resulting mixture was heated at 70° C. overnight. The reaction mixture was concentrated and the residue was passed through a 1 g of Silicycle (carbonate) cartridge eluting with MeOH (35 mL) to afford (E)-10-(ethyl(tetrahydro-2H-pyran-4-yl)amino)-3,5-dimethyl-5,6,15,16-tetrahydrobenzo[c]pyrido[4,3-j][1]azacyclododecine-1,14(2H,9H)-dione (73 mg, 0.162 mmol, 69.4% yield) as an off-white solid. LC-MS (ES) m/z=450.5 [M+H]⁺. ¹H NMR (600 MHz, DMSO-d₆) δ: 11.27 (br. s., 1H), 8.20 (d, J=5.3 Hz, 1H), 7.20 (q, J=7.7 Hz, 2H), 6.96 (d, J=6.4 Hz, 1H), 5.96 (s, 1H), 5.07-5.16 (m, 1H), 5.00 (dt, J=14.82, 7.1 Hz, 1H), 4.57 (d, J=13.6 Hz, 1H), 3.84-3.95 (m, 2H), 3.80 (br. s., 2H), 3.11-3.24 (m, 3H), 3.06 (dd, J=14.2, 6.6 Hz, 1H), 2.97 (br. s., 3H), 2.14 (s, 3H), 2.11 (d, J=6.8 Hz, 2H), 1.30-1.80 (m, 4H), 1.08 (d, J=6.8 Hz, 3H), 0.75 (t, J=7.0 Hz, 3H).

### Example 24

### 10-(ethyl(tetrahydro-2H-pyran-4-yl)amino)-3,5-dimethyl-6,7,8,9,15,16-hexahydrobenzo[c]pyrido[4,3-j][1]azacyclododecine-1,14(2H,5H)-dione

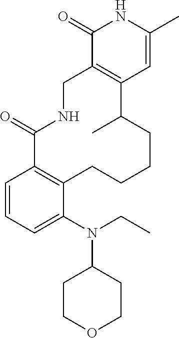 [SMILES: CCN(c1cccc2c1CCCCC(C)c1cc(C)[nH]c(=O)c1CNC2=O)C1CCOCC1]

A solution of (Z)-10-(ethyl(tetrahydro-2H-pyran-4-yl)amino)-3,5-dimethyl-5,6,15,16-tetrahydrobenzo[c]pyrido[4,3-j][1]azacyclododecine-1,14(2H,9H)-dione (35 mg, 0.078 mmol) in MeOH (6 mL) was degassed for 5 min with nitrogen, then Pd/C (10 wt % on active carbon, 10 mg) was added, and the solution was purged with nitrogen for another 5 min then placed under an atmosphere of hydrogen (balloon) and stirred for 10 h. The mixture was filtered and the filtrate was concentrated to afford 10-(ethyl(tetrahydro-2H-pyran-4-yl)amino)-3,5-dimethyl-6,7,8,9,15,16-hexahydrobenzo[c]pyrido[4,3-j][1]azacyclododecine-1,14(2H,5H)-dione (35 mg, 0.078 mmol, 100% yield) as a white solid. LC-MS (ES) m/z=452.5 [M+H]⁺. ¹H NMR (400 MHz, DMSO-d₆) δ: 11.42 (br. s., 1H), 8.39 (d, J=8.8 Hz, 1H), 7.10-7.23 (m, 2H), 7.01 (dd, J=1.8, 7.1 Hz, 1H), 5.94 (s, 1H), 4.61 (dd, J=9.1, 13.6 Hz, 1H), 4.23 (d, J=13.4 Hz, 1H), 3.77-3.85 (m, 2H), 3.13-3.26 (m, 2H), 2.85-3.04 (m, 5H), 2.55-2.70 (m, 1H), 2.10-2.19 (m, 3H), 1.56-1.72 (m, 5H), 1.22-1.50 (m, 5H), 1.01-1.14 (m, 3H), 0.75 (t, J=7.0 Hz, 3H).

### Example 25

### (E)-10-(ethyl(tetrahydro-2H-pyran-4-yl)amino)-6-(hydroxymethyl)-3-methyl-5,6,15,16-tetrahydrobenzo[c]pyrido[4,3-j][1]azacyclododecine-1,14(2H,9H)-dione

### (a) 4-(3-hydroxypent-4-en-1-yl)-2-methoxy-6-methylnicotinonitrile and 4-(2-(hydroxymethyl)but-3-en-1-yl)-2-methoxy-6-methylnicotinonitrile

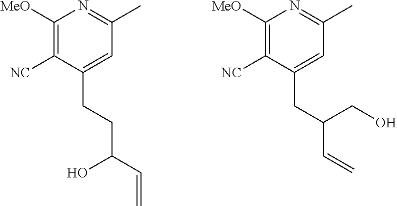 [SMILES: C=CC(O)CCc1cc(C)nc(OC)c1C#N ; C=CC(CO)Cc1cc(C)nc(OC)c1C#N]

To a solution of 2-methoxy-4,6-dimethylnicotinonitrile (2.8 g, 17.26 mmol) in THF (85 mL) was added LHMDS (1 M in THF, 18.13 mL, 18.13 mmol) at 0° C. dropwise via dropping funnel over 10 min, and the reaction mixture turned an orange color. The mixture was stirred at 0° C. for 50 min, 2-vinyloxirane (1.703 mL, 20.72 mmol) was added dropwise via syringe and the mixture was stirred from 0° C. to room temperature for 4 h. The reaction mixture was quenched with saturated aqueous ammonium chloride (40 mL) and the layers were separated, the aqueous layer was extracted with EtOAc (3×). The combined organics were concentrated and the residue was adsorbed onto silica, and purified by flash chromatography (CombiFlash®, 0-40% EtOAc in hexane, 80 g column) to afford 4-(3-hydroxypent-4-en-1-yl)-2-methoxy-6-methylnicotinonitrile (512 mg, 2.204 mmol, 12.8% yield) as a yellow oil. LC-MS (ES) m/z=233.3 [M+H]⁺.

Also isolated was 4-(2-(hydroxymethyl)but-3-en-1-yl)-2-methoxy-6-methylnicotinonitrile (1.08 g, 4.65 mmol, 26.9% yield) as a yellow oil. LC-MS (ES) m/z=233.3 [M+H]⁺.

### (b) 2-((3-(aminomethyl)-2-methoxy-6-methylpyridin-4-yl)methyl)but-3-en-1-ol

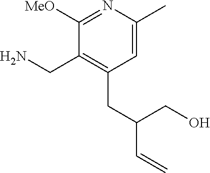 [SMILES: C=CC(CO)Cc1cc(C)nc(OC)c1CN]

To a solution of 4-(2-(hydroxymethyl)but-3-en-1-yl)-2-methoxy-6-methylnicotinonitrile (1.08 g, 4.65 mmol) in Et₂O (23 mL) at 0° C. was added LiAlH₄ (2 M in THF, 4.65 mL, 9.30 mmol) dropwise. The reaction was allowed to warm to room temperature overnight under nitrogen. The reaction was slowly quenched with water (˜0.45 mL) until all hydrogen production ceased. The solution was then diluted with DCM (50 mL) and stirred for 15 min. The precipitate was filtered through a Celite® pad, and the filtrate was concentrated in-vacuo. The residue was purified by flash chromatography (CombiFlash®, 30 g column, 0-100% 90:9:1 CHCl₃:MeOH:NH₄OH in CHCl₃) to afford 2-((3-(aminomethyl)-2-methoxy-6-methylpyridin-4-yl)methyl)but-3-en-1-ol (508 mg, 2.150 mmol, 46.2% yield) as a yellow oil. LC-MS (ES) m/z=237.1 [M+H]⁺ (minor), 220.0 [M+H—NH₃]⁺ (major) and 202.0 (minor).

### (c) 2-allyl-3-(ethyl(tetrahydro-2H-pyran-4-yl)amino)-N-((4-(2-(hydroxymethyl)but-3-en-1-yl)-2-methoxy-6-methylpyridin-3-yl)methyl)benzamide

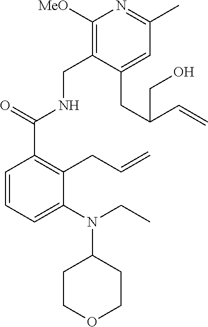 [SMILES: C=CCc1c(C(=O)NCc2c(CC(C=C)CO)cc(C)nc2OC)cccc1N(CC)C1CCOCC1]

The reaction mixture of 2-allyl-3-(ethyl(tetrahydro-2H-pyran-4-yl)amino)benzoic acid (230 mg, 0.795 mmol) and 2-((3-(aminomethyl)-2-methoxy-6-methylpyridin-4-yl)methyl)but-3-en-1-ol (188 mg, 0.795 mmol), EDC (229 mg, 1.192 mmol), HOAt (162 mg, 1.192 mmol) and N-methylmorpholine (0.262 mL, 2.384 mmol) in DCM (7 mL) was stirred at room temperature overnight. The reaction mixture was quenched with water and the layers were separated. The aqueous layer was extracted with DCM. The combined organics were washed with water and brine, dried over Na₂SO₄, concentrated and the residue purified by flash chromatography (CombiFlash®, 12 g column, 0-50% EtOAc in hexane) to afford 2-allyl-3-(ethyl(tetrahydro-2H-pyran-4-yl)amino)-N-((4-(2-(hydroxymethyl)but-3-en-1-yl)-2-methoxy-6-methylpyridin-3-yl)methyl)benzamide (271 mg, 0.534 mmol, 67.2% yield) as a white foam solid. LC-MS (ES) m/z=508.5 [M+H]⁺.

### (d) (E)-10-(ethyl(tetrahydro-2H-pyran-4-yl)amino)-6-(hydroxymethyl)-1-methoxy-3-methyl-5,6,15,16-tetrahydrobenzo[c]pyrido[4,3-j][1]azacyclododecin-14(9H)-one trifluoroacetate

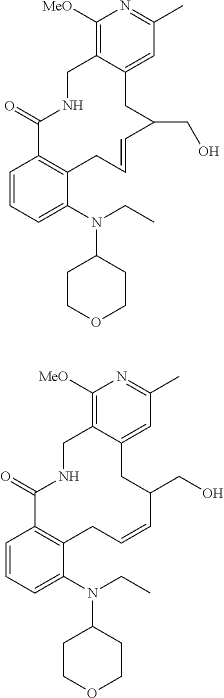 [SMILES: CCN(c1cccc2c1C/C=C/C(CO)Cc1cc(C)nc(OC)c1CNC2=O)C1CCOCC1 ; CCN(c1cccc2c1C/C=C\C(CO)Cc1cc(C)nc(OC)c1CNC2=O)C1CCOCC1]

To a degassed solution of 2-allyl-3-(ethyl(tetrahydro-2H-pyran-4-yl)amino)-N-((4-(2-(hydroxymethyl)but-3-en-1-yl)-2-methoxy-6-methylpyridin-3-yl)methyl)benzamide (270 mg, 0.532 mmol) in DCM (40 mL) was added Grubbs II (90 mg, 0.106 mmol), the reaction mixture was stirred at room temperature overnight under nitrogen. The reaction mixture was concentrated, and purified by flash chromatography (CombiFlash®, 12 g column, 20-70% EtOAc in hexane) to afford a mixture of E and Z isomers. The mixture was further purified by HPLC (0.1% TFA in mobile phase; 5-35% CH₃CN in water) to afford (E)-10-(ethyl(tetrahydro-2H-pyran-4-yl)amino)-6-(hydroxymethyl)-1-methoxy-3-methyl-5,6,15,16-tetrahydrobenzo[c]pyrido[4,3-j][1]azacyclododecin-14(9H)-one, trifluoroacetate (220 mg). LC-MS (ES) m/z=480.4 [M+H]⁺. LC-MS (ES) indicated the presence of a ca. 12% impurity.

Also isolated was (Z)-10-(ethyl(tetrahydro-2H-pyran-4-yl)amino)-6-(hydroxymethyl)-1-methoxy-3-methyl-5,6,15,16-tetrahydrobenzo[c]pyrido[4,3-j][1]azacyclododecin-14(9H)-one trifluoroacetate (72 mg). LC-MS (ES) m/z=480.4 [M+H]⁺. LC-MS (ES) indicated the presence of a ca. 4% impurity.

### (e) (E)-10-(ethyl(tetrahydro-2H-pyran-4-yl)amino)-6-(hydroxymethyl)-3-methyl-5,6,15,16-tetrahydrobenzo[c]pyrido[4,3-j][1]azacyclododecine-1,14(2H,9H)-dione

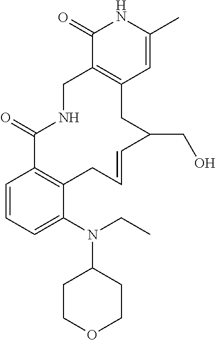 [SMILES: CCN(c1cccc2c1C/C=C/C(CO)Cc1cc(C)[nH]c(=O)c1CNC2=O)C1CCOCC1]

To a solution of (E)-10-(ethyl(tetrahydro-2H-pyran-4-yl)amino)-6-(hydroxymethyl)-1-methoxy-3-methyl-5,6,15,16-tetrahydrobenzo[c]pyrido[4,3-j][1]azacyclododecin-14(9H)-one, trifluoroacetate (220 mg, ca. 88% purity) in dioxane (6 mL) and MeOH (2 mL) was added HCl (4 M, dioxane, 2.5 mL, 10.00 mmol). The resulting mixture was heated at 70° C. overnight. The reaction mixture was concentrated and the residue was passed through a 1 g of Silicycle (carbonate) cartridge eluting with MeOH (35 mL) to afford (E)-10-(ethyl(tetrahydro-2H-pyran-4-yl)amino)-6-(hydroxymethyl)-3-methyl-5,6,15,16-tetrahydrobenzo[c]pyrido[4,3-j][1]azacyclododecine-1,14(2H,9H)-dione (120 mg, 0.258 mmol) as an off-white solid. LC-MS (ES) m/z=466.5 [M+H]⁺. ¹H NMR (600 MHz, DMSO-d6) δ: 11.27 (br. s., 1H), 7.90 (br. s., 1H), 7.21 (d, J=3.8 Hz, 2H), 6.89-7.02 (m, 1H), 5.77 (s, 1H), 5.04-5.14 (m, 1H), 4.92-5.04 (m, 1H), 4.65 (br. s., 1H), 4.36 (br. s., 2H), 3.79 (br. s., 2H), 3.51 (dd, J=15.5, 4.9 Hz, 2H), 3.23-3.30 (m, 2H), 3.12-3.21 (m, 2H), 2.87-3.06 (m, 3H), 2.47 (br. s., 2H), 2.21 (br. s., 1H), 2.04-2.16 (m, 3H), 1.53 (br. s., 2H), 1.32-1.50 (m, 2H), 0.75 (t, J=6.2 Hz, 3H).

### Example 26

### (Z)-10-(ethyl(tetrahydro-2H-pyran-4-yl)amino)-6-(hydroxymethyl)-3-methyl-5,6,15,16-tetrahydrobenzo[c]pyrido[4,3-j][1]azacyclododecine-1,14(2H,9H)-dione

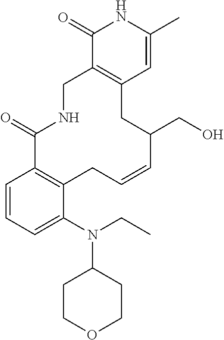 [SMILES: CCN(c1cccc2c1C/C=C\C(CO)Cc1cc(C)[nH]c(=O)c1CNC2=O)C1CCOCC1]

To a solution of (Z)-10-(ethyl(tetrahydro-2H-pyran-4-yl)amino)-6-(hydroxymethyl)-1-methoxy-3-methyl-5,6,15,16-tetrahydrobenzo[c]pyrido[4,3-j][1]azacyclododecin-14(9H)-one, trifluoroacetate (69 mg, ca. 93% purity) in dioxane (3 mL) and MeOH (1 mL) was added HCl (4 M in dioxane, 1.3 mL, 5.20 mmol). The resulting mixture was heated at 70° C. overnight. The reaction mixture was concentrated and the residue was passed through a 500 mg Silicycle (carbonate) cartridge eluting with MeOH (35 mL) to afford (Z)-10-(ethyl(tetrahydro-2H-pyran-4-yl)amino)-6-(hydroxymethyl)-3-methyl-5,6,15,16-tetrahydrobenzo[c]pyrido[4,3-j][1]azacyclododecine-1,14(2H,9H)-dione (43 mg, 0.092 mmol) as an off-white solid. LC-MS (ES) m/z=466.5 [M+H]⁺. ¹H NMR (400 MHz, DMSO-d₆) δ: 11.48 (br. s., 1H), 7.84 (br. s., 1H), 7.15-7.28 (m, 2H), 7.02 (dd, J=1.8, 7.1 Hz, 1H), 5.91 (s, 1H), 5.03-5.20 (m, 2H), 4.73 (br. s., 1H), 4.26 (dd, J=3.5, 13.1 Hz, 1H), 4.15 (dd, J=5.4, 13.3 Hz, 1H), 3.75-3.90 (m, 3H), 3.37-3.46 (m, 2H), 3.16-3.27 (m, 3H), 2.99 (q, J=7.1 Hz, 3H), 2.68 (d, J=1.8 Hz, 1H), 2.59 (dd, J=6.4, 13.0 Hz, 1H), 2.41 (dd, J=3.66, 13.0 Hz, 1H), 2.08-2.15 (m, 3H), 1.60 (br. s., 2H), 1.28-1.50 (m, 2H), 0.76 (t, J=7.0 Hz, 3H).

### Example 27

### 10-(ethyl(tetrahydro-2H-pyran-4-yl)amino)-6-(hydroxymethyl)-3-methyl-6,7,8,9,15,16-hexahydrobenzo[c]pyrido[4,3-j][1]azacyclododecine-1,14(2H,5H)-dione

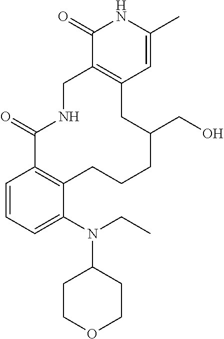 [SMILES: CCN(c1cccc2c1CCCC(CO)Cc1cc(C)[nH]c(=O)c1CNC2=O)C1CCOCC1]

A solution of (E)-10-(ethyl(tetrahydro-2H-pyran-4-yl)amino)-6-(hydroxymethyl)-3-methyl-5,6,15,16-tetrahydrobenzo[c]pyrido[4,3-j][1]azacyclododecine-1,14(2H,9H)-dione (70 mg, 0.150 mmol) in MeOH (7 mL) was degassed for 5 min with nitrogen, then Pd/C (10 wt % on active carbon, 10 mg) was added, and the solution was purged with nitrogen for another 5 min then placed under an atmosphere of hydrogen (balloon) and stirred overnight. The mixture was filtered and concentrated. The residue was re-dissolved in MeOH (8 mL), Pd/C (10 wt % on active carbon, 10 mg) and placed under a hydrogen atmosphere (balloon) overnight. The mixture was filtered and the filtrate was concentrated, and purified by flash chromatography (CombiFlash®, 4 g column, 0-100% (1% NH₄OH+9% MeOH+90% CHCl₃) in CHCl₃) to afford 10-(ethyl(tetrahydro-2H-pyran-4-yl)amino)-6-(hydroxymethyl)-3-methyl-6,7,8,9,15,16-hexahydrobenzo[c]pyrido[4,3-j][1]azacyclododecine-1,14(2H,5H)-dione (23 mg, 0.049 mmol, 32.7% yield) as a white solid. LC-MS (ES) m/z=468.5 [M+H]⁺. ¹H NMR (400 MHz, DMSO-d₆) δ: 11.42 (br. s., 1H), 8.24-8.38 (m, 1H), 7.12-7.24 (m, 2H), 7.04 (dd, J=1.9, 7.0 Hz, 1H), 5.92 (s, 1H), 5.86-5.97 (m, 1H), 4.60 (t, J=5.2 Hz, 1H), 4.49 (dd, J=8.0, 13.8 Hz, 1H), 4.22-4.35 (m, 1H), 3.80 (d, J=4.6 Hz, 2H), 3.05-3.40 (m, 5H), 2.85-3.02 (m, 3H), 2.34 (dd, J=5.2, 14.0 Hz, 1H), 2.13 (s, 3H), 1.93 (br. s., 1H), 1.60 (br. s., 2H), 1.14-1.48 (m, 7H), 0.75 (t, J=7.0 Hz, 3H).

### Example 28

### 11-(ethyl(tetrahydro-2H-pyran-4-yl)amino)-7-hydroxy-3-methyl-5,6,7,8,9,10,16,17-octahydro-1H-benzo[c]pyrido[4,3-k][1]azacyclotridecine-1,15(2H)-dione

### (a) 5-(3-(aminomethyl)-2-methoxy-6-methylpyridin-4-yl)pent-1-en-3-ol

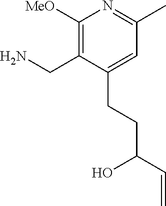 [SMILES: C=CC(O)CCc1cc(C)nc(OC)c1CN]

To a solution of 4-(3-hydroxypent-4-en-1-yl)-2-methoxy-6-methylnicotinonitrile (510 mg, 2.196 mmol) in Et₂O (11 mL) at 0° C. was added LiAlH₄ (2 M in THF, 2.196 mL, 4.39 mmol) dropwise. The reaction was allowed to warm to room temperature overnight under nitrogen. The reaction was slowly quenched with water (˜0.2 mL) until all hydrogen production ceased. The solution was then diluted with DCM (30 mL) and stirred for 15 min. The precipitate was filtered through a Celite® pad, and the filtrate was concentrated in-vacuo. The residue was dissolved in DCM and purified by flash chromatography (CombiFlash®, 15 g column, 0-100% (90:9:1 of CHCl₃:MeOH:NH₄OH) in CHCl₃) to afford 5-(3-(aminomethyl)-2-methoxy-6-methylpyridin-4-yl)pent-1-en-3-ol (342 mg, 1.447 mmol, 65.9% yield) as a light yellow oil. LC-MS (ES) m/z=220 [M+H—NH₃]⁺ (major), 237.1 [M+H]⁺ (minor).

### (b) -allyl-3-(ethyl(tetrahydro-2H-pyran-4-yl)amino)-N-((4-(3-hydroxypent-4-en-1-yl)-2-methoxy-6-methylpyridin-3-yl)methyl)benzamide

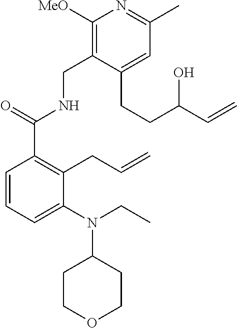 [SMILES: C=CCc1c(C(=O)NCc2c(CCC(O)C=C)cc(C)nc2OC)cccc1N(CC)C1CCOCC1]

The reaction mixture of 2-allyl-3-(ethyl(tetrahydro-2H-pyran-4-yl)amino)benzoic acid (200 mg, 0.691 mmol) and 5-(3-(aminomethyl)-2-methoxy-6-methylpyridin-4-yl)pent-1-en-3-ol (163 mg, 0.691 mmol), EDC (199 mg, 1.037 mmol), HOAt (141 mg, 1.037 mmol) and N-methylmorpholine (0.228 mL, 2.073 mmol) in DCM (6 mL) was stirred at room temperature overnight. The reaction mixture was quenched with water and the layers were separated. The aqueous layer was extracted with DCM (2×). The combined organics were washed with water and brine, dried over Na₂SO₄, concentrated and purified by flash chromatography (CombiFlash®, 12 g column, 0-50% EtOAc in hexane) to afford 2-allyl-3-(ethyl(tetrahydro-2H-pyran-4-yl)amino)-N-((4-(3-hydroxypent-4-en-1-yl)-2-methoxy-6-methylpyridin-3-yl)methyl)benzamide (215 mg, 0.424 mmol, 61.3% yield) as a colorless thick wax. LC-MS (ES) m/z=508.6 [M+H]⁺ (minor), 424.4 (minor), 254.8 (major).

### (c) (E)-11-(ethyl(tetrahydro-2H-pyran-4-yl)amino)-7-hydroxy-1-methoxy-3-methyl-6,7,16,17-tetrahydro-5H-benzo[c]pyrido[4,3-k][1]azacyclotridecin-15(10H)-one

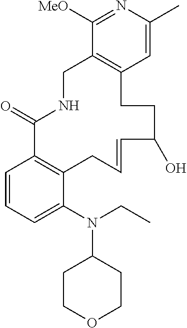 [SMILES: CCN(c1cccc2c1C/C=C/C(O)CCc1cc(C)nc(OC)c1CNC2=O)C1CCOCC1]

To a degassed solution of 2-allyl-3-(ethyl(tetrahydro-2H-pyran-4-yl)amino)-N-((4-(3-hydroxypent-4-en-1-yl)-2-methoxy-6-methylpyridin-3-yl)methyl)benzamide (210 mg, 0.414 mmol) in DCM (35 mL) was added Grubbs II (70.2 mg, 0.083 mmol), the reaction mixture was stirred at room temperature overnight under nitrogen. The reaction mixture was concentrated, and purified by flash chromatography (CombiFlash®, 12 g column, 20-70% EtOAc in hexane) to afford a mixture of isomers. The resulting mixture was further purified by HPLC (0.1% TFA in mobile phases; 10-50% CH₃CN in water), and the product fractions concentrated and the residue was passed through 1 g of Silicycle (carbonate) cartridge eluting with MeOH (45 mL) to afford (E)-11-(ethyl(tetrahydro-2H-pyran-4-yl)amino)-7-hydroxy-1-methoxy-3-methyl-6,7,16,17-tetrahydro-5H-benzo[c]pyrido[4,3-k][1]azacyclotridecin-15(10H)-one (140 mg, 0.292 mmol, 70.6% yield) as a white solid. LC-MS (ES) m/z=480.5 [M+H]⁺ (minor), 396.3 (minor), 240.7 (major).

### (d) 11-(ethyl(tetrahydro-2H-pyran-4-yl)amino)-7-hydroxy-1-methoxy-3-methyl-7,8,9,10,16,17-hexahydro-5H-benzo[c]pyrido[4,3-k][1]azacyclotridecin-15(6H)-one

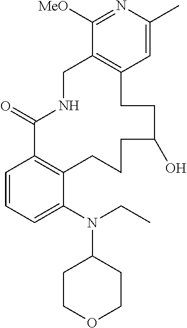 [SMILES: CCN(c1cccc2c1CCCC(O)CCc1cc(C)nc(OC)c1CNC2=O)C1CCOCC1]

A solution of (E)-11-(ethyl(tetrahydro-2H-pyran-4-yl)amino)-7-hydroxy-1-methoxy-3-methyl-6,7,16,17-tetrahydro-5H-benzo[c]pyrido[4,3-k][1]azacyclotridecin-15(10H)-one (80 mg, 0.167 mmol) in MeOH (6 mL) was degassed for 5 min with nitrogen, then Pd/C (10 wt % on active carbon, 15 mg) was added, and the solution was purged with nitrogen for another 5 min then placed under an atmosphere of hydrogen (balloon) and stirred overnight. The mixture was filtered and concentrated to afford 11-(ethyl(tetrahydro-2H-pyran-4-yl)amino)-7-hydroxy-1-methoxy-3-methyl-7,8,9,10,16,17-hexahydro-5H-benzo[c]pyrido[4,3-k][1]azacyclotridecin-15(6H)-one (79 mg, 0.164 mmol, 98% yield) as a white solid. LC-MS (ES) m/z=482.5 [M+H]⁺.

### (e) 11-(ethyl(tetrahydro-2H-pyran-4-yl)amino)-7-hydroxy-3-methyl-5,6,7,8,9,10,16,17-octahydro-1H-benzo[c]pyrido[4,3-k][1]azacyclotridecine-1,15(2H)-dione

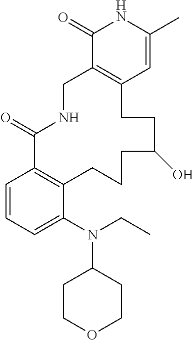 [SMILES: CCN(c1cccc2c1CCCC(O)CCc1cc(C)[nH]c(=O)c1CNC2=O)C1CCOCC1]

To a mixture of 11-(ethyl(tetrahydro-2H-pyran-4-yl)amino)-7-hydroxy-1-methoxy-3-methyl-7,8,9,10,16,17-hexahydro-5H-benzo[c]pyrido[4,3-k][1]azacyclotridecin-15(6H)-one (79 mg, 0.164 mmol) in dioxane (8 mL) was added HCl (4 M in dioxane, 1.5 mL, 6.00 mmol). The resulting mixture was heated at 70° C. for 4 h. The reaction mixture was concentrated and the residue was neutralized with 20% NH₄OH in MeOH, then concentrated and the residue was purified by flash chromatography (CombiFlash®, 4 g column, 0-80% (1% NH₄OH+9% MeOH+90% CHCl₃) in CHCl₃) to afford 11-(ethyl(tetrahydro-2H-pyran-4-yl)amino)-7-hydroxy-3-methyl-5,6,7,8,9,10,16,17-octahydro-1H-benzo[c]pyrido[4,3-k][1]azacyclotridecine-1,15(2H)-dione (15 mg, 0.032 mmol, 19.6% yield) as an off-white solid. LC-MS (ES) m/z=468.5 [M+H]⁺. ¹H NMR (400 MHz, DMSO-d₆) δ: 11.42 (s, 1H), 8.23 (t, J=4.4 Hz, 1H), 7.10-7.22 (m, 2H), 6.98 (dd, J=1.4, 7.2 Hz, 1H), 5.86 (s, 1H), 4.27-4.45 (m, 3H), 3.78-3.88 (m, 2H), 3.59 (d, J=4.0 Hz, 1H), 3.19-3.29 (m, 3H), 2.88-3.06 (m, 3H), 2.71-2.88 (m, 2H), 2.36-2.49 (m, 2H), 2.11 (s, 3H), 1.11-1.77 (m, 9H), 0.78 (t, J=7.0 Hz, 3H).

### Example 29

### (E)-10-(ethyl(tetrahydro-2H-pyran-4-yl)amino)-15-(2-hydroxyethyl)-3-methyl-5,6,15,16-tetrahydrobenzo[c]pyrido[4,3-j][1]azacyclododecine-1,14(2H,9H)-dione

### (a) (E)-15-(2-((tert-butyldimethylsilyl)oxy)ethyl)-10-(ethyl(tetrahydro-2H-pyran-4-yl)amino)-1-methoxy-3-methyl-5,6,15,16-tetrahydrobenzo[c]pyrido[4,3-j][1]azacyclododecin-14(9H)-one

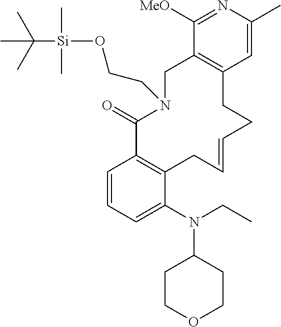 [SMILES: CCN(c1cccc2c1C/C=C/CCc1cc(C)nc(OC)c1CN(CCO[Si](C)(C)C(C)(C)C)C2=O)C1CCOCC1]

To a solution of (E)-10-(ethyl(tetrahydro-2H-pyran-4-yl)amino)-1-methoxy-3-methyl-5,6,15,16-tetrahydrobenzo[c]pyrido[4,3-j][1]azacyclododecin-14(9H)-one (80 mg, 0.178 mmol) in DMF (2 mL) was added NaH (95 wt %, 13.48 mg, 0.534 mmol), after 15 min stirring, tert-butyl(2-iodoethoxy)dimethylsilane (0.083 mL, 0.356 mmol) was added. The resulting mixture was stirred at room temperature for 2.5 h. NaH (95 wt %, 13.48 mg, 0.534 mmol) and tert-butyl(2-iodoethoxy)dimethylsilane (0.083 mL, 0.356 mmol) were added and the reaction was stirred overnight. The reaction mixture was quenched with water and extracted with EtOAc (3×). The combined organics were washed with water (2×) and brine, adsorbed onto silica and purified by flash chromatography (CombiFlash®, 12 g column, 0-30% EtOAc in hexane) to afford (E)-15-(2-((tert-butyldimethylsilyl)oxy)ethyl)-10-(ethyl(tetrahydro-2H-pyran-4-yl)amino)-1-methoxy-3-methyl-5,6,15,16-tetrahydrobenzo[c]pyrido[4,3-j][1]azacyclododecin-14(9H)-one (51 mg, 0.084 mmol, 47.1% yield) as an off white solid. LC-MS (ES) m/z=608.7 [M+H]⁺ (minor), 305.0 (major).

### (b) (E)-10-(ethyl(tetrahydro-2H-pyran-4-yl)amino)-15-(2-hydroxyethyl)-3-methyl-5,6,15,16-tetrahydrobenzo[c]pyrido[4,3-j][1]azacyclododecine-1,14(2H,9H)-dione

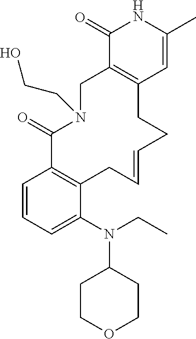 [SMILES: CCN(c1cccc2c1C/C=C/CCc1cc(C)[nH]c(=O)c1CN(CCO)C2=O)C1CCOCC1]

To a solution of (E)-15-(2-((tert-butyldimethylsilyl)oxy)ethyl)-10-(ethyl(tetrahydro-2H-pyran-4-yl)amino)-1-methoxy-3-methyl-5,6,15,16-tetrahydrobenzo[c]pyrido[4,3-j][1]azacyclododecin-14(9H)-one (50 mg, 0.082 mmol) in dioxane (3 mL) and MeOH (1 mL) was added HCl (4 M in dioxane, 1.5 mL, 6.00 mmol). The resulting mixture was heated at 70° C. overnight, then HCl (4 M in dioxane, 1 mL) was added and the reaction was stirred for another 4 h. The reaction mixture was concentrated and the residue was purified by HPLC (0.1% TFA in mobile phase; 5-50% CH₃CN in water) and the product fractions were concentrated and the residue was passed through a 500 mg of Silicycle (carbonate) cartridge eluting with MeOH (30 mL) to afford (E)-10-(ethyl(tetrahydro-2H-pyran-4-yl)amino)-15-(2-hydroxyethyl)-3-methyl-5,6,15,16-tetrahydrobenzo[c]pyrido[4,3-j][1]azacyclododecine-1,14(2H,9H)-dione (16 mg, 0.033 mmol, 40.6% yield) as a white solid. LC-MS (ES) m/z=480.4 [M+H]⁺. ¹H NMR (400 MHz, DMSO-d₆) δ: 7.18-7.30 (m, 2H), 6.93 (t, J=4.4 Hz, 1H), 6.04 (s, 1H), 5.18-5.33 (m, 2H), 5.00-5.11 (m, 1H), 4.39 (br. s., 1H), 4.02-4.11 (m, 1H), 3.78 (br. s., 3H), 3.56 (br. s., 1H), 3.48, (br. s., 1H), 3.12-3.24 (m, 6H), 2.92-3.07 (m, 5H), 2.65-2.79 (m, 1H), 2.39-2.46 (m, 1H), 2.20-2.35 (m, 1H), 2.17 (s, 3H), 1.13-1.44 (m, 3H), 0.75 (br. s., 3H).

### Example 30

### (E)-10-((4,4-difluorocyclohexyl)(ethyl)amino)-3-methyl-5,6,15,16-tetrahydrobenzo[c]pyrido[4,3-j][1]azacyclododecine-1,14(2H,9H)-dione

### (a) methyl 2-bromo-3-((4,4-difluorocyclohexyl)amino)benzoate

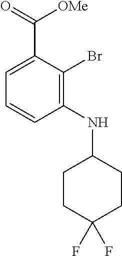 [SMILES: COC(=O)c1cccc(NC2CCC(F)(F)CC2)c1Br]

To a solution of 4,4-difluorocyclohexanone (1.312 g, 9.78 mmol) and methyl 3-amino-2-bromobenzoate (1.5 g, 6.52 mmol) in DCM (30 mL), was added AcOH (0.560 mL, 9.78 mmol), then Na(OAc)₃BH (4.15 g, 19.56 mmol) portionwise. The reaction solution was stirred at room temperature for 2 days. The reaction solution was quenched with saturated aqueous NaHCO₃ solution slowly and the layers were separated, then the aqueous layer was extracted with DCM. The combined organics were adsorbed onto silica, and purified by flash chromatography (CombiFlash®, 40 g column, 0-10% EtOAc in hexane) to afford methyl 2-bromo-3-((4,4-difluorocyclohexyl)amino)benzoate (1.23 g, 3.53 mmol, 54.2% yield) as a colorless oil. LC-MS (ES) m/z=348.1, 350.1 [M+H]⁺.

### (b) methyl 2-bromo-3-((4,4-difluorocyclohexyl)(ethyl)amino)benzoate

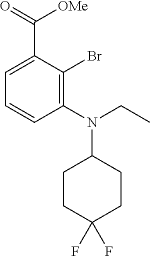 [SMILES: CCN(c1cccc(C(=O)OC)c1Br)C1CCC(F)(F)CC1]

To a solution of acetaldehyde (1.982 mL, 35.3 mmol) and methyl 2-bromo-3-((4,4-difluorocyclohexyl)amino)benzoate (1.23 g, 3.53 mmol) in DCM (20 mL), was added AcOH (0.303 mL, 5.30 mmol), then Na(OAc)₃BH (3.74 g, 17.66 mmol) portionwise, The reaction solution was stirred at room temperature overnight. The reaction was quenched with saturated aqueous NaHCO₃ solution slowly and the layers were separated. The aqueous layer was extracted with DCM (3×). The combined organics were washed with brine and adsorbed onto silica and purified by flash chromatography (CombiFlash®, 40 g column, 0-10% EtOAc in hexane) to afford methyl 2-bromo-3-((4,4-difluorocyclohexyl)(ethyl)amino)benzoate (603 mg, 1.603 mmol, 45.4% yield) as a yellow thick oil. LC-MS (ES) m/z=376.1, 378.1 [M+H]⁺.

### (c) methyl 2-allyl-3-((4,4-difluorocyclohexyl)(ethyl)amino)benzoate

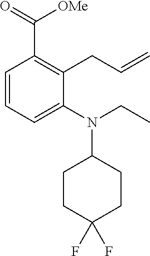 [SMILES: C=CCc1c(C(=O)OC)cccc1N(CC)C1CCC(F)(F)CC1]

A mixture of methyl 2-bromo-3-((4,4-difluorocyclohexyl)(ethyl)amino)benzoate (600 mg, 1.595 mmol), allyltributylstannane (0.895 mL, 2.87 mmol) and lithium chloride (220 mg, 5.18 mmol) in 1,4-dioxane (15 mL) was purged with nitrogen, then PdCl₂(dppf).CH₂Cl₂ adduct (130 mg, 0.159 mmol) was added. The reaction mixture was heated at 90° C. under nitrogen overnight. The reaction mixture was diluted with EtOAc, and added saturated aqueous CsF, stirred for 30 min then filtered through a pad of Celite®. The organic layer was adsorbed onto silica and purified by flash chromatography (CombiFlash®, 2×12 g column, 0-10% EtOAc hexane) to afford methyl 2-allyl-3-((4,4-difluorocyclohexyl)(ethyl)amino)benzoate (298 mg, 0.883 mmol, 55.4% yield) as a light yellow oil. LC-MS (ES) m/z=338.2 [M+H]⁺.

### (d) 2-allyl-3-((4,4-difluorocyclohexyl)(ethyl)amino)benzoic acid

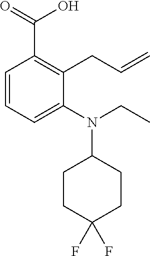 [SMILES: C=CCc1c(C(=O)O)cccc1N(CC)C1CCC(F)(F)CC1]

A mixture of methyl 2-allyl-3-((4,4-difluorocyclohexyl)(ethyl)amino)benzoate (298 mg, 0.883 mmol) and aqueous NaOH (6 M, 1.472 mL, 8.83 mmol) in MeOH (6 mL) was heated at 50° C. under nitrogen overnight. The reaction mixture was concentrated, then diluted with water and washed with hexane. The aqueous layer was acidified with aqueous HCl (6 N, 1.472 mL, 8.83 mmol) to pH 4, extracted with EtOAc (3×). The combined organic extracts were washed with brine and dried over Na₂SO₄, filtered and concentrated to afford 2-allyl-3-((4,4-difluorocyclohexyl)(ethyl)amino)benzoic acid (276 mg, 0.853 mmol, 97% yield) as a colorless wax/oil. LC-MS (ES) m/z=324.2 [M+H]⁺.

### (e) 2-allyl-N-((4-(but-3-en-1-yl)-2-methoxy-6-methylpyridin-3-yl)methyl)-3-((4,4-difluorocyclohexyl)(ethyl)amino)benzamide

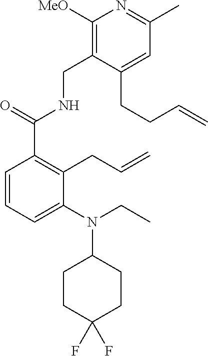 [SMILES: C=CCCc1cc(C)nc(OC)c1CNC(=O)c1cccc(N(CC)C2CCC(F)(F)CC2)c1CC=C]

A mixture of 2-allyl-3-((4,4-difluorocyclohexyl)(ethyl)amino)benzoic acid (274 mg, 0.847 mmol) and (4-(but-3-en-1-yl)-2-methoxy-6-methylpyridin-3-yl)methanamine (184 mg, 0.890 mmol), EDC (211 mg, 1.101 mmol), HOAt (150 mg, 1.101 mmol) and N-methylmorpholine (0.279 mL, 2.54 mmol) in DCM (5 mL) was stirred at room temperature overnight. The reaction mixture was quenched with water and the layers were separated. The aqueous layer was extracted with DCM (2×). The combined organics were washed with water and brine, dried over Na₂SO₄, filtered, then concentrated and purified by flash chromatography (CombiFlash®, 12 g column, using 0-20% EtOAc in hexane) to afford 2-allyl-N-((4-(but-3-en-1-yl)-2-methoxy-6-methylpyridin-3-yl)methyl)-3-((4,4-difluorocyclohexyl)(ethyl)amino)benzamide (328 mg, 0.641 mmol, 76% yield) as a thick colorless oil. LC-MS (ES) m/z=512.6 [M+H]⁺.

### (f) (E)-10-((4,4-difluorocyclohexyl)(ethyl)amino)-1-methoxy-3-methyl-5,6,15,16-tetrahydrobenzo[c]pyrido[4,3-j][1]azacyclododecin-14(9H)-onetrifluoroacetate

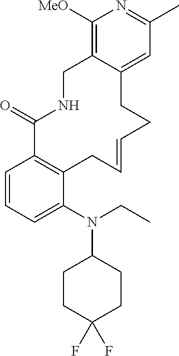 [SMILES: CCN(c1cccc2c1C/C=C/CCc1cc(C)nc(OC)c1CNC2=O)C1CCC(F)(F)CC1]

To a degassed solution of 2-allyl-N-((4-(but-3-en-1-yl)-2-methoxy-6-methylpyridin-3-yl)methyl)-3-((4,4-difluorocyclohexyl)(ethyl)amino)benzamide (328 mg, 0.641 mmol) in DCM (20 mL) was added Grubbs II (82 mg, 0.096 mmol), the reaction mixture was stirred at room temperature overnight under nitrogen. The reaction mixture concentrated and purified by flash chromatography (CombiFlash®, 10 g column, 0-30% EtOAc in hexane) to afford a mixture of E and Z isomers. The resulting mixture was purified by HPLC (0.1% TFA in mobile phase; 15-55% CH₃CN in water) to afford (E)-10-((4,4-difluorocyclohexyl)(ethyl)amino)-1-methoxy-3-methyl-5,6,15,16-tetrahydrobenzo[c]pyrido[4,3-j][1]azacyclododecin-14(9H)-one, trifluoroacetate (238 mg, 62%). LC-MS (ES) m/z=484.5 [M+H]⁺ (minor), 242.7 (major).

### (g) (E)-10-((4,4-difluorocyclohexyl)(ethyl)amino)-3-methyl-5,6,15,16-tetrahydrobenzo[c]pyrido[4,3-j][1]azacyclododecine-1,14(2H,9H)-dione

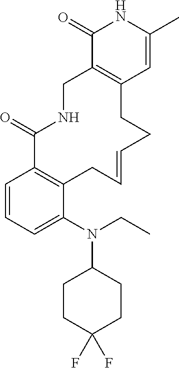 [SMILES: CCN(c1cccc2c1C/C=C/CCc1cc(C)[nH]c(=O)c1CNC2=O)C1CCC(F)(F)CC1]

To a solution of (E)-10-((4,4-difluorocyclohexyl)(ethyl)amino)-1-methoxy-3-methyl-5,6,15,16-tetrahydrobenzo[c]pyrido[4,3-j][1]azacyclododecin-14(9H)-one, trifluoroacetate (165 mg, 0.277 mmol) in dioxane (3 mL) and MeOH (1 mL) was added HCl (4 M, dioxane, 1.5 mL, 6.00 mmol). The resulting mixture was heated at 70° C. overnight. The reaction mixture was concentrated and the residue was passed through a 1 g Silicycle (carbonate) cartridge eluting with MeOH (35 mL). The resulting residue was triturated with EtOAc to afford (E)-10-((4,4-difluorocyclohexyl)(ethyl)amino)-3-methyl-5,6,15,16-tetrahydrobenzo[c]pyrido[4,3-j][1]azacyclododecine-1,14(2H,9H)-dione (92 mg, 0.196 mmol, 70.8% yield) as an off-white solid. LC-MS (ES) m/z=470.5 [M+H]⁺. ¹H NMR (600 MHz, DMSO-d₆) δ: 11.31 (br. s., 1H), 8.00 (br. s., 1H), 7.17-7.28 (m, 2H), 6.97 (d, J=6.8 Hz, 1H), 5.84 (s, 1H), 5.09-5.22 (m, 2H), 4.17 (br. s., 2H), 3.49-3.61 (m, 2H), 3.03 (br. s., 1H), 2.96 (br. s., 2H), 2.52-2.62 (m, 2H), 2.22 (br. s., 2H), 2.11 (s, 3H), 1.88-2.02 (m, 2H), 1.72 (d, J=7.9 Hz, 4H), 1.52 (br. s., 2H), 0.76 (t, J=7.0 Hz, 3H).

### Example 31

### (E)-10-(ethyl(piperidin-4-yl)amino)-3-methyl-5,6,15,16-tetrahydrobenzo[c]pyrido[4,3-j][1]azacyclododecine-1,14(2H,9H)-dione

### (a) tert-butyl 4-((2-bromo-3-(methoxycarbonyl)phenyl)amino)piperidine-1-carboxylate

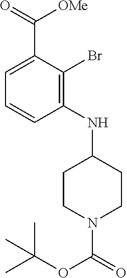 [SMILES: COC(=O)c1cccc(NC2CCN(C(=O)OC(C)(C)C)CC2)c1Br]

To a flask containing tert-butyl 4-oxopiperidine-1-carboxylate (10.59 g, 53.1 mmol) and methyl 3-amino-2-bromobenzoate (4.89 g, 21.26 mmol) in DCM (100 mL), was added AcOH (2.434 mL, 42.5 mmol), then Na(OAc)₃BH (18.02 g, 85 mmol) portionwise, and the reaction solution was stirred at room temperature for 4 days. The reaction solution was quenched with saturated aqueous NaHCO₃ solution slowly and the layers separated. The aqueous layer was extracted with DCM (3×). The combined organics were washed with brine and adsorbed onto silica and purified by flash chromatography (CombiFlash®, 120 g column, 0-20% EtOAc in hexane) to afford tert-butyl 4-((2-bromo-3-(methoxycarbonyl)phenyl)amino)piperidine-1-carboxylate (5.5 g, 13.31 mmol, 62.6% yield) as a yellow waxy solid. LC-MS (ES) m/z=357.1, 359.1 [M+H-tBu]⁺ (major), 413.2 415.2 [M+H]⁺ (minor).

### (b) tert-butyl 4-((2-bromo-3-(methoxycarbonyl)phenyl)(ethyl)amino)piperidine-1-carboxylate

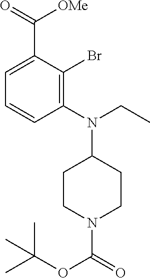 [SMILES: CCN(c1cccc(C(=O)OC)c1Br)C1CCN(C(=O)OC(C)(C)C)CC1]

Into a flask containing acetaldehyde (6.79 mL, 121 mmol) and tert-butyl 4-((2-bromo-3-(methoxycarbonyl)phenyl)amino)piperidine-1-carboxylate (5 g, 12.10 mmol) in DCM (60 mL), was added AcOH (1.039 mL, 18.15 mmol), then Na(OAc)₃BH (12.82 g, 60.5 mmol) portionwise, and the reaction solution was stirred at room temperature overnight. Acetaldehyde (6.79 mL, 121 mmol), was added and the reaction was stirred overnight. The reaction was quenched by the addition of saturated aqueous NaHCO₃ solution slowly and layers were separated. The aqueous layer was extracted with DCM (3×). The combined organics were washed with brine and adsorbed onto silica, and purified by flash chromatography (CombiFlash®, 120 g column, using 0-20% EtOAc in hexane) to afford tert-butyl 4-((2-bromo-3-(methoxycarbonyl)phenyl)(ethyl)amino)piperidine-1-carboxylate (3.08 g, 6.98 mmol, 57.7% yield) as a yellow thick oil. LC-MS (ES) m/z=441.3, 443.3 [M+H]⁺.

### (c) tert-butyl 4-((2-allyl-3-(methoxycarbonyl)phenyl)(ethyl)amino)piperidine-1-carboxylate

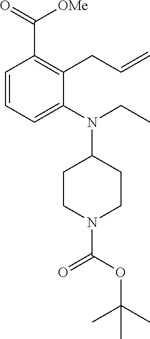 [SMILES: C=CCc1c(C(=O)OC)cccc1N(CC)C1CCN(C(=O)OC(C)(C)C)CC1]

A mixture of tert-butyl 4-((2-bromo-3-(methoxycarbonyl)phenyl)(ethyl)amino)piperidine-1-carboxylate (2.8 g, 6.34 mmol), allyltributylstannane (2.97 mL, 9.52 mmol) and lithium chloride (0.874 g, 20.62 mmol) in 1,4-dioxane (65 mL) was purged with nitrogen, then PdCl₂(dppf).CH₂Cl₂ adduct (0.518 g, 0.634 mmol) was added. The reaction mixture was heated at 90° C. under nitrogen for 3 days. The reaction mixture was diluted with EtOAc, and then saturated aqueous CsF solution was added and the mixture was stirred for 30 min then filtered through a pad of Celite®. The organic layer was separated, then adsorbed onto silica and purified by silica column (CombiFlash®, 80 g column, 0-10% EtOAc in hexane) to afford tert-butyl 4-((2-allyl-3-(methoxycarbonyl)phenyl)(ethyl)amino)piperidine-1-carboxylate (1.79 g, 4.45 mmol, 70.1% yield) as a yellow oil. LC-MS (ES) m/z=403.4 [M+H]⁺.

### (d) 2-allyl-34(1-(tert-butoxycarbonyl)piperidin-4-yl)(ethyl)amino)benzoic acid

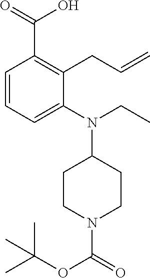 [SMILES: C=CCc1c(C(=O)O)cccc1N(CC)C1CCN(C(=O)OC(C)(C)C)CC1]

A mixture of tert-butyl 4-((2-allyl-3-(methoxycarbonyl)phenyl)(ethyl)amino)piperidine-1-carboxylate (1.79 g, 4.45 mmol) and aqueous NaOH (6 M, 7.41 mL, 44.5 mmol) in MeOH (28 mL) was heated at 50° C. under nitrogen overnight. The reaction mixture was concentrated, then diluted with water and extracted with hexane. The aqueous layer was acidified with aqueous HCl (6 N, 7.41 mL, 44.5 mmol) to pH 4, extracted with EtOAc (3×). The combined organic extracts were washed with brine and dried over Na₂SO₄, filtered and concentrated to afford 2-allyl-3-((1-(tert-butoxycarbonyl)piperidin-4-yl)(ethyl)amino)benzoic acid (1.43 g, 3.68 mmol, 83% yield) as a yellow solid. LC-MS (ES) m/z=389.3 [M+H]⁺.

### (e) tert-butyl 4-((2-allyl-3-(44-(but-3-en-1-yl)-2-methoxy-6-methylpyridin-3-yl)methyl)carbamoyl)phenyl)(ethyl)amino)piperidine-1-carboxylate

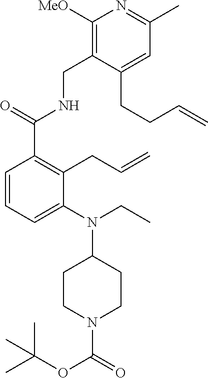 [SMILES: C=CCCc1cc(C)nc(OC)c1CNC(=O)c1cccc(N(CC)C2CCN(C(=O)OC(C)(C)C)CC2)c1CC=C]

A mixture of 2-allyl-3-((1-(tert-butoxycarbonyl)piperidin-4-yl)(ethyl)amino)benzoic acid (540 mg, 1.390 mmol) and (4-(but-3-en-1-yl)-2-methoxy-6-methylpyridin-3-yl)methanamine (301 mg, 1.459 mmol), EDC (346 mg, 1.807 mmol), HOAt (246 mg, 1.807 mmol) and N-methylmorpholine (0.458 mL, 4.17 mmol) in DCM (8 mL) was stirred at room temperature overnight. The reaction mixture was quenched with water and the layers were separated. The aqueous layer was extracted with DCM (2×). The combined organics were washed with water and brine, dried over Na₂SO₄, concentrated and the residue was purified by flash chromatography (CombiFlash®, 12 g column, 0-20% EtOAc in hexane) to afford tert-butyl 4-((2-allyl-3-(((4-(but-3-en-1-yl)-2-methoxy-6-methylpyridin-3-yl)methyl)carbamoyl)phenyl)(ethyl)amino)piperidine-1-carboxylate (564 mg, 0.978 mmol, 70.4% yield) as a thick colorless wax/oil. LC-MS (ES) m/z=577.7 [M+H]⁺.

### (f) (E)-10-(ethyl(piperidin-4-yl)amino)-3-methyl-5,6,15,16-tetrahydrobenzo[c]pyrido[4,3-j][1]azacyclododecine-1,14(2H,9H)-dione

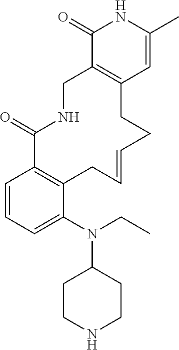 [SMILES: CCN(c1cccc2c1C/C=C/CCc1cc(C)[nH]c(=O)c1CNC2=O)C1CCNCC1]

To a degassed solution of tert-butyl 4-((2-allyl-3-(((4-(but-3-en-1-yl)-2-methoxy-6-methylpyridin-3-yl)methyl)carbamoyl)phenyl)(ethyl)amino)piperidine-1-carboxylate (560 mg, 0.971 mmol) in DCM (40 mL) was added Grubbs II (124 mg, 0.146 mmol), and the reaction mixture was stirred at room temperature for 2 days under nitrogen. The reaction mixture was concentrated, and purified by flash chromatography (CombiFlash®, 10 g column, 0-30% EtOAc in hexane) to afford a mixture of E and Z isomers. The resulting mixture was purified by HPLC (0.1% TFA in mobile phase; 25-55% CH₃CN in water) to afford 567 mg of a residue. The residue was dissolved in 1,4-dioxane (5 mL) and MeOH (4 mL), then HCl (4 M in dioxane, 3 mL, 12.00 mmol) was added and the reaction mixture was heated at 70° C. overnight, forming a white precipitate. The reaction mixture was concentrated and the residue was treated with 30% NH₄OH in MeOH and then concentrated, and this procedure was repeated two more times. The residue was then adsorbed onto silica and purified by flash chromatography (CombiFlash®, 4 g column, 0-100% (1% NH₄OH+9% MeOH+90% CHCl₃) in CHCl₃) to afford (E)-10-(ethyl(piperidin-4-yl)amino)-3-methyl-5,6,15,16-tetrahydrobenzo[c]pyrido[4,3-j][1]azacyclododecine-1,14(2H,9H)-dione (233 mg, 0.536 mmol, 55.2% yield) as a white solid. LC-MS (ES) m/z=435.4 [M+H]⁺. ¹H NMR (600 MHz, DMSO-d₆) δ: 11.27 (br. s., 1H), 8.00 (br. s., 1H), 7.18 (d, J=4.5 Hz, 2H), 6.94 (t, J=4.3 Hz, 1H), 5.83 (s, 1H), 5.06-5.24 (m, 2H), 3.86-4.52 (m, 2H), 3.53 (br. s., 2H), 2.98 (br. s., 2H), 2.87 (d, J=11.3 Hz, 2H), 2.70 (t, J=10.6 Hz, 1H), 2.54 (br. s., 2H), 2.29 (t, J=11.5 Hz, 2H), 2.22 (br. s., 2H), 2.11 (s, 3H), 1.58 (br. s., 2H), 1.21-1.34 (m, 2H), 0.75 (t, J=7.0 Hz, 3H). One H not observed.

### Example 32

### (E)-10-(ethyl(1-isopropylpiperidin-4-yl)amino)-3-methyl-5,6,15,16-tetrahydrobenzo[c]pyrido[4,3-j][1]azacyclododecine-1,14(2H,9H)-dione

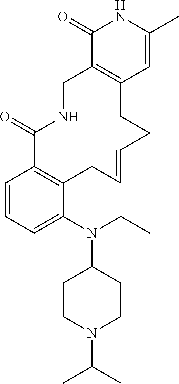 [SMILES: CCN(c1cccc2c1C/C=C/CCc1cc(C)[nH]c(=O)c1CNC2=O)C1CCN(C(C)C)CC1]

To a solution of (E)-10-(ethyl(piperidin-4-yl)amino)-3-methyl-5,6,15,16-tetrahydrobenzo[c]pyrido[4,3-j][1]azacyclododecine-1,14(2H,9H)-dione (100 mg, 0.230 mmol) in MeOH (3 mL) was added NaBH₃CN (72.3 mg, 1.151 mmol), acetone (0.135 mL, 1.841 mmol) and AcOH (0.026 mL, 0.460 mmol). The reaction mixture was stirred at room temperature overnight. NaBH₃CN (72.3 mg, 1.151 mmol), and acetone (0.135 mL, 1.841 mmol) were added and the reaction was stirred for another day. The reaction mixture was adsorbed onto silica and purified by flash chromatography (CombiFlash®, 4 g column, using 0-100% (1% NH₄OH+9% MeOH+90% CHCl₃) in CHCl₃) to afford (E)-10-(ethyl(1-isopropylpiperidin-4-yl)amino)-3-methyl-5,6,15,16-tetrahydrobenzo[c]pyrido[4,3-j][1]azacyclododecine-1,14(2H,9H)-dione (82 mg, 0.172 mmol, 74.8% yield) as a white solid. LC-MS (ES) m/z=477.5 [M+H]⁺ (minor), 239.3 (major). ¹H NMR (400 MHz, METHANOL-d₄) δ: 7.21-7.27 (m, 1H), 7.15-7.21 (m, 1H), 7.03 (dd, J=7.2, 1.4 Hz, 1H), 6.05 (s, 1H), 5.20-5.34 (m, 1H), 5.14 (dt, J=15.2, 5.9 Hz, 1H), 4.31 (br. s., 2H), 3.63 (br. s., 2H), 2.92-3.05 (m, 4H), 2.89 (br. s., 2H), 2.64 (br. s., 2H), 2.36 (d, J=7.6 Hz, 2H), 2.27 (br. s., 2H), 2.19 (s, 3H), 1.67-1.97 (m, 2H), 1.57 (br. s., 2H), 1.07 (d, J=6.6 Hz, 6H), 0.77 (t, J=7.1 Hz, 3H). 2H not observed.

### Example 33

### (E)-10-(ethyl(1-methylpiperidin-4-yl)amino)-3-methyl-5,6,15,16-tetrahydrobenzo[c]pyrido[4,3-j][1]azacyclododecine-1,14(2H,9H)-dione

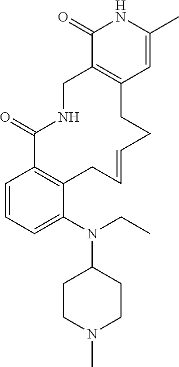 [SMILES: CCN(c1cccc2c1C/C=C/CCc1cc(C)[nH]c(=O)c1CNC2=O)C1CCN(C)CC1]

To a solution of (E)-10-(ethyl(piperidin-4-yl)amino)-1-methoxy-3-methyl-5,6,15,16-tetrahydrobenzo[c]pyrido[4,3-j][1]azacyclododecin-14(9H)-one (100 mg, 0.223 mmol) in MeOH (3 mL) was added NaBH₃CN (70.0 mg, 1.115 mmol), formaldehyde (0.205 mL, 2.229 mmol) and AcOH (0.026 mL, 0.446 mmol). The reaction mixture was stirred at room temperature for 3 h. The reaction mixture was adsorbed onto silica, then purified by flash chromatography (CombiFlash®, 4 g column, 0-100% (1% NH₄OH+9% MeOH+90% CHCl₃) in CHCl₃) to afford 71 mg of a white solid. The solid was dissolved in 1,4-dioxane (3.00 mL) and MeOH (1 mL), then was HCl (4 M in dioxane, 1.5 mL, 6.00 mmol) was added and the reaction mixture was heated at 70° C. overnight. The reaction mixture was concentrated and the residue was passed through a 1 g of Silicycle (carbonate) cartridge eluting with MeOH (30 mL) to afford (E)-10-(ethyl(1-methylpiperidin-4-yl)amino)-3-methyl-5,6,15,16-tetrahydrobenzo[c]pyrido[4,3-j][1]azacyclododecine-1,14(2H,9H)-dione (48 mg, 0.107 mmol, 48.0% yield) as an off-white solid. LC-MS (ES) m/z=449.4 [M+H]⁺. ¹H NMR (400 MHz, METHANOL-d₄) δ: 7.20-7.35 (m, 2H), 7.08 (dd, J=1.5, 7.3 Hz, 1H), 6.12 (s, 1H), 5.29-5.41 (m, 1H), 5.16-5.26 (m, 1H), 4.38 (s, 2H), 3.66-3.73 (m, 2H), 3.06 (q, J=7.1 Hz, 2H), 2.82 (br. s., 3H), 2.72 (br. s., 2H), 2.31 (br. s., 2H), 2.26 (s, 3H), 2.23 (s, 3H), 1.93-2.05 (m, 2H), 1.66-1.88 (m, 2H), 1.51-1.65 (br. s., 2H), 0.83 (t, J=7.1 Hz, 3H). 2H not observed.

### Example 34

### (E)-10-(ethyl(1-(methylsulfonyl)piperidin-4-yl)amino)-3-methyl-5,6,15,16-tetrahydrobenzo[c]pyrido[4,3-j][1]azacyclododecine-1,14(2H,9H)-dione

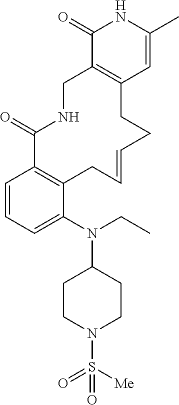 [SMILES: CCN(c1cccc2c1C/C=C/CCc1cc(C)[nH]c(=O)c1CNC2=O)C1CCN(S(C)(=O)=O)CC1]

To a solution of (E)-10-(ethyl(piperidin-4-yl)amino)-1-methoxy-3-methyl-5,6,15,16-tetrahydrobenzo[c]pyrido[4,3-j][1]azacyclododecin-14(9H)-one (100 mg, 0.223 mmol) in DCM (4 mL) was added Et₃N (0.093 mL, 0.669 mmol), and methanesulfonyl chloride (0.026 mL, 0.334 mmol). The reaction mixture was stirred at room temperature for 3 h. The reaction mixture was quenched with 0.1 mL of water, then adsorbed onto silica, purified by flash chromatography (CombiFlash®, 4 g column, using 0-20% (1% NH₄OH+9% MeOH+90% CHCl₃ in CHCl₃) to afford 84 mg of a white solid. The solid was dissolved in 1,4-dioxane (3.00 mL) and MeOH (1 mL), then HCl (4 M in dioxane, 1.5 mL, 6.00 mmol) was added and the reaction mixture was heated at 70° C. overnight. The reaction mixture was concentrated and purified by HPLC (0.1% TFA in mobile phase; 5-40 CH₃CN in water) and product fractions were concentrated and the residue was passed through a 1 g of Silicycle (carbonate) cartridge eluting with MeOH (30 mL) to afford (E)-10-(ethyl(1-(methylsulfonyl)piperidin-4-yl)amino)-3-methyl-5,6,15,16-tetrahydrobenzo[c]pyrido[4,3-j][1]azacyclododecine-1,14(2H,9H)-dione (49 mg, 0.096 mmol, 42.9% yield) as a white solid. LC-MS (ES) m/z=513.4 [M+H]⁺. ¹H NMR (400 MHz, METHANOL-d₄) δ: 7.28-7.35 (m, 1H), 7.22-7.28 (m, 1H), 7.10 (dd, J=1.5, 7.3 Hz, 1H), 6.12 (s, 1H), 5.29-5.40 (m, 1H), 5.17-5.27 (m, 1H), 4.39 (s, 2H), 3.53-3.76 (m, 4H), 3.03-3.13 (m, 2H), 2.92-3.02 (m, 1H), 2.65-2.83 (m, 7H), 2.34 (br. s., 2H), 2.26 (s, 3H), 1.75-2.01 (m, 2H), 1.52-1.68 (m, 2H), 0.84 (t, J=7.1 Hz, 3H). 2H not observed.

### Example 35

### (E)-10-(ethyl(1-(2-hydroxyethyl)piperidin-4-yl)amino)-3-methyl-5,6,15,16-tetrahydrobenzo[c]pyrido[4,3-j][1]azacyclododecine-1,14(2H,9H)-dione

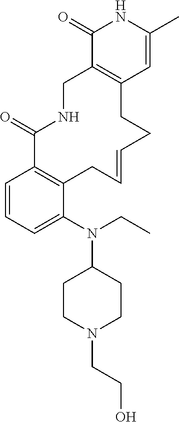 [SMILES: CCN(c1cccc2c1C/C=C/CCc1cc(C)[nH]c(=O)c1CNC2=O)C1CCN(CCO)CC1]

To a solution of (E)-10-(ethyl(piperidin-4-yl)amino)-1-methoxy-3-methyl-5,6,15,16-tetrahydrobenzo[c]pyrido[4,3-j][1]azacyclododecin-14(9H)-one (105 mg, 0.234 mmol) in MeOH (3 mL) was added 2-((tert-butyldimethylsilyl)oxy)acetaldehyde (0.111 mL, 0.585 mmol), AcOH (0.027 mL, 0.468 mmol) and then NaBH₃CN (73.5 mg, 1.170 mmol). The reaction mixture was stirred at room temperature for 3 h. The reaction mixture was adsorbed onto silica, purified by flash chromatography (CombiFlash®, 4 g column, 0-80% (1% NH₄OH+9% MeOH+90% CHCl₃) in CHCl₃) to afford 90 mg of a waxy solid. The solid was dissolved in 1,4-dioxane (3.00 mL) and MeOH (1 mL), then HCl (4 M dioxane, 1.5 mL, 6.00 mmol) was added and the reaction mixture was heated at 70° C. overnight. The reaction mixture was concentrated to dryness and the residue was treated with 30% NH₄OH in MeOH and concentrated (this procedure was repeated twice). The residue was purified by flash chromatography (CombiFlash®, 4 g column, 0-100% (1% NH₄OH+9% MeOH+90% CHCl₃) in CHCl₃) to afford (E)-10-(ethyl(1-(2-hydroxyethyl)piperidin-4-yl)amino)-3-methyl-5,6,15,16-tetrahydrobenzo[c]pyrido[4,3-j][1]azacyclododecine-1,14(2H,9H)-dione (31 mg, 0.065 mmol, 27.7% yield) as an off-white solid. LC-MS (ES) m/z=479.4 [M+H]⁺ (minor), 240.3 (major). ¹H NMR (400 MHz, METHANOL-d₄) δ: 7.20-7.32 (m, 2H), 7.08 (dd, J=1.4, 7.2 Hz, 1H), 6.12 (s, 1H), 5.28-5.41 (m, 1H), 5.16-5.26 (m, 1H), 4.38 (s, 2H), 3.60-3.76 (m, 4H), 3.07 (q, J=7.1 Hz, 2H), 2.89-3.00 (m, 2H), 2.76-2.88 (m, 1H), 2.72 (br. s., 2H), 2.50 (t, J=6.2 Hz, 2H), 2.34 (br. s., 2H), 2.26 (s, 3H), 2.00-2.13 (m, 2H), 1.66-1.89 (m, 2H), 1.52-1.67 (m, 2H), 0.83 (t, J=7.1 Hz, 3H). 3H not observed.

### Example 36

### (E)-10-((trans-4-aminocyclohexyl)(ethyl)amino)-3-methyl-5,6,15,16-tetrahydrobenzo[c]pyrido[4,3-j][1]azacyclododecine-1,14(2H,9H)-dione

### (a) methyl 2-bromo-3-nitrobenzoate

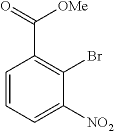 [SMILES: COC(=O)c1cccc([N+](=O)[O-])c1Br]

To a solution of 2-bromo-3-nitrobenzoic acid (33.9 g, 138 mmol) in DMF (240 mL) were added Na₂CO₃ (58.5 g, 552 mmol) and iodomethane (34 mL, 544 mmol). The reaction mixture was heated at 60° C. for 4 h. The reaction mixture was diluted with water (1.5 L) and extracted with Et₂O (2×750 mL). The combined organic layers were washed with brine (500 mL) and concentrated to afford methyl 2-bromo-3-nitrobenzoate (36.78 g, 137 mmol, 100% yield) as a yellow solid. LC-MS (ES) m/z=259.9, 261.9 [M+H]⁺.

### (b) methyl 2-allyl-3-nitrobenzoate

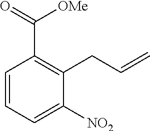 [SMILES: C=CCc1c(C(=O)OC)cccc1[N+](=O)[O-]]

1,2-Dimethoxyethane (300 mL) was added to a 600 mL Parr reaction vessel containing methyl 2-bromo-3-nitrobenzoate (14.0 g, 53.8 mmol), potassium allyltrifluoroborate (12.0 g, 81 mmol), CsF (26.2 g, 172 mmol) and Pd(Ph₃P)₄ (2.7 g, 2.337 mmol). The vessel was flushed with argon and placed in a 175° C. oil bath which was immediately turned down to 120° C. to increase the rate at which the reaction achieved 120° C. Once this temperature was achieved (˜30 min), the reaction was heated for 6 h. After 6 h the reaction mixture was filtered, adsorbed onto silica and purified via flash chromatography (CombiFlash®, 0-5% EtOAc in hexanes; 220 g column) to afford methyl 2-allyl-3-nitrobenzoate (7.84 g, 35.4 mmol, 65.8% yield) as a yellow oil. LC-MS (ES) m/z=221.9 [M+H]⁺.

### (c) ethyl 2-allyl-3-aminobenzoate

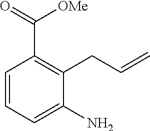

Zinc (51.29 g, 784 mmol) was added to a solution of methyl 2-allyl-3-nitrobenzoate (11.56 g, 52.3 mmol) in EtOH (300 mL) at −9° C. with stirring. While at −9° C., AcOH (40 mL, 699 mmol) was added slowly with vigorous stirring and monitoring of internal temperature, after about 20 mL was added and about 5 min the reaction rapidly exothermed to 26° C. and then cooled back to 0° C. at which point the rest of the AcOH was added and after several min the ice bath removed. After 1 h the reaction mixture was filtered, concentrated under vacuum to remove EtOH and a saturated aqueous solution of NaHCO₃ added. The aqueous layer was extracted with EtOAc (2×250 mL), the combined organics were washed with brine and dried over Na₂SO₄ and the dried solution filtered and concentrated to afford methyl 2-allyl-3-aminobenzoate (9.96 g, 52.1 mmol, 100% yield) as an orange oil. LC-MS (ES) m/z=192 [M+H]⁺.

### (d) methyl 2-allyl-3-((trans-4-((tert-butoxycarbonyl)amino)cyclohexyl)amino)benzoate

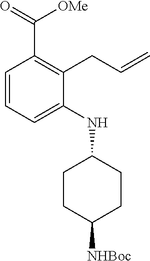 [SMILES: C=CCc1c(N[C@H]2CC[C@H](NC(=O)OC(C)(C)C)CC2)cccc1C(=O)OC]

To a stirred solution of methyl 2-allyl-3-aminobenzoate (2 g, 10.46 mmol) and tert-butyl(4-oxocyclohexyl)carbamate (4.46 g, 20.92 mmol) in DCE (50 mL) was added AcOH (0.599 mL, 10.46 mmol). The reaction was stirred for 2 h at room temperature, then Na(OAc)₃BH (4.43 g, 20.92 mmol) was added portionwise and the reaction was stirred overnight at room temperature. The reaction was diluted with DCM (200 mL) and washed sequentially with saturated aqueous NaHCO₃ solution (50 mL) and saturated aqueous sodium chloride solution (50 mL). The organic layer was dried over Na₂SO₄, filtered, concentrated and the residue was purified by flash chromatography (0-15% EtOAc in hexanes, 400-g column, mixed fractions re-columned, product is second eluting peak by flash chromatography) to afford methyl 2-allyl-3-((trans-4-((tert-butoxycarbonyl)amino)cyclohexyl)amino)benzoate (1.45 g, 3.73 mmol, 35.7% yield) as a white solid. LC-MS (ES) m/z=389.0 [M+H]⁺.

### (e) methyl 2-allyl-3-((trans-4-((tert-butoxycarbonyl)amino)cyclohexyl)(ethyl)amino)benzoate

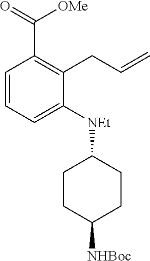

To a stirred solution of methyl 2-allyl-3-((trans-4-((tert-butoxycarbonyl)amino)cyclohexyl)amino)benzoate (1.45 g, 3.73 mmol), acetaldehyde (1.265 mL, 22.39 mmol) and AcOH (0.534 mL, 9.33 mmol) in DCE (10 mL) was added Na(OAc)₃BH (2.69 g, 12.69 mmol). The reaction was stirred at room temperature for one h. The reaction was diluted with DCM (100 mL) and washed with saturated aqueous NaHCO₃ solution (50 mL), then saturated aqueous sodium chloride solution (50 mL). The organic layer was dried over Na₂SO₄ and concentrated to afford methyl 2-allyl-3-((trans-4-((tert-butoxycarbonyl)amino)cyclohexyl)(ethyl)amino)benzoate (1.555 g, 3.73 mmol, 100% yield) as a white solid. LC-MS (ES) m/z=417.0 [M+H]⁺.

### (f) 2-allyl-3-((trans-4-((tert-butoxycarbonyl)amino)cyclohexyl)(ethyl)amino)benzoic acid

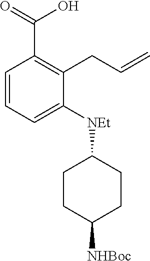

To a stirred solution of methyl 2-allyl-3-((trans-4-((tert-butoxycarbonyl)amino)cyclohexyl)(ethyl)amino)benzoate (1.55 g, 3.72 mmol) in MeOH (40 mL) was added aqueous NaOH (6 M, 3.10 mL, 18.61 mmol). The reaction was heated at 60° C. overnight. The reaction was concentrated, neutralized with aqueous HCl (1 M, 18.61 mL, 18.61 mmol) which resulted in a precipitate, adjusted to pH ca. 4 (tested with pH paper), then extracted with EtOAc (4×100 mL). The combined organics were washed with saturated aqueous sodium chloride solution, then dried over Na₂SO₄, filtered, concentrated to afford 2-allyl-3-((trans-4-((tert-butoxycarbonyl)amino)cyclohexyl)(ethyl)amino)benzoic acid (1.498 g, 3.72 mmol, 100% yield) as a yellow oil. LC-MS (ES) m/z=403.0 [M+H]⁺.

### (g) tert-butyl(trans-4-((2-allyl-3-(((4-(but-3-en-1-yl)-2-methoxy-6-methylpyridin-3-yl)methyl)carbamoyl)phenyl)(ethyl)amino)cyclohexyl)carbamate

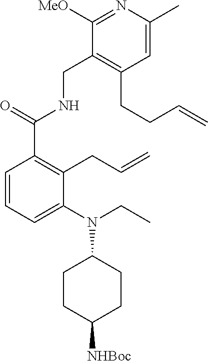 [SMILES: C=CCCc1cc(C)nc(OC)c1CNC(=O)c1cccc(N(CC)[C@H]2CC[C@H](NC(=O)OC(C)(C)C)CC2)c1CC=C]

A mixture of 2-allyl-3-((trans-4-((tert-butoxycarbonyl)amino)cyclohexyl)(ethyl)amino)benzoic acid (1.498 g, 3.72 mmol), (4-(but-3-en-1-yl)-2-methoxy-6-methylpyridin-3-yl)methanamine (0.921 g, 4.47 mmol), HOAt (0.760 g, 5.58 mmol), EDC (1.070 g, 5.58 mmol), N-methylmorpholine (1.227 mL, 11.16 mmol) was stirred at room temperature overnight. The reaction mixture was poured into water and stirred for one h. The precipitate was collected by filtration, and dried at the pump overnight. The filter cake was dissolved in EtOAc (100 mL) and washed with saturated aqueous sodium chloride solution (20 mL), then the organic layer was dried over Na₂SO₄, filtered, concentrated and the residue was purified by flash chromatography (0-30% EtOAc in hexanes) to afford tert-butyl(trans-4-((2-allyl-3-(((4-(but-3-en-1-yl)-2-methoxy-6-methylpyridin-3-yl)methyl)carbamoyl)phenyl)(ethyl)amino)cyclohexyl)carbamate (2.13 g, 3.61 mmol, 97% yield) as a colorless oil. LC-MS (ES) m/z=591.1 [M+H]⁺.

### (h) tert-butyl(trans-4-(ethyl((E)-1-methoxy-3-methyl-14-oxo-5,6,9,14,15,16-hexahydrobenzo[c]pyrido[4,3-j][1]azacyclododecin-10-yl)amino)cyclohexyl)carbamate

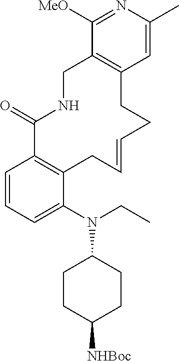 [SMILES: CCN(c1cccc2c1C/C=C/CCc1cc(C)nc(OC)c1CNC2=O)[C@H]1CC[C@H](NC(=O)OC(C)(C)C)CC1]

To a solution of tert-butyl(trans-4-((2-allyl-3-(((4-(but-3-en-1-yl)-2-methoxy-6-methylpyridin-3-yl)methyl)carbamoyl)phenyl)(ethyl)amino)cyclohexyl)carbamate (2.08 g, 3.52 mmol) in DCM (60 mL) was added Grubbs II (0.299 g, 0.352 mmol). The reaction was stirred overnight at room temperature. An additional portion of Grubbs II (0.299 g, 0.352 mmol) was added, and the reaction was stirred overnight at room temperature. The reaction was concentrated and purified by flash chromatography (0-40% EtOAc in hexanes, 60-g column) to afford a mixture of E and Z isomers (ca. 1.2 g) and impure starting material (600 mg). The impure starting material was dissolved in DCM (40 mL) and Grubbs II (120 mg) was added. The reaction was stirred for 4 days. The reaction mixture was concentrated, then purified by flash chromatography (0-40% EtOAc in hexanes, 40-g column) to afford a mixture of E and Z isomers (ca. 0.2 g). The combined mixtures (ca. 1.4 g total) was purified by RP-HPLC (30-45% CH₃CN in water, 0.1% TFA) to afford tert-butyl(trans-4-(ethyl((E)-1-methoxy-3-methyl-14-oxo-5,6,9,14,15,16-hexahydrobenzo[c]pyrido[4,3-j][1]azacyclododecin-10-yl)amino)cyclohexyl)carbamate (842 mg) as a colorless oil. LC-MS (ES)=563 [M+H]⁺ (minor), 365.9 (major). LC-MS (ES) and ¹H NMR both indicated a ca. 50% impurity present consistent with boc-deprotected material.

### (i) (E)-10-((trans-4-aminocyclohexyl)(ethyl)amino)-3-methyl-5,6,15,16-tetrahydrobenzo[c]pyrido[4,3-j][1]azacyclododecine-1,14(2H,9H)-dione

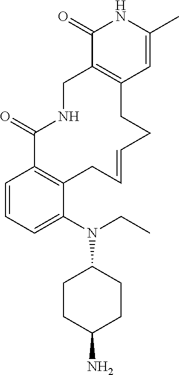 [SMILES: CCN(c1cccc2c1C/C=C/CCc1cc(C)[nH]c(=O)c1CNC2=O)[C@H]1CC[C@H](N)CC1]

Tert-butyl(trans-4-(ethyl((E)-1-methoxy-3-methyl-14-oxo-5,6,9,14,15,16-hexahydrobenzo[c]pyrido[4,3-j][1]azacyclododecin-10-yl)amino)cyclohexyl)carbamate (112 mg, containing ca. 50% boc-deprotected material) was dissolved in 1,4-dioxane (2 mL) and MeOH (0.5 mL). HCl (4 M, dioxane, 2 mL, 8.00 mmol) was added and the reaction was stirred at 60° C. overnight and the reaction was concentrated. The residue was dissolved in DCM (3 mL) and MeOH (1 mL) and concentrated NH₄OH was added (0.5 mL). The mixture was stirred for 30 min, then diluted with DCM (50 mL), adsorbed onto silica and purified by flash chromatography (0-100% (90:10:1 DCM:MeOH:NH₄OH) in DCM, 12-g column) to afford (E)-10-((trans-4-aminocyclohexyl)(ethyl)amino)-3-methyl-5,6,15,16-tetrahydrobenzo[c]pyrido[4,3-j][1]azacyclododecine-1,14(2H,9H)-dione (45 mg, 0.100 mmol) as a white solid. LC-MS (ES) m/z=449.0 [M+H]⁺. ¹H NMR (400 MHz, DMSO-d₆) δ: 7.99 (t, J=4.7 Hz, 1H), 7.12-7.24 (m, 2H), 6.88-6.96 (m, 1H), 5.84 (s, 1H), 5.02-5.24 (m, 2H), 4.17 (br. s., 2H), 3.52 (br. s., 2H), 2.98 (d, J=6.6 Hz, 2H), 2.53-2.66 (m, 2H), 2.43 (t, J=10.7 Hz, 1H), 2.22 (br. s., 2H), 2.12 (s, 3H), 1.58-1.83 (m, 4H), 1.13-1.41 (m, 2H), 0.82-1.00 (m, 2H), 0.75 (t, J=6.95 Hz, 3H). Note: exchangeable NHs not observed as distinct peaks. One methine proton not clearly observed.

### Example 37

### (E)-10-((trans-4-(dimethylamino)cyclohexyl)(ethyl)amino)-3-methyl-5,6,15,16-tetrahydrobenzo[c]pyrido[4,3-j][1]azacyclododecine-1,14(2H,9H)-dione

### (a) (E)-10-((trans-4-aminocyclohexyl)(ethyl)amino)-3-methyl-5,6,15,16-tetrahydrobenzo[c]pyrido[4,3-j][1]azacyclododecine-1,14(2H,9H)-dione hydrochloride

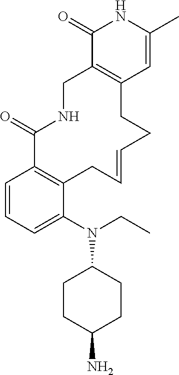 [SMILES: CCN(c1cccc2c1C/C=C/CCc1cc(C)[nH]c(=O)c1CNC2=O)[C@H]1CC[C@H](N)CC1]

Tert-butyl(trans-4-(ethyl((E)-1-methoxy-3-methyl-14-oxo-5,6,9,14,15,16-hexahydrobenzo[c]pyrido[4,3-j][1]azacyclododecin-10-yl)amino)cyclohexyl)carbamate (730 mg, containing ca. 50% boc-deprotected material) was dissolved in 1,4-dioxane (4 mL) and MeOH (1 mL). HCl (4 M in dioxane, 2 mL, 8.00 mmol) was added and the reaction was stirred at 60° C. overnight. The reaction was concentrated to afford (E)-10-((trans-4-aminocyclohexyl)(ethyl)amino)-3-methyl-5,6,15,16-tetrahydrobenzo[c]pyrido[4,3-j][1]azacyclododecine-1,14(2H,9H)-dione, hydrochloride (561 mg, 1.157 mmol) as a white solid. LC-MS (ES) m/z=449.0 [M+H]⁺.

### (b) (E)-10-((trans-4-(dimethylamino)cyclohexyl)(ethyl)amino)-3-methyl-5,6,15,16-tetrahydrobenzo[c]pyrido[4,3-j][1]azacyclododecine-1,14(2H,9H)-dione

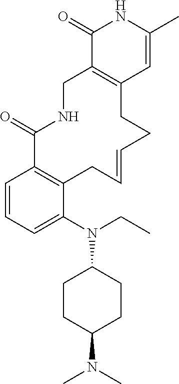 [SMILES: CCN(c1cccc2c1C/C=C/CCc1cc(C)[nH]c(=O)c1CNC2=O)[C@H]1CC[C@H](N(C)C)CC1]

To a solution of (E)-10-((trans-4-aminocyclohexyl)(ethyl)amino)-3-methyl-5,6,15,16-tetrahydrobenzo[c]pyrido[4,3-j][1]azacyclododecine-1,14(2H,9H)-dione, hydrochloride (150 mg, 0.309 mmol) and formaldehyde (0.092 mL, 3.34 mmol) in MeOH (3 mL) was added NaBH₃CN (210 mg, 3.34 mmol) in one portion. The reaction mixture was stirred at room temperature for 1 h, then the reaction mixture was concentrated. The reaction was re-dissolved in MeOH and DCM, NH₄OH and the mixture was concentrated onto silica gel, then purified by flash chromatography (100% DCM to 90:10:1 DCM:MeOH:NH₄OH) to afford (E)-10-((trans-4-(dimethylamino)cyclohexyl)(ethyl)amino)-3-methyl-5,6,15,16-tetrahydrobenzo[c]pyrido[4,3-j][1]azacyclododecine-1,14(2H,9H)-dione (65 mg, 0.136 mmol, 44.1% yield) as a white solid. LC-MS (ES) m/z=477.0 [M+H]⁺. ¹H NMR (400 MHz, DMSO-d₆) δ: 11.29 (s, 1H), 7.98 (t, J=4.8 Hz, 1H), 7.13-7.24 (m, 2H), 6.88-6.98 (m, 1H), 5.84 (s, 1H), 5.04-5.24 (m, 2H), 4.00-4.34 (m, 2H), 3.53 (br. s., 2H), 2.99 (d, J=6.6 Hz, 2H), 2.53-2.69 (m, 3H), 2.17-2.29 (m, 2H), 2.14 (s, 6H), 2.12 (s, 3H), 2.04-2.10 (m, 1H), 1.76 (d, J=10.1 Hz, 4H), 1.24 (q, J=11.5 Hz, 2H), 1.07 (q, J=11.6 Hz, 2H), 0.75 (t, J=7.0 Hz, 3H).

### Example 38

### (E)-11-(ethyl(tetrahydro-2H-pyran-4-yl)amino)-3-methyl-5,6,7,10,16,17-hexahydro-1H-benzo[h]pyrido[4,3-c][1,6]diazacyclotridecine-1,15(2H)-dione

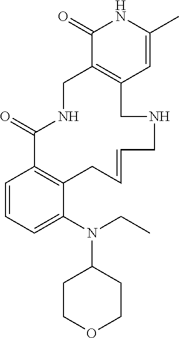 [SMILES: CCN(c1cccc2c1C/C=C/CNCc1cc(C)[nH]c(=O)c1CNC2=O)C1CCOCC1]

A mixture of (E)- and (Z)-tert-butyl 11-(ethyl(tetrahydro-2H-pyran-4-yl)amino)-3-methyl-1,15-dioxo-5,7,10,15,16,17-hexahydro-1H-benzo[h]pyrido[4,3-c][1,6]diazacyclotridecine-6(2H)-carboxylate (31 mg, 0.056 mmol) in MeOH (0.5 mL) at room temperature was added HCl (4 M in dioxane, 0.5 mL, 2.00 mmol). The reaction mixture was then stirred at room temperature for 2 h then the reaction mixture was then concentrated. The resulting oil was then taken up in CH₃CN and 172 μL of isopropylamine was added. The mixture was then purified by reverse phase HPLC (Gilson®, 30 mm Gemini Column, NH₄OH modifier) to afford (E)-11-(ethyl(tetrahydro-2H-pyran-4-yl)amino)-3-methyl-5,6,7,10,16,17-hexahydro-1H-benzo[h]pyrido[4,3-c][1,6]diazacyclotridecine-1,15(2H)-dione (18.3 mg, 0.041 mmol) as a white solid. LC-MS (ES) m/z=451 [M+H]⁺. ¹H NMR (400 MHz, DMSO-d₆) δ: 11.46 (br. s., 1H), 7.77 (t, J=4.6 Hz, 1H), 7.15-7.31 (m, 2H), 7.05 (dd, J=7.2, 1.4 Hz, 1H), 6.12 (s, 1H), 5.31-5.43 (m, 1H), 5.01-5.16 (m, 1H), 4.30 (d, J=4.8 Hz, 2H), 4.09 (q, J=5.3 Hz, 1H), 3.75-3.85 (m, 4H), 3.47 (s, 2H), 3.10-3.26 (m, 4H), 2.88-3.03 (m, 4H), 2.12 (s, 3H), 1.53-1.67 (m, 1H), 1.26-1.52 (m, 2H), 0.75 (t, J=7.0 Hz, 3H).

### Example 39

### (Z)-11-(ethyl(tetrahydro-2H-pyran-4-yl)amino)-3-methyl-5,6,7,10,16,17-hexahydro-1H-benzo[h]pyrido[4,3-c][1,6]diazacyclotridecine-1,15(2H)-dione

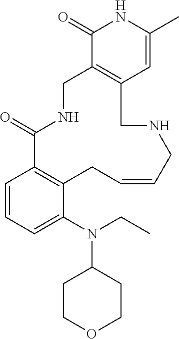 [SMILES: CCN(c1cccc2c1C/C=C\CNCc1cc(C)[nH]c(=O)c1CNC2=O)C1CCOCC1]

Also isolated from the above purification was (Z)-11-(ethyl(tetrahydro-2H-pyran-4-yl)amino)-3-methyl-5,6,7,10,16,17-hexahydro-1H-benzo[h]pyrido[4,3-c][1,6]diazacyclotridecine-1,15(2H)-dione (6.8 mg, 0.015 mmol) as an off-white solid. LC-MS (ES) m/z=451 [M+H]⁺. ¹H NMR (400 MHz, DMSO-d₆) δ: 11.45 (br. s., 1H), 7.92 (t, J=4.6 Hz, 1H), 7.14-7.31 (m, 2H), 7.04 (dd, J=7.0, 1.9 Hz, 1H), 6.22 (s, 1H), 5.07-5.23 (m, 2H), 4.26 (d, J=5.05 Hz, 2H), 3.73-3.88 (m, 2H), 3.54-3.71 (m, 4H), 3.15-3.27 (m, 2H), 3.05-3.15 (m, 2H), 2.92-3.05 (m, 3H), 2.15 (s, 3H), 1.49-1.70 (m, 2H), 1.31-1.49 (m, 2H), 0.76 (t, J=7.0 Hz, 3H). 1H not observed.

### Example 40

### (E)-11-(ethyl(piperidin-4-yl)amino)-3-methyl-5,6,7,10,16,17-hexahydro-1H-benzo[c]pyrido[4,3-k][1]azacyclotridecine-1,15(2H)-dione

### (a) tert-butyl 4-((2-allyl-3-(42-methoxy-6-methyl-4-(pent-4-en-1-yl)pyridin-3-yl)methyl)carbamoyl)phenyl)(ethyl)amino)piperidine-1-carboxylate

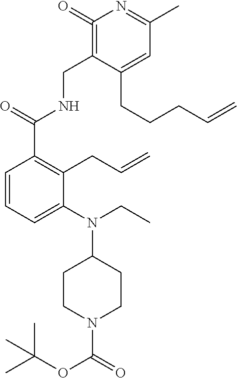

The reaction mixture of 2-allyl-3-((1-(tert-butoxycarbonyl)piperidin-4-yl)(ethyl)amino)benzoic acid (800 mg, 2.059 mmol) and (2-methoxy-6-methyl-4-(pent-4-en-1-yl)pyridin-3-yl)methanamine (454 mg, 2.059 mmol), EDC (513 mg, 2.68 mmol), HOAt (364 mg, 2.68 mmol) and N-methylmorpholine (0.679 mL, 6.18 mmol) in DCM (11 mL) was stirred at room temperature for 2 h. The reaction mixture was quenched with water and the layers were separated. The aqueous layer was extracted with DCM (2×). The combined organics were washed with water and brine, dried over Na₂SO₄, concentrated and purified by silica (CombiFlash®, 30 g column, 0-20% EtOAc in hexane) to afford tert-butyl 4-((2-allyl-3-(((2-methoxy-6-methyl-4-(pent-4-en-1-yl)pyridin-3-yl)methyl)carbamoyl)phenyl)(ethyl)amino)piperidine-1-carboxylate (1.03 g, 1.743 mmol, 85% yield) as a white foam solid. LC-MS (ES) m/z=591.7 [M+H]⁺.

### (b) (E)-tert-butyl 4-(ethyl(1-methoxy-3-methyl-15-oxo-6,7,10,15,16,17-hexahydro-5H-benzo[c]pyrido[4,3-k][1]azacyclotridecin-11-yl)amino)piperidine-1-carboxylate

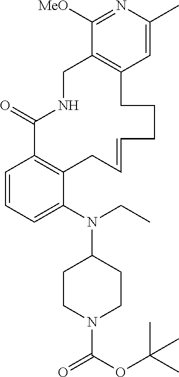 [SMILES: CCN(c1cccc2c1C/C=C/CCCc1cc(C)nc(OC)c1CNC2=O)C1CCN(C(=O)OC(C)(C)C)CC1]

To a degassed solution of tert-butyl 4-((2-allyl-3-(((2-methoxy-6-methyl-4-(pent-4-en-1-yl)pyridin-3-yl)methyl)carbamoyl)phenyl)(ethyl)amino)piperidine-1-carboxylate (1.03 g, 1.743 mmol) in DCM (80 mL) was added Grubbs II (0.296 g, 0.349 mmol). The reaction mixture was stirred at room temperature for 20 h under nitrogen. The reaction mixture was adsorbed onto silica, and purified by silica column (CombiFlash®, 30 g column, 0-30% EtOAc in hexane) to afford a mixture of E and Z isomers. The mixture was purified by HPLC (0.1% TFA in mobile phase; 30-60% CH₃CN in water) to afford (E)-tert-butyl 4-(ethyl(1-methoxy-3-methyl-15-oxo-6,7,10,15,16,17-hexahydro-5H-benzo[c]pyrido[4,3-k][1]azacyclotridecin-11-yl)amino)piperidine-1-carboxylate (683 mg) as a white solid. LC-MS (ES) m/z=563.6 [M+H]⁺.

### (c) (E)-11-(ethyl(piperidin-4-yl)amino)-3-methyl-5,6,7,10,16,17-hexahydro-1H-benzo[c]pyrido[4,3-k][1]azacyclotridecine-1,15(2H)-dione

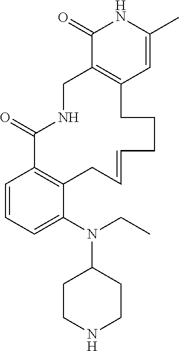 [SMILES: CCN(c1cccc2c1C/C=C/CCCc1cc(C)[nH]c(=O)c1CNC2=O)C1CCNCC1]

To a solution of (E)-tert-butyl 4-(ethyl(1-methoxy-3-methyl-15-oxo-6,7,10,15,16,17-hexahydro-5H-benzo[c]pyrido[4,3-k][1]azacyclotridecin-11-yl)amino)piperidine-1-carboxylate (660 mg, 1.173 mmol) in dioxane (20 mL) and MeOH (10 mL) was added HCl (4 M, dioxane, 5 mL, 20.00 mmol). The resulting mixture was heated at 70° C. overnight. A white precipitate formed and the reaction mixture was concentrated. The residue was basified with saturated aqueous NaHCO₃ solution, and the solid was filtered and washed with water to afford (E)-11-(ethyl(piperidin-4-yl)amino)-3-methyl-5,6,7,10,16,17-hexahydro-1H-benzo[c]pyrido[4,3-k][1]azacyclotridecine-1,15(2H)-dione (478 mg, 1.066 mmol, 91% yield) as a white solid. LC-MS (ES) m/z=449.4 [M+H]⁺. ¹H NMR (METHANOL-d₄) δ: 7.33 (dd, J=8.1, 1.3 Hz, 1H), 7.24 (t, J=7.7 Hz, 1H), 7.13-7.19 (m, 1H), 6.16 (s, 1H), 5.41-5.50 (m, 1H), 5.15-5.25 (m, 1H), 4.53 (s, 2H), 3.87-3.94 (m, 2H), 2.99-3.14 (m, 4H), 2.90-2.99 (m, 1H), 2.43-2.59 (m, 4H), 2.25 (s, 3H), 1.97-2.05 (m, 2H), 1.78 (br. s., 2H), 1.59-1.70 (m, 2H), 1.43-1.57 (m, 2H), 0.86 (t, J=6.9 Hz, 3H). Note 3H not observed.

### Example 41

### (E)-10-((1-(cyclopropylmethyl)piperidin-4-yl)(ethyl)amino)-3-methyl-5,6,15,16-tetrahydrobenzo[c]pyrido[4,3-j][1]azacyclododecine-1,14(2H,9H)-dione

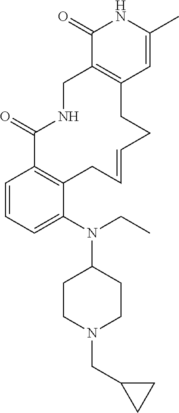 [SMILES: CCN(c1cccc2c1C/C=C/CCc1cc(C)[nH]c(=O)c1CNC2=O)C1CCN(CC2CC2)CC1]

To a solution of (E)-10-(ethyl(piperidin-4-yl)amino)-3-methyl-5,6,15,16-tetrahydrobenzo[c]pyrido[4,3-j][1]azacyclododecine-1,14(2H,9H)-dione (100 mg, 0.230 mmol) in MeOH (3 mL) was added cyclopropanecarbaldehyde (0.103 mL, 1.381 mmol), AcOH (0.026 mL, 0.460 mmol) and NaBH₃CN (116 mg, 1.841 mmol). The reaction mixture was stirred overnight, then quenched with saturated aqueous NaHCO₃(3 mL), adsorbed onto silica, then purified by silica column (CombiFlash®, 4 g column, 0-100% (1% NH₄OH+9% MeOH+90% CHCl₃)/CHCl₃) to afford (E)-10-((1-(cyclopropylmethyl)piperidin-4-yl)(ethyl)amino)-3-methyl-5,6,15,16-tetrahydrobenzo[c]pyrido[4,3-j][1]azacyclododecine-1,14(2H,9H)-dione (85 mg, 0.174 mmol, 76% yield) as a white solid. LC-MS (ES) m/z=245.2 (major), 489.5 [M+H]⁺ (minor). ¹H NMR (METHANOL-d₄) δ: 7.27-7.33 (m, 1H), 7.20-7.27 (m, 1H), 7.08 (dd, J=7.2, 1.4 Hz, 1H), 6.12 (s, 1H), 5.28-5.40 (m, 1H), 5.15-5.27 (m, 1H), 4.38 (s, 2H), 3.70 (d, J=5.6 Hz, 2H), 3.07 (q, J=7.0 Hz, 4H), 2.84 (br. s., 1H), 2.72 (br. s., 2H), 2.34 (br. s., 2H), 2.26 (s, 3H), 2.22 (d, J=6.8 Hz, 2H), 2.00 (t, J=11.0 Hz, 2H), 1.68-1.91 (m, 2H), 1.52-1.67 (m, 2H), 0.78-0.91 (m, 4H), 0.48-0.57 (m, 2H), 0.12 (q, J=4.8 Hz, 2H). Note 2H not observed.

### Example 42

### (E)-11-(ethyl(1-isopropylpiperidin-4-yl)amino)-3-methyl-5,6,7,10,16,17-hexahydro-1H-benzo[c]pyrido[4,3-k][1]azacyclotridecine-1,15(2H)-dione

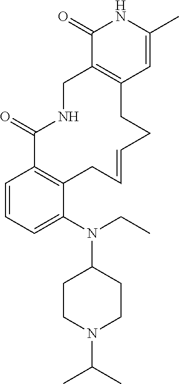 [SMILES: CCN(c1cccc2c1C/C=C/CCc1cc(C)[nH]c(=O)c1CNC2=O)C1CCN(C(C)C)CC1]

To a solution of (E)-11-(ethyl(piperidin-4-yl)amino)-3-methyl-5,6,7,10,16,17-hexahydro-1H-benzo[c]pyrido[4,3-k][1]azacyclotridecine-1,15(2H)-dione (145 mg, 0.323 mmol) in MeOH (3 mL) was added NaBH₃CN (162 mg, 2.59 mmol), acetone (0.356 mL, 4.85 mmol) and AcOH (0.037 mL, 0.646 mmol). The reaction mixture was stirred at room temperature overnight. Added more NaBH₃CN (72.3 mg, 1.151 mmol), and acetone (0.135 mL, 1.841 mmol), and stirred for another day. The reaction mixture was quenched with saturated aqueous NaHCO₃ (3 mL), adsorbed onto silica, then purified by silica column (CombiFlash®, 4 g column, using 0-100% (1% NH₄OH+9% MeOH+90% CHCl₃)/CHCl₃) to afford (E)-11-(ethyl(1-isopropylpiperidin-4-yl)amino)-3-methyl-5,6,7,10,16,17-hexahydro-1H-benzo[c]pyrido[4,3-k][1]azacyclotridecine-1,15(2H)-dione (118 mg, 0.240 mmol, 74.4% yield) as an off-white solid. LC-MS (ES) m/z=491.5 [M+H]⁺. ¹H NMR (METHANOL-d₄) δ: 7.33 (dd, J=8.0, 1.4 Hz, 1H), 7.24 (t, J=7.7 Hz, 1H), 7.14-7.19 (m, 1H), 6.16 (s, 1H), 5.45 (dt, J=15.3, 5.5 Hz, 1H), 5.14-5.24 (m, 1H), 4.52 (s, 2H), 3.89-3.94 (m, 2H), 3.09 (q, J=7.1 Hz, 2H), 2.82-2.94 (m, 3H), 2.69 (dt, J=13.1, 6.6 Hz, 1H), 2.49-2.58 (m, 2H), 2.25 (s, 3H), 2.10-2.20 (m, 2H), 1.95-2.05 (m, 2H), 1.80 (br. s., 2H), 1.54-1.69 (m, 4H), 1.06 (d, J=6.6 Hz, 6H), 0.86 (t, J=7.1 Hz, 3H). Note 2H not observed.

### Example 43

### (E)-10-(ethyl(1-(3,3,3-trifluoropropyl)piperidin-4-yl)amino)-3-methyl-5,6,15,16-tetrahydrobenzo[c]pyrido[4,3-j][1]azacyclododecine-1,14(2H,9H)-dione

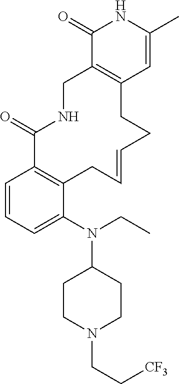 [SMILES: CCN(c1cccc2c1C/C=C/CCc1cc(C)[nH]c(=O)c1CNC2=O)C1CCN(CCC(F)(F)F)CC1]

To a solution of (E)-10-(ethyl(piperidin-4-yl)amino)-3-methyl-5,6,15,16-tetrahydrobenzo[c]pyrido[4,3-j][1]azacyclododecine-1,14(2H,9H)-dione (90 mg, 0.207 mmol) in MeOH (3 mL) was added 3,3,3-trifluoropropanal (0.124 mL, 1.035 mmol), AcOH (0.024 mL, 0.414 mmol) and NaBH₃CN (104 mg, 1.657 mmol). The reaction mixture was stirred overnight then quenched with saturated aqueous NaHCO₃ (3 mL), adsorbed onto silica, then purified by silica column (CombiFlash®, 4 g column, 0-100% (1% NH₄OH+9% MeOH+90% CHCl₃)/CHCl₃) to afford (E)-10-(ethyl(1-(3,3,3-trifluoropropyl)piperidin-4-yl)amino)-3-methyl-5,6,15,16-tetrahydrobenzo[c]pyrido[4,3-j][1]azacyclododecine-1,14(2H,9H)-dione (59 mg, 0.111 mmol, 53.7% yield) as a white solid. LC-MS (ES) m/z=531.5 [M+H]⁺. ¹H NMR (METHANOL-d₄) δ: 7.27-7.31 (m, 1H), 7.21-7.27 (m, 1H), 7.08 (dd, J=7.2, 1.4 Hz, 1H), 6.12 (s, 1H), 5.29-5.39 (m, 1H), 5.16-5.26 (m, 1H), 4.38 (s, 2H), 3.66-3.74 (m, 2H), 3.06 (q, J=7.1 Hz, 2H), 2.78-2.94 (m, 3H), 2.72 (br. s., 2H), 2.53-2.60 (m, 2H), 2.29-2.45 (m, 4H), 2.26 (s, 3H), 1.96-2.07 (m, 2H), 1.79 (br. s., 2H), 1.50-1.63 (m, 2H), 0.83 (t, J=6.9 Hz, 3H). Note 2H not observed.

### Example 44

### (E)-10-(ethyl(1-ethylpiperidin-4-yl)amino)-3-methyl-5,6,15,16-tetrahydrobenzo[c]pyrido[4,3-j][1]azacyclododecine-1,14(2H,9H)-dione

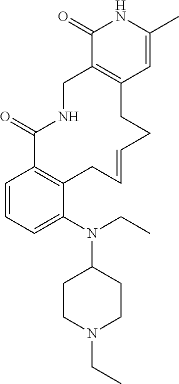 [SMILES: CCN1CCC(N(CC)c2cccc3c2C/C=C/CCc2cc(C)[nH]c(=O)c2CNC3=O)CC1]

To a solution of (E)-10-(ethyl(piperidin-4-yl)amino)-3-methyl-5,6,15,16-tetrahydrobenzo[c]pyrido[4,3-j][1]azacyclododecine-1,14(2H,9H)-dione (94 mg, 0.216 mmol) in MeOH (3 mL) was added acetaldehyde (0.182 mL, 3.24 mmol), AcOH (0.025 mL, 0.433 mmol) and NaBH₃CN (136 mg, 2.163 mmol). The reaction mixture was stirred for 4 h. The reaction mixture was quenched with saturated aqueous NaHCO₃ (3 mL), adsorbed onto silica, then purified by silica column (CombiFlash®, 4 g column, 0-100% (1% NH₄OH+9% MeOH+90% CHCl₃)/CHCl₃). The resulting solid was not pure and was further purified by HPLC (0.1% TFA in mobile phase; 10-40% CH₃CN in water). The resulting fractions were concentrated and the residue was passed through a 1 g of Silicycle (carbonate) cartridge eluting with MeOH (30 mL) to afford (E)-10-(ethyl(1-ethylpiperidin-4-yl)amino)-3-methyl-5,6,15,16-tetrahydrobenzo[c]pyrido[4,3-j][1]azacyclododecine-1,14(2H,9H)-dione (11 mg, 0.024 mmol, 10.99% yield) as a white solid. LC-MS (ES) m/z=232.2 (major), 463.4 [M+H]⁺ (minor). ¹H NMR (METHANOL-d₄) δ: 7.19-7.33 (m, 2H), 7.08 (dd, J=7.3, 1.5 Hz, 1H), 6.12 (s, 1H), 5.28-5.41 (m, 1H), 5.14-5.27 (m, 1H), 4.39 (s, 2H), 3.64-3.74 (m, 2H), 3.07 (q, J=7.1 Hz, 2H), 2.77-2.98 (m, 3H), 2.66-2.76 (m, 2H), 2.30-2.44 (m, 4H), 2.26 (s, 3H), 1.89-1.99 (m, 2H), 1.66-1.88 (m, 2H), 1.45-1.65 (m, 2H), 1.08 (t, J=7.2 Hz, 3H), 0.83 (t, J=6.9 Hz, 3H). 2H not observed.

### Example 45

### (E)-10-(ethyl(14(1-methyl-1H-pyrazol-3-yl)methyl)piperidin-4-yl)amino)-3-methyl-5,6,15,16-tetrahydrobenzo[c]pyrido[4,3-j][1]azacyclododecine-1,14(2H,9H)-dione

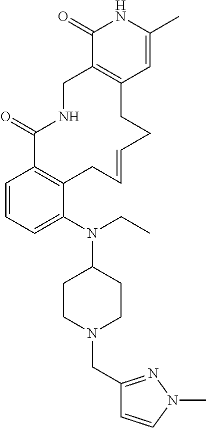 [SMILES: CCN(c1cccc2c1C/C=C/CCc1cc(C)[nH]c(=O)c1CNC2=O)C1CCN(Cc2ccn(C)n2)CC1]

To a solution of (E)-10-(ethyl(piperidin-4-yl)amino)-3-methyl-5,6,15,16-tetrahydrobenzo[c]pyrido[4,3-j][1]azacyclododecine-1,14(2H,9H)-dione (100 mg, 0.230 mmol) in MeOH (3 mL) was added 1-methyl-1H-pyrazole-3-carbaldehyde (101 mg, 0.920 mmol), AcOH (0.026 mL, 0.460 mmol) and NaBH₃CN (116 mg, 1.841 mmol). The reaction mixture was stirred overnight. The reaction mixture was quenched with saturated aqueous NaHCO₃ (3 mL), concentrated down with silica, then purified by silica column (CombiFlash®, 4 g column, 0-100% (1% NH₄OH+9% MeOH+90% CHCl₃)/CHCl₃). The resulting solid was not pure enough and was further purified by HPLC (0.1% TFA in mobile phase; 10-40% CH₃CN in water). The resulting fractions were concentrated and the residue was passed through a 1 g of Silicycle (carbonate) cartridge eluting with MeOH (30 mL) to afford to afford (E)-10-(ethyl(1-((l-methyl-1H-pyrazol-3-yl)methyl)piperidin-4-yl)amino)-3-methyl-5,6,15,16-tetrahydrobenzo[c]pyrido[4,3-j][1]azacyclododecine-1,14(2H,9H)-dione (56 mg, 0.106 mmol, 46.0% yield) as a white solid. LC-MS (ES) m/z=265.4 (major), 529.5 [M+H]⁺ (minor). ¹H NMR (400 MHz, METHANOL-d₄) δ: 7.51 (d, J=2.02 Hz, 1H), 7.19-7.31 (m, 2H), 7.07 (dd, J=1.52, 7.33 Hz, 1H), 6.25 (d, J=2.27 Hz, 1H), 6.12 (s, 1H), 5.27-5.39 (m, 1H), 5.12-5.26 (m, 1H), 4.38 (s, 2H), 3.85 (s, 3H), 3.60-3.72 (m, 2H), 3.50 (s, 2H), 3.05 (q, J=6.91 Hz, 2H), 2.64-2.94 (m, 5H), 2.34 (br. s., 2H), 2.27 (s, 3H), 2.02 (t, J=10.61 Hz, 2H), 1.75 (br. s., 2H), 1.59 (br. s., 2H), 0.82 (t, J=7.07 Hz, 3H). 2H not observed.

### Example 46

### (E)-ethyl 2-(4-(ethyl(3-methyl-1,14-dioxo-1,2,5,6,9,14,15,16-octahydrobenzo[c]pyrido[4,3-j][1]azacyclododecin-10-yl)amino)piperidin-1-yl)-2-methylpropanoate

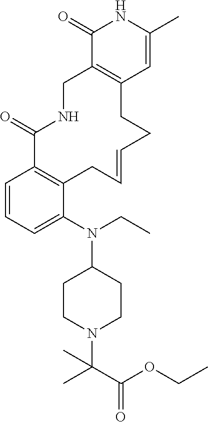 [SMILES: CCOC(=O)C(C)(C)N1CCC(N(CC)c2cccc3c2C/C=C/CCc2cc(C)[nH]c(=O)c2CNC3=O)CC1]

To a solution of (E)-10-(ethyl(piperidin-4-yl)amino)-3-methyl-5,6,15,16-tetrahydrobenzo[c]pyrido[4,3-j][1]azacyclododecine-1,14(2H,9H)-dione (100 mg, 0.230 mmol) in DMF (3 mL) was added ethyl 2-bromo-2-methylpropanoate (180 mg, 0.920 mmol), ethyl 2-bromo-2-methylpropanoate (180 mg, 0.920 mmol) and K₂CO₃ (95 mg, 0.690 mmol). The reaction mixture was stirred at 60° C. for 4 days. The reaction mixture was quenched with water, and the solid was filtered and purified by HPLC (0.1% TFA in mobile phase; 10-40% CH₃CN in water). The resulting fractions were concentrated and the residue was passed through a 1 g Silicycle (carbonate) cartridge eluting with MeOH (30 mL) to afford (E)-ethyl 2-(4-(ethyl(3-methyl-1,14-dioxo-1,2,5,6,9,14,15,16-octahydrobenzo[c]pyrido[4,3-j][1]azacyclododecin-10-yl)amino)piperidin-1-yl)-2-methylpropanoate (45 mg, 0.082 mmol, 35.6% yield) as a white solid. LC-MS (ES) m/z=275.4 (major), 549.5 [M+H]⁺ (minor). ¹H NMR (400 MHz, METHANOL-d₄) δ: 7.17-7.34 (m, 2H), 7.07 (dd, J=1.39, 7.20 Hz, 1H), 6.12 (s, 1H), 5.28-5.40 (m, 1H), 5.16-5.26 (m, 1H), 4.38 (s, 2H), 4.15 (q, J=7.07 Hz, 2H), 3.69 (d, J=5.31 Hz, 2H), 3.06 (q, J=6.91 Hz, 2H), 2.86-2.97 (m, 2H), 2.66-2.82 (m, 3H), 2.34 (br. s., 2H), 2.26 (s, 3H), 2.10-2.20 (m, 2H), 1.74 (br. s., 2H), 1.50-1.63 (m, 2H), 1.24-1.30 (m, 9H), 0.82 (t, J=7.07 Hz, 3H). 2H not observed.

### Example 47

### (E)-10-(ethyl(1-(2,2,2-trifluoroethyl)piperidin-4-yl)amino)-3-methyl-5,6,15,16-tetrahydrobenzo[c]pyrido[4,3-j][1]azacyclododecine-1,14(2H,9H)-dione

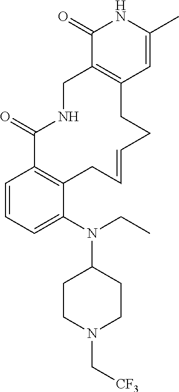 [SMILES: CCN(c1cccc2c1C/C=C/CCc1cc(C)[nH]c(=O)c1CNC2=O)C1CCN(CC(F)(F)F)CC1]

To a sealable tube containing (E)-10-(ethyl(piperidin-4-yl)amino)-3-methyl-5,6,15,16-tetrahydrobenzo[c]pyrido[4,3-j][1]azacyclododecine-1,14(2H,9H)-dione (120 mg, 0.276 mmol) in THF (3 mL) was added 2,2,2-trifluoroethyl trifluoromethanesulfonate (0.048 mL, 0.331 mmol), 2,2,2-trifluoroethyl trifluoromethanesulfonate (0.048 mL, 0.331 mmol) and Et₃N (0.192 mL, 1.381 mmol). The reaction vessel was sealed and stirred at 75° C. overnight. The reaction mixture was quenched with water, and the solid was filtered and to afford (E)-10-(ethyl(1-(2,2,2-trifluoroethyl)piperidin-4-yl)amino)-3-methyl-5,6,15,16-tetrahydrobenzo[c]pyrido[4,3-j][1]azacyclododecine-1,14(2H,9H)-dione (115 mg, 0.223 mmol, 81% yield) as a white solid. LC-MS (ES) m/z=517.4 [M+H]⁺. ¹H NMR (400 MHz, METHANOL-d₄) δ: 7.31-7.48 (m, 2H), 7.18-7.27 (m, 1H), 6.13 (s, 1H), 5.29-5.42 (m, 1H), 5.14-5.25 (m, 1H), 4.39 (br. s., 2H), 3.68 (br. s., 2H), 3.39-3.55 (m, 2H), 3.05-3.30 (m, 5H), 2.59-2.82 (m, 4H), 2.36 (br. s., 2H), 2.27 (s, 3H), 1.95 (s, 1H), 1.73 (br. s., 3H), 0.88 (t, J=6.95 Hz, 3H). 2H not observed.

### Example 48

### (E)-10-(ethyl(1-((6-methylpyridin-2-yl)methyl)piperidin-4-yl)amino)-3-methyl-5,6,15,16-tetrahydrobenzo[c]pyrido[4,3-j][1]azacyclododecine-1,14(2H,9H)-dione

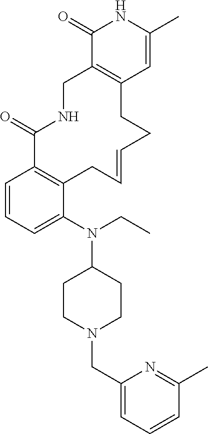 [SMILES: CCN(c1cccc2c1C/C=C/CCc1cc(C)[nH]c(=O)c1CNC2=O)C1CCN(Cc2cccc(C)n2)CC1]

To a solution of (E)-10-(ethyl(piperidin-4-yl)amino)-3-methyl-5,6,15,16-tetrahydrobenzo[c]pyrido[4,3-j][1]azacyclododecine-1,14(2H,9H)-dione (140 mg, 0.322 mmol) in MeOH (3 mL) was added 6-methylpicolinaldehyde (117 mg, 0.966 mmol), AcOH (0.037 mL, 0.644 mmol) and NaBH₃CN (162 mg, 2.58 mmol). The reaction mixture was stirred overnight then quenched with saturated aqueous NaHCO₃ (3 mL), concentrated down with silica, then purified by silica column (CombiFlash®, 4 g column, 0-100% (1% NH₄OH+9% MeOH+90% CHCl₃)/CHCl₃). The resulting solid was not pure and was further purified by HPLC (0.1% TFA in mobile phase; 10-40% CH₃CN in water). The resulting fractions were concentrated and the residue was passed through a 1 g Silicycle (carbonate) cartridge eluting with MeOH (30 mL) to afford (E)-10-(ethyl(1-((6-methylpyridin-2-yl)methyl)piperidin-4-yl)amino)-3-methyl-5,6,15,16-tetrahydrobenzo[c]pyrido[4,3-j][1]azacyclododecine-1,14(2H,9H)-dione (45 mg, 0.083 mmol, 25.9% yield) as a white solid. LC-MS (ES) m/z=270.8 (major), [M+H]⁺ (minor). ¹H NMR (400 MHz, METHANOL-d₄) δ: 8.07 (br. s., 1H), 7.74 (t, J=7.71 Hz, 1H), 7.24-7.36 (m, 4H), 7.12 (dd, J=1.52, 7.33 Hz, 1H), 6.12 (s, 1H), 5.27-5.40 (m, 1H), 5.15-5.26 (m, 1H), 4.39 (br. s., 2H), 4.15 (br. s., 2H), 3.70 (br. s., 2H), 3.22-3.31 (m, 2H), 3.11-3.18 (m, 1H), 3.07 (q, J=6.7 Hz, 2H), 2.77-2.92 (m, 2H), 2.65-2.77 (m, 2H), 2.54-2.59 (m, 3H), 2.34 (br. s., 2H), 2.26 (s, 3H), 1.79 (br. s., 3H), 0.84 (t, J=6.95 Hz, 3H). 2H not observed.

### Example 49

### (E)-10-((trans-4-(diethylamino)cyclohexyl)(ethyl)amino)-3-methyl-5,6,15,16-tetrahydrobenzo[c]pyrido[4,3-j][1]azacyclododecine-1,14(2H,9H)-dione

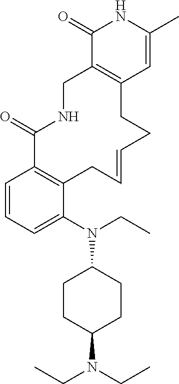 [SMILES: CCN(CC)[C@H]1CC[C@H](N(CC)c2cccc3c2C/C=C/CCc2cc(C)[nH]c(=O)c2CNC3=O)CC1]

To a solution of (E)-10-((trans-4-aminocyclohexyl)(ethyl)amino)-3-methyl-5,6,15,16-tetrahydrobenzo[c]pyrido[4,3-j][1]azacyclododecine-1,14(2H,9H)-dione, hydrochloride (130 mg, 0.268 mmol), AcOH (0.015 mL, 0.268 mmol), and acetaldehyde (0.151 mL, 2.68 mmol) in MeOH (3 mL) was added NaBH₃CN (168 mg, 2.68 mmol) in one portion. The reaction mixture was stirred at room temperature overnight, and then concentrated. The residue was purified by flash chromatography (100% DCM to 90:10:1 DCM:MeOH:NH₄OH, 4-g column) to afford a white solid. The solid was purified by reverse-phase HPLC (8-25% CH₃CN in water, 0.1% TFA) and the product fractions pooled and concentrated to ca. 20 mL of aqueous. The solution was basified with NaHCO₃ (20 mL) and extracted with EtOAc (3×50 mL). The combined organics were dried over Na₂SO₄, filtered, and concentrated to afford (E)-10-((trans-4-(diethylamino)cyclohexyl)(ethyl)amino)-3-methyl-5,6,15,16-tetrahydrobenzo[c]pyrido[4,3-j][1]azacyclododecine-1,14(2H,9H)-dione (37 mg, 0.073 mmol, 27.4% yield) as a white solid. LC-MS (ES) m/z=504.9 [M+H]⁺. ¹H NMR (DMSO-d₆) δ: 11.29 (br. s., 1H), 7.99 (t, J=4.7 Hz, 1H), 7.14-7.21 (m, 2H), 6.90-6.97 (m, 1H), 5.84 (s, 1H), 5.07-5.22 (m, 2H), 4.18 (br. s., 2H), 3.51 (br. s., 2H), 2.93-3.05 (m, 2H), 2.53-2.69 (m, 3H), 2.31-2.47 (m, 5H), 2.22 (br. s., 2H), 2.11 (s, 3H), 1.62-1.80 (m, 4H), 1.18-1.33 (m, 2H), 1.04-1.18 (m, 2H), 0.91 (t, J=6.9 Hz, 6H), 0.75 (t, J=6.9 Hz, 3H).

### Example 50

### (E)-10-(ethyl(trans-4-morpholinocyclohexyl)amino)-3-methyl-5,6,15,16-tetrahydrobenzo[c]pyrido[4,3-j][1]azacyclododecine-1,14(2H,9H)-dione

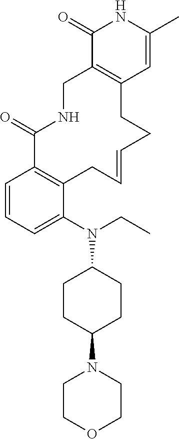 [SMILES: CCN(c1cccc2c1C/C=C/CCc1cc(C)[nH]c(=O)c1CNC2=O)[C@H]1CC[C@H](N2CCOCC2)CC1]

A solution of (E)-10-((trans-4-aminocyclohexyl)(ethyl)amino)-3-methyl-5,6,15,16-tetrahydrobenzo[c]pyrido[4,3-j][1]azacyclododecine-1,14(2H,9H)-dione, hydrochloride (65 mg, 0.134 mmol), 1-bromo-2-(2-bromoethoxy)ethane (0.046 mL, 0.270 mmol), DIEA (0.5 mL, 2.86 mmol) in CH₃CN (4 mL) was stirred at 60° C. overnight. The reaction mixture was concentrated, then purified by reverse-phase HPLC (25-55% CH₃CN in 0.1% aqueous NH₄OH) then concentrated to ca. 20 mL and extracted with EtOAc (3×50 mL), the combined organics were dried over Na₂SO₄, filtered, concentrated to afford (E)-10-(ethyl(trans-4-morpholinocyclohexyl)amino)-3-methyl-5,6,15,16-tetrahydrobenzo[c]pyrido[4,3-j][1]azacyclododecine-1,14(2H,9H)-dione (27 mg, 0.052 mmol, 38.8% yield) as a white solid. LC-MS (ES) m/z=260.3 (major), 519.5 [M+H]⁺ (minor). ¹H NMR (DMSO-d₆) δ: 11.29 (s, 1H), 7.99 (t, J=4.8 Hz, 1H), 7.18 (d, J=4.3 Hz, 2H), 6.93 (t, J=4.3 Hz, 1H), 5.84 (s, 1H), 5.07-5.22 (m, 2H), 4.15 (br. s., 2H), 3.52 (br. s., 6H), 2.94-3.03 (m, 2H), 2.56-2.71 (m, 2H), 2.40 (br. s., 4H), 2.21 (br. s., 2H), 2.11 (s, 3H), 2.02-2.10 (m, 1H), 1.77 (br. s., 4H), 1.18-1.33 (m, 2H), 1.01-1.15 (m, 2H), 0.75 (t, J=6.9 Hz, 3H).

### Example 51

### (E)-10-((1-(1,3-dihydroxypropan-2-yl)piperidin-4-yl)(ethyl)amino)-3-methyl-5,6,15,16-tetrahydrobenzo[c]pyrido[4,3-j][1]azacyclododecine-1,14(2H,9H)-dione

### (a) (E)-10-(ethyl(piperidin-4-yl)amino)-1-methoxy-3-methyl-5,6,15,16-tetrahydrobenzo[c]pyrido[4,3-j][1]azacyclododecin-14(9H)-one

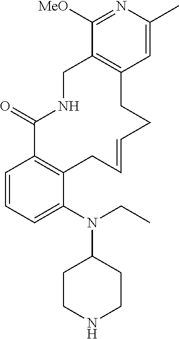 [SMILES: CCN(c1cccc2c1C/C=C/CCc1cc(C)nc(OC)c1CNC2=O)C1CCNCC1]

To a degassed solution of tert-butyl 4-((2-allyl-3-(((4-(but-3-en-1-yl)-2-methoxy-6-methylpyridin-3-yl)methyl)carbamoyl)phenyl)(ethyl)amino)piperidine-1-carboxylate (1.04 g, 1.803 mmol) in DCM (80 mL) was added Grubbs II (0.230 g, 0.270 mmol), and the reaction mixture was stirred at room temperature overnight under nitrogen. Additional Grubbs II (50 mg) was added and the reaction was stirred for another 5 h. The reaction mixture was concentrated, and purified by silica (CombiFlash®, 30 g column, 0-30% EtOAc in hexane) to afford a mixture of olefin isomers. The resulting mixture was purified by HPLC (0.1% TFA in mobile phase; 25-55% CH₃CN in water). The purified material was dissolved in DCM (10 mL) and TFA (3 mL, 38.9 mmol) was added and the reaction mixture was stirred at room temperature for 2 h. The reaction mixture was concentrated, neutralized with saturated aqueous NaHCO₃ and a white precipitate formed. The solid was filtered and washed with water to afford (E)-10-(ethyl(piperidin-4-yl)amino)-1-methoxy-3-methyl-5,6,15,16-tetrahydrobenzo[c]pyrido[4,3-j][1]azacyclododecin-14(9H)-one (529 mg, 1.179 mmol, 65.4% yield) as a white solid. LC-MS (ES) m/z=449.3 [M+H]⁺.

### (b) (E)-10-((1-(1,3-dihydroxypropan-2-yl)piperidin-4-yl)(ethyl)amino)-3-methyl-5,6,15,16-tetrahydrobenzo[c]pyrido[4,3-j][1]azacyclododecine-1,14(2H,9H)-dione

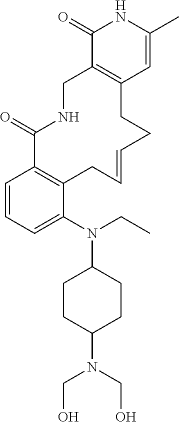 [SMILES: CCN(c1cccc2c1C/C=C/CCc1cc(C)[nH]c(=O)c1CNC2=O)C1CCC(N(CO)CO)CC1]

To a solution of (E)-10-(ethyl(piperidin-4-yl)amino)-1-methoxy-3-methyl-5,6,15,16-tetrahydrobenzo[c]pyrido[4,3-j][1]azacyclododecin-14(9H)-one (100 mg, 0.223 mmol) in DCM (3 mL) was added Et₃N (0.093 mL, 0.669 mmol) and dimethyl 2-bromomalonate (0.044 mL, 0.334 mmol). The reaction mixture was stirred at room temperature overnight. The reaction mixture was diluted with DCM, washed with water, brine and dried over Na₂SO₄, concentrated to dryness to afford a light brown wax. The residue was dissolved in THF (4 mL), cooled to 0° C. in an ice-bath, then LiAlH₄ (2 M, THF, 0.334 mL, 0.669 mmol) dropwise. After 10 min, the ice-bath was removed and the reaction was allowed to warm to room temperature under nitrogen, and stirred over the weekend. The reaction mixture was quenched with water (0.2 mL), filtered through a pad of Celite® and rinsed with MeOH and concentrated. The residue was purified by HPLC (0.1% TFA in mobile phase; 10-35% CH₃CN in water) to afford a residue. The residue was dissolved in 1,4-dioxane (3 mL) and MeOH (1 mL), then HCl (4 M, dioxane, 1 mL) was added. The reaction mixture was heated at 70° C. overnight, then concentrated down to dryness and the residue was passed through a 500 mg of Silicycle (carbonate) cartridge eluting with MeOH (20 mL) to afford (E)-10-((1-(1,3-dihydroxypropan-2-yl)piperidin-4-yl)(ethyl)amino)-3-methyl-5,6,15,16-tetrahydrobenzo[c]pyrido[4,3-j][1]azacyclododecine-1,14(2H,9H)-dione (18 mg, 0.035 mmol, 15.9% yield) as an off-white solid. LC-MS (ES) m/z=255.3 (major), 509.5 [M+H]⁺ (minor). ¹H NMR (400 MHz, METHANOL-d₄) δ: 7.19-7.32 (m, 2H), 7.07 (dd, J=1.5, 7.3 Hz, 1H), 6.12 (s, 1H), 5.27-5.42 (m, 1H), 5.15-5.27 (m, 1H), 4.38 (s, 2H), 3.54-3.75 (m, 6H), 3.07 (q, J=6.9 Hz, 2H), 2.84-2.94 (m, 2H), 2.66-2.83 (m, 3H), 2.59 (quin., J=6.1 Hz, 1H), 2.29-2.49 (m, 4H), 2.26 (s, 3H), 1.75 (br. s., 2H), 1.46-1.62 (m, 2H), 0.82 (t, J=7.0 Hz, 3H). Note: exchangeable Hs not observed.

### Example 52

### (Z)-10-(ethyl(piperidin-4-yl)amino)-3-methyl-5,6,15,16-tetrahydrobenzo[c]pyrido[4,3-j][1]azacyclododecine-1,14(2H,9H)-dione

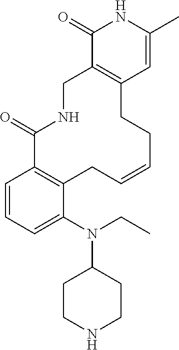 [SMILES: CCN(c1cccc2c1C/C=C\CCc1cc(C)[nH]c(=O)c1CNC2=O)C1CCNCC1]

To a degassed solution of tert-butyl 4-((2-allyl-3-(((4-(but-3-en-1-yl)-2-methoxy-6-methylpyridin-3-yl)methyl)carbamoyl)phenyl)(ethyl)amino)piperidine-1-carboxylate (6.7 g, 11.62 mmol) in DCM (400 mL) was added Grubbs II (1.479 g, 1.742 mmol), the reaction mixture was stirred at room temperature overnight under nitrogen. The reaction mixture was adsorbed onto Celite®, and purified by silica (CombiFlash®, 50 g column, 0-30% EtOAc in hexane) to afford a mixture of olefin isomers. The resulting mixture was triturated with EtOAc to afford a white solid which was further purified by HPLC (0.1% TFA in mobile phase; 25-55% CH₃CN in water) to afford 95 mg of a residue. The residue was dissolved in dioxane (3 mL) and MeOH (1.5 mL), and then HCl (4 M, dioxane, 1.5 mL, 6.00 mmol) was added. The resulting mixture was heated at 70° C. over the weekend. The reaction mixture was concentrated and the residue was basified with 15 mL of 30% NH₄OH/MeOH and concentrated, and purified by silica (CombiFlash®, 4 g column, 60-100% (1% NH₄OH+9% MeOH+90% CHCl₃)/CHCl₃) to afford (Z)-10-(ethyl(piperidin-4-yl)amino)-3-methyl-5,6,15,16-tetrahydrobenzo[c]pyrido[4,3-j][1]azacyclododecine-1,14(2H,9H)-dione (61 mg, 0.140 mmol) as a white solid. LC-MS (ES) m/z=435.4 [M+H]⁺. ¹H NMR (DMSO-d₆) δ: 10.87-11.97 (m, 1H), 8.15 (t, J=5.4 Hz, 1H), 7.13-7.28 (m, 2H), 7.0 (dd, J=6.8, 1.8 Hz, 1H), 5.94 (s, 1H), 5.02-5.25 (m, 2H), 4.35 (br. s., 2H), 3.63 (br. s., 2H), 3.01 (q, J=6.9 Hz, 2H), 2.89 (d, J=12.1 Hz, 2H), 2.54-2.79 (m, 3H), 2.22-2.43 (m, 4H), 2.12 (s, 3H), 1.57-1.68 (m, 2H), 1.17-1.39 (m, 2H), 0.78 (t, J=6.9 Hz, 3H). 1H not observed.

### Example 53

### (E)-11-((trans-4-(dimethylamino)cyclohexyl)(ethyl)amino)-3-methyl-5,6,7,10,16,17-hexahydro-1H-benzo[c]pyrido[4,3-k][1]azacyclotridecine-1,15(2H)-dione

### (a) tert-butyl(trans-4-((2-allyl-3-(((2-methoxy-6-methyl-4-(pent-4-en-1-yl)pyridin-3-yl)methyl)carbamoyl)phenyl)(ethyl)amino)cyclohexyl)carbamate

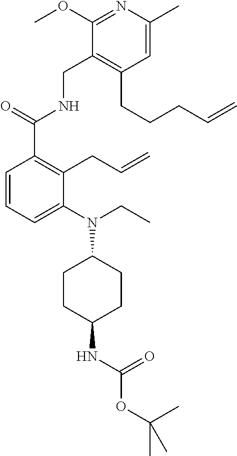 [SMILES: C=CCCCc1cc(C)nc(OC)c1CNC(=O)c1cccc(N(CC)[C@H]2CC[C@H](NC(=O)OC(C)(C)C)CC2)c1CC=C]

EDC (441 mg, 2.300 mmol) and HOAt (321 mg, 2.358 mmol) were added to a solution of 2-allyl-3-((trans-4-((tert-butoxycarbonyl)amino)cyclohexyl)(ethyl)amino)benzoic acid (618 mg, 1.535 mmol), (2-methoxy-6-methyl-4-(pent-4-en-1-yl)pyridin-3-yl)methanamine (405 mg, 1.838 mmol) and NMM (0.5 mL, 4.60 mmol) in DCM (7 mL) at room temperature and stirred for 2 h at room temperature. Water (50 mL) and CHCl₃ (50 mL) were added, the layers separated, and the organic layer dried over Na₂SO₄, adsorbed onto silica and purified via column chromatography (CombiFlash® Rf, 0-40% EtOAc in hexanes, 40 g column) to afford tert-butyl(trans-4-((2-allyl-3-(((2-methoxy-6-methyl-4-(pent-4-en-1-yl)pyridin-3-yl)methyl)carbamoyl)phenyl)(ethyl)amino)cyclohexyl)carbamate (685 mg, 1.133 mmol, 73.8% yield) as a colorless oil. LC-MS (ES) m/z=605.5 [M+H]⁺.

### (b) tert-butyl(trans-4-(ethyl((E)-1-methoxy-3-methyl-15-oxo-6,7,10,15,16,17-hexahydro-5H-benzo[c]pyrido[4,3-k][1]azacyclotridecin-11-yl)amino)cyclohexyl)carbamate

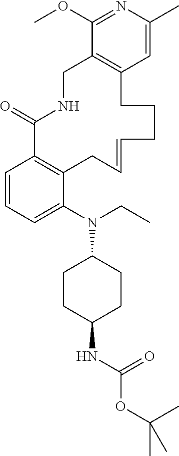 [SMILES: CCN(c1cccc2c1C/C=C/CCCc1cc(C)nc(OC)c1CNC2=O)[C@H]1CC[C@H](NC(=O)OC(C)(C)C)CC1]

To a solution of tert-butyl(trans-4-((2-allyl-3-(((2-methoxy-6-methyl-4-(pent-4-en-1-yl)pyridin-3-yl)methyl)carbamoyl)phenyl)(ethyl)amino)cyclohexyl)carbamate (612 mg, 1.012 mmol) in DCM (101.00 mL) degassed with argon was added Grubbs II (172 mg, 0.202 mmol) and the reaction was stirred for 24 h at room temperature. Water (500 mL) and CHCl₃ (2×500 mL) were added, the layers separated, and the organic layer dried over Na₂SO₄, adsorbed onto silica and purified via column chromatography (CombiFlash® Rf, 0-40% EtOAc in hexanes; 24 g column) to afford tert-butyl(trans-4-(ethyl((E)-1-methoxy-3-methyl-15-oxo-6,7,10,15,16,17-hexahydro-5H-benzo[c]pyrido[4,3-k][1]azacyclotridecin-11-yl)amino)cyclohexyl)carbamate (217 mg, 0.376 mmol, 37.2% yield) as an off-white solid. LC-MS (ES) m/z=141.9 (major), 380.2 (minor), 577.4 [M+H]⁺ (minor).

### (c) (E)-11-((trans-4-aminocyclohexyl)(ethyl)amino)-3-methyl-5,6,7,10,16,17-hexahydro-1H-benzo[c]pyrido[4,3-k][1]azacyclotridecine-1,15(2H)-dione

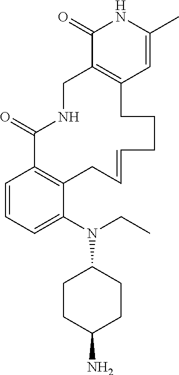 [SMILES: CCN(c1cccc2c1C/C=C/CCCc1cc(C)[nH]c(=O)c1CNC2=O)[C@H]1CC[C@H](N)CC1]

HCl (4 M, dioxane, 5 mL, 20.00 mmol) was added to a reaction vessel containing tert-butyl(trans-4-(ethyl((E)-1-methoxy-3-methyl-15-oxo-6,7,10,15,16,17-hexahydro-5H-benzo[c]pyrido[4,3-k][1]azacyclotridecin-11-yl)amino)cyclohexyl)carbamate (217 mg, 0.376 mmol) in MeOH (1 mL). The mixture was stirred for 48 h at 60° C. The mixture was concentrated to dryness, re-dissolved in MeOH (1 mL), adsorbed onto silica and purified via column chromatography (CombiFlash® Rf, 0-50% [80:20:2 CHCl₃/MeOH/NH₄OH] in CHCl₃; 4 g column) to afford (E)-11-((trans-4-aminocyclohexyl)(ethyl)amino)-3-methyl-5,6,7,10,16,17-hexahydro-1H-benzo[c]pyrido[4,3-k][1]azacyclotridecine-1,15(2H)-dione (150 mg, 0.324 mmol, 86% yield) as a white solid. LC-MS (ES) m/z=463.3 [M+H]⁺.

### (d) (E)-11-((trans-4-(dimethylamino)cyclohexyl)(ethyl)amino)-3-methyl-5,6,7,10,16,17-hexahydro-1H-benzo[c]pyrido[4,3-k][1]azacyclotridecine-1,15(2H)-dione

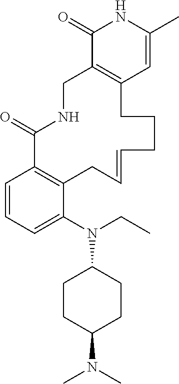 [SMILES: CCN(c1cccc2c1C/C=C/CCCc1cc(C)[nH]c(=O)c1CNC2=O)[C@H]1CC[C@H](N(C)C)CC1]

AcOH (15 μL, 0.262 mmol) and then Na(OAc)₃BH (114 mg, 0.538 mmol) were added to a solution of (E)-11-((trans-4-aminocyclohexyl)(ethyl)amino)-3-methyl-5,6,7,10,16,17-hexahydro-1H-benzo[c]pyrido[4,3-k][1]azacyclotridecine-1,15(2H)-dione (75 mg, 0.162 mmol) and formaldehyde (130 μL, 1.746 mmol) in MeOH (1.4 mL) at room temperature and stirred for 1 h. Caution: fairly vigorous bubbling! NaHCO₃ was added until the mixture was basic; the mixture was extracted with CHCl₃ and dried over Na₂SO₄. The solvent was removed and the resulting oil placed under high vacuum overnight to afford (E)-11-((trans-4-(dimethylamino)cyclohexyl)(ethyl)amino)-3-methyl-5,6,7,10,16,17-hexahydro-1H-benzo[c]pyrido[4,3-k][1]azacyclotridecine-1,15(2H)-dione (70.3 mg, 0.136 mmol, 84% yield) as a white solid. LC-MS (ES) m/z=246.2 (major), 366.1 (minor), 491.3 [M+H]⁺ (minor). ¹H NMR (400 MHz, DMSO-d6) δ: 11.42 (s, 1H), 7.94 (t, J=4.2 Hz, 1H), 7.13-7.26 (m, 2H), 6.99-7.06 (m, 1H), 5.87 (s, 1H), 5.40 (dt, J=15.4, 5.3 Hz, 1H), 4.94-5.06 (m, 1H), 4.32 (d, J=4.0 Hz, 2H), 3.79 (br. s., 2H), 2.97-3.05 (m, 2H), 2.64 (t, J=11.5 Hz, 1H), 2.25-2.35 (m, 2H), 1.99-2.18 (m, 10H), 1.91 (br. s., 2H), 1.76 (d, J=9.6 Hz, 4H), 1.50 (br. s., 2H), 1.20-1.35 (m, 2H), 1.04 (q, J=11.5 Hz, 2H), 0.77 (t, J=6.9 Hz, 3H).

### Example 54

### 9-(ethyl(piperidin-4-yl)amino)-3-methyl-5,8,14,15-tetrahydro-1H-benzo[c]pyrido[4,3-i][1]azacycloundecine-1,13(2H)-dione

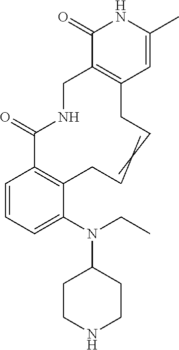 [SMILES: CCN(c1cccc2c1CC=CCc1cc(C)[nH]c(=O)c1CNC2=O)C1CCNCC1]

To the degassed solution of tert-butyl 4-((2-allyl-3-(((4-(but-3-en-1-yl)-2-methoxy-6-methylpyridin-3-yl)methyl)carbamoyl)phenyl)(ethyl)amino)piperidine-1-carboxylate (6.7 g, 11.62 mmol) in DCM (400 mL) was added Grubbs II (1.479 g, 1.742 mmol), the reaction mixture was stirred at room temperature overnight under nitrogen. The reaction mixture was concentrated down with Celite®, and purified by silica (CombiFlash®, 50 g column, 0-30% EtOAc in hexane) to afford a mixture. The resulting mixture was triturated with EtOAc to afford 2.8 g of a white solid, which was further purified by Gilson® HPLC (25-55% CH₃CN in water, 0.1% TFA) to afford 50 mg of a residue. To a solution of the residue in 1,4-dioxane (3 mL) and MeOH (1.5 mL) was added HCl (4 M in 1,4-dioxane, 1.5 mL, 6.00 mmol). The resulting mixture was heated at 70° C. overnight. The reaction mixture was concentrated down to dryness. The residue was passed through a 1 g of Silicycle® (carbonate) cartridge eluting with MeOH (35 mL). The resulting residue was triturated with EtOAc to afford 9-(ethyl(piperidin-4-yl)amino)-3-methyl-5,8,14,15-tetrahydro-1H-benzo[c]pyrido[4,3-i][1]azacycloundecine-1,13(2H)-dione (10 mg, 0.024 mmol) as an off white solid. ¹H NMR (400 MHz, DMSO-d₆) δ: 11.43 (br. s., 1H), 8.19 (t, J=4.93 Hz, 1H), 7.13-7.25 (m, 2H), 6.91 (dd, J=1.77, 6.82 Hz, 1H), 5.94 (s, 1H), 5.31-5.43 (m, 1H), 5.08-5.31 (m, 1H), 4.45 (d, J=5.05 Hz, 2H), 3.60 (d, J=8.08 Hz, 2H), 2.98-3.11 (m, 3H), 2.86-2.96 (m, 2H), 2.69-2.86 (m, 1H), 2.27-2.41 (m, 2H), 2.11 (s, 3H), 1.64 (br. s., 2H), 1.37 (br. s., 2H), 0.81 (t, J=6.95 Hz, 3H). 2H not observed. Stereochemistry of the olefin was not clearly identified.

### Example 55

### (E)-10-((cis-4-(dimethylamino)cyclohexyl)(ethyl)amino)-3-methyl-5,6,15,16-tetrahydrobenzo[c]pyrido[4,3-j][1]azacyclododecine-1,14(2H,9H)-dione

### (a) methyl 2-allyl-3-((cis-4-((tert-butoxycarbonyl)amino)cyclohexyl)amino)benzoate

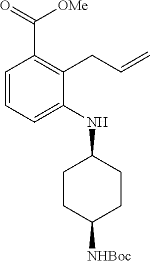 [SMILES: C=CCc1c(N[C@H]2CC[C@@H](NC(=O)OC(C)(C)C)CC2)cccc1C(=O)OC]

To a stirred solution of methyl 2-allyl-3-aminobenzoate (2 g, 10.46 mmol) and tert-butyl(4-oxocyclohexyl)carbamate (4.46 g, 20.92 mmol) in DCE (50 mL) was added AcOH (0.599 mL, 10.46 mmol). The reaction was stirred for 2 h at room temperature, then Na(OAc)₃BH (4.43 g, 20.92 mmol) was added portionwise and the reaction was stirred overnight at room temperature. The reaction was diluted with DCM (200 mL) and washed sequentially with saturated aqueous NaHCO₃ solution (50 mL) and brine (50 mL). The organic layer was dried over Na₂SO₄, filtered, concentrated and the residue was purified by flash column chromatography (0-15% EtOAc in hexanes, 400-g column, mixed fractions re-columned) to afford methyl 2-allyl-3-((cis-4-((tert-butoxycarbonyl)amino)cyclohexyl)amino)benzoate (1.43 g, 3.68 mmol, 35.2% yield) as a white solid. LC-MS (ES) m/z=389.0 [M+H]⁺.

### (b) methyl 2-allyl-3-((cis-4-((tertbutoxycarbonyl)amino)cyclohexyl)(ethyl)amino)benzoate

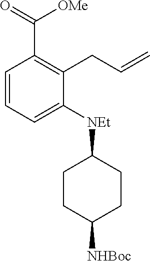

To methyl 2-allyl-3-((cis-4-((tert-butoxycarbonyl)amino)cyclohexyl)amino)benzoate (1.43 g, 3.68 mmol) in DCE (50 mL) were added acetaldehyde (1.247 mL, 22.08 mmol), AcOH (0.527 mL, 9.20 mmol) and Na(OAc)₃BH (2.65 g, 12.51 mmol) in that order. The reaction was then let stir at room temperature. After 3 h more acetaldehyde (0.1 g) and Na(OAc)₃BH (0.3 g) were added and let stir. After 1 h the reaction was diluted with DCM (50 mL) then washed with saturated aqueous NaHCO₃ solution then brine. The organic layer was dried over Na₂SO₄ then filtered and concentrated to give a light brown reddish oil. The oil was then purified by flash column chromatography (40 g Analogix® column conditioned with hexane then 2 min at 100% hexane then a gradient of 0-25% EtOAc in hexanes for 26 min) to afford methyl 2-allyl-3-((cis-4-((tert-butoxycarbonyl)amino)cyclohexyl)(ethyl)amino)benzoate (1.42 g, 3.41 mmol, 93% yield) as a clear oil upon concentration. LC-MS (ES) m/z=416.8 [M+H]⁺.

### (c) 2-allyl-3-((cis-4-((tert-butoxycarbonyl)amino)cyclohexyl)(ethyl)amino)benzoic acid

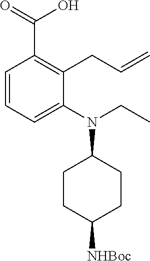 [SMILES: C=CCc1c(C(=O)O)cccc1N(CC)[C@H]1CC[C@@H](NC(=O)OC(C)(C)C)CC1]

To methyl 2-allyl-3-((cis-4-((tert-butoxycarbonyl)amino)cyclohexyl)(ethyl)amino)benzoate (1.42 g, 3.41 mmol) in MeOH (50 mL) was added aqueous NaOH (2.84 mL, 17.04 mmol). The reaction was then heated at 60° C. overnight. Additional aqueous NaOH (6 N, 500 uL) and THF (10 mL) were added to the reaction and the reaction was let stir overnight at 60° C. The reaction was concentrated then diluted with water (30 mL) then the pH was adjusted with aqueous HCl (6 N) until slightly acidic, and a white precipitate formed. The solid was then isolated by filtration to afford 2-allyl-3-((cis-4-((tert-butoxycarbonyl)amino)cyclohexyl)(ethyl)amino)benzoic acid (1.45 g). LC-MS (ES) m/z=403.0 [M+H]⁺.

### (d) tert-butyl(cis-4-((2-allyl-3-(04-(but-3-en-1-yl)-2-methoxy-6-methylpyridin-3-yl)methyl)carbamoyl)phenyl)(ethyl)amino)cyclohexyl)carbamate

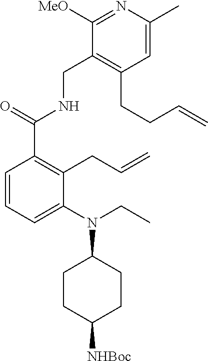 [SMILES: C=CCCc1cc(C)nc(OC)c1CNC(=O)c1cccc(N(CC)[C@H]2CC[C@@H](NC(=O)OC(C)(C)C)CC2)c1CC=C]

To 2-allyl-3-((cis-4-((tert-butoxycarbonyl)amino)cyclohexyl)(ethyl)amino)benzoic acid (908 mg, 2.256 mmol) in DMSO (15 mL) were added HOBt (518 mg, 3.38 mmol), EDC (649 mg, 3.38 mmol), N-methylmorpholine (0.744 mL, 6.77 mmol) and (4-(but-3-en-1-yl)-2-methoxy-6-methylpyridin-3-yl)methanamine (558 mg, 2.71 mmol). The reaction was then stirred overnight at room temperature. The reaction was transferred into water (50 mL) and a precipitate formed which was then isolated by filtration and dried by vacuum. The solid was then redissolved in DCM and then purified by flash column chromatography (hexanes for 3 min then 0-35% EtOAc in hexanes over 33 min, 50 g column) to afford tert-butyl(cis-4-((2-allyl-3-(((4-(but-3-en-1-yl)-2-methoxy-6-methylpyridin-3-yl)methyl)carbamoyl)phenyl)(ethyl)amino)cyclohexyl)carbamate (990 mg, 1.676 mmol, 74.3% yield) as a clear oil. LC-MS (ES) m/z=591.0 [M+H]⁺.

### (e) a mixture of (E) and (Z)-isomers of tert-butyl(cis-4-(ethyl(1-methoxy-3-methyl-14-oxo-5,6,9,14,15,16-hexahydrobenzo[c]pyrido[4,3-j][1]azacyclododecin-10-yl)amino)cyclohexyl)carbamate

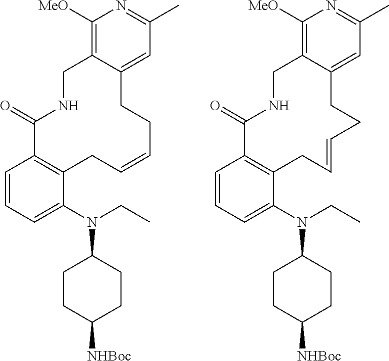 [SMILES: CCN(c1cccc2c1C/C=C\CCc1cc(C)nc(OC)c1CNC2=O)[C@H]1CC[C@@H](NC(=O)OC(C)(C)C)CC1 ; CCN(c1cccc2c1C/C=C/CCc1cc(C)nc(OC)c1CNC2=O)[C@H]1CC[C@@H](NC(=O)OC(C)(C)C)CC1]

Tert-butyl(cis-4-((2-allyl-3-(((4-(but-3-en-1-yl)-2-methoxy-6-methylpyridin-3-yl)methyl)carbamoyl)phenyl)(ethyl)amino)cyclohexyl)carbamate (706 mg, 1.195 mmol) was diluted with DCM (30 mL) then Hoveyda-Grubbs 2nd generation catalyst (150 mg, 0.239 mmol) was added. A condenser was fitted and the reaction heated at 55° C. overnight. After 48 h additional Hoveyda-Grubbs 2nd generation catalyst (150 mg, 0.239 mmol) was added and the reaction was allowed to stir. After a total of 72 h the reaction was concentrated then purified by flash column chromatography (hexane 3 min, DCM 2 min, 0 to 50% DCM:MeOH:NH₄OH (80:20:2) in DCM for 35 min, 40 g column) to afford a mixture of (E)- and (Z)-tert-butyl(cis-4-(ethyl(1-methoxy-3-methyl-14-oxo-5,6,9,14,15,16-hexahydrobenzo[c]pyrido[4,3-j][1]azacyclododecin-10-yl)amino)cyclohexyl)carbamate (389 mg, 0.691 mmol, 57.8% yield) as a solid. LC-MS (ES) m/z=563.0 [M+H]⁺.

### (f) (E)-10-((cis-4-aminocyclohexyl)(ethyl)amino)-3-methyl-3,4,5,6,15,16-hexahydrobenzo[c]pyrido[4,3-j][1]azacyclododecine-1,14(2H,9H)-dione

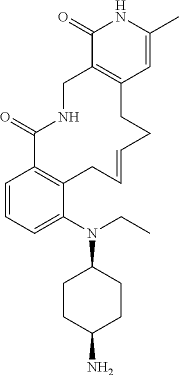 [SMILES: CCN(c1cccc2c1C/C=C/CCc1cc(C)[nH]c(=O)c1CNC2=O)[C@H]1CC[C@@H](N)CC1]

An E/Z mixture of tert-butyl(cis-4-(ethyl(1-methoxy-3-methyl-14-oxo-5,6,9,14,15,16-hexahydrobenzo[c]pyrido[4,3-j][1]azacyclododecin-10-yl)amino)cyclohexyl)carbamate (363 mg, 0.645 mmol) was dissolved in 1,4-dioxane (5 mL) and MeOH (2 mL), and then HCl (4 M in 1,4-dioxane, 2.5 mL) was added. The reaction mixture was heated at 70° C. overnight forming a white precipitate. The reaction mixture was concentrated down to dryness, and the residue was treated with NH₄OH (30% in MeOH, 10 mL), then concentrated, and this procedure was repeated one more time. The residue was then concentrated down with Celite® and purified by flash column chromatography (50-100% (1% NH₄OH+9% MeOH+90% CHCl₃) in CHCl₃, 4 g column) to afford (E)-10-((cis-4-aminocyclohexyl)(ethyl)amino)-3-methyl-3,4,5,6,15,16-hexahydrobenzo[c]pyrido[4,3-j][1]azacyclododecine-1,14(2H,9H)-dione (164 mg, 0.364 mmol, 51.2% yield) as a white solid. LC-MS (ES) m/z=449.4 [M+H]⁺.

### (g) (E)-10-((cis-4-(dimethylamino)cyclohexyl)(ethyl)amino)-3-methyl-5,6,15,16-tetrahydrobenzo[c]pyrido[4,3-j][1]azacyclododecine-1,14(2H,9H)-dione

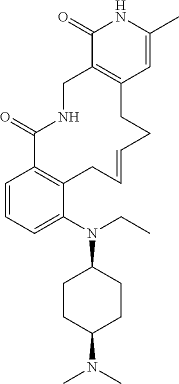 [SMILES: CCN(c1cccc2c1C/C=C/CCc1cc(C)[nH]c(=O)c1CNC2=O)[C@H]1CC[C@@H](N(C)C)CC1]

To (E)-10-((cis-4-aminocyclohexyl)(ethyl)amino)-3-methyl-5,6,15,16-tetrahydrobenzo[c]pyrido[4,3-j][1]azacyclododecine-1,14(2H,9H)-dione (95 mg, 0.212 mmol) in MeOH (3 mL) was added formaldehyde (37 wt. % solution in water, 0.236 mL, 3.18 mmol), AcOH (0.024 mL, 0.424 mmol) and NaBH₃CN (133 mg, 2.118 mmol). The reaction mixture was stirred for 1 h. The reaction mixture was treated with saturated aqueous NaHCO₃ (2 mL) for 10 min, adsorbed onto silica, then purified by flash column chromatography (50-100% (1% NH₄OH+9% MeOH+90% CHCl₃) in CHCl₃, 4 g column) to afford a solid that was further purified by Gilson® HPLC (10-40% CH₃CN in water, 0.1% TFA in mobile phase). The resulting fractions were concentrated and the residue was passed through a 1 g Silicycle® (carbonate) cartridge eluting with MeOH (30 mL) to afford (E)-10-((cis-4-(dimethylamino)cyclohexyl)(ethyl)amino)-3-methyl-5,6,15,16-tetrahydrobenzo[c]pyrido[4,3-j][1]azacyclododecine-1,14(2H,9H)-dione (68 mg, 0.143 mmol, 67.4% yield) as a white solid. LC-MS (ES) m/z=477.4 [M+H]⁺. ¹H NMR (400 MHz, METHANOL-d₄) δ: 7.22-7.33 (m, 2H), 7.12 (dd, J=1.52, 7.07 Hz, 1H), 6.04-6.18 (m, 1H), 5.18-5.41 (m, 2H), 4.43 (br. s., 1H), 4.35 (br. s., 1H), 3.66-3.83 (m, 2H), 3.46-3.59 (m, 1H), 3.07-3.22 (m, 1H), 2.95-3.05 (m, 1H), 2.79-2.94 (m, 1H), 2.64-2.80 (m, 8H), 2.34 (br. s., 2H), 2.26 (s, 4H), 1.85-2.03 (m, 1H), 1.48-1.89 (m, 4H), 1.42 (br. s., 2H), 0.80-0.95 (m, 3H). 2H not observed.

### Example 56

### (E)-10-((trans-4-((2,2-difluoroethyl)amino)cyclohexyl)(ethyl)amino)-3-methyl-5,6,15,16-tetrahydrobenzo[c]pyrido[4,3-j][1]azacyclododecine-1,14(2H,9H)-dione

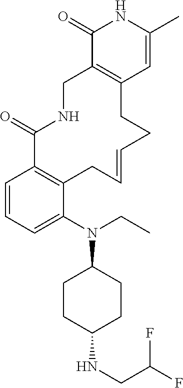 [SMILES: CCN(c1cccc2c1C/C=C/CCc1cc(C)[nH]c(=O)c1CNC2=O)[C@H]1CC[C@H](NCC(F)F)CC1]

To the reaction mixture of (E)-10-((trans-4-aminocyclohexyl)(ethyl)amino)-3-methyl-5,6,15,16-tetrahydrobenzo[c]pyrido[4,3-j][1]azacyclododecine-1,14(2H,9H)-dione (130 mg, 0.290 mmol) in CH₃CN (3.00 mL) was added 2,2-difluoroethyl trifluoromethanesulfonate (186 mg, 0.869 mmol) and DIPEA (0.202 mL, 1.159 mmol). The reaction mixture was stirred at 70° C. for 2 h. The reaction mixture was concentrated and purified by Gilson® HPLC (5-40% CH₃CN in water, 0.1% TFA in mobile phase). The resulting fractions were concentrated and the residue was passed through a 1 g Silicycle® (carbonate) cartridge eluting with MeOH (30 mL) to afford (E)-10-((trans-4-((2,2-difluoroethyl)amino)cyclohexyl)(ethyl)amino)-3-methyl-5,6,15,16-tetrahydrobenzo[c]pyrido[4,3-j][1]azacyclododecine-1,14(2H,9H)-dione (70 mg, 0.137 mmol, 47.1% yield) as a white solid. LC-MS (ES) m/z=513.4 [M+H]⁺. ¹H NMR (400 MHz, DMSO-d₆) δ: 11.29 (s, 1H), 7.98 (t, J=4.93 Hz, 1H), 7.11-7.24 (m, 2H), 6.87-6.99 (m, 1H), 5.83 (s, 2H), 5.05-5.24 (m, 2H), 4.18 (br. s., 1H), 3.85-4.14 (m, 1H), 3.52 (br. s., 2H), 2.94-3.05 (m, 2H), 2.78-2.93 (m, 2H), 2.57-2.69 (m, 2H), 2.51-2.57 (m, 2H), 2.33 (br. s., 1H), 2.16-2.24 (m, 2H), 2.11 (s, 3H), 1.79-1.91 (m, 2H), 1.63-1.78 (m, 2H), 1.17-1.31 (m, 2H), 0.83-1.00 (m, 2H), 0.75 (t, J=6.95 Hz, 3H).

### Example 57

### (E)-10-((trans-4-((2,2-difluoroethyl)(methyl)amino)cyclohexyl)(ethyl)amino)-3-methyl-5,6,15,16-tetrahydrobenzo[c]pyrido[4,3-j][1]azacyclododecine-1,14(2H,9H)-dione

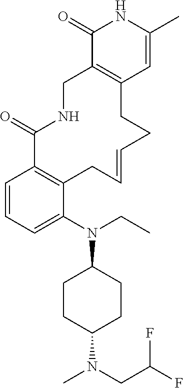 [SMILES: CCN(c1cccc2c1C/C=C/CCc1cc(C)[nH]c(=O)c1CNC2=O)[C@H]1CC[C@H](N(C)CC(F)F)CC1]

To a microwave tube containing (E)-10-((trans-4-aminocyclohexyl)(ethyl)amino)-3-methyl-5,6,15,16-tetrahydrobenzo[c]pyrido[4,3-j][1]azacyclododecine-1,14(2H,9H)-dione (130 mg, 0.290 mmol) in THF (3 mL) was added 2,2-difluoroethyl trifluoromethanesulfonate (124 mg, 0.580 mmol), and TEA (0.162 mL, 1.159 mmol). The reaction vessel was capped and stirred at 70° C. for 2 h. The reaction mixture was concentrated to afford an off white solid. To this solid in MeOH (3.00 mL) was added Na(OAc)₃BH (184 mg, 0.869 mmol), formaldehyde (37 wt % in water, 0.080 mL, 2.90 mmol) and AcOH (0.050 mL, 0.869 mmol). The reaction mixture was stirred at room temperature for 20 min. The reaction mixture was concentrated and purified by Gilson® HPLC (5-40% CH₃CN in water, 0.1% TFA in mobile phase). The resulting fractions were concentrated and the residue was passed through a 1 g of Silicycle® (carbonate) cartridge eluting with MeOH (30 mL) to afford (E)-10-((trans-4-((2,2-difluoroethyl)(methyl)amino)cyclohexyl)(ethyl)amino)-3-methyl-5,6,15,16-tetrahydrobenzo[c]pyrido[4,3-j][1]azacyclododecine-1,14(2H,9H)-dione (103 mg, 0.196 mmol, 67.5% yield) as a white solid. LC-MS (ES) m/z=527.4 [M+H]⁺ (minor), 264.3 (major). ¹H NMR (400 MHz, DMSO-d₆) δ: 11.31 (br. s., 1H), 10.24 (br. s., 1H), 7.96 (br. s., 1H), 7.21 (d, J=4.55 Hz, 2H), 6.88-7.06 (m, 1H), 6.15-6.78 (m, 1H), 5.84 (s, 1H), 5.02-5.26 (m, 2H), 4.17 (br. s., 2H), 3.54 (br. s., 3H), 3.09-3.30 (m, 1H), 2.99 (br. s., 2H), 2.61-2.88 (m, 4H), 2.54 (br. s., 1H), 2.22 (br. s., 2H), 2.11 (s, 3H), 1.90-2.01 (m, 2H), 1.86 (br. s., 2H), 1.40 (br. s., 2H), 1.21-1.37 (m, 3H), 0.75 (t, J=6.95 Hz, 3H).

### Example 58

### (E)-10-(ethyl(trans-4-((2,2,2-trifluoroethyl)amino)cyclohexyl)amino)-3-methyl-5,6,15,16-tetrahydrobenzo[c]pyrido[4,3-j][1]azacyclododecine-1,14(2H,9H)-dione

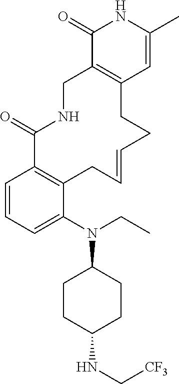 [SMILES: CCN(c1cccc2c1C/C=C/CCc1cc(C)[nH]c(=O)c1CNC2=O)[C@H]1CC[C@H](NCC(F)(F)F)CC1]

To a microwave tube containing (E)-10-((trans-4-aminocyclohexyl)(ethyl)amino)-3-methyl-5,6,15,16-tetrahydrobenzo[c]pyrido[4,3-j][1]azacyclododecine-1,14(2H,9H)-dione (100 mg, 0.223 mmol) in THF (3 mL) and N-methyl-2-pyrrolidone (0.3 mL) was added 2,2,2-trifluoroethyl trifluoromethanesulfonate (0.058 mL, 0.401 mmol) and TEA (0.124 mL, 0.892 mmol). The reaction vessel was capped and stirred at 70° C. overnight. The reaction mixture was concentrated and purified by Gilson® HPLC (5-40% CH₃CN in water, 0.1% TFA in mobile phase). The resulting fractions were concentrated and the residue was passed through a 1 g Silicycle® (carbonate) cartridge eluting with MeOH (30 mL) to afford (E)-10-(ethyl(trans-4-((2,2,2-trifluoroethyl)amino)cyclohexyl)amino)-3-methyl-5,6,15,16-tetrahydrobenzo[c]pyrido[4,3-j][1]azacyclododecine-1,14(2H,9H)-dione (105 mg, 0.198 mmol, 89% yield) as a white solid. LC-MS (ES) m/z=531.4 [M+H]⁺. ¹H NMR (400 MHz, DMSO-d₆) δ: 11.31 (br. s., 1H), 8.81-10.18 (m, 1H), 7.98 (br. s., 1H), 7.22 (br. s., 2H), 6.94-7.01 (m, 1H), 5.84 (s, 1H), 5.04-5.24 (m, 2H), 4.17 (br. s., 2H), 3.95 (br. s., 2H), 3.53 (br. s., 2H), 2.98 (br. s., 3H), 2.64 (br. s., 1H), 2.51-2.59 (m, 2H), 2.21 (br. s., 2H), 2.11 (s, 3H), 2.04 (br. s., 2H), 1.66-1.90 (m, 2H), 1.18-1.40 (m, 4H), 0.75 (t, J=6.95 Hz, 3H).

### Example 59

### (E)-10-(ethyl(trans-4-(methyl(2,2,2-trifluoroethyl)amino)cyclohexyl)amino)-3-methyl-5,6,15,16-tetrahydrobenzo[c]pyrido[4,3-j][1]azacyclododecine-1,14(2H,9H)-dione

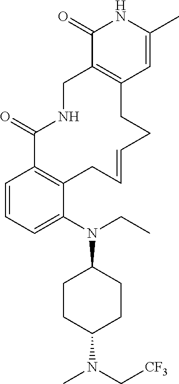 [SMILES: CCN(c1cccc2c1C/C=C/CCc1cc(C)[nH]c(=O)c1CNC2=O)[C@H]1CC[C@H](N(C)CC(F)(F)F)CC1]

To (E)-10-(ethyl(trans-4-((2,2,2-trifluoroethyl)amino)cyclohexyl)amino)-3-methyl-5,6,15,16-tetrahydrobenzo[c]pyrido[4,3-j][1]azacyclododecine-1,14(2H,9H)-dione (130 mg, 0.245 mmol) in MeOH (5 mL) was added Na(OAc)₃BH (208 mg, 0.980 mmol), formaldehyde (37 wt % in water, 0.182 mL, 2.450 mmol) and AcOH (0.042 mL, 0.735 mmol). The reaction mixture was stirred at room temperature for 90 min. The reaction mixture was concentrated and purified by silica column (0-50% (1% NH₄OH+9% MeOH+90% CHCl₃) in CHCl₃, 4 g column) to afford (E)-10-(ethyl(trans-4-(methyl(2,2,2-trifluoroethyl)amino)cyclohexyl)amino)-3-methyl-5,6,15,16-tetrahydrobenzo[c]pyrido[4,3-j][1]azacyclododecine-1,14(2H,9H)-dione (90 mg, 0.165 mmol, 67.4% yield) as a white solid. LC-MS (ES) m/z=545.4 [M+H]⁺ (minor), 273.3 (major). ¹H NMR (400 MHz, DMSO-d₆) δ: 11.30 (s, 1H), 7.96 (t, J=5.05 Hz, 1H), 7.13-7.28 (m, 2H), 6.85-7.01 (m, 1H), 5.84 (s, 1H), 5.03-5.25 (m, 2H), 4.17 (br. s., 2H), 3.52 (br. s., 2H), 3.07 (q, J=10.02 Hz, 2H), 2.92-3.02 (m, 2H), 2.51-2.70 (m, 3H), 2.31-2.41 (m, 1H), 2.29 (s, 3H), 2.22 (br. s., 2H), 2.11 (s, 3H), 1.73-1.82 (m, 2H), 1.64-1.73 (m, 2H), 1.06-1.32 (m, 4H), 0.75 (t, J=6.95 Hz, 3H).

### Example 60

### (E)-10-((trans-4-(azetidin-1-yl)cyclohexyl)(ethyl)amino)-3-methyl-5,6,15,16-tetrahydrobenzo[c]pyrido[4,3-j][1]azacyclododecine-1,14(2H,9H)-dione

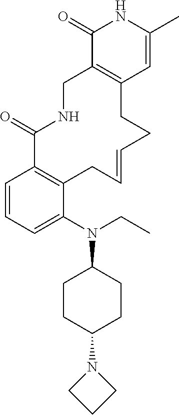 [SMILES: CCN(c1cccc2c1C/C=C/CCc1cc(C)[nH]c(=O)c1CNC2=O)[C@H]1CC[C@H](N2CCC2)CC1]

To a microwave tube containing (E)-10-((trans-4-aminocyclohexyl)(ethyl)amino)-3-methyl-5,6,15,16-tetrahydrobenzo[c]pyrido[4,3-j][1]azacyclododecine-1,14(2H,9H)-dione (130 mg, 0.290 mmol) in THF (3 mL) and N-methyl-2-pyrrolidone (0.5 mL) was added 1,3,2-dioxathiane 2,2-dioxide (120 mg, 0.869 mmol), and TEA (0.202 mL, 1.449 mmol). The reaction vessel was capped and stirred at 70° C. over the weekend. The reaction mixture was concentrated down and triturated with EtOAc to afford a crude yellow solid. To a microwave reaction vessel containing the above crude solid in water (15 mL) was added aqueous NaOH (1 N, 0.869 mL, 0.869 mmol). The reaction vessel was capped and heated at 150° C. for a 40 min in a Biotage Initiator® microwave reactor with the absorption level set to normal. After cooling, the cap was removed and the solution was transferred to a separatory funnel and extracted three times with DCM. The combined organic layers were dried over Na₂SO₄ and concentrated, then purified by flash column chromatography (0-100% (1% NH₄OH+9% MeOH+90% CHCl₃) in CHCl₃, 12 g column). The resulting fractions were concentrated and purified by Gilson® HPLC (5-40% CH₃CN in water, 0.1% TFA in mobile phase), and the resulting fractions were concentrated and the residue was passed through a 500 mg Silicycle® (carbonate) cartridge eluting with MeOH (20 mL) to afford (E)-10-((trans-4-(azetidin-1-yl)cyclohexyl)(ethyl)amino)-3-methyl-5,6,15,16-tetrahydrobenzo[c]pyrido[4,3-j][1]azacyclododecine-1,14(2H,9H)-dione (57 mg, 0.117 mmol, 40.3% yield) as a white solid. LC-MS (ES) m/z=489.4 [M+H]⁺ (minor), 245.3 (major). ¹H NMR (400 MHz, DMSO-d₆) δ: 11.30 (s, 1H), 10.14 (br. s., 1H), 7.99 (t, J=4.67 Hz, 1H), 7.13-7.28 (m, 2H), 6.97 (dd, J=2.53, 6.06 Hz, 1H), 5.83 (s, 1H), 5.01-5.24 (m, 2H), 4.17 (br. s., 2H), 3.90-4.07 (m, 4H), 3.52-3.74 (m, 2H), 2.87-3.11 (m, 3H), 2.58-2.76 (m, 1H), 2.54 (br. s., 1H), 2.30-2.43 (m, 1H), 2.06-2.27 (m, 6H), 1.84-1.94 (m, 3H), 1.81 (br. s., 1H), 1.13-1.30 (m, 2H), 0.92-1.11 (m, 2H), 0.75 (t, J=6.95 Hz, 3H).

### Example 61

### (Z)-9-((trans-4-(dimethylamino)cyclohexyl)(ethyl)amino)-3-methyl-5,8,14,15-tetrahydro-1H-benzo[c]pyrido[4,3-i][1]azacycloundecine-1,13(2H)-dione

### (a) tert-butyl(trans-4-(ethyl((Z)-1-methoxy-3-methyl-13-oxo-8,13,14,15-tetrahydro-5Hbenzo[c]pyrido[4,3-i][1]azacycloundecin-9-yl)amino)cyclohexyl)carbamate

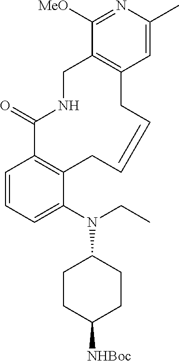 [SMILES: CCN(c1cccc2c1C/C=C\Cc1cc(C)nc(OC)c1CNC2=O)[C@H]1CC[C@H](NC(=O)OC(C)(C)C)CC1]

Hoveyda-Grubbs catalyst (920 mg, 1.468 mmol) was added to an argon degassed solution of tert-butyl(trans-4-((2-allyl-3-(((4-(but-3-en-1-yl)-2-methoxy-6-methylpyridin-3-yl)methyl)carbamoyl)phenyl)(ethyl)amino)cyclohexyl)carbamate (4.95 g, 8.38 mmol) in DCE (524 mL) at room temperature and stirred for 24 h at 50° C. H₂O (500 mL) and CHCl₃ (10 mL) were added, the layers separated organic layer dried over Na₂SO₄, adsorbed onto silica and purified via column chromatography (Isco CombiFlash® Rf, 0-50% EtOAc:hexanes; 12 g column), then further purified by HPLC (Chiralpak IF, 5 microns, 30 mm×250 mm, 80:20:0.1 n-heptane:EtOH:isopropylamine (isocratic)) to afford tert-butyl(trans-4-(ethyl((Z)-1-methoxy-3-methyl-13-oxo-8,13,14,15-tetrahydro-5H-benzo[c]pyrido[4,3-i][1]azacycloundecin-9-yl)amino)cyclohexyl)carbamate (631 mg, 1.150 mmol, 13.73% yield) as a white solid. LC-MS (ES) m/z=549.4 [M+H]⁺ (minor), 352.1 (major).

### (b) (Z)-9-((trans-4-aminocyclohexyl)(ethyl)amino)-3-methyl-5,8,14,15-tetrahydro-1H-benzo[c]pyrido[4,3-i][1]azacycloundecine-1,13(2H)-dione

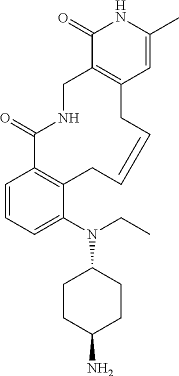 [SMILES: CCN(c1cccc2c1C/C=C\Cc1cc(C)[nH]c(=O)c1CNC2=O)[C@H]1CC[C@H](N)CC1]

HCl (4 M in 1,4-dioxane, 4 mL, 16.00 mmol) was added to a reaction vessel containing tert-butyl(trans-4-(ethyl((Z)-1-methoxy-3-methyl-13-oxo-8,13,14,15-tetrahydro-5Hbenzo[c]pyrido[4,3-i][1]azacycloundecin-9-yl)amino)cyclohexyl)carbamate (107 mg, 0.195 mmol) in MeOH (1 mL). The mixture was stirred for 48 h at 60° C., then concentrated to dryness, redissolved in MeOH (20 mL), adsorbed onto silica and purified via column chromatography (Isco CombiFlash® Rf, 0-50% (80:20:2 CHCl₃:MeOH:NH₄OH) in CHCl₃; 4 g column) to afford (Z)-9-((trans-4-aminocyclohexyl)(ethyl)amino)-3-methyl-5,8,14,15-tetrahydro-1H-benzo[c]pyrido[4,3-i][1]azacycloundecine-1,13(2H)-dione (74.7 mg, 0.172 mmol, 88% yield) as a white solid. LC-MS (ES) m/z=435 [M+H]⁺.

### (c) (Z)-9-((trans-4-(dimethylamino)cyclohexyl)(ethyl)amino)-3-methyl-5,8,14,15-tetrahydro-1H-benzo[c]pyrido[4,3-i][1]azacycloundecine-1,13(2H)-dione

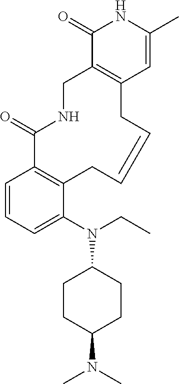 [SMILES: CCN(c1cccc2c1C/C=C\Cc1cc(C)[nH]c(=O)c1CNC2=O)[C@H]1CC[C@H](N(C)C)CC1]

AcOH (20 μL, 0.349 mmol) and then Na(OAc)₃BH (118 mg, 0.557 mmol) were added to a solution of (Z)-9-((trans-4-aminocyclohexyl)(ethyl)amino)-3-methyl-5,8,14,15-tetrahydro-1H-benzo[c]pyrido[4,3-i][1]azacycloundecine-1,13(2H)-dione (74.7 mg, 0.172 mmol) and formaldehyde (37 wt % in water, 130 μL, 1.746 mmol) in MeOH (1.2 mL) at room temperature and stirred for 1 h. (Caution: fairly vigorous bubbling!) NaHCO₃ was added until slightly basic, the mixture extracted with CHCl₃ (100 mL) and dried over Na₂SO₄. The mixture was concentrated to dryness, re-dissolved in MeOH (2 mL), adsorbed onto silica and purified via column chromatography (Isco CombiFlash® Rf, 0-50% 80:20:2 (CHCl₃:MeOH:NH₄OH) in CHCl₃; 4 g column) to afford (Z)-9-((trans-4-(dimethylamino)cyclohexyl)(ethyl)amino)-3-methyl-5,8,14,15-tetrahydro-1H-benzo[c]pyrido[4,3-i][1]azacycloundecine-1,13(2H)-dione (69.3 mg, 0.147 mmol, 85% yield) as a white solid. LC-MS (ES) m/z=463 [M+H]⁺ (minor), 232.2 (major). ¹H NMR (400 MHz, DMSO-d₆) δ: 11.43 (br. s., 1H) 8.20 (t, J=5.18 Hz, 1H) 7.07-7.30 (m, 2H) 6.91 (dd, J=6.82, 1.52 Hz, 1H) 5.94 (s, 1H) 5.31-5.43 (m, 1H) 5.19-5.31 (m, 1H) 4.42-4.47 (m, 2H) 3.54-3.62 (m, 2H) 3.34 (s, 2H) 3.99-3.08 (m, 2H) 2.60-2.70 (m, 1H) 1.98-2.19 (m, 10H) 1.68-1.89 (m, 4H) 1.30-1.44 (d, J=10.61 Hz, 2H) 0.98-1.22 (m, 2H) 0.81 (t, J=6.95 Hz, 3H).

### Example 62

### 9-((trans-4-(dimethylamino)cyclohexyl)(ethyl)amino)-3-methyl-5,6,7,8,14,15-hexahydro-1H-benzo[c]pyrido[4,3-i][1]azacycloundecine-1,13(2H)-dione

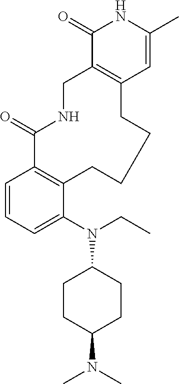 [SMILES: CCN(c1cccc2c1CCCCc1cc(C)[nH]c(=O)c1CNC2=O)[C@H]1CC[C@H](N(C)C)CC1]

Pd/C (10 wt %, 90 mg, 0.085 mmol) was added to a solution of (Z)-9-((trans-4-(dimethylamino)cyclohexyl)(ethyl)amino)-3-methyl-5,8,14,15-tetrahydro-1H-benzo[c]pyrido[4,3-i][1]azacycloundecine-1,13(2H)-dione (36 mg, 0.078 mmol) in EtOH (2 mL). The reaction was degassed several times by evacuating the flask and refilling with argon then placed under a balloon of hydrogen (excess) at room temperature and stirred for 24 h. After 24 h the reaction filtered, concentrated to dryness, re-dissolved in MeOH (2 mL), adsorbed onto silica and purified via column chromatography (Isco CombiFlash® Rf, 0-50% 80:20:2 [CHCl₃:MeOH:NH₄OH] in CHCl₃, 4 g column) to afford 9-((trans-4-(dimethylamino)cyclohexyl)(ethyl)amino)-3-methyl-5,6,7,8,14,15-hexahydro-1H-benzo[c]pyrido[4,3-i][1]azacycloundecine-1,13(2H)-dione (20 mg, 0.039 mmol, 49.8% yield) as a white solid. LC-MS (ES) m/z=465 [M+H]⁺ (minor), 233.2 (major). ¹H NMR (400 MHz, DMSO-d₆) δ: 11.37 (br. s., 1H) 8.27 (t, J=4.93 Hz, 1H) 7.08-7.23 (m, 2H) 6.92 (dd, J=7.07, 1.52 Hz, 1H) 5.83 (s, 1H) 4.43 (br. s., 2H) 2.93-3.09 (m, 2H) 2.83 (br. s., 2H) 2.54-2.68 (m, 2H) 2.22-2.45 (m, 1H) 2.07-2.12 (m, 10H) 1.68-1.88 (m, 4H) 1.58 (br. s., 2H) 1.27-1.51 (m, 4H) 0.99-1.18 (m, 2H) 0.76 (t, J=6.95 Hz, 3H).

### Example 63

### (E)-10-(ethyl(tetrahydro-2H-pyran-4-yl)amino)-3-methyl-5,6,15,16-tetrahydrodipyrido[3,4-c:3′,4′-j][1]azacyclododecine-1,14(2H,9H)-dione

### a) N-((4-(but-3-en-1-yl)-2-methoxy-6-methylpyridin-3-yl)methyl)-2-chloroisonicotinamide

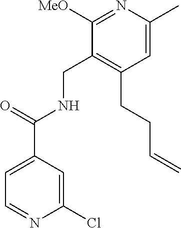 [SMILES: C=CCCc1cc(C)nc(OC)c1CNC(=O)c1ccnc(Cl)c1]

To a mixture of 2-chloroisonicotinic acid (0.62 g, 3.94 mmol), (4-(but-3-en-1-yl)-2-methoxy-6-methylpyridin-3-yl)methanamine (0.834 mL, 4.13 mmol), HOAt (0.643 g, 4.72 mmol), and EDC (0.905 g, 4.72 mmol) in DMF (20 mL) was added N-methylmorpholine (1.731 mL, 15.74 mmol) and all solids slowly dissolved. The reaction was allowed to stir at room temperature overnight. The reaction was diluted into water (100 mL) with stirring and then partitioned with 20% EtOAc in Et₂O (50 mL, 2×). The organics were combined and washed with brine, dried over MgSO₄, filtered and concentrated in vacuo to a residue which was purified via flash column chromatography (Isco®, Rf, 40 gram Gold silica, 5-60% EtOAc in heptane), to afford N-((4-(but-3-en-1-yl)-2-methoxy-6-methylpyridin-3-yl)methyl)-2-chloroisonicotinamide (1.24 g, 3.51 mmol, 89% yield) as a residue that solidified on standing. LC-MS (ES) m/z=346.1 [M+H]⁺.

### b) 3-allyl-N-((4-(but-3-en-1-yl)-2-methoxy-6-methylpyridin-3-yl)methyl)-2-chloroisonicotinamide

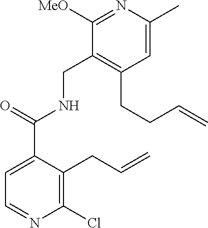 [SMILES: C=CCCc1cc(C)nc(OC)c1CNC(=O)c1ccnc(Cl)c1CC=C]

To N-((4-(but-3-en-1-yl)-2-methoxy-6-methylpyridin-3-yl)methyl)-2-chloroisonicotinamide (1.24 g, 3.59 mmol) was added THF (40 mL). The mixture was cooled to about −78° C. in a dry ice/acetone bath, n-BuLi (3.16 mL, 7.89 mmol) was added dropwise over 5 min. The reaction was allowed to stir for 30 min and then copper(I) bromide (0.514 g, 3.59 mmol) was added and the reaction was allowed to stir for 15 min then allyl bromide (0.341 mL, 3.94 mmol) was added. The reaction was held at −78° C. for 1 h and then allowed to warm to room temperature. The reaction was allowed to stir overnight, then poured into ice/(sat)NH₄Cl/1 M HCl (pH 3-4) and this was stirred for 15 min then EtOAc was added and stirred for 10 min. The mixture was partitioned and back extracted with EtOAc, then the combined organics were dried over MgSO₄ and filtered and concentrated in vacuo to a residue. This was dissolved in DCM and purified via flash column chromatography (Isco® Rf, 40 gram column, 5-60% EtOAc in heptane). Purification was repeated again as described above except using a 80 gram column to afford 3-allyl-N-((4-(but-3-en-1-yl)-2-methoxy-6-methylpyridin-3-yl)methyl)-2-chloroisonicotinamide (820 mg, 1.806 mmol, 50.4% yield) as a white solid. LC-MS (ES) m/z=386.1 [M+H]⁺.

### c) (E) and (Z)-10-chloro-1-methoxy-3-methyl-5,6,15,16-tetrahydrodipyrido[3,4-c:3′,4′-j][1]azacyclododecin-14(9H)-one

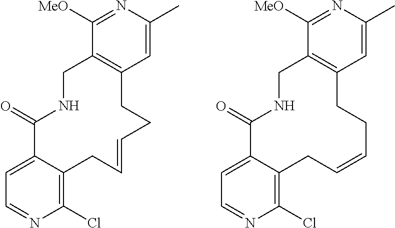 [SMILES: COc1nc(C)cc2c1CNC(=O)c1ccnc(Cl)c1C/C=C/CC2 ; COc1nc(C)cc2c1CNC(=O)c1ccnc(Cl)c1C/C=C\CC2]

A solution of 3-allyl-N-((4-(but-3-en-1-yl)-2-methoxy-6-methylpyridin-3-yl)methyl)-2-chloroisonicotinamide (820 mg, 2.125 mmol) in DCM (200 mL) was degassed with a stream of argon for 20 min, then Grubbs II (180 mg, 0.212 mmol) was added. This was capped and allowed to stir overnight. Silica gel was added and the mixture was concentrated in vacuo to a free-flowing solid, and purified via flash column chromatography (Isco®, Rf-12 gram column, 8-65% EtOAc in heptane) to afford a solid which was further purified by HPLC (Gilson®, Sunfire 30×75 mm column, 20-65% CH₃CN in water, 0.1% TFA) to afford a residue. The residue was dissolved in DCM and MeOH and concentrated NH₄OH was added and then passed quickly through a silica column (50% (90% DCM 10% MeOH, 1% NH₄OH) in DCM) to afford (E)-10-chloro-1-methoxy-3-methyl-5,6,15,16-tetrahydrodipyrido[3,4-c:3′,4′-j][1]azacyclododecin-14(9H)-one (284 mg, 0.794 mmol, 37.3%) as a solid. LC-MS (ES) m/z=358.1 [M+H]⁺.

Also isolated was (Z)-10-chloro-1-methoxy-3-methyl-5,6,15,16-tetrahydrodipyrido[3,4-c:3′,4′-j][1]azacyclododecin-14(9H)-one (139 mg, 0.388 mmol, 18.28% yield) as a solid. LC-MS (ES) m/z=358.1 [M+H]⁺.

### d) 1-methoxy-3-methyl-10-((tetrahydro-2H-pyran-4-yl)amino)-5,6,15,16-tetrahydrodipyrido[3,4-c:3′,4′-j][1]azacyclododecin-14(9H)-one

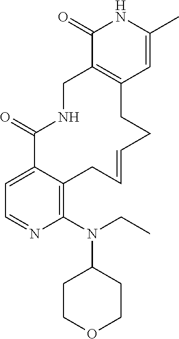 [SMILES: CCN(c1nccc2c1C/C=C/CCc1cc(C)[nH]c(=O)c1CNC2=O)C1CCOCC1]

Ca. 70 mg of (E)-10-chloro-1-methoxy-3-methyl-5,6,15,16-tetrahydrodipyrido[3,4-c:3′,4′-j][1]azacyclododecin-14(9H)-one, and 40 mg of (Z)-10-chloro-1-methoxy-3-methyl-5,6,15,16-tetrahydrodipyrido[3,4-c:3′,4′-j][1]azacyclododecin-14(9H)-one, were combined (total mass: 110 mg, 0.307 mmol) in a reaction vessel. Tetrahydro-2H-pyran-4-amine (622 mg, 6.15 mmol) was added, then the vessel was capped and sealed then placed into a heat block at 130° C. and stirred slowly for 96 h. This was then allowed to cool to about 35° C. and then diluted with DCM and MeOH was added, and the mixture adsorbed onto silica gel and purified via flash column chromatography (Isco® Rf-4 gram column, 5-65% (90:10:1 DCM:MeOH:NH₄OH) in DCM) to afford a residue. The residue was dissolved in DCE (5.0 mL) and the solution stirred. Then added in acetaldehyde (estimated, 0.134 mL, 2.367 mmol) and stirred for 10 min, then added in Na(OAc)₃BH (251 mg, 1.183 mmol) and then AcOH (0.041 mL, 0.710 mmol) and stirred vigorously at room temperature for 24 h. Then added in more reagents and capped and allowed to stir for 48 h. Diluted the reaction with DCM and then quenched with water and saturated aqueous NaHCO₃ with stirring for 30 min. Then separated and back extracted with DCM (2×) combined organics and dried over MgSO₄, filtered and concentrated in vacuo to a residue that was purified via flash column chromatography using (Isco® Rf, 4 gram column, 5-60% (90:10:1 DCM:MeOH:NH₄OH) in DCM) to afford a residue. This was dissolved in DMSO and purified using reverse phase HPLC (Gilson®, Sunfire 30×75 mm, 10-60% CH₃CN in water, 0.1% TFA) to afford a residue which was purified by via flash column (Isco® Rf-4 gram column, 5-60% (90:10:1 DCM:MeOH:NH₄OH) in DCM) to afford a residue. To the residue was added MeOH (0.30 mL) and then HCl (4 M in 1,4-dioxane, 0.738 mL, 2.95 mmol). The mixture was stirred for 15 min at room temperature then sealed and was placed into a heat block at 60° C. and stirred for 3 h. The temperature was lowered to 40° C. and allowed to stir over the weekend. HCl (4 M in 1,4-dioxane, 0.2 mL) was added, the vessel capped and placed into heat block for 4 h. Lowered the heat to 50° C. and stirred overnight. Then allowed to cool to room temperature and then blown down with nitrogen stream for 3 h. The residue was taken up in DMSO and TFA and purified using reverse phase HPLC (Gilson®, Sunfire 30×75 mm, 8-50% CH₃CN in water, 0.1% TFA) to afford a residue which was purified via flash column chromatography (Isco® Rf-4 gram column, 10-100% (90:10:1 DCM:MeOH:NH₄OH) to afford (E)-10-(ethyl(tetrahydro-2H-pyran-4-yl)amino)-3-methyl-5,6,15,16-tetrahydrodipyrido[3,4-c:3′,4′-j][1]azacyclododecine-1,14(2H,9H)-dione (4 mg, 8.98 μmol) as a white solid. LC-MS (ES) m/z=437.2 [M+H]⁺. ¹H NMR (400 MHz, DMSO-d₆) δ: 11.35 (s, 1H), 8.26 (t, J=5.1 Hz, 1H), 8.22 (d, J=4.8 Hz, 1H), 6.89 (d, J=5.1 Hz, 1H), 5.87 (s, 1H), 5.13-5.25 (m, 2H), 4.21 (br. s., 2H), 3.82 (d, J=11.4 Hz, 2H), 3.43 (br. s., 2H), 3.08-3.26 (m, 5H), 2.45-2.60 (m, 2H), 2.23 (br. s., 2H), 2.12 (s, 3H), 1.46-1.63 (m, 4H), 0.79 (t, J=6.9 Hz, 3H).

### Example 64

### (E)-10-((2-hydroxyethyl)(tetrahydro-2H-pyran-4-yl)amino)-3-methyl-5,6,15,16-tetrahydrobenzo[c]pyrido[4,3-j][1]azacyclododecine-1,14(2H,9H)-dione

### (a) 2-allyl-3-nitrobenzoic acid

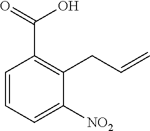

To a solution of methyl 2-allyl-3-nitrobenzoate (2.46 g, 11.12 mmol) in THF (30 mL) was added a solution of LiOH (1.598 g, 66.7 mmol) in water (9 mL). The reaction mixture was stirred at room temperature overnight. Aqueous HCl (1 M, 66.7 mL, 66.7 mmol) followed by EtOAc (50 mL) were added to the reaction mixture, which was then separated, then the aqueous layer was extracted with additional EtOAc (2×50 mL). The combined organics were dried over Na₂SO₄, filtered and concentrated to afford 2-allyl-3-nitrobenzoic acid (2.3 g, 11.10 mmol, 100% yield) as an orange solid. LC-MS (ES) m/z=208.0 [M+H]⁺.

### (b) 2-allyl-N-((4-(but-3-en-1-yl)-2-methoxy-6-methylpyridin-3-yl)methyl)-3-nitrobenzamide

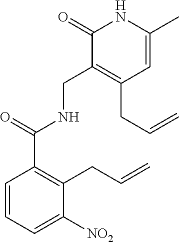 [SMILES: C=CCc1cc(C)[nH]c(=O)c1CNC(=O)c1cccc([N+](=O)[O-])c1CC=C]

A mixture of 2-allyl-3-nitrobenzoic acid (2.3 g, 11.10 mmol), (4-(but-3-en-1-yl)-2-methoxy-6-methylpyridin-3-yl)methanamine (2.75 g, 13.32 mmol), HOAt (2.267 g, 16.65 mmol), EDC (3.19 g, 16.65 mmol), N-methylmorpholine (3.66 mL, 33.3 mmol) in DMSO (40 mL) was stirred at room temperature over the weekend. The reaction mixture was poured into water (250 mL) and stirred for 1 h. The precipitate was collected by filtration, washed with water, then dried at the pump for 2 h, then in a vacuum oven overnight to afford 2-allyl-N-((4-(but-3-en-1-yl)-2-methoxy-6-methylpyridin-3-yl)methyl)-3-nitrobenzamide (4.4 g, 11.13 mmol, 100% yield) as a pale brown solid. LC-MS (ES) m/z=396.2 [M+H]⁺.

### (c) (E)-1-methoxy-3-methyl-10-nitro-5,6,15,16-tetrahydrobenzo[c]pyrido[4,3-j][1]azacyclododecin-14(9H)-one

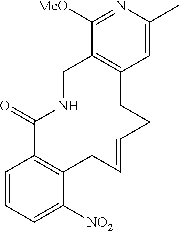 [SMILES: COc1nc(C)cc2c1CNC(=O)c1cccc([N+](=O)[O-])c1C/C=C/CC2]

A solution of 2-allyl-N-((4-(but-3-en-1-yl)-2-methoxy-6-methylpyridin-3-yl)methyl)-3-nitrobenzamide (3.65 g, 9.23 mmol) and Grubbs II (1.567 g, 1.846 mmol) in DCM (500 mL) in a 1 L RB flask was sparged with nitrogen for 10 min, then allowed to stir overnight at room temperature. The reaction mixture was concentrated, and the residue was triturated with EtOAc (30 mL). The filtrate was concentrated then triturated with EtOAc (10 mL). The solids were combined to afford a mixture of (E) and (Z)-isomers (2.47 g) as a white solid. A 300 mg portion of the mixture was dissolved in 100 mL refluxing EtOAc, filtered whilst hot (only a trace of solid remained), then allowed to cool and a solid formed over the weekend. Ca. 180 mg of a white solid was recovered by filtration. A separate 2 g portion of the mixture was dissolved in 300 mL refluxing EtOAc, filtered whilst hot (affording 231 mg of a white solid), then allowed to cool. The solution rapidly became turbid, and a white precipitate formed as the mixture cooled to room temperature and allowed to stand for the weekend. Ca. 860 mg of a white solid was recovered by filtration. The filtrate from the reaction was purified by flash column chromatography (0-80% EtOAc in hexanes, 40-g column) and product fractions were pooled with the filtrates, washings from above crystallizations and concentrated. This material was recrystallized from refluxing EtOAc (250 mL) to afford 100 mg of a white solid. The solids were combined to afford (E)-1-methoxy-3-methyl-10-nitro-5,6,15,16-tetrahydrobenzo[c]pyrido[4,3-j][1]azacyclododecin-14(9H)-one (1.37 g) as a white solid. LC-MS (ES) m/z=368.2 [M+H]⁺.

### (d) (E)-10-amino-1-methoxy-3-methyl-5,6,15,16-tetrahydrobenzo[c]pyrido[4,3-j][1]azacyclododecin-14(9H)-one

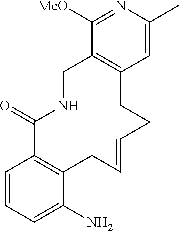 [SMILES: COc1nc(C)cc2c1CNC(=O)c1cccc(N)c1C/C=C/CC2]

To a suspension of (E)-1-methoxy-3-methyl-10-nitro-5,6,15,16-tetrahydrobenzo[c]pyrido[4,3-j][1]azacyclododecin-14(9H)-one (1.37 g, 3.73 mmol) in EtOH (50 mL) was added zinc dust (<10 micron, 3.66 g, 55.9 mmol), followed by the slow addition of AcOH (3.20 mL, 55.9 mmol). The reaction mixture was stirred for 2 h. The reaction mixture was concentrated, and the residue treated with saturated NaHCO₃ solution (100 mL) and allowed to stand overnight. 9:1 DCM:MeOH (500 mL) was added and the resulting mixture was mixed thoroughly, then filtered and the residue was sonicated with additional 9:1 DCM:MeOH (200 mL) and filtered. The combined filtrates were separated, and the organic layer was washed with brine (200 mL), then dried over Na₂SO₄, filtered and concentrated to afford (E)-10-amino-1-methoxy-3-methyl-5,6,15,16-tetrahydrobenzo[c]pyrido[4,3-j][1]azacyclododecin-14(9H)-one (1.23 g, 3.65 mmol, 98% yield) as a white solid. LC-MS (ES) m/z=338.2 [M+H]+.

### (e) (E)-10-((2-hydroxyethyl)(tetrahydro-2H-pyran-4-yl)amino)-3-methyl-5,6,15,16-tetrahydrobenzo[c]pyrido[4,3-j][1]azacyclododecine-1,14(2H,9H)-dione

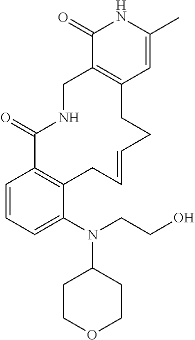 [SMILES: Cc1cc2c(c(=O)[nH]1)CNC(=O)c1cccc(N(CCO)C3CCOCC3)c1C/C=C/CC2]

To a flask containing (E)-10-amino-1-methoxy-3-methyl-5,6,15,16-tetrahydrobenzo[c]pyrido[4,3-j][1]azacyclododecin-14(9H)-one (160 mg, 0.474 mmol) in DCM (10 mL) was added dihydro-2H-pyran-4(3H)-one (142 mg, 1.423 mmol) and AcOH (0.054 mL, 0.948 mmol), then Na(OAc)₃BH (302 mg, 1.423 mmol). The reaction mixture was stirred at room temperature overnight. The reaction mixture was diluted with DCM then neutralized with saturated aqueous NaHCO₃. The layers were separated and the aqueous layer was extracted with DCM one more time. The combined organics were washed with brine, dried over Na₂SO₄, and concentrated to afford a white solid. To the solid in CH₃CN (10 mL) was added 2-iodoethanol (0.055 mL, 0.711 mmol) and DIPEA (0.124 mL, 0.711 mmol). The reaction mixture was stirred at 70° C. for 4 h. Additional 2-iodoethanol (0.165 mL) was added and the reaction was stirred over the weekend at 70° C. The reaction mixture was concentrated, and the residue was redissolved in CH₃CN (4 mL) in a microwave tube, then 2-iodoethanol (0.333 mL, 4.27 mmol) and DIPEA (0.248 mL, 1.423 mmol) were added. The reaction mixture was heated under microwave irradiation at 150° C. for 1.5 h. The reaction mixture was concentrated and purified by flash column chromatography (0-100% (1% NH₄OH+9% MeOH+90% CHCl₃) in CHCl₃, 4 g column). The resulting fractions were concentrated down and purified by Gilson® HPLC (5-40% CH₃CN in water, 0.1% TFA in mobile phase), and the resulting fractions were concentrated and the residue was passed through a 500 mg Silicycle® (carbonate) cartridge eluting with MeOH (20 mL) to afford (E)-10-((2-hydroxyethyl)(tetrahydro-2H-pyran-4-yl)amino)-3-methyl-5,6,15,16-tetrahydrobenzo[c]pyrido[4,3-j][1]azacyclododecine-1,14(2H,9H)-dione (65 mg, 0.144 mmol, 30.4% yield) as a white solid. LC-MS (ES) m/z=452.3 [M+H]⁺. ¹H NMR (400 MHz, DMSO-d₆) δ: 11.30 (s., 1H), 7.98 (t, J=5.05 Hz, 1H), 7.27 (dd, J=1.26, 8.08 Hz, 1H), 7.20 (t, J=7.71 Hz, 1H), 6.97 (dd, J=1.01, 7.33 Hz, 1H), 5.84 (s, 1H), 5.07-5.22 (m, 2H), 4.18 (br. s., 2H), 3.78-3.85 (m, 2H), 3.53 (br. s., 2H), 3.18-3.22 (m, 4H), 3.00-3.03-3.08 (m, 2H), 2.88-2.98 (m, 1H), 2.45-2.60 (m, 2H), 2.22 (br. s., 2H), 2.12 (s, 3H), 1.60 (br. s., 2H), 1.31-1.48 (m, 2H). 1H not observed.

### Example 65

### (E)-10-((1-(dimethylamino)piperidin-4-yl)(ethyl)amino)-3-methyl-5,6,15,16-tetrahydrobenzo[c]pyrido[4,3-j][1]azacyclododecine-1,14(2H,9H)-dione

### (a) (E)-10-((1-(dimethylamino)piperidin-4-yl)(ethyl)amino)-1-methoxy-3-methyl-5,6,15,16-tetrahydrobenzo[c]pyrido[4,3-j][1]azacyclododecin-14(9H)-one

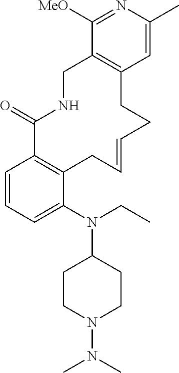 [SMILES: CCN(c1cccc2c1C/C=C/CCc1cc(C)nc(OC)c1CNC2=O)C1CCN(N(C)C)CC1]

To a stirred suspension of (E)-10-amino-1-methoxy-3-methyl-5,6,15,16-tetrahydrobenzo[c]pyrido[4,3-j][1]azacyclododecin-14(9H)-one (87 mg, 0.258 mmol) and 1-(dimethylamino)piperidin-4-one (73.3 mg, 0.516 mmol) in DCE (3 mL) was added AcOH (0.015 mL, 0.258 mmol). The suspension was stirred for 2 h at room temperature, then Na(OAc)₃BH (109 mg, 0.516 mmol) was added in one portion and the reaction was stirred overnight at room temperature. The reaction was diluted with DCM (100 mL) and washed sequentially with saturated aqueous Na₂CO₃ solution (20 mL) and brine (20 mL). The organic layer was dried over Na₂SO₄, filtered, concentrated to afford a white solid. The solid was dissolved in DCE (10 mL) at room temperature and acetaldehyde (0.044 mL, 0.774 mmol) was added followed by AcOH (0.074 mL, 1.290 mmol). The reaction was stirred for 5 min, then Na(OAc)₃BH (164 mg, 0.774 mmol) was added, and the reaction was stirred at room temperature overnight. The reaction mixture was poured into saturated aqueous NaHCO₃ (30 mL) and extracted with DCM (3×500 mL). The pooled organics were washed with brine (30 mL), then dried over Na₂SO₄, filtered, concentrated, and the residue purified by flash column chromatography (0-10% MeOH in DCM, 12-g column) to afford (E)-10-((1-(dimethylamino)piperidin-4-yl)(ethyl)amino)-1-methoxy-3-methyl-5,6,15,16-tetrahydrobenzo[c]pyrido[4,3-j][1]azacyclododecin-14(9H)-one (100 mg, 0.203 mmol, 79% yield) as an off-white solid. LC-MS (ES) m/z=492.4 [M+H]⁺ (minor), 366.2 (major).

### (b) (E)-10-((1-(dimethylamino)piperidin-4-yl)(ethyl)amino)-3-methyl-5,6,15,16-tetrahydrobenzo[c]pyrido[4,3-j][1]azacyclododecine-1,14(2H,9H)-dione

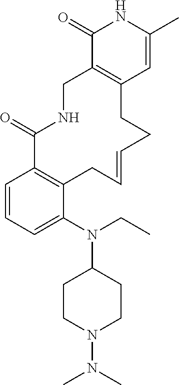 [SMILES: CCN(c1cccc2c1C/C=C/CCc1cc(C)[nH]c(=O)c1CNC2=O)C1CCN(N(C)C)CC1]

A suspension of (E)-10-((1-(dimethylamino)piperidin-4-yl)(ethyl)amino)-1-methoxy-3-methyl-5,6,15,16-tetrahydrobenzo[c]pyrido[4,3-j][1]azacyclododecin-14(9H)-one (100 mg, 0.203 mmol) in HCl (4 M, 1,4-dioxane, 3 mL, 12.00 mmol) and MeOH (1 mL) was stirred at room temperature overnight, then at 70° C. overnight. The reaction mixture was concentrated, then redissolved in DCM, MeOH and NH₄OH, adsorbed onto silica then purified by flash column chromatography (0-20% MeOH in DCM, 12-g column) to afford (E)-10-((1-(dimethylamino)piperidin-4-yl)(ethyl)amino)-3-methyl-5,6,15,16-tetrahydrobenzo[c]pyrido[4,3-j][1]azacyclododecine-1,14(2H,9H)-dione (86 mg, 0.180 mmol, 89% yield) as a white solid. LC-MS (ES) m/z=478.4 [M+H]⁺ (minor), 239.8 (major). ¹H NMR (400 MHz, DMSO-d₆) δ: 11.29 (s, 1H), 8.00 (t, J=4.9 Hz, 1H), 7.11-7.27 (m, 2H), 6.87-7.05 (m, 1H), 5.84 (s, 1H), 5.00-5.24 (m, 2H), 3.92-4.35 (m, 2H), 3.54 (br. s., 2H), 3.32 (s, 3H), 2.91-3.02 (m, 2H), 2.73-2.83 (m, 2H), 2.61-2.71 (m, 1H), 2.15-2.27 (m, 9H), 2.11 (s, 3H), 1.66 (br. s., 2H), 1.31-1.50 (m, 2H), 0.75 (t, J=6.9 Hz, 3H).

### Example 66

### (E)-10-(ethyl(2-azaspiro[3.5]nonan-7-yl)amino)-3-methyl-5,6,15,16-tetrahydrobenzo[c]pyrido[4,3-j][1]azacyclododecine-1,14(2H,9H)-dione

### (a) (E)-tert-butyl 7-(ethyl(1-methoxy-3-methyl-14-oxo-5,6,9,14,15,16-hexahydrobenzo[c]pyrido[4,3-j][1]azacyclododecin-10-yl)amino)-2-azaspiro[3.5]nonane-2-carboxylate

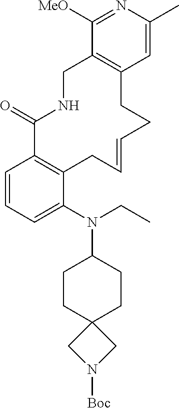 [SMILES: CCN(c1cccc2c1C/C=C/CCc1cc(C)nc(OC)c1CNC2=O)C1CCC2(CC1)CN(C(=O)OC(C)(C)C)C2]

To a stirred suspension of (E)-10-amino-1-methoxy-3-methyl-5,6,15,16-tetrahydrobenzo[c]pyrido[4,3-j][1]azacyclododecin-14(9H)-one (200 mg, 0.593 mmol) and tert-butyl 7-oxo-2-azaspiro[3.5]nonane-2-carboxylate (284 mg, 1.185 mmol) in DCE (10 mL) was added AcOH (0.05 mL, 0.873 mmol). The suspension was stirred for 1 h at room temperature, then Na(OAc)₃BH (251 mg, 1.185 mmol) was added in one portion and the reaction was stirred overnight at room temperature. LCMS analysis indicated complete conversion, so AcOH (0.170 mL, 2.96 mmol), acetaldehyde (0.167 mL, 2.96 mmol), Na(OAc)₃BH (377 mg, 1.778 mmol) were added and the reaction mixture was stirred overnight at room temperature. The reaction mixture was poured into aqueous NaHCO₃ (50 mL) then extracted with DCM (3×50 mL). The combined organics were washed with brine (30 mL), dried over Na₂SO₄, filtered, concentrated and the residue was purified by flash column chromatography (0-50% EtOAc in hexanes, 24 g column) to afford (E)-tert-butyl 7-(ethyl(1-methoxy-3-methyl-14-oxo-5,6,9,14,15,16-hexahydrobenzo[c]pyrido[4,3-j][1]azacyclododecin-10-yl)amino)-2-azaspiro[3.5]nonane-2-carboxylate (308 mg) as a white solid. LC-MS (ES) m/z=589.5 [M+H]⁺ (minor), 267.3 (major). ¹H NMR indicated tert-butyl 7-oxo-2-azaspiro[3.5]nonane-2-carboxylate was present in the product in a ca. 3:2 mol ratio, suggesting 79% purity by mass.

### (b) (E)-10-(ethyl(2-azaspiro[3.5]nonan-7-yl)amino)-3-methyl-5,6,15,16-tetrahydrobenzo[c]pyrido[4,3-j][1]azacyclododecine-1,14(2H,9H)-dione

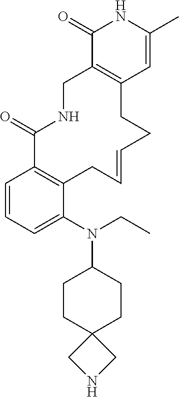 [SMILES: CCN(c1cccc2c1C/C=C/CCc1cc(C)[nH]c(=O)c1CNC2=O)C1CCC2(CC1)CNC2]

A suspension of (E)-tert-butyl 7-(ethyl(1-methoxy-3-methyl-14-oxo-5,6,9,14,15,16-hexahydrobenzo[c]pyrido[4,3-j][1]azacyclododecin-10-yl)amino)-2-azaspiro[3.5]nonane-2-carboxylate (308 mg) in HCl (4 M, 1,4-dioxane, 3 mL, 12.00 mmol) and MeOH (1 mL) was stirred at 70° C. overnight. LCMS analysis indicated complete conversion. The reaction mixture was concentrated, then redissolved in DMSO and purified by reverse-phase HPLC (8-28% CH₃CN in water; 0.1% TFA). The product fractions were pooled, basified and concentrated to minimal aqueous, then extracted with EtOAc (3×100 mL) and then 9:1 DCM:MeOH (3×100 mL). The combined organics were dried over Na₂SO₄, filtered, and concentrated to afford (E)-10-(ethyl(2-azaspiro[3.5]nonan-7-yl)amino)-3-methyl-5,6,15,16-tetrahydrobenzo[c]pyrido[4,3-j][1]azacyclododecine-1,14(2H,9H)-dione (35 mg, 0.074 mmol) as an off-white solid. LC-MS (ES) m/z=475.3 [M+H]⁺ (minor), 238.4 (major). ¹H NMR (400 MHz, DMSO-d₆) δ: 11.29 (br. s., 1H), 7.89-8.05 (m, 1H), 7.09-7.31 (m, 2H), 6.86-7.01 (m, 1H), 5.83 (s, 1H), 5.04-5.24 (m, 2H), 4.15 (br. s., 2H), 3.41-3.70 (m, 4H), 3.29 (s, 1H), 2.85-3.21 (m, 5H), 2.62-2.70 (m, 1H), 2.53-2.57 (m, 1H), 2.30-2.43 (m, 2H), 2.22 (br. s., 2H), 2.11 (s, 3H), 1.77-1.89 (m, 1H), 1.40-1.67 (m, 4H), 0.65-0.82 (m, 3H). 1H not observed.

### Example 67

### (E)-10-(ethyl(2-methyl-2-azaspiro[3.5]nonan-7-yl)amino)-3-methyl-5,6,15,16-tetrahydrobenzo[c]pyrido[4,3-j][1]azacyclododecine-1,14(2H,9H)-dione

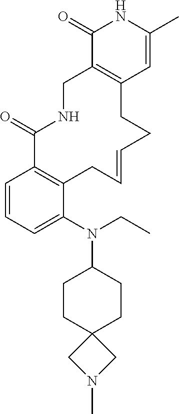 [SMILES: CCN(c1cccc2c1C/C=C/CCc1cc(C)[nH]c(=O)c1CNC2=O)C1CCC2(CC1)CN(C)C2]

To a stirred solution of (E)-10-(ethyl(2-azaspiro[3.5]nonan-7-yl)amino)-3-methyl-5,6,15,16-tetrahydrobenzo[c]pyrido[4,3-j][1]azacyclododecine-1,14(2H,9H)-dione (30 mg, 0.063 mmol) and formaldehyde (37 wt % in water, 0.1 mL, 1.343 mmol) in MeOH (2 mL) was added AcOH (5.43 μL, 0.095 mmol) followed by Na(OAc)₃BH (46.9 mg, 0.221 mmol) in one portion and the reaction was stirred overnight at room temperature. The reaction was quenched with saturated aqueous NaHCO₃ solution until basic (caution, effervesence!), then the resulting mixture was extracted with CHCl₃ (3×30 mL). The combined organics were dried over Na₂SO₄, filtered, and concentrated to afford an off-white solid. The crude was purified by flash column chromatography (400 mg SiO₂, 100% DCM, then 10% MeOH in DCM, then 80:20:2 DCM:MeOH:NH₄OH) to afford (E)-10-(ethyl(2-methyl-2-azaspiro[3.5]nonan-7-yl)amino)-3-methyl-5,6,15,16-tetrahydrobenzo[c]pyrido[4,3-j][1]azacyclododecine-1,14(2H,9H)-dione (8 mg, 0.016 mmol, 25.9% yield) as a white solid. LC-MS (ES) m/z=489.4 [M+H]⁺ (minor), 245.5 (major). ¹H NMR (400 MHz, DMSO-d₆) δ: 11.25-11.36 (m, 1H), 7.93-8.00 (m, 1H), 7.17-7.24 (m, 2H), 6.90-6.99 (m, 1H), 5.77-5.87 (m, 1H), 5.03-5.24 (m, 2H), 4.00-4.32 (m, 2H), 3.61-3.93 (m, 4H), 3.44-3.57 (m, 2H), 2.86-3.03 (m, 2H), 2.77 (s, 3H), 2.62-2.71 (m, 1H), 2.45-2.60 (m, 2H), 2.15-2.26 (m, 2H), 2.11 (s, 3H), 1.88-2.01 (m, 2H), 1.54-1.70 (m, 2H), 1.30-1.42 (m, 2H), 1.07-1.29 (m, 2H), 0.75 (s, 3H).

### Example 68

### (E)-10-(ethyl(7-azaspiro[3.5]nonan-2-yl)amino)-3-methyl-5,6,15,16-tetrahydrobenzo[c]pyrido[4,3-j][1]azacyclododecine-1,14(2H,9H)-dione

### (a) (E)-tert-butyl 2-(ethyl(1-methoxy-3-methyl-14-oxo-5,6,9,14,15,16-hexahydrobenzo[c]pyrido[4,3-j][1]azacyclododecin-10-yl)amino)-7-azaspiro[3.5]nonane-7-carboxylate

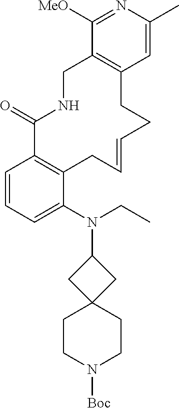 [SMILES: CCN(c1cccc2c1C/C=C/CCc1cc(C)nc(OC)c1CNC2=O)C1CC2(CCN(C(=O)OC(C)(C)C)CC2)C1]

To a flask containing (E)-tert-butyl 2-(ethyl(1-methoxy-3-methyl-14-oxo-5,6,9,14,15,16-hexahydrobenzo[c]pyrido[4,3-j][1]azacyclododecin-10-yl)amino)-7-azaspiro[3.5]nonane-7-carboxylate (170 mg, 0.289 mmol, 64.9% yield) in DCE (10 mL) was added tert-butyl 2-oxo-7-azaspiro[3.5]nonane-7-carboxylate (213 mg, 0.889 mmol) and AcOH (0.051 mL, 0.889 mmol), then Na(OAc)₃BH (283 mg, 1.334 mmol). The reaction mixture was stirred at room temperature overnight. Additional tert-butyl 2-oxo-7-azaspiro[3.5]nonane-7-carboxylate (213 mg, 0.889 mmol) and Na(OAc)₃BH (142 mg, 0.445 mmol) were added and the reaction was stirred for another 5 h. To the above reaction mixture was added acetaldehyde (0.089 mL, 4.45 mmol) and AcOH (0.051 mL, 0.889 mmol) then Na(OAc)₃BH (283 mg, 1.334 mmol). The reaction mixture was stirred at room temperature overnight. The reaction mixture was diluted with DCM then neutralized with saturated aqueous NaHCO₃, and the aqueous layer was extracted with DCM one more time. The combined organics were washed with brine and dried over Na₂SO₄, concentrated and purified by flash column chromatography (0-50% EtOAc in hexane, 10 g column) to afford (E)-tert-butyl 2-(ethyl(1-methoxy-3-methyl-14-oxo-5,6,9,14,15,16-hexahydrobenzo[c]pyrido[4,3-j][1]azacyclododecin-10-yl)amino)-7-azaspiro[3.5]nonane-7-carboxylate (170 mg, 0.289 mmol, 64.9% yield) as a white solid. LC-MS (ES) m/z=589.5 [M+H]⁺ (minor), 533.5 (major).

### (b) (E)-10-(ethyl(7-azaspiro[3.5]nonan-2-yl)amino)-3-methyl-5,6,15,16-tetrahydrobenzo[c]pyrido[4,3-j][1]azacyclododecine-1,14(2H,9H)-dione

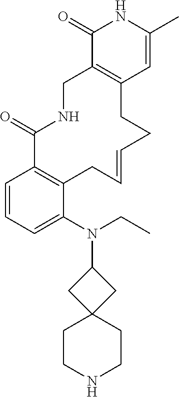 [SMILES: CCN(c1cccc2c1C/C=C/CCc1cc(C)[nH]c(=O)c1CNC2=O)C1CC2(CCNCC2)C1]

(E)-tert-butyl 2-(ethyl(1-methoxy-3-methyl-14-oxo-5,6,9,14,15,16-hexahydrobenzo[c]pyrido[4,3-j][1]azacyclododecin-10-yl)amino)-7-azaspiro[3.5]nonane-7-carboxylate (165 mg, 0.280 mmol) was dissolved in 1,4-dioxane (6 mL) and MeOH (3 mL). HCl (4 M, 1,4-dioxane, 2 mL, 8.00 mmol) was added and the reaction was stirred at 70° C. overnight. LCMS analysis indicated complete conversion, and the reaction was concentrated down. The residue was neutralized with 20% NH₄OH in MeOH and the mixture was adsorbed onto Celite® and purified by flash column chromatography (0-100% (1% NH₄OH+9% MeOH+90% CHCl₃) in CHCl₃, 4 g column) to afford (E)-10-(ethyl(7-azaspiro[3.5]nonan-2-yl)amino)-3-methyl-5,6,15,16-tetrahydrobenzo[c]pyrido[4,3-j][1]azacyclododecine-1,14(2H,9H)-dione (115 mg, 0.242 mmol, 86% yield) as a white solid. LC-MS (ES) m/z=475.7 [M+H]⁺ (minor), 238.3 (major). ¹H NMR (400 MHz, DMSO-d₆) δ: 8.01 (br. s., 1H), 7.13-7.21 (m, 1H), 7.05 (d, J=7.07 Hz, 1H), 6.94 (dd, J=1.01, 7.33 Hz, 1H), 5.84 (s, 1H), 5.05-5.22 (m, 2H), 3.91-4.41 (m, 2H), 2.94-3.79 (m, 5H), 2.74-2.88 (m, 2H), 2.65 (br. s., 2H), 2.56 (br. s., 2H), 2.02-2.31 (m, 6H), 1.73-1.99 (m, 2H), 1.23-1.58 (m, 6H), 0.73 (t, J=6.9 Hz, 3H). 1H not observed.

### Example 69

### (E)-10-(ethyl(7-methyl-7-azaspiro[3.5]nonan-2-yl)amino)-3-methyl-5,6,15,16-tetrahydrobenzo[c]pyrido[4,3-j][1]azacyclododecine-1,14(2H,9H)-dione

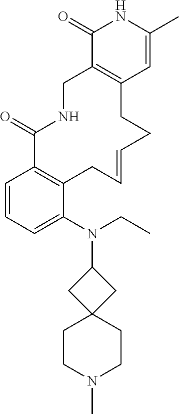 [SMILES: CCN(c1cccc2c1C/C=C/CCc1cc(C)[nH]c(=O)c1CNC2=O)C1CC2(CCN(C)CC2)C1]

To (E)-10-(ethyl(7-azaspiro[3.5]nonan-2-yl)amino)-3-methyl-5,6,15,16-tetrahydrobenzo[c]pyrido[4,3-j][1]azacyclododecine-1,14(2H,9H)-dione (95 mg, 0.200 mmol) in MeOH (4 mL) was added Na(OAc)₃BH (170 mg, 0.801 mmol), formaldehyde (37 wt % in water, 0.149 mL, 2.002 mmol) and AcOH (0.034 mL, 0.600 mmol). The reaction mixture was stirred at room temperature for 3 h. The reaction mixture was adsorbed onto Celite® and purified by flash column chromatography (0-80% (1% NH₄OH+9% MeOH+90% CHCl₃) in CHCl₃, 4 g column) to afford (E)-10-(ethyl(7-methyl-7-azaspiro[3.5]nonan-2-yl)amino)-3-methyl-5,6,15,16-tetrahydrobenzo[c]pyrido[4,3-j][1]azacyclododecine-1,14(2H,9H)-dione (70 mg, 0.143 mmol, 71.6% yield) as a white solid. LC-MS (ES) m/z=489.4 [M+H]⁺ (minor), 245.3 (major). ¹H NMR (400 MHz, DMSO-d₆) δ: 11.29 (br. s., 1H), 7.88-8.19 (m, 1H), 7.12-7.21 (m, 1H), 7.05 (dd, J=1.14, 7.96 Hz, 1H), 6.94 (dd, J=1.14, 7.45 Hz, 1H), 5.84 (s, 1H), 5.08-5.20 (m, 2H), 3.96-4.41 (m, 2H), 3.69 (quin, J=7.33 Hz, 1H), 3.46-3.64 (m, 2H), 2.81 (q, J=7.07 Hz, 2H), 2.52-2.67 (m, 2H), 1.99-2.31 (m, 12H), 1.73-1.95 (m, 2H), 1.28-1.59 (m, 6H), 0.73 (t, J=7.1 Hz, 3H).

### Example 70

### (E)-10-((6-aminospiro[3.3]heptan-2-yl)(ethyl)amino)-3-methyl-5,6,15,16-tetrahydrobenzo[c]pyrido[4,3-j][1]azacyclododecine-1,14(2H,9H)-dione

### (a) (E)-tert-butyl(6-(ethyl(1-methoxy-3-methyl-14-oxo-5,6,9,14,15,16-hexahydrobenzo[c]pyrido[4,3-j][1]azacyclododecin-10-yl)amino)spiro[3.3]heptan-2-yl)carbamate

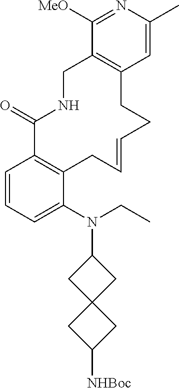 [SMILES: CCN(c1cccc2c1C/C=C/CCc1cc(C)nc(OC)c1CNC2=O)C1CC2(CC(NC(=O)OC(C)(C)C)C2)C1]

To a flask containing (E)-tert-butyl(6-(ethyl(1-methoxy-3-methyl-14-oxo-5,6,9,14,15,16-hexahydrobenzo[c]pyrido[4,3-j][1]azacyclododecin-10-yl)amino)spiro[3.3]heptan-2-yl)carbamate (200 mg, 0.348 mmol, 49.5% yield) in DCE (12 mL) was added tert-butyl(6-oxospiro[3.3]heptan-2-yl)carbamate (500 mg, 2.107 mmol) and AcOH (0.080 mL, 1.405 mmol), then Na(OAc)₃BH (447 mg, 2.107 mmol). The reaction mixture was stirred at room temperature overnight. To the above reaction mixture was added acetaldehyde (0.140 mL, 7.02 mmol), AcOH (0.080 mL, 1.405 mmol) then Na(OAc)₃BH (447 mg, 2.107 mmol) and the reaction mixture was stirred at room temperature overnight. The reaction mixture was diluted with DCM then neutralized with saturated aqueous NaHCO₃. The aqueous layer was extracted with DCM one more time. The combined organics were washed with brine, concentrated and purified by flash column chromatography (0-40% EtOAc in hexane, 10 g column) to afford a white solid. The solid was further purified by Gilson® HPLC (25-55% CH₃CN in water, 0.1% TFA in mobile phase) to afford (E)-tert-butyl(6-(ethyl(1-methoxy-3-methyl-14-oxo-5,6,9,14,15,16-hexahydrobenzo[c]pyrido[4,3-j][1]azacyclododecin-10-yl)amino)spiro[3.3]heptan-2-yl)carbamate (200 mg, 0.348 mmol, 49.5% yield) as a white solid. LC-MS (ES) m/z=575.5 [M+H]⁺ (minor), 366.2 (major).

### (b) (E)-10-((6-aminospiro[3.3]heptan-2-yl)(ethyl)amino)-3-methyl-5,6,15,16-tetrahydrobenzo[c]pyrido[4,3-j][1]azacyclododecine-1,14(2H,9H)-dione

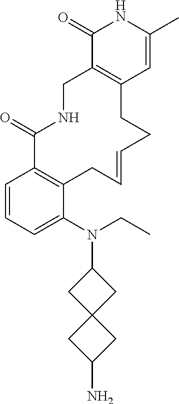 [SMILES: CCN(c1cccc2c1C/C=C/CCc1cc(C)[nH]c(=O)c1CNC2=O)C1CC2(CC(N)C2)C1]

A mixture of (E)-tert-butyl(6-(ethyl(1-methoxy-3-methyl-14-oxo-5,6,9,14,15,16-hexahydrobenzo[c]pyrido[4,3-j][1]azacyclododecin-10-yl)amino)spiro[3.3]heptan-2-yl)carbamate (200 mg, 0.348 mmol) was dissolved in 1,4-dioxane (4 mL) and MeOH (2 mL). HCl (4 M, 1,4-dioxane, 1.5 mL, 6.00 mmol) was added and the reaction was stirred at 70° C. overnight. The reaction was concentrated then treated with 20% NH₄OH in MeOH and the mixture was adsorbed onto Celite® and purified by flash column chromatography (0-100% (1% NH₄OH+9% MeOH+90% CHCl₃) in CHCl₃, 4 g column) to afford (E)-10-((6-aminospiro[3.3]heptan-2-yl)(ethyl)amino)-3-methyl-5,6,15,16-tetrahydrobenzo[c]pyrido[4,3-j][1]azacyclododecine-1,14(2H,9H)-dione (160 mg, 0.347 mmol, 100% yield) as a white solid. LC-MS (ES) m/z=575.5 [M+H]⁺ (minor), 366.2 (major). ¹H NMR (400 MHz, DMSO-d₆) δ: 11.38 (br. s., 1H), 8.01 (br. s., 1H), 7.13-7.20 (m, 1H), 7.01-7.07 (m, 1H), 6.93 (dd, J=1.01, 7.33 Hz, 1H), 5.84 (s, 1H), 5.06-5.21 (m, 2H), 4.00-4.32 (m, 2H), 3.42-3.64 (m, 3H), 3.19-3.41 (m, 3H), 3.04-3.19 (m, 1H), 2.78 (q, J=7.07 Hz, 2H), 2.51 (br. s., 2H), 2.16-2.29 (m, 3H), 2.12 (s, 3H), 1.97-2.08 (m, 2H), 1.79-1.95 (m, 1H), 1.46-1.70 (m, 4H), 0.72 (t, J=7.07 Hz, 3H).

### Example 71

### (E)-10-((6-(dimethylamino)spiro[3.3]heptan-2-yl)(ethyl)amino)-3-methyl-5,6,15,16-tetrahydrobenzo[c]pyrido[4,3-j][1]azacyclododecine-1,14(2H,9H)-dione

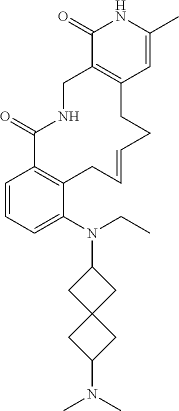 [SMILES: CCN(c1cccc2c1C/C=C/CCc1cc(C)[nH]c(=O)c1CNC2=O)C1CC2(CC(N(C)C)C2)C1]

To (E)-104(6-aminospiro[3.3]heptan-2-yl)(ethyl)amino)-3-methyl-5,6,15,16-tetrahydrobenzo[c]pyrido[4,3-j][1]azacyclododecine-1,14(2H,9H)-dione (145 mg, 0.315 mmol) in MeOH (4 mL) was added Na(OAc)₃BH (267 mg, 1.259 mmol), formaldehyde (37 wt % in water, 0.234 mL, 3.15 mmol) and AcOH (0.054 mL, 0.944 mmol). The reaction mixture was stirred at room temperature for 3 h. The reaction mixture was concentrated onto Celite® and purified by flash column chromatography (0-100% (1% NH₄OH+9% MeOH+90% CHCl₃) in CHCl₃, 4 g column) to afford (E)-1046-(dimethylamino)spiro[3.3]heptan-2-yl)(ethyl)amino)-3-methyl-5,6,15,16-tetrahydrobenzo[c]pyrido[4,3-j][1]azacyclododecine-1,14(2H,9H)-dione (129 mg, 0.264 mmol, 84% yield) as a white solid. LC-MS (ES) m/z=489.4 [M+H]⁺ (minor), 245.3 (major). ¹H NMR (400 MHz, DMSO-d₆) δ: 11.29 (s, 1H), 8.02 (t, J=4.80 Hz, 1H), 7.16 (t, J=7.71 Hz, 1H), 7.04 (dd, J=1.26, 8.08 Hz, 1H), 6.93 (dd, J=1.01, 7.33 Hz, 1H), 5.84 (s, 1H), 5.06-5.20 (m, 2H), 4.02-4.30 (m, 2H), 3.44-3.67 (m, 3H), 2.79 (q, J=6.91 Hz, 2H), 2.51-2.61 (m, 2H), 2.42 (quin, J=7.58 Hz, 1H), 2.22 (br. s., 2H), 2.03-2.15 (m, 5H), 1.82-1.98 (m, 8H), 1.53-1.77 (m, 4H), 0.72 (t, J=7.07 Hz, 3H).

### Example 72

### (E)-10-(ethyl(2-methyl-2-azaspiro[3.3]heptan-6-yl)amino)-3-methyl-5,6,15,16-tetrahydrobenzo[c]pyrido[4,3-j][1]azacyclododecine-1,14(2H,9H)-dione

### (a) (E)-tert-butyl 6-(ethyl(1-methoxy-3-methyl-14-oxo-5,6,9,14,15,16-hexahydrobenzo[c]pyrido[4,3-j][1]azacyclododecin-10-yl)amino)-2-azaspiro[3.3]heptane-2-carboxylate

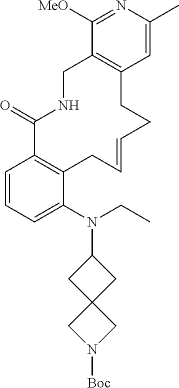 [SMILES: CCN(c1cccc2c1C/C=C/CCc1cc(C)nc(OC)c1CNC2=O)C1CC2(C1)CN(C(=O)OC(C)(C)C)C2]

To a stirred suspension of (E)-10-amino-1-methoxy-3-methyl-5,6,15,16-tetrahydrobenzo[c]pyrido[4,3-j][1]azacyclododecin-14(9H)-one (200 mg, 0.593 mmol) and tert-butyl 6-oxo-2-azaspiro[3.3]heptane-2-carboxylate (250 mg, 1.185 mmol) in DCE (6 mL) was added AcOH (0.034 mL, 0.593 mmol). The suspension was stirred for 1 h at room temperature, then Na(OAc)₃BH (251 mg, 1.185 mmol) was added in one portion and the reaction was stirred overnight at room temperature. AcOH (0.170 mL, 2.96 mmol), acetaldehyde (0.167 mL, 2.96 mmol), Na(OAc)₃BH (377 mg, 1.778 mmol) were added and the reaction mixture was stirred overnight at room temperature. The reaction mixture was poured into aqueous NaHCO₃ (50 mL) then extracted with DCM (3×50 mL). The combined organics were washed with brine (30 mL), dried over Na₂SO₄, filtered, concentrated and the residue was purified by flash column chromatography (0-50% EtOAc in hexanes, 24 g column) to afford (E)-tert-butyl 6-(ethyl(1-methoxy-3-methyl-14-oxo-5,6,9,14,15,16-hexahydrobenzo[c]pyrido[4,3-j][1]azacyclododecin-10-yl)amino)-2-azaspiro[3.3]heptane-2-carboxylate (210 mg, 0.375 mmol, 63.2% yield) as a white solid. LC-MS (ES) m/z=561.5 [M+H]⁺ (minor), 505.4 (major).

### (b) (E)-10-(ethyl(2-methyl-2-azaspiro[3.3]heptan-6-yl)amino)-3-methyl-5,6,15,16-tetrahydrobenzo[c]pyrido[4,3-j][1]azacyclododecine-1,14(2H,9H)-dione

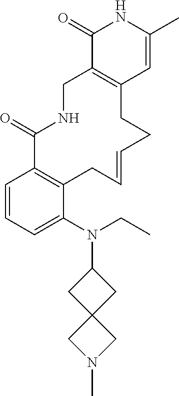 [SMILES: CCN(c1cccc2c1C/C=C/CCc1cc(C)[nH]c(=O)c1CNC2=O)C1CC2(C1)CN(C)C2]

A suspension of (E)-tert-butyl 6-(ethyl(1-methoxy-3-methyl-14-oxo-5,6,9,14,15,16-hexahydrobenzo[c]pyrido[4,3-j][1]azacyclododecin-10-yl)amino)-2-azaspiro[3.3]heptane-2-carboxylate (210 mg, 0.375 mmol) in HCl (4 M, 1,4-dioxane, 3 mL, 12.00 mmol) and MeOH (1 mL) was stirred at 70° C. overnight. The mixture was concentrated, dissolved in DMSO and purified by reverse-phase HPLC (8-28% CH₃CN in water; 0.1% TFA). The product fractions were pooled, basified and concentrated to minimal aqueous, then extracted with EtOAc (3×100 mL) and 9:1 DCM:MeOH (3×100 mL). The product remained in the aqueous layer. The aqueous layer was concentrated to a solid residue and triturated with MeOH (100 mL). The mixture was filtered, then the filtrate concentrated to afford a white solid, likely containing inorganic salts. To the residue and formaldehyde (37 wt % in water, 0.467 mL, 6.27 mmol) in MeOH (3 mL) was added AcOH (0.054 mL, 0.940 mmol) followed by Na(OAc)₃BH (465 mg, 2.194 mmol) in one portion and the reaction was stirred overnight at room temperature. The reaction was quenched with saturated aqueous NaHCO₃ solution until basic (caution, effervesence!), then the resulting mixture was extracted with CHCl₃ (3×30 mL). The combined organics were dried over Na₂SO₄, filtered, and concentrated to afford an off-white solid. The crude was purified by flash column chromatography (200 mg SiO₂, 10% MeOH in DCM, then 80:20:2 DCM:MeOH:NH₄OH) to afford (E)-10-(ethyl(2-methyl-2-azaspiro[3.3]heptan-6-yl)amino)-3-methyl-5,6,15,16-tetrahydrobenzo[c]pyrido[4,3-j][1]azacyclododecine-1,14(2H,9H)-dione (7 mg, 0.015 mmol) as a white solid. LC-MS (ES) m/z=461.4 [M+H]⁺. ¹H NMR (400 MHz, DMSO-d₆) δ: 11.29 (br. s., 1H), 8.02 (br. s., 1H), 7.13-7.25 (m, 1H), 7.04 (d, J=8.1 Hz, 1H), 6.96 (d, J=7.3 Hz, 1H), 5.84 (s, 1H), 5.03-5.22 (m, 2H), 4.18 (br. s., 2H), 3.44-3.71 (m, 7H), 2.80 (q, J=7.1 Hz, 2H), 2.45-2.60 (m, 2H) 2.22 (br. s., 4H), 2.12 (s, 4H), 1.79 (br. s., 1H), 0.72 (t, J=7.1 Hz, 3H). 3H not observed.

### Example 73

### (E)-10-(ethyl(trans-4-(methylamino)cyclohexyl)amino)-3-methyl-5,6,15,16-tetrahydrobenzo[c]pyrido[4,3-j][1]azacyclododecine-1,14(2H,9H)-dione formic acid salt

### a) ((E)-tert-butyl(4-(ethyl(1-methoxy-3-methyl-14-oxo-5,6,9,14,15,16-hexahydrobenzo[c]pyrido[4,3-j][1]azacyclododecin-10-yl)amino)cyclohexyl)(methyl)carbamate

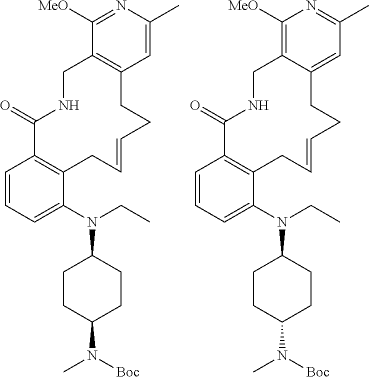 [SMILES: CCN(c1cccc2c1C/C=C/CCc1cc(C)nc(OC)c1CNC2=O)[C@H]1CC[C@@H](N(C)C(=O)OC(C)(C)C)CC1 ; CCN(c1cccc2c1C/C=C/CCc1cc(C)nc(OC)c1CNC2=O)[C@H]1CC[C@H](N(C)C(=O)OC(C)(C)C)CC1]

(E)-10-amino-1-methoxy-3-methyl-5,6,15,16-tetrahydrobenzo[c]pyrido[4,3-j][1]azacyclododecin-14(9H)-one (0.150 g, 0.445 mmol) and tert-butyl methyl(4-oxocyclohexyl)carbamate (0.121 g, 0.533 mmol) were dissolved in DCE (8 mL), then AcOH (0.076 mL, 1.334 mmol) and Na(OAc)₃BH (0.283 g, 1.334 mmol) were added and the mixture was stirred vigorously at room temperature overnight. Zinc chloride (0.061 g, 0.445 mmol) was added and the mixture was stirred for 1 h and then tert-butyl methyl(4-oxocyclohexyl)carbamate (0.14 g), Na(OAc)₃BH (0.4 g) and a few drops of AcOH were added. The reaction was placed into a heat block at 30° C. and allowed to stir for 2 h. Then acetaldehyde (0.126 mL, 2.223 mmol) was added, the reaction capped and allowed to stir overnight at room temperature. The reaction was diluted with DCM (30 mL) and then water and saturated aqueous NaHCO₃ solution were added to achieve a pH of about 8. The biphasic system was stirred well for 30 min, then partitioned, separated and back extracted with DCM. The combined organics were dried over MgSO₄, filtered and concentrated in vacuo to a residue that was dissolved in a small amount of DCM and adsorbed onto silica gel, then purified using flash column chromatography (Isco® Rf, 12 gram, 5-50% EtOAc in heptane) to afford a mixture of cis and trans cyclohexane isomers of (E)-tert-butyl(4-(ethyl(1-methoxy-3-methyl-14-oxo-5,6,9,14,15,16-hexahydrobenzo[c]pyrido[4,3-j][1]azacyclododecin-10-yl)amino)cyclohexyl)(methyl)carbamate (180 mg, 0.306 mmol, 68.8% yield) as a glassy solid. LC-MS (ES) 577.4 [M+H]⁺ (minor), 366.2 (major).

### b) (E)-10-(ethyl((trans)-4-(methylamino)cyclohexyl)amino)-3-methyl-5,6,15,16-tetrahydrobenzo[c]pyrido[4,3-j][1]azacyclododecine-1,14(2H,9H)-dione formic acid salt

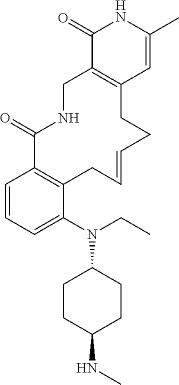 [SMILES: CCN(c1cccc2c1C/C=C/CCc1cc(C)[nH]c(=O)c1CNC2=O)[C@H]1CC[C@H](NC)CC1]

To (E)-tert-butyl(4-(ethyl(1-methoxy-3-methyl-14-oxo-5,6,9,14,15,16-hexahydrobenzo[c]pyrido[4,3-j][1]azacyclododecin-10-yl)amino)cyclohexyl)(methyl)carbamate (152 mg, 0.264 mmol) was added MeOH (1.0 mL) and then HCl (4 M in 1,4-dioxane, 3.95 mL, 15.81 mmol) to form a solution. The reaction vial was sealed and placed in a heat block at 60° C. and was stirred overnight (20 h). The volatiles were removed in vacuo to a residue, which was then dissolved in MeOH and adsorbed onto Biotage® resin (Celite®) and purified using reverse phase (Isco® Rf, 50 gram GOLD C18 aq column, 5-45% MeOH in water with 0.1% formic acid) to afford (E)-10-(ethyl(trans-4-(methylamino)cyclohexyl)amino)-3-methyl-5,6,15,16-tetrahydrobenzo[c]pyrido[4,3-j][1]azacyclododecine-1,14(2H,9H)-dione formic acid salt (23 mg, 0.044 mmol, 16.81% yield) as a white solid. LC-MS (ES) m/z=463.6 [M+H]⁺ (minor), 232.2 (minor). ¹H NMR (400 MHz, DMSO-d₆) δ: 11.29 (br. s., 1H), 8.37 (br. s., 1H), 7.99 (br. s., 1H), 7.10-7.27 (m, 2H), 6.94 (dd, J=5.3, 3.0 Hz, 1H), 5.83 (s, 1H), 5.04-5.22 (m, 2H), 4.16 (br. s., 2H), 3.52 (br. s., 2H), 2.89-3.05 (m, 2H), 2.54-2.70 (m, 4H), 2.36 (s, 3H), 2.21 (br. s., 2H), 2.05-2.15 (m, 3H), 1.94 (d, J=9.9 Hz, 2H), 1.75 (br. s., 2H), 1.26 (q, J=11.9 Hz, 2H), 1.04-1.16 (m, 2H), 0.75 (t, J=6.9 Hz, 3H). 2H not observed.

### Example 74

### (E)-10-(ethyl(cis-4-(methylamino)cyclohexyl)amino)-3-methyl-5,6,15,16-tetrahydrobenzo[c]pyrido[4,3-j][1]azacyclododecine-1,14(2H,9H)-dione formic acid salt

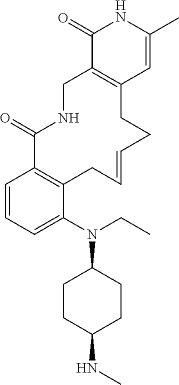 [SMILES: CCN(c1cccc2c1C/C=C/CCc1cc(C)[nH]c(=O)c1CNC2=O)[C@H]1CC[C@@H](NC)CC1]

Also isolated from the purification of Example 73(b) was (E)-10-(ethyl(cis-4-(methylamino)cyclohexyl)amino)-3-methyl-5,6,15,16-tetrahydrobenzo[c]pyrido[4,3-j][1]azacyclododecine-1,14(2H,9H)-dione formic acid salt (45 mg, 0.087 mmol, 32.9% yield) as a white solid. LC-MS (ES) m/z=463.3 [M+H]⁺ (minor), 232.2 (major). ¹H NMR (400 MHz, DMSO-d₆) δ: 11.30 (br. s., 1H), 8.36 (br. s., 1H), 7.97 (br. s., 1H), 7.13-7.25 (m, 2H), 6.87-7.02 (m, 1H), 5.84 (s, 1H), 5.09-5.25 (m, 2H), 4.17 (br. s., 2H), 3.57 (br. s., 2H), 3.13 (br. s., 1H), 2.93 (br. s., 2H), 2.77 (br. s., 1H), 2.54-2.63 (m, 2H), 2.39 (s, 3H), 2.22 (br. s., 2H), 2.11 (s, 3H), 1.25-1.85 (m, 8H), 0.76 (t, J=6.9 Hz, 3H). 2H not observed.

### Example 75

### (E)-10-(ethyl(cis-4-(3-fluoroazetidin-1-yl)cyclohexyl)amino)-3-methyl-5,6,15,16-tetrahydrobenzo[c]pyrido[4,3-j][1]azacyclododecine-1,14(2H,9H)-dione

### a) (E)-10-(ethyl(4-hydroxycyclohexyl)amino)-1-methoxy-3-methyl-5,6,15,16-tetrahydrobenzo[c]pyrido[4,3-j][1]azacyclododecin-14(9H)-one

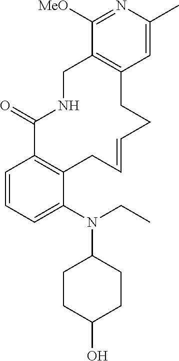 [SMILES: CCN(c1cccc2c1C/C=C/CCc1cc(C)nc(OC)c1CNC2=O)C1CCC(O)CC1]

To (E)-10-amino-1-methoxy-3-methyl-5,6,15,16-tetrahydrobenzo[c]pyrido[4,3-j][1]azacyclododecin-14(9H)-one (0.177 g, 0.525 mmol) and 4-hydroxycyclohexanone (0.120 g, 1.049 mmol) was added DCE (7 mL) and the suspension was stirred for 10 min. AcOH (0.120 mL, 2.098 mmol) and Na(OAc)₃BH (0.445 g, 2.098 mmol) were added and the reaction was stirred vigorously at room temperature overnight. Acetaldehyde (0.148 mL, 2.62 mmol) was added and the reaction was allowed to stir for 1 h. The reaction was diluted into DCM (25 mL) with stirring, then water and saturated aqueous NaHCO₃ solution were added and the biphasic system was stirred for 30 min. This was then separated and back extracted with DCM. The combined organics were dried over MgSO₄, filtered and concentrated in vacuo to a residue that was adsorbed onto silica gel and purified via flash column chromatography using (Isco® Rf-12 gram column, 10-60% (3:1 EtOAc:EtOH) in heptane) to afford a mixture of cis and trans cyclohexyl isomers of (E)-10-(ethyl(4-hydroxycyclohexyl)amino)-1-methoxy-3-methyl-5,6,15,16-tetrahydrobenzo[c]pyrido[4,3-j][1]azacyclododecin-14(9H)-one (185 mg, 0.391 mmol, 74.5% yield) as a solid. LC-MS (ES) 464.2 [M+H]⁺ (minor), 183.5 (major).

### b) (E)-10-(ethyl(4-hydroxycyclohexyl)amino)-3-methyl-5,6,15,16-tetrahydrobenzo[c]pyrido[4,3-j][1]azacyclododecine-1,14(2H,9H)-dione

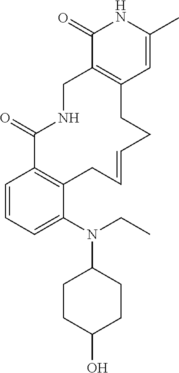 [SMILES: CCN(c1cccc2c1C/C=C/CCc1cc(C)[nH]c(=O)c1CNC2=O)C1CCC(O)CC1]

To (E)-10-(ethyl(4-hydroxycyclohexyl)amino)-1-methoxy-3-methyl-5,6,15,16-tetrahydrobenzo[c]pyrido[4,3-j][1]azacyclododecin-14(9H)-one (152 mg, 0.328 mmol) was added MeOH (0.50 mL) and then HCl (4 M in 1,4-dioxane, 4.92 mL, 19.67 mmol). The solids dissolved and the reaction was stirred for 5 min at room temperature then the reaction vessel was sealed and placed into a heat block at 70° C. and stirred overnight (20 h). The volatiles were removed and the residue was diluted with water and basified to pH 10 with concentrated NH₄OH, stirred well then partitioned with 10% MeOH in DCM (3×) The organics were combined and concentrated in vacuo to a solid that was dissolved in DCM and adsorbed onto silica gel and purified via flash column chromatography (Isco®)-Rf-12 gram GOLD column, 6-80% 90:10:1 of DCM:MeOH:NH₄OH in DCM) to afford a mixture of cis- and trans-isomers of (E)-10-(ethyl(4-hydroxycyclohexyl)amino)-3-methyl-5,6,15,16-tetrahydrobenzo[c]pyrido[4,3-j][1]azacyclododecine-1,14(2H,9H)-dione (130 mg, 0.283 mmol, 86% yield) as a white solid.

LC-MS (ES) m/z=450.2 [M+H]⁺.

### c) (E)-10-(ethyl(4-oxocyclohexyl)amino)-3-methyl-5,6,15,16-tetrahydrobenzo[c]pyrido[4,3-j][1]azacyclododecine-1,14(2H,9H)-dione

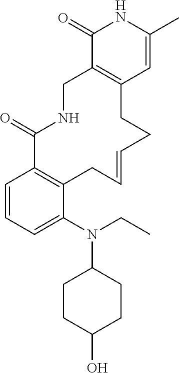 [SMILES: CCN(c1cccc2c1C/C=C/CCc1cc(C)[nH]c(=O)c1CNC2=O)C1CCC(O)CC1]

A mixture of cis- and trans-(E)-10-(ethyl(4-hydroxycyclohexyl)amino)-3-methyl-5,6,15,16-tetrahydrobenzo[c]pyrido[4,3-j][1]azacyclododecine-1,14(2H,9H)-dione (125 mg, 0.278 mmol) in DCM (4 mL) was stirred for 10 min and then Dess-Martin periodinane (142 mg, 0.334 mmol) was added and the reaction mixture was stirred for 1 h. Silica gel was then added and the mixture concentrated to dryness, and then purified via flash column chromatography using (Isco® Rf-4 gram column, 5-65% (10% MeOH in DCM) in DCM) to afford (E)-10-(ethyl(4-oxocyclohexyl)amino)-3-methyl-5,6,15,16-tetrahydrobenzo[c]pyrido[4,3-j][1]azacyclododecine-1,14(2H,9H)-dione (103 mg, 0.219 mmol, 79% yield) as a solid. LC-MS (ES) m/z=448.2 [M+H]⁺ (minor), 176.5 (major).

### d) (E)-10-(ethyl(cis-4-(3-fluoroazetidin-1-yl)cyclohexyl)amino)-3-methyl-5,6,15,16-tetrahydrobenzo[c]pyrido[4,3-j][1]azacyclododecine-1,14(2H,9H)-dione

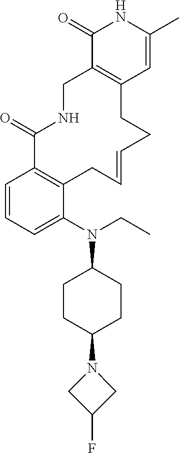 [SMILES: CCN(c1cccc2c1C/C=C/CCc1cc(C)[nH]c(=O)c1CNC2=O)[C@H]1CC[C@@H](N2CC(F)C2)CC1]

To (E)-10-(ethyl(4-oxocyclohexyl)amino)-3-methyl-5,6,15,16-tetrahydrobenzo[c]pyrido[4,3-j][1]azacyclododecine-1,14(2H,9H)-dione (100 mg, 0.223 mmol) and 3-fluoroazetidine hydrochloride (100 mg, 0.894 mmol) were added DCE (4 mL), MeOH (1 mL) and DIPEA (0.160 ml, 0.916 mmol). The resulting solution was stirred and then AcOH (0.051 mL, 0.894 mmol) was added and stirred for 10 min. Na(OAc)₃BH (189 mg, 0.894 mmol) was then added and stirred for 1 h. The reaction mixture was diluted into DCM (20 mL) with stirring and saturated aqueous NaHCO₃ was added and a few drops of concentrated NH₄OH to achieve a pH of about 10 and the mixture was stirred for 30 min. This was partitioned and back extracted with DCM (1×). The combined organics were dried over MgSO₄, filtered, and concentrated in vacuo to a solid which was then dried under high vacuum for 1 h. The solid was dissolved in DCM, adsorbed onto silica gel, and then purified via flash column chromatography using (Isco®, Rf-4 gram Gold silica column, 5-80% 90:10:1 (DCM:MeOH:NH₄OH) in DCM) to afford (E)-10-(ethyl(cis-4-(3-fluoroazetidin-1-yl)cyclohexyl)amino)-3-methyl-5,6,15,16-tetrahydrobenzo[c]pyrido[4,3-j][1]azacyclododecine-1,14(2H,9H)-dione (55 mg, 0.100 mmol, 44.7% yield) as a solid. LC-MS (ES) m/z=507.4 [M+H]⁺ (minor), 254.3 (major). ¹H NMR (400 MHz, DMSO-d₆) δ: 11.29 (s, 1H), 7.97 (t, J=4.9 Hz, 1H), 7.12-7.22 (m, 2H), 6.88-6.95 (m, 1H), 5.83 (s, 1H), 4.96-5.24 (m, 4H), 4.16 (br. s., 2H), 3.42-3.58 (m, 4H), 2.79-3.03 (m, 5H), 2.45-2.60 (m, 2H), 2.21 (br. s., 2H), 2.08-2.16 (m, 4H), 1.52-1.66 (m, 2H), 1.43 (br. s., 2H), 1.36 (br. s., 2H), 0.75 (t, J=6.9 Hz, 3H).

### Example 76

### (E)-10-(ethyl(trans-4-(3-fluoroazetidin-1-yl)cyclohexyl)amino)-3-methyl-5,6,15,16-tetrahydrobenzo[c]pyrido[4,3-j][1]azacyclododecine-1,14(2H,9H)-dione

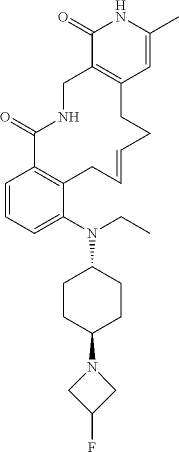 [SMILES: CCN(c1cccc2c1C/C=C/CCc1cc(C)[nH]c(=O)c1CNC2=O)[C@H]1CC[C@H](N2CC(F)C2)CC1]

Also isolated from the purification of Example 75(d) was (E)-10-(ethyl(trans-4-(3-fluoroazetidin-1-yl)cyclohexyl)amino)-3-methyl-5,6,15,16-tetrahydrobenzo[c]pyrido[4,3-j][1]azacyclododecine-1,14(2H,9H)-dione (19 mg, 0.037 mmol, 16.45% yield) as a solid. LC-MS (ES) m/z=507.4 [M+H]⁺ (minor), 254.3 (major). ¹H NMR (400 MHz, DMSO-d₆) δ: 11.28 (s, 1H), 7.98 (t, J=4.8 Hz, 1H), 7.09-7.23 (m, 2H), 6.85-6.97 (m, 1H), 5.83 (s, 1H), 4.94-5.21 (m, 3H), 4.14 (br. s., 2H), 3.38-3.61 (m, 4H), 2.87-3.05 (m, 4H), 2.56-2.68 (m, 3H), 2.21 (br. s., 2H), 2.11 (s, 3H), 1.90 (t, J=10.7 Hz, 1H), 1.62-1.77 (m, 4H), 1.13-1.36 (m, 2H), 0.65-0.88 (m, 5H).

### Example 77

### (E)-10-(azepan-4-yl(ethyl)amino)-3-methyl-5,6,15,16-tetrahydrobenzo[c]pyrido[4,3-j][1]azacyclododecine-1,14(2H,9H)-dione

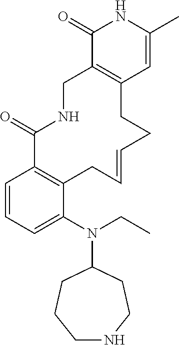 [SMILES: CCN(c1cccc2c1C/C=C/CCc1cc(C)[nH]c(=O)c1CNC2=O)C1CCCNCC1]

A suspension of (E)-10-amino-1-methoxy-3-methyl-5,6,15,16-tetrahydrobenzo[c]pyrido[4,3-j][1]azacyclododecin-14(9H)-one (0.12 g, 0.356 mmol) and tert-butyl 4-oxoazepane-1-carboxylate (0.121 g, 0.569 mmol) in DCE (6 mL) was stirred for 10 min. Then AcOH (0.081 mL, 1.423 mmol) was added followed by Na(OAc)₃BH (0.302 g, 1.423 mmol) and the suspension was stirred vigorously at room temperature overnight. Additional tert-butyl 4-oxoazepane-1-carboxylate (0.1 g) was added and the reaction mixture was heated to 37° C. and allowed to stir for 5 h. To the reaction was then added acetaldehyde (0.100 mL, 1.778 mmol) via pipette and the reaction mixture was allowed to stir overnight. The reaction was diluted into DCM (20 mL) with stirring and saturated NaHCO₃ was added and stirred for 20 min. The layers were separated and back extracted with DCM (1×). The combined organics were dried over MgSO₄, filtered and concentrated in vacuo to a residue that was dried on high vacuum for 1 h. Diluted with DCM and added in silica gel and concentrated in vacuo to dryness and dried on high vacuum for 30 min, then purified by flash column chromatography (12 gram Isco® GOLD silica column, 8-85% (3 to 1 EtOAc to EtOH) in heptane) to afford a liquid. The liquid was dissolved in MeOH (1.2 mL) and HCl (4 M in 1,4-dioxane, 4.76 mL, 19.06 mmol) was added, the reaction was stirred for 5 min at room temperature then placed into a heat block at 70° C. and stirred overnight (20 h). Allowed to cool in ice bath then vented reaction with needle then transferred to a 50 mL RB Flask with MeOH/DCM. Removed volatiles in vacuo to a residue that was dried on high vacuum for 1 h. Dissolved in 10% MeOH/DCM and then added in concentrated NH₄OH (0.4 mL) and swirled for 5 min, then adsorbed onto silica gel and purified by flash column chromatography (4 gram Isco® GOLD silica column, 15-100% (90:10:1 of DCM:MeOH:NH₄OH) in DCM, then 80:20:2 DCM:MeOH:NH₄OH) to afford a residue that was triturated with TBME to afford a white solid. This solid was treated with water (dissolves) pH of about 5. Then added in 2 drops of NH₄OH and solids precipitated. This was then extracted 10% MeOH/DCM (3×), combined organics and dried over MgSO₄ then filtered and rinsed with (90:10:1 DCM:MeOH:NH₄OH) and concentrated under nitrogen stream. The residue was triturated with TBME to obtain a solid then dried solid in vacuum oven overnight to afford (E)-10-(azepan-4-yl)ethyl)amino)-3-methyl-5,6,15,16-tetrahydrobenzo[c]pyrido[4,3-j][1]azacyclododecine-1,14(2H,9H)-dione (32 mg, 0.070 mmol, 20.17% yield) as a solid. LC-MS (ES) m/z=449.3 [M+H]⁺ (minor), 352.2 (major). ¹H NMR (400 MHz, DMSO-d₆) δ: 11.23 (br. s., 1H), 8.01 (br. s., 1H), 7.03-7.27 (m, 2H), 6.82-6.96 (m, 1H), 5.84 (s, 1H), 5.08-5.22 (m, 2H), 4.16 (br. s., 2H), 3.48 (br. s., 3H), 2.93-3.06 (m, 2H), 2.78-2.90 (m, 1H), 2.39-2.77 (m, 5H), 2.22 (br. s., 2H), 2.11 (s, 3H), 1.75 (br. s., 2H), 1.49-1.68 (m, 3H), 1.18-1.32 (m, 2H), 0.78 (t, J=6.9 Hz, 3H).

### Example 78

### (E)-10-(ethyl(cis-4-hydroxycyclohexyl)amino)-3-methyl-5,6,15,16-tetrahydrobenzo[c]pyrido[4,3-j][1]azacyclododecine-1,14(2H,9H)-dione

### a) (E)-10-(ethyl(cis-4-hydroxycyclohexyl)amino)-1-methoxy-3-methyl-5,6,15,16-tetrahydrobenzo[c]pyrido[4,3-j][1]azacyclododecin-14(9H)-one and (E)-10-(ethyl(trans-4-hydroxycyclohexyl)amino)-1-methoxy-3-methyl-5,6,15,16-tetrahydrobenzo[c]pyrido[4,3-j][1]azacyclododecin-14(9H)-one

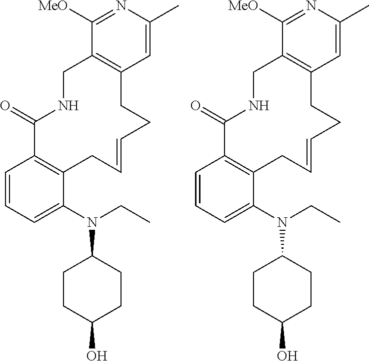 [SMILES: CCN(c1cccc2c1C/C=C/CCc1cc(C)nc(OC)c1CNC2=O)[C@H]1CC[C@@H](O)CC1 ; CCN(c1cccc2c1C/C=C/CCc1cc(C)nc(OC)c1CNC2=O)[C@H]1CC[C@H](O)CC1]

A suspension of (E)-10-amino-1-methoxy-3-methyl-5,6,15,16-tetrahydrobenzo[c]pyrido[4,3-j][1]azacyclododecin-14(9H)-one (0.66 g, 1.956 mmol), and 4-hydroxycyclohexanone (0.447 g, 3.91 mmol) in DCE (20 mL) was stirred for 10 min. Then added in AcOH (0.448 mL, 7.82 mmol) then Na(OAc)₃BH (1.658 g, 7.82 mmol) and stirred vigorously at room temperature overnight. Then added to reaction acetaldehyde (0.552 mL, 9.78 mmol) via pipette, and capped and allowed to stir for 1 h. The reaction was diluted into DCM (100 mL) with stirring then added in water and saturated NaHCO₃ and stirred for 30 min. The layers were separated and back extracted with DCM, and the combined organics were dried with MgSO₄, filtered and concentrated in vacuo. The resulting residue was dissolved in DCM and concentrated onto silica and dried on high vacuum for 1 h, then purified by flash column chromatography (40 gram Isco® GOLD silica column, 8-55% (3 to 1 EtOAc to EtOH) in heptane) to afford a residue that was triturated with TBME to afford (E)-10-(ethyl(cis-4-hydroxycyclohexyl)amino)-1-methoxy-3-methyl-5,6,15,16-tetrahydrobenzo[c]pyrido[4,3-j][1]azacyclododecin-14(9H)-one (150 mg, 0.324 mmol, 16.54% yield) as a white solid. LC-MS (ES) m/z=464.3 [M+H]⁺ (minor), 183.6 (major).

Also isolated was (E)-10-(ethyl(trans-4-hydroxycyclohexyl)amino)-1-methoxy-3-methyl-5,6,15,16-tetrahydrobenzo[c]pyrido[4,3-j][1]azacyclododecin-14(9H)-one (172 mg, 0.371 mmol, 18.97% yield) as a white solid. LC-MS (ES) m/z=464.3 [M+H]⁺ (minor), 183.6 (major).

### b) (E)-10-(ethyl(cis-4-hydroxycyclohexyl)amino)-3-methyl-5,6,15,16-tetrahydrobenzo[c]pyrido[4,3-j][1]azacyclododecine-1,14(2H,9H)-dione

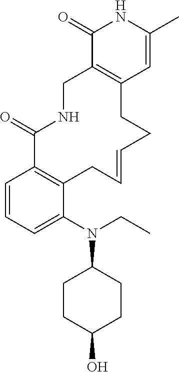 [SMILES: CCN(c1cccc2c1C/C=C/CCc1cc(C)[nH]c(=O)c1CNC2=O)[C@H]1CC[C@@H](O)CC1]

To (E)-10-(ethyl(cis-4-hydroxycyclohexyl)amino)-1-methoxy-3-methyl-5,6,15,16-tetrahydrobenzo[c]pyrido[4,3-j][1]azacyclododecin-14(9H)-one (150 mg, 0.324 mmol) in MeOH (0.50 mL) was added HCl (4 M in 1,4-dioxane, 5.26 mL, 21.03 mmol). The solid dissolved, the reaction vessel was sealed and stirred for 5 min at room temperature then placed into a heat block at 70° C. and stirred overnight (20 h). The reaction was transferred to a 50 mL RB flask, rinsed with 10% MeOH/DCM and then removed volatiles in vacuo and high vacuum to a residue that was then re-dissolved in DCM/MeOH and concentrated NH₄OH, then adsorbed onto silica gel and purified via flash column chromatography (4 gram Isco® GOLD silica column, 10-85% 90:10:1 DCM:MeOH:NH₄OH in DCM) to afford (E)-10-(ethyl(cis-4-hydroxycyclohexyl)amino)-3-methyl-5,6,15,16-tetrahydrobenzo[c]pyrido[4,3-j][1]azacyclododecine-1,14(2H,9H)-dione (95 mg, 0.205 mmol, 63.3% yield) as a solid. ¹H NMR (400 MHz, DMSO-d₆) δ: 11.30 (s, 1H), 8.01 (t, J=4.9 Hz, 1H), 7.12-7.24 (m, 2H), 6.92 (dd, J=5.3, 3.3 Hz, 1H), 5.84 (s, 1H), 5.06-5.23 (m, 2H), 4.30 (d, J=3.3 Hz, 1H), 4.16 (br. s., 2H), 3.58-3.77 (m, 1H), 3.54 (br. s., 2H), 2.97 (br. s., 2H), 2.80 (br. s., 1H), 2.45-2.61 (m, 2H), 2.21 (br. s., 2H), 2.11 (s, 3H), 1.50-1.72 (m, 4H), 1.40 (br. s., 2H), 1.21-1.36 (m, 2H), 0.76 (t, J=6.9 Hz, 3H). LC-MS (ES) m/z=450.2 [M+H]⁺.

### Example 79

### (E)-10-(ethyl(trans-4-hydroxycyclohexyl)amino)-3-methyl-5,6,15,16-tetrahydrobenzo[c]pyrido[4,3-j][1]azacyclododecine-1,14(2H,9H)-dione

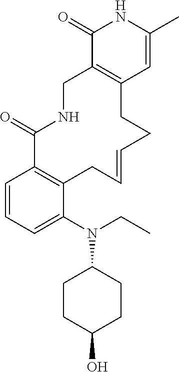 [SMILES: CCN(c1cccc2c1C/C=C/CCc1cc(C)[nH]c(=O)c1CNC2=O)[C@H]1CC[C@H](O)CC1]

To (E)-10-(ethyl(trans-4-hydroxycyclohexyl)amino)-1-methoxy-3-methyl-5,6,15,16-tetrahydrobenzo[c]pyrido[4,3-j][1]azacyclododecin-14(9H)-one (172 mg) in MeOH (0.50 mL) was added HCl (4 M in 1,4-dioxane, 5.26 mL, 21.03 mmol). The solid dissolved, the reaction vessel was sealed and stirred for 5 min at room temperature then placed into a heat block at 70° C. and stirred overnight (20 h). The reaction was transferred to a 50 mL RB flask, rinsed with 10% MeOH/DCM and then removed volatiles in vacuo and high vacuum to a residue that was then re-dissolved in DCM/MeOH and concentrated NH₄OH, then adsorbed onto silica gel and purified via flash column chromatography (4 gram Isco® GOLD silica column, 10-85% 90:10:1 DCM:MeOH:NH₄OH in DCM) to afford (E)-10-(ethyl(trans-4-hydroxycyclohexyl)amino)-3-methyl-5,6,15,16-tetrahydrobenzo[c]pyrido[4,3-j][1]azacyclododecine-1,14(2H,9H)-dione (110 mg, 0.240 mmol, 74.1% yield) as a solid. LC-MS (ES) m/z=450.2 [M+H]⁺. ¹H NMR (400 MHz, DMSO-d₆) δ: 11.30 (s, 1H), 8.01 (t, J=4.8 Hz, 1H), 7.10-7.25 (m, 2H), 6.83-6.97 (m, 1H), 5.83 (s, 1H), 5.05-5.23 (m, 2H), 4.46 (d, J=4.3 Hz, 1H), 4.16 (br. s., 2H), 3.51 (br. s., 2H), 3.23-3.33 (m, 1H), 2.88-3.05 (m, 2H), 2.40-2.70 (m, 3H), 2.21 (br. s., 2H), 2.11 (s, 3H), 1.73-1.81 (m, 2H), 1.57-1.71 (m, 2H), 1.19-1.34 (m, 2H), 0.95-1.09 (m, 2H), 0.74 (t, J=6.9 Hz, 3H).

### Example 80

### (E)-10-((cis-4-(3,3-difluoroazetidin-1-yl)cyclohexyl)(ethyl)amino)-3-methyl-5,6,15,16-tetrahydrobenzo[c]pyrido[4,3-j][1]azacyclododecine-1,14(2H,9H)-dione

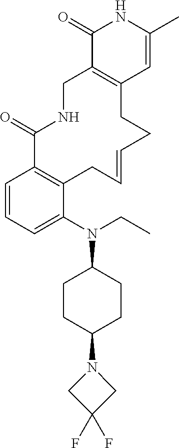 [SMILES: CCN(c1cccc2c1C/C=C/CCc1cc(C)[nH]c(=O)c1CNC2=O)[C@H]1CC[C@@H](N2CC(F)(F)C2)CC1]

To a solution of (E)-10-(ethyl(4-oxocyclohexyl)amino)-3-methyl-5,6,15,16-tetrahydrobenzo[c]pyrido[4,3-j][1]azacyclododecine-1,14(2H,9H)-dione (102 mg, 0.228 mmol) and 3,3-difluoroazetidine hydrochloride (118 mg, 0.912 mmol) in DCE (4 mL) was added AcOH (0.052 mL, 0.912 mmol) and the mixture stirred for 10 min then Na(OAc)₃BH (193 mg, 0.912 mmol) was added and stirred overnight. Diluted into DCM (20 mL) with stirring and added in saturated NaHCO₃ and stirred for 30 min. The layers were separated and back extracted with DCM (1×). The combined organics were dried over MgSO₄ and filtered and concentrated in vacuo to a solid which was dried on high vacuum for 1 h. The solid was dissolved in MeOH and adsorbed onto Biotage® Solid load and purified by reverse phase chromatography (30 gram Isco® C18aq column, 5-55% MeOH in water with 0.1% formic acid) to afford a residue that was dissolved in MeOH and 7 M NH₃ in MeOH and adsorbed onto silica gel and purified by flash column chromatography (4 gram Isco® silica column, 8-60% (90:10:1 DCM:MeOH:NH₄OH in DCM) to afford a solid which was triturated with TBME to afford (E)-10-((cis-4-(3,3-difluoroazetidin-1-yl)cyclohexyl)(ethyl)amino)-3-methyl-5,6,15,16-tetrahydrobenzo[c]pyrido[4,3-j][1]azacyclododecine-1,14(2H,9H)-dione (52 mg, 0.097 mmol, 42.6% yield) as a white solid. ¹H NMR (400 MHz, DMSO-d₆) δ: 11.30 (s, 1H), 7.99 (t, J=4.9 Hz, 1H), 7.07-7.23 (m, 2H), 6.84-7.01 (m, 1H), 5.83 (s, 1H), 5.06-5.23 (m, 2H), 4.16 (br. s., 2H), 3.37-3.67 (m, 6H), 2.81-3.04 (m, 3H), 2.45-2.60 (m, 2H), 2.16-2.30 (m, 3H), 2.05-2.13 (m, 3H), 1.52-1.77 (m, 2H), 1.44 (br. s., 2H), 1.38 (br. s., 2H), 1.16-1.32 (m, 2H), 0.76 (t, J=6.9 Hz, 3H). LC-MS (ES) m/z=525.3[M+H]⁺ (minor), 263.2 (major).

### Example 81

### (E)-10-((trans-4-(3,3-difluoroazetidin-1-yl)cyclohexyl)(ethyl)amino)-3-methyl-5,6,15,16-tetrahydrobenzo[c]pyrido[4,3-j][1]azacyclododecine-1,14(2H,9H)-dione

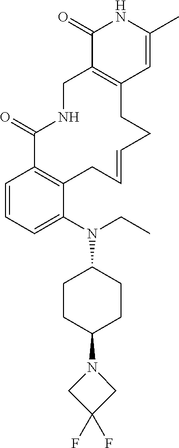 [SMILES: CCN(c1cccc2c1C/C=C/CCc1cc(C)[nH]c(=O)c1CNC2=O)[C@H]1CC[C@H](N2CC(F)(F)C2)CC1]

Also obtained from the purification of Example 80 was (E)-10-((trans-4-(3,3-difluoroazetidin-1-yl)cyclohexyl)(ethyl)amino)-3-methyl-5,6,15,16-tetrahydrobenzo[c]pyrido[4,3-j][1]azacyclododecine-1,14(2H,9H)-dione (22 mg, 0.041 mmol, 18.03% yield) as a white solid. LC-MS (ES) m/z=525.3[M+H]⁺ (minor), 173.9 (major). ¹H NMR (400 MHz, DMSO-d₆) δ: 11.29 (s, 1H), 8.00 (t, J=4.7 Hz, 1H), 7.10-7.26 (m, 2H), 6.79-7.00 (m, 1H), 5.83 (s, 1H), 5.05-5.23 (m, 2H), 4.16 (br. s., 2H), 3.42-3.58 (m, 6H), 2.98 (br. s., 2H), 2.46-2.69 (m, 3H), 2.21 (br. s., 2H), 2.11 (s, 3H), 1.93-2.07 (m, 1H), 1.57-1.78 (m, 4H), 1.14-1.32 (m, 2H), 0.78-0.93 (m, 2H), 0.75 (t, J=6.9 Hz, 3H).

### Example 82

### (E)-10-(ethyl(cis-4-((3,3,3-trifluoropropyl)amino)cyclohexyl)amino)-3-methyl-5,6,15,16-tetrahydrobenzo[c]pyrido[4,3-j][1]azacyclododecine-1,14(2H,9H)-dione

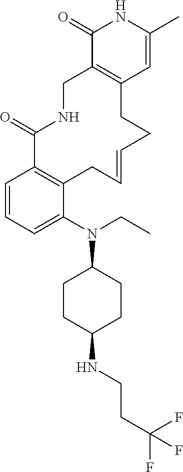 [SMILES: CCN(c1cccc2c1C/C=C/CCc1cc(C)[nH]c(=O)c1CNC2=O)[C@H]1CC[C@@H](NCCC(F)(F)F)CC1]

To a solution of (E)-10-(ethyl(4-oxocyclohexyl)amino)-3-methyl-5,6,15,16-tetrahydrobenzo[c]pyrido[4,3-j][1]azacyclododecine-1,14(2H,9H)-dione (115 mg, 0.257 mmol) and 3,3,3-trifluoropropan-1-amine (116 mg, 1.028 mmol) in DCE (4 mL) was added AcOH (0.059 mL, 1.028 mmol) then after 10 min stirring Na(OAc)₃BH (218 mg, 1.028 mmol) was added and stirred for 1 h. The mixture was diluted into DCM (20 mL) with stirring and added in saturated NaHCO₃ and stirred for 30 min. The layers were separated and back extracted with DCM (1×). The combined organics were dried over MgSO₄ and filtered and concentrated in vacuo to a solid which was dissolved in MeOH and adsorbed onto a Biotage® Solid load and purified via reverse phase chromatography (30 gram Isco® C18aq column, 5-55% MeOH in water with 0.1% formic acid) to afford a residue that was dissolved in MeOH and 7 M NH₃ in MeOH and adsorbed onto silica gel and purified by flash column chromatography (8-60% (90:10:1 DCM:MeOH:NH₄OH in DCM) to afford a solid which was triturated with TBME to afford (E)-10-(ethyl(cis-4-((3,3,3-trifluoropropyl)amino)cyclohexyl)amino)-3-methyl-5,6,15,16-tetrahydrobenzo[c]pyrido[4,3-j][1]azacyclododecine-1,14(2H,9H)-dione (51 mg, 0.091 mmol, 35.3% yield) as a solid. LC-MS (ES) m/z=545.3 [M+H]⁺ (minor), 273.3 (major). ¹H NMR (400 MHz, DMSO-d₆) δ: 11.30 (br. s., 1H), 7.99 (t, J=4.9 Hz, 1H), 7.11-7.28 (m, 2H), 6.88-7.00 (m, 1H), 5.83 (s, 1H), 5.03-5.25 (m, 2H), 4.16 (br. s., 2H), 3.53 (br. s., 2H), 2.85-3.03 (m, 3H), 2.66 (t, J=7.5 Hz, 2H), 2.50-2.60 (m, 2H), 2.29-2.43 (m, 3H), 2.16-2.24 (m, 2H), 2.11 (s, 3H), 1.65 (br. s., 2H), 1.58 (br. s., 1H), 1.50 (br. s., 2H), 1.20-1.43 (m, 4H), 0.76 (t, J=6.9 Hz, 3H).

### Example 83

### (E)-10-(ethyl(trans-4-((3,3,3-trifluoropropyl)amino)cyclohexyl)amino)-3-methyl-5,6,15,16-tetrahydrobenzo[c]pyrido[4,3-j][1]azacyclododecine-1,14(2H,9H)-dione

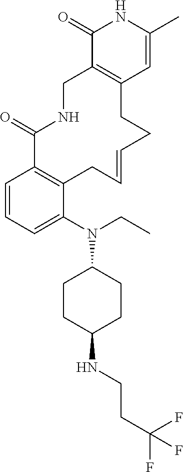 [SMILES: CCN(c1cccc2c1C/C=C/CCc1cc(C)[nH]c(=O)c1CNC2=O)[C@H]1CC[C@H](NCCC(F)(F)F)CC1]

Also obtained from the purification of Example 82 was (E)-10-(ethyl(trans-4-((3,3,3-trifluoropropyl)amino)cyclohexyl)amino)-3-methyl-5,6,15,16-tetrahydrobenzo[c]pyrido[4,3-j][1]azacyclododecine-1,14(2H,9H)-dione (14 mg, 0.025 mmol, 9.70% yield) as a solid. LC-MS (ES) m/z=545.3 [M+H]⁺ (minor), 193.9 (major). ¹H NMR (400 MHz, DMSO-d₆) δ: 11.29 (br. s., 1H), 8.00 (t, J=4.8 Hz, 1H), 7.18 (d, J=4.5 Hz, 2H), 6.93 (t, J=4.3 Hz, 1H), 5.83 (s, 1H), 5.05-5.22 (m, 2H), 4.16 (br. s., 2H), 3.51 (br. s., 2H), 2.98 (d, J=5.3 Hz, 2H), 2.50-2.72 (m, 5H), 2.15-2.41 (m, 5H), 2.11 (s, 3H), 1.82 (d, J=11.6 Hz, 2H), 1.69 (br. s., 2H), 1.60 (br. s., 1H), 1.18-1.35 (m, 2H), 0.79-0.93 (m, 2H), 0.75 (t, J=6.9 Hz, 3H).

### Example 84

### (E)-10-((trans-4-(dimethylamino)cyclohexyl)oxy)-3-methyl-5,6,15,16-tetrahydrobenzo[c]pyrido[4,3-j][1]azacyclododecine-1,14(2H,9H)-dione

### (a) 2-bromo-N-((4-(but-3-en-1-yl)-2-methoxy-6-methylpyridin-3-yl)methyl)-3-hydroxybenzamide

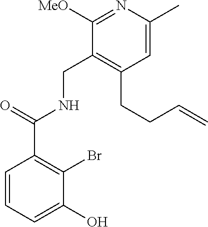 [SMILES: C=CCCc1cc(C)nc(OC)c1CNC(=O)c1cccc(O)c1Br]

A mixture of 2-bromo-3-hydroxybenzoic acid (1 g, 4.61 mmol), (4-(but-3-en-1-yl)-2-methoxy-6-methylpyridin-3-yl)methanamine (1.141 g, 5.53 mmol), HOAt (0.941 g, 6.91 mmol), EDC (1.325 g, 6.91 mmol), and N-methylmorpholine (1.520 mL, 13.82 mmol) was stirred at room temperature over the weekend. The reaction mixture was poured into water and stirred for 1 h. The precipitate was collected by filtration, and dried at the pump overnight. The residue was dissolved in EtOAc (200 mL) and washed with water (30 mL), then brine (30 mL), then the organic layer was dried over Na₂SO₄, filtered, and concentrated to afford an orange solid. The solid was purified by flash column chromatography (0-50% EtOAc in hexanes, 40 g column) to afford 2-bromo-N-((4-(but-3-en-1-yl)-2-methoxy-6-methylpyridin-3-yl)methyl)-3-hydroxybenzamide (1.3 g, 3.21 mmol, 69.6% yield) as a pale orange oil. LC-MS (ES) m/z=405.2, 407.2 [M+H]⁺.

### (b) tert-butyl(trans-4-(2-bromo-3-(((4-(but-3-en-1-yl)-2-methoxy-6-methylpyridin-3-yl)methyl)carbamoyl)phenoxy)cyclohexyl)carbamate

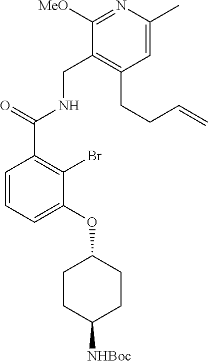 [SMILES: C=CCCc1cc(C)nc(OC)c1CNC(=O)c1cccc(O[C@H]2CC[C@H](NC(=O)OC(C)(C)C)CC2)c1Br]

To a solution of 2-bromo-N4(4-(but-3-en-1-yl)-2-methoxy-6-methylpyridin-3-yl)methyl)-3-hydroxybenzamide (1.06 g, 2.62 mmol) in DMF (15 mL) was added cis-4-((tert-butoxycarbonyl)amino)cyclohexyl methanesulfonate (1.535 g, 5.23 mmol) and Cs₂CO₃ (3.41 g, 10.46 mmol). The mixture was heated at 60° C. overnight. More cis-4-((tert-butoxycarbonyl)amino)cyclohexyl methanesulfonate (384 mg, 1.308 mmol) was added, and the reaction mixture was heated for another day. The reaction mixture was diluted with water and extracted with EtOAc (3×). The combined organic extracts were washed with water (2×), brine (ix), dried over Na₂SO₄, concentrated, and purified by flash column chromatography (0-40% EtOAc in hexane, 30 g column) to afford tert-butyl(trans-4-(2-bromo-3-(((4-(but-3-en-1-yl)-2-methoxy-6-methylpyridin-3-yl)methyl)carbamoyl)phenoxy)cyclohexyl)carbamate (1.27 g, 2.108 mmol, 81% yield) as a white solid. LC-MS (ES) m/z=602.4, 604.4 [M+H]⁺.

### (c) tert-butyl(trans-4-(2-allyl-3-(((4-(but-3-en-1-yl)-2-methoxy-6-methylpyridin-3-yl)methyl)carbamoyl)phenoxy)cyclohexyl)carbamate

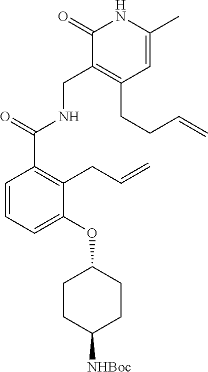 [SMILES: C=CCCc1cc(C)[nH]c(=O)c1CNC(=O)c1cccc(O[C@H]2CC[C@H](NC(=O)OC(C)(C)C)CC2)c1CC=C]

To a solution of tert-butyl(trans-4-(2-bromo-3-(((4-(but-3-en-1-yl)-2-methoxy-6-methylpyridin-3-yl)methyl)carbamoyl)phenoxy)cyclohexyl)carbamate (1.25 g, 2.074 mmol) in DME (12 mL) were added allyltrifluoroborate potassium salt (1.074 g, 7.26 mmol), CsF (1.260 g, 8.30 mmol) and Pd(PPh₃)₄ (0.240 g, 0.207 mmol). The reaction mixture was heated at 120° C. for 30 min in a microwave reactor. The reaction mixture was adsorbed onto Celite® and purified by flash column chromatography (CombiFlash®, 40 g column, 0-40% EtOAc in hexane) to afford tert-butyl(trans-4-(2-allyl-3-(((4-(but-3-en-1-yl)-2-methoxy-6-methylpyridin-3-yl)methyl)carbamoyl)phenoxy)cyclohexyl)carbamate (961 mg, 1.705 mmol, 82% yield) as an off-white foam solid. LC-MS (ES) m/z=564.5 [M+H]⁺.

### (d) tert-butyl(trans-4-4(E)-1-methoxy-3-methyl-14-oxo-5,6,9,14,15,16-hexahydrobenzo[c]pyrido[4,3-j][1]azacyclododecin-10-yl)oxy)cyclohexyl)carbamate

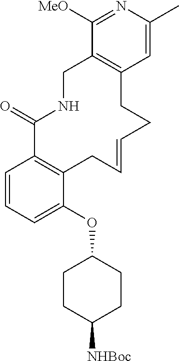 [SMILES: COc1nc(C)cc2c1CNC(=O)c1cccc(O[C@H]3CC[C@H](NC(=O)OC(C)(C)C)CC3)c1C/C=C/CC2]

To a degassed solution of tert-butyl(trans-4-(2-allyl-3-(((4-(but-3-en-1-yl)-2-methoxy-6-methylpyridin-3-yl)methyl)carbamoyl)phenoxy)cyclohexyl)carbamate (961 mg, 1.705 mmol) in DCM (80 mL) was added Grubbs II (289 mg, 0.341 mmol) and the reaction mixture was stirred at room temperature overnight under nitrogen. Additional Grubbs II (50 mg) was added and the reaction was stirred for another 5 h. The reaction mixture was concentrated, and purified by flash column chromatography (0-40% EtOAc in hexane, 30 g column) to afford a mixture of isomers. The resulting mixture was purified by Gilson® HPLC (40-80% CH₃CN in water, 0.1% TFA in mobile phase) to afford tert-butyl(trans-4-(((E)-1-methoxy-3-methyl-14-oxo-5,6,9,14,15,16-hexahydrobenzo[c]pyrido[4,3-j][1]azacyclododecin-10-yl)oxy)cyclohexyl)carbamate (132 mg) as a white solid. LC-MS (ES) m/z=536.4 [M+H]⁺.

Also isolated was tert-butyl(trans-4-(((Z)-1-methoxy-3-methyl-14-oxo-5,6,9,14,15,16-hexahydrobenzo[c]pyrido[4,3-j][1]azacyclododecin-10-yl)oxy)cyclohexyl)carbamate (120 mg) as a white solid. LC-MS (ES) m/z=536.4 [M+H]⁺.

### (f) (E)-10-((trans-4-(dimethylamino)cyclohexyl)oxy)-3-methyl-5,6,15,16-tetrahydrobenzo[c]pyrido[4,3-j][1]azacyclododecine-1,14(2H,9H)-dione

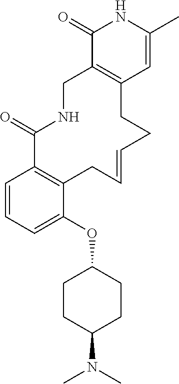 [SMILES: Cc1cc2c(c(=O)[nH]1)CNC(=O)c1cccc(O[C@H]3CC[C@H](N(C)C)CC3)c1C/C=C/CC2]

A suspension of tert-butyl(trans-4-(((E)-1-methoxy-3-methyl-14-oxo-5,6,9,14,15,16-hexahydrobenzo[c]pyrido[4,3-j][1]azacyclododecin-10-yl)oxy)cyclohexyl)carbamate (132 mg, 0.246 mmol) in HCl (4 M, 1,4-dioxane, 3 mL, 12.00 mmol) and MeOH (1 mL) was stirred at 70° C. overnight. The reaction mixture was concentrated to afford an off-white solid. To a solution of the solid and formaldehyde (37 wt % in water, 0.183 mL, 2.460 mmol) in MeOH (2 mL) was added AcOH (0.021 mL, 0.369 mmol) followed by Na(OAc)₃BH (182 mg, 0.861 mmol) in one portion and the reaction was stirred overnight at room temperature. Additional formaldehyde (37 wt % in water, 0.183 mL, 2.460 mmol) and Na(OAc)₃BH (182 mg, 0.861 mmol) were added, and the reaction stirred over the weekend. The reaction was concentrated, treated with 10 mL of (80:20:2, DCM:MeOH:NH₄OH), adsorbed onto silica, and purified by flash column chromatography (0-50% 80:20:2, DCM:MeOH:NH₄OH in DCM) to afford (E)-10-((trans-4-(dimethylamino)cyclohexyl)oxy)-3-methyl-5,6,15,16-tetrahydrobenzo[c]pyrido[4,3-j][1]azacyclododecine-1,14(2H,9H)-dione (93 mg, 0.207 mmol, 84% yield) as a white solid. LC-MS (ES) m/z=450.3 [M+H]⁺ (minor), 225.9 (major). ¹H NMR (400 MHz, DMSO-d₆) δ: 11.28 (s, 1H), 7.97 (t, J=4.9 Hz, 1H), 7.11-7.21 (m, 1H), 7.04 (d, J=7.6 Hz, 1H), 6.80 (dd, J=7.5, 0.9 Hz, 1H), 5.84 (s, 1H), 4.99-5.22 (m, 2H), 4.17 (br. s., 3H), 3.36 (br. s., 2H), 2.53-2.57 (m, 2H), 2.19-2.25 (m, 3H), 2.17 (s, 6H), 2.11 (s, 3H), 1.99-2.06 (m, 2H), 1.75-1.83 (m, 2H), 1.22-1.41 (m, 4H).

### Example 85

### (Z)-10-((trans-4-(dimethylamino)cyclohexyl)oxy)-3-methyl-5,6,15,16-tetrahydrobenzo[c]pyrido[4,3-j][1]azacyclododecine-1,14(2H,9H)-dione

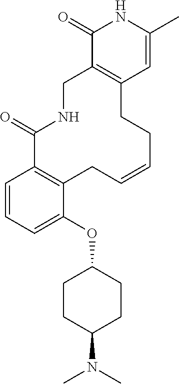 [SMILES: Cc1cc2c(c(=O)[nH]1)CNC(=O)c1cccc(O[C@H]3CC[C@H](N(C)C)CC3)c1C/C=C\CC2]

A suspension of tert-butyl(trans-4-((l-methoxy-3-methyl-14-oxo-5,6,9,14,15,16-hexahydrobenzo[c]pyrido[4,3-j][1]azacyclododecin-10-yl)oxy)cyclohexyl)carbamate (120 mg, 0.224 mmol) in HCl (4 M, 1,4-dioxane, 3 mL, 12.00 mmol) and MeOH (1 mL) was stirred at 70° C. overnight. The reaction mixture was concentrated to afford an off-white solid. To a stirred solution of the solid and formaldehyde (37 wt % in water, 0.167 mL, 2.240 mmol) in MeOH (2 mL) was added AcOH (0.019 mL, 0.336 mmol) followed by Na(OAc)₃BH (166 mg, 0.784 mmol) in one portion and the reaction was stirred overnight at room temperature. Additional formaldehyde (37 wt % in water, 0.167 mL, 2.240 mmol) and Na(OAc)₃BH (166 mg, 0.784 mmol) were added, and the reaction stirred over the weekend. The reaction was concentrated, treated with 10 mL of (80:20:2, DCM:MeOH:NH₄OH), adsorbed onto silica, and purified by flash column chromatography (0-50% 80:20:2, DCM:MeOH:NH₄OH in DCM) to afford (Z)-10-((trans-4-(dimethylamino)cyclohexyl)oxy)-3-methyl-5,6,15,16-tetrahydrobenzo[c]pyrido[4,3-j][1]azacyclododecine-1,14(2H,9H)-dione (36 mg, 0.080 mmol, 35.7% yield) as a white solid. LC-MS (ES) m/z=450.3 [M+H]⁺ (minor), 225.9 (major). ¹H NMR (400 MHz, DMSO-d₆) δ: 11.40 (s, 1H), 8.08 (t, J=5.4 Hz, 1H), 7.12-7.20 (m, 1H), 7.04 (d, J=7.8 Hz, 1H), 6.79 (dd, J=7.5, 0.9 Hz, 1H), 5.93 (s, 1H), 5.18-5.18 (m, 2H), 4.21-4.45 (m, 3H), 3.41 (br. s., 2H), 2.56-2.70 (m, 2H), 2.30-2.39 (m, 2H), 2.18 (s, 7H), 2.11 (s, 3H), 2.06 (br. s., 2H), 1.81 (br. s., 2H), 1.28-1.44 (m, 4H).

### Example 86

### (E)-10-(ethyl(cis-4-morpholinocyclohexyl)amino)-3-methyl-5,6,15,16-tetrahydrobenzo[c]pyrido[4,3-j][1]azacyclododecine-1,14(2H,9H)-dione

### a) (E)-tert-butyl(4-(ethyl(1-methoxy-3-methyl-14-oxo-5,6,9,14,15,16-hexahydrobenzo[c]pyrido[4,3-j][1]azacyclododecin-10-yl)amino)cyclohexyl)carbamate

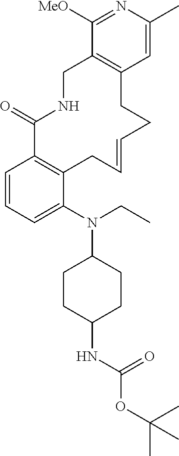 [SMILES: CCN(c1cccc2c1C/C=C/CCc1cc(C)nc(OC)c1CNC2=O)C1CCC(NC(=O)OC(C)(C)C)CC1]

A suspension of (E)-10-amino-1-methoxy-3-methyl-5,6,15,16-tetrahydrobenzo[c]pyrido[4,3-j][1]azacyclododecin-14(9H)-one (0.66 g, 1.956 mmol) and tert-butyl(4-oxocyclohexyl)carbamate (0.834 g, 3.91 mmol) in DCE (25 mL) was stirred for 10 min. Then AcOH (0.448 mL, 7.82 mmol) and Na(OAc)₃BH (1.658 g, 7.82 mmol) were added and the suspension stirred vigorously at room temperature over the weekend. Acetaldehyde (0.552 mL, 9.78 mmol) was added, and the reaction was capped and allowed to stir for 1 h. The reaction was diluted into DCM (100 mL) with stirring then added in water and saturated NaHCO₃ and stirred for 30 min. The layers were separated and back extracted with DCM. The combined organics were dried over MgSO₄, filtered and concentrated in vacuo. The resulting residue was dissolved in MeOH, added in small amount of water then formic acid and adsorbed onto a Biotage® Isolute solid load then purified by reverse phase chromatography (100 gram Isco® C18aq column, 12-95% MeOH in water with 0.1% formic acid) to afford a solid that was triturated with TBME to afford a mixture of cis- and trans-cyclochexyl isomers of (E)-tert-butyl(4-(ethyl(1-methoxy-3-methyl-14-oxo-5,6,9,14,15,16-hexahydrobenzo[c]pyrido[4,3-j][1]azacyclododecin-10-yl)amino)cyclohexyl)carbamate (934 mg, 1.627 mmol, 83% yield) as a solid. LC-MS (ES) m/z=563.5 [M+H]⁺ (minor), 254.2 (major).

### b) (E)-10-((cis-4-aminocyclohexyl)(ethyl)amino)-1-methoxy-3-methyl-5,6,15,16-tetrahydrobenzo[c]pyrido[4,3-j][1]azacyclododecin-14(9H)-one formic acid salt

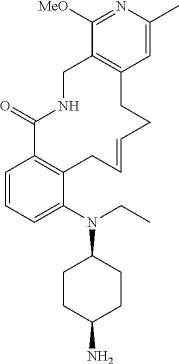 [SMILES: CCN(c1cccc2c1C/C=C/CCc1cc(C)nc(OC)c1CNC2=O)[C@H]1CC[C@@H](N)CC1]

A mixture of cis- and trans-cyclochexyl isomers of (E)-tert-butyl(4-(ethyl(1-methoxy-3-methyl-14-oxo-5,6,9,14,15,16-hexahydrobenzo[c]pyrido[4,3-j][1]azacyclododecin-10-yl)amino)cyclohexyl)carbamate (934 mg, 1.660 mmol) was dissolved in DCM (20 mL) and stirred at room temperature under nitrogen. To this was added TFA (4.48 mL, 58.1 mmol) and the reaction was allowed to stir for 1 h. The reaction mixture was concentrated in vacuo, and the resulting residue was dissolved in CH₃CN (10 mL) and formic acid (1 mL) was added and the resulting mixture concentrated in vacuo and dried overnight. The residue was dissolved in MeOH and pre-absorbed onto Biotage® Isolute resin, then purified via reverse phase chromatography (Isco® Rf-100 gram C18 aq column, 15-50% MeOH in water with 0.1% formic acid) to afford (E)-10-((cis-4-aminocyclohexyl)(ethyl)amino)-1-methoxy-3-methyl-5,6,15,16-tetrahydrobenzo[c]pyrido[4,3-j][1]azacyclododecin-14(9H)-one formic acid salt (402 mg, 0.775 mmol, 46.7% yield) as a white solid. LC-MS (ES) m/z=563.5 [M+H]⁺ (minor), 232.2 (major).

### c) (E)-10-(ethyl(cis-4-morpholinocyclohexyl)amino)-1-methoxy-3-methyl-5,6,15,16-tetrahydrobenzo[c]pyrido[4,3-j][1]azacyclododecin-14(9H)-one

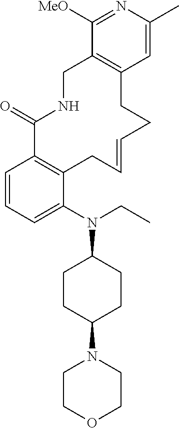 [SMILES: CCN(c1cccc2c1C/C=C/CCc1cc(C)nc(OC)c1CNC2=O)[C@H]1CC[C@@H](N2CCOCC2)CC1]

To (E)-10-((cis-4-aminocyclohexyl)(ethyl)amino)-1-methoxy-3-methyl-5,6,15,16-tetrahydrobenzo[c]pyrido[4,3-j][1]azacyclododecin-14(9H)-one formic acid salt (420 mg, 0.826 mmol) in MeOH was added HCl (4 M in 1,4-dioxane, 3 mL) and the mixture was concentrated in vacuo, repeated once. To the resulting solid was added CH₃CN (25 mL) then DIPEA (0.361 mL, 2.064 mmol), the mixture was stirred and then K₂CO₃ (228 mg, 1.651 mmol) was added then 1-bromo-2-(2-bromoethoxy)ethane (0.325 mL, 0.991 mmol) and the mixture was stirred well for 10 min, fitted with a condenser and nitrogen inlet and heated at 85° C. for 6 h. The reaction was then diluted with DCM (100 mL) and then water (30 mL) and stirred for 30 min. The mixture was partitioned and back extracted with DCM and the combined organics were dried over MgSO₄, filtered and concentrated in vacuo. The residue was adsorbed onto silica gel and purified via flash column chromatography (Isco® Rf-12 gram silica column, 10-65% (3:1 EtOAc to EtOH+1% NH₄OH) in heptane) to afford E)-10-(ethyl(cis-4-morpholinocyclohexyl)amino)-1-methoxy-3-methyl-5,6,15,16-tetrahydrobenzo[c]pyrido[4,3-j][1]azacyclododecin-14(9H)-one (290 mg, 0.533 mmol, 64.6% yield) as a solid. LC-MS (ES) m/z=533.4 [M+H]⁺ (minor), 267.3 (major).

### d) (E)-10-(ethyl(cis-4-morpholinocyclohexyl)amino)-3-methyl-5,6,15,16-tetrahydrobenzo[c]pyrido[4,3-j][1]azacyclododecine-1,14(2H,9H)-dione

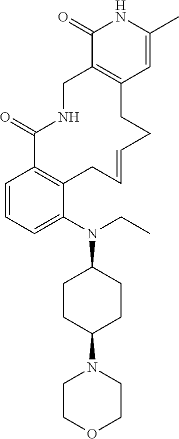 [SMILES: CCN(c1cccc2c1C/C=C/CCc1cc(C)[nH]c(=O)c1CNC2=O)[C@H]1CC[C@@H](N2CCOCC2)CC1]

A solution of (E)-10-(ethyl(cis-4-morpholinocyclohexyl)amino)-1-methoxy-3-methyl-5,6,15,16-tetrahydrobenzo[c]pyrido[4,3-j][1]azacyclododecin-14(9H)-one (290 mg, 0.544 mmol) in MeOH (1.0 mL) and HCl (4 M in 1,4-dioxane, 6.80 mL, 27.2 mmol) was stirred for 5 min at room temperature then placed into a heat block at 70° C. and stirred overnight (18 h). The reaction was allowed to cool in an ice bath then vented with a needle and transferred to a 50 mL RB flask with MeOH/DCM. Volatiles were removed in vacuo to a residue that was dried on high vacuum for 1 h. The resulting residue was dissolved in 10% MeOH/DCM and added in 4 mL of 7 M NH₃ in MeOH then adsorbed onto silica gel and purified by flash column chromatography (12 gram Isco® GOLD silica column, 10-70% (90:10:1 DCM:MeOH:NH₄OH) in DCM) to afford a residue that was triturated with TBME to afford (E)-10-(ethyl(cis-4-morpholinocyclohexyl)amino)-3-methyl-5,6,15,16-tetrahydrobenzo[c]pyrido[4,3-j][1]azacyclododecine-1,14(2H,9H)-dione (224 mg, 0.423 mmol, 78% yield) as a solid. LC-MS (ES) m/z=519.3 [M+H]⁺ (minor), 260.2 (major). ¹H NMR (400 MHz, DMSO-d₆) δ: 11.29 (s, 1H), 8.02 (t, J=4.9 Hz, 1H), 7.18 (d, J=4.5 Hz, 2H), 6.87-7.01 (m, 1H), 5.83 (s, 1H), 5.05-5.24 (m, 2H), 4.16 (br. s., 2H), 3.47-3.64 (m, 6H), 3.16 (br. s., 1H), 2.75-3.05 (m, 2H), 2.45-2.60 (m, 2H), 2.37 (br. s., 4H), 2.21 (br. s., 2H), 2.04-2.18 (m, 4H), 1.61 (br. s., 4H), 1.17-1.46 (m, 4H), 0.76 (t, J=6.9 Hz, 3H).

### Assay Protocol 1

Compounds contained herein were evaluated for their ability to inhibit the methyltransferase activity of EZH2 within the PRC2 complex. Human PRC2 complex was prepared by co-expressing each of the 5 member proteins (FLAG-EZH2, EED, SUZ12, RbAp48, AEBP2) in Sf9 cells followed by co-purification. Enzyme activity was measured in a scintillation proximity assay (SPA) where a tritiated methyl group is transferred from 3H-SAM to a lysine residue on Histone H3 of a mononucleosome, purified from HeLa cells. Mononucleosomes were captured on SPA beads and the resulting signal is read on a ViewLux plate reader.

### Part A. Compound Preparation

1. Prepare 10 mM stock of compounds from solid in 100% DMSO.
2. Set up an 11-point serial dilution (1:3 dilution, top concentration 10 mM) in 100% DMSO for each test compound in a 384 well plate leaving columns 6 and 18 for DMSO controls.
3. Dispense 100 nL of compound from the dilution plate into reaction plates (Grenier Bio-One, 384-well, Cat#784075).

### Part B. Reagent Preparation

Prepare the following solutions:
1. 50 mM Tris-HCl, pH 8: Per 1 L of base buffer, combine 1 M Tris-HCl, pH 8 (50 mL) and distilled water (950 mL).
2. 1× Assay Buffer: Per 10 mL of 1× Assay Buffer, combine 50 mM Tris-HCl, pH 8 (9958 uL), 1 M MgCl₂ (20 uL), 2 M DTT (20 uL), and 10% Tween-20 (2 uL) to provide a final concentration of 50 mM Tris-HCl, pH 8, 2 mM MgCl₂, 4 mM DTT, 0.002% Tween-20.
3. 2× Enzyme Solution: Per 10 mL of 2× Enzyme Solution, combine 1× Assay Buffer and PRC2 complex to provide a final enzyme concentration of 10 nM.
4. SPA Bead Suspension: Per 1 mL of SPA Bead Suspension, combine PS-PEI coated LEADSeeker beads (40 mg) and ddH2O (1 mL) to provide a final concentration of 40 mg/mL.
5. 2× Substrate Solution: Per 10 mL of 2× Substrate Solution, combine 1× Assay Buffer (9728.55 uL), 800 ug/mL mononucleosomes (125 uL), 1 mM cold SAM (4 uL), and 7.02 uM 3H-SAM (142.45 uL; 0.55 mCi/mL) to provide a final concentration of 5 ug/mL nucleosomes, 0.2 uM cold SAM, and 0.05 uM 3H-SAM.
6. 2.67× Quench/Bead Mixture: Per 10 mL of 2.67× Quench/Bead Mixture, combine ddH₂O (9358 uL), 10 mM cold SAM (267 uL), 40 mg/mL Bead Suspension (375 uL) to provide a final concentration of 100 uM cold SAM and 0.5 mg/mL SPA beads.

### Part C. Assay Reaction in 384-Well Grenier Bio-One Plates

### Compound Addition

1. Dispense 100 nL/well of 100× Compound to test wells (as noted above).
2. Dispense 100 nL/well of 100% DMSO to columns 6 & 18 for high and low controls, respectively.

### Assay

1. Dispense 5 uL/well of 1× Assay Buffer to column 18 (low control reactions).
2. Dispense 5 uL/well of 2× Enzyme Solution to columns 1-17, 19-24.
3. Spin assay plates for ˜1 minute at 500 rpm.
4. Stack the assay plates, covering the top plate.
5. Incubate the compound/DMSO with the enzyme for 30 minutes at room temperature.
6. Dispense 5 uL/well of 2× Substrate Solution to columns 1-24.
7. Spin assay plates for ˜1 minute at 500 rpm.
8. Stack the assay plates, covering the top plate.
9. Incubate the assay plates at room temperature for 1 hour.

### Quench/Bead Addition

1. Dispense 5 uL/well of the 3× Quench/Bead Mixture to columns 1-24.
2. Seal the top of each assay plate with adhesive TopSeal.
3. Spin assay plates for ˜1 minute at 500 rpm.
4. Equilibrate the plates for >20 min.

### Read Plates

1. Read the assay plates on the Viewlux Plate Reader utilizing the 613 nm emission filter with a 300 s read time.
   Reagent addition can be done manually or with automated liquid handler.
   The final DMSO concentration in this assay is 1%.
   The positive control is in column 6; negative control is in column 18.
   Final starting concentration of compounds is 100 μM.

### Results

Percent inhibition was calculated relative to the DMSO control for each compound concentration and the resulting values were fit using standard IC₅₀ fitting parameters within the ABASE data fitting software package.

Exemplified compounds of the present invention were generally tested according to the above or an analogous assay and were found to be inhibitors of EZH2. Specific biological activities tested according to assays described herein are listed in the following table. Repeating the assay run(s) may result in somewhat different IC₅₀ values.

| | EZH2 IC₅₀ |
|---|---|
| Example | (nM) |
| 1 | 32 |
| 2 | 25 |
| 3 | 100 |
| 4 | 2,512 |
| 5 | 2,512 |
| 6 | 100 |
| 7 | 1,585 |
| 8 | 63 |
| 9 | 79 |
| 10 | 1,000 |
| 11 | 501 |
| 12 | 126 |
| 13 | 794 |
| 14 | 50 |
| 15 | 501 |
| 16 | 501 |
| 17 | 32 |
| 18 | 100 |
| 19 | 40 |
| 20 | 25 |
| 21 | 100 |
| 22 | 6,310 |
| 23 | 126 |
| 24 | 631 |
| 25 | 158 |
| 26 | 12,589 |
| 27 | 316 |
| 28 | 631 |
| 29 | 1,259 |
| 30 | 16 |
| 31 | 13 |
| 32 | 50 |
| 33 | 40 |
| 34 | 63 |
| 35 | 40 |
| 36 | 16 |
| 37 | 50 |
| 38 | 100 |
| 39 | 1,259 |
| 40 | 32 |
| 41 | 40 |
| 42 | 20 |
| 43 | 63 |
| 51 | 200 |
| 52 | 631 |
| 54 | 3,981 |

### Assay Protocol 2

Compounds contained herein were evaluated for their ability to inhibit the methyltransferase activity of EZH2 within the PRC2 complex. Human PRC2 complex was prepared by co-expressing each of the 5 member proteins (FLAG-EZH2, EED, SUZ12, RbAp48, AEBP2) in Sf9 cells followed by co-purification. Enzyme activity was measured in a scintillation proximity assay (SPA) where a tritiated methyl group is transferred from 3H-SAM to a lysine residue on a biotinylated, unmethylated peptide substrate derived from histone H3. The peptides were captured on streptavidin-coated SPA beads and the resulting signal was read on a ViewLux plate reader.

### Part A. Compound Preparation

4. Prepare 10 mM stock of compounds from solid in 100% DMSO.
5. Set up an 11-point serial dilution (1:4 dilution, top concentration 10 mM) in 100% DMSO for each test compound in a 384 well plate leaving columns 6 and 18 for DMSO controls.
6. Dispense 10 nL of compound from the dilution plate into reaction plates (Corning, 384-well polystyrene NBS, Cat#3673).

### Part B. Reagent Preparation

Prepare the following solutions:
7. 1× Base Buffer, 50 mM Tris-HCl, pH 8, 2 mM MgCl₂: Per 1 L of base buffer, combine 1 M Tris-HCl, pH 8 (50 mL), 1 M MgCl₂ (2 mL), and distilled water (948 mL).
8. 1× Assay Buffer: Per 10 mL of 1× Assay Buffer, combine 1× Base Buffer (9.96 mL), 1 M DTT (40 uL), and 10% Tween-20 (1 uL) to provide a final concentration of 50 mM Tris-HCl, pH 8, 2 mM MgCl₂, 4 mM DTT, 0.001% Tween-20.
9. 2× Enzyme Solution: Per 10 mL of 2× Enzyme Solution, combine 1× Assay Buffer (9.99 mL) and 3.24 uM EZH2 5 member complex (6.17 uL) to provide a final enzyme concentration of 1 nM.
10. SPA Bead Solution: Per 1 mL of SPA Bead Solution, combine Streptavidin coated SPA beads (PerkinElmer, Cat# RPNQ0261, 40 mg) and 1× Assay Buffer (1 mL) to provide a working concentration of 40 mg/mL.
11. 2× Substrate Solution: Per 10 mL of 2× Substrate Solution, combine 40 mg/mL SPA Bead Solution (375 uL), 1 mM biotinylated histone H3K27 peptide (200 uL), 12.5 uM 3H-SAM (240 uL; 1 mCi/mL), 1 mM cold SAM (57 uL), and 1× Assay Buffer (9.13 mL) to provide a final concentration of 0.75 mg/mL SPA Bead Solution, 10 uM biotinylated histone H3K27 peptide, 0.15 uM 3H-SAM (˜12 uCi/mL 3H-SAM), and 2.85 uM cold SAM.
12. 2.67× Quench Solution: Per 10 mL of 2.67× Quench Solution, combine 1× Assay Buffer (9.73 mL) and 10 mM cold SAM (267 uL) to provide a final concentration of 100 uM cold SAM.

### Part C. Assay Reaction in 384-Well Grenier Bio-One Plates

### Compound Addition

3. Stamp 10 nL/well of 1000× Compound to test wells (as noted above).
4. Stamp 10 nL/well of 100% DMSO to columns 6 & 18 (high and low controls, respectively).

### Assay

10. Dispense 5 uL/well of 1× Assay Buffer to column 18 (low control reactions).
11. Dispense 5 uL/well of 2× Substrate Solution to columns 1-24 (note: substrate solution should be mixed to ensure homogeneous bead suspension before dispensing into matrix reservoir).
12. Dispense 5 uL/well of 2× Enzyme Solution to columns 1-17, 19-24.
13. Incubate the reaction for 60 min at room temperature.

### Quench

5. Dispense 6 uL/well of the 2.67× Quench Solution to columns 1-24.
6. Seal assay plates and spin for ˜1 min at 500 rpm.
7. Dark adapt plates in the ViewLux instrument for 15-60 min.

### Read Plates

2. Read the assay plates on the Viewlux Plate Reader utilizing the 613 nm emission filter or clear filter (300 s exposure).
   Reagent addition can be done manually or with automated liquid handler.

### Results

Percent inhibition was calculated relative to the DMSO control for each compound concentration and the resulting values were fit using standard IC₅₀ fitting parameters within the ABASE data fitting software package.

Several of the exemplified compounds were generally tested according to the above or an analogous assay and were found to be inhibitors of EZH2. Specific biological activities tested according to such assays are listed in the following table. Repeating the assay run(s) may result in somewhat different IC₅₀ values.

| | EZH2 IC₅₀ |
|---|---|
| Example | (nM) |
| 1 | 100 |
| 2 | 126 |
| 3 | 158 |
| 6 | 158 |
| 8 | 251 |
| 9 | 126 |
| 12 | 200 |
| 14 | 158 |
| 17 | 126 |
| 18 | 316 |
| 19 | 79 |
| 20 | 1,000 |
| 21 | 398 |
| 30 | 79 |
| 31 | 200 |
| 32 | 158 |
| 33 | 200 |
| 34 | 126 |
| 35 | 200 |
| 36 | 126 |
| 37 | 126 |
| 38 | 158 |
| 39 | 1,995 |
| 40 | 100 |
| 41 | 40 |
| 42 | 158 |
| 43 | 200 |
| 44 | 79 |
| 45 | 50 |
| 46 | 251 |
| 47 | 200 |
| 48 | 316 |
| 49 | 158 |
| 50 | 126 |
| 51 | 398 |
| 52 | 1,995 |
| 53 | 79 |
| 54 | 10,000 |
| 55 | 251 |
| 56 | 126 |
| 57 | 100 |
| 58 | 158 |
| 59 | 316 |
| 60 | 1,259 |
| 61 | 158 |
| 62 | 251 |
| 63 | 251 |
| 64 | 251 |
| 65 | 316 |
| 66 | 251 |
| 67 | 100 |
| 68 | 126 |
| 69 | 158 |
| 70 | 126 |
| 71 | 251 |
| 72 | 79 |
| 73 | 126 |
| 74 | 158 |
| 75 | 251 |
| 76 | 126 |
| 77 | 158 |
| 78 | 158 |
| 79 | 126 |
| 80 | 200 |
| 81 | 200 |
| 82 | 251 |
| 83 | 158 |
| 84 | 200 |
| 85 | 5,012 |
| 86 | 200 |

1. A compound according to Formula (I): 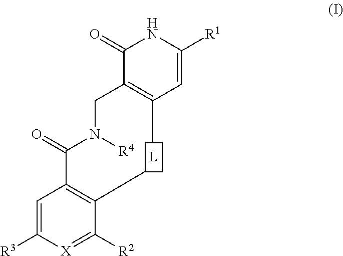
wherein:
X is CH or N;
L is (C₂-C₈)alkylenyl or (C₂-C₈)alkenylenyl, each optionally substituted by hydroxyl, wherein any one methylene unit of said (C₂-C₈)alkylenyl or (C₂-C₈)alkenylenyl is optionally replaced by —O—, —NH—, or —N(C₁-C₄)alkyl-;
R¹ is hydrogen, halogen, (C₁-C₆)alkyl, (C₂-C₆)alkenyl, (C₂-C₆)alkynyl, halo(C₁-C₄)alkyl, (C₃-C₆)cycloalkyl, (C₃-C₆)cycloalkyl(C₁-C₆)alkyl, (C₃-C₆)cycloalkyl(C₂-C₆)alkenyl, (C₅-C₆)cycloalkenyl, (C₅-C₆)cycloalkenyl(C₁-C₆)alkyl, (C₅-C₆)cycloalkenyl(C₂-C₆)alkenyl, (C₆-C₁₀)bicycloalkyl, heterocycloalkyl, heterocycloalkyl(C₁-C₆)alkyl-, heterocycloalkyl(C₂-C₆)alkenyl, phenyl, phenyl(C₁-C₆)alkyl, phenyl(C₂-C₆)alkenyl, heteroaryl, heteroaryl(C₁-C₆)alkyl, heteroaryl(C₂-C₆)alkenyl, cyano, —C(O)R^{a}, —CO₂R^{a}, —C(O)NR^{a}R^{b}, —C(O)NR^{a}NR^{a}R^{b}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OC(O)R^{a}, or —OC(O)NR^{a}R^{b}, wherein each cycloalkyl, cycloalkenyl, bicycloalkyl, heterocycloalkyl, phenyl, or heteroaryl group is optionally substituted 1, 2, or 3 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;
R² is (C₄-C₈)alkyl, (C₁-C₈)alkoxy, (C₄-C₈)cycloalkyl, (C₃-C₈)cycloalkyloxy-, heterocycloalkyl, heterocycloalkyloxy-, aryl, heteroaryl, or —NR^{a}R^{b}, wherein said (C₄-C₈)alkyl, (C₃-C₈)alkoxy, (C₄-C₈)cycloalkyl, (C₃-C₈)cycloalkyloxy-, heterocycloalkyl, heterocycloalkyloxy-, aryl, or heteroaryl is optionally substituted 1, 2, or 3 times, independently, by halogen, —OR^{a}, —NR^{a}R^{b}, —NHCO₂R^{a}, nitro, (C₁-C₃)alkyl, R^{a}R^{b}N(C₁-C₃)alkyl-, R^{a}O(C₁-C₃)alkyl-, (C₃-C₈)cycloalkyl, cyano, —CO₂R^{a}, —C(O)NR^{a}R^{b}, —SO₂NR^{a}R^{b}, aryl, or heteroaryl;
R³ is selected from the group consisting of hydrogen, halogen, (C₁-C₆)alkyl, (C₂-C₆)alkenyl, (C₂-C₆)alkynyl, (C₁-C₄)alkoxy, —B(OH)₂, (C₃-C₆)cycloalkyl, (C₃-C₆)cycloalkyl(C₁-C₄)alkyl-, (C₆-C₁₀)bicycloalkyl, heterocycloalkyl, heterocycloalkyl(C₁-C₄)alkyl-, phenyl, phenyl(C₁-C₂)alkyl, heteroaryl, heteroaryl(C₁-C₂)alkyl, cyano, —C(O)R^{a}, —CO₂R^{a}, —C(O)NR^{a}R^{b}, —C(O)NR^{a}NR^{a}R^{b}, —S(O)R^{a}, —SO₂R^{a}, —SO₂NR^{a}R^{b}, nitro, —NR^{a}R^{b}, R^{a}R^{b}N(C₁-C₄)alkyl-, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, R^{a}O(C₁-C₄)alkyl-, R^{a}O(C₃-C₆)alkynyl-, —OC(O)R^{a}, and —OC(O)NR^{a}R^{b}, wherein each cycloalkyl, bicycloalkyl, heterocycloalkyl, phenyl, or heteroaryl group is optionally substituted 1, 2, or 3 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;
R⁴ is hydrogen, (C₁-C₄)alkyl, or hydroxy(C₂-C₄)alkyl-;
each R^{c} is independently —S(O)R^{a}, —SO₂R^{a}, —NR^{a}R^{b}, —NR^{a}C(O)OR^{a}, —NR^{a}SO₂R^{b}, or —CO₂R^{a}; and
R^{a} and R^{b} are each independently hydrogen, (C₁-C₄)alkyl, hydroxy(C₁-C₄)alkyl-, (C₁-C₄)alkoxy(C₁-C₄)alkyl-, (C₃-C₆)cycloalkyl, (C₆-C₁₀)bicycloalkyl, heterocycloalkyl, phenyl, phenyl(C₁-C₂)alkyl-, heteroaryl(C₁-C₄)alkyl-, or heteroaryl, wherein any said cycloalkyl, bicycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, —NH(halo(C₁-C₄)alkyl), —N(halo(C₁-C₄)alkyl)₂, —N((C₁-C₄)alkyl)(halo(C₁-C₄)alkyl), (C₁-C₄)alkyl, halo(C₁-C₄)alkyl, hydroxy(C₁-C₄)alkyl-, (C₁-C₄)alkoxy(C₁-C₄)alkyl-, (C₃-C₆)cycloalkyl, (C₃-C₆)cycloalkyl(C₁-C₄)alkyl-, heterocycloalkyl optionally substituted by one or two halogens, heterocycloalkyl(C₁-C₄)alkyl-, heteroaryl optionally substituted by (C₁-C₄)alkyl, heteroaryl(C₁-C₄)alkyl- optionally substituted by (C₁-C₄)alkyl, (C₁-C₄)alkoxycarbonyl(C₁-C₄)alkyl-, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂;
or R^{a} and R^{b} taken together with the nitrogen to which they are attached represent a 5- or 6-membered saturated or unsaturated ring, optionally containing an additional heteroatom selected from oxygen, nitrogen, and sulfur, wherein said ring is optionally substituted 1, 2, or 3 times, independently, by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-, wherein said ring is optionally fused to a (C₃-C₆)cycloalkyl, heterocycloalkyl, phenyl, or heteroaryl ring;
or R^{a} and R^{b} taken together with the nitrogen to which they are attached represent a 6- to 10-membered bridged bicyclic ring system optionally fused to a (C₃-C₆)cycloalkyl, heterocycloalkyl, phenyl, or heteroaryl ring;
or a pharmaceutically acceptable salt thereof.

2. The compound or pharmaceutically acceptable salt according to claim 1, wherein X is CH.

3. The compound or pharmaceutically acceptable salt according to claim 1, wherein R¹ is hydrogen, halogen, (C₁-C₆)alkyl, halo(C₁-C₄)alkyl, (C₃-C₆)cycloalkyl, (C₃-C₆)cycloalkyl(C₁-C₄)alkyl, phenyl, or phenyl(C₁-C₂)alkyl.

4. The compound or pharmaceutically acceptable salt according to claim 1, wherein R¹ is (C₁-C₄)alkyl.

5. The compound or pharmaceutically acceptable salt according to claim 1, wherein R² is (C₃-C₆)alkoxy, (C₃-C₆)cycloalkyloxy-, heterocycloalkyloxy-, heterocycloalkyl, —NH((C₃-C₆)cycloalkyl), —N((C₁-C₃)alkyl)((C₃-C₆)cycloalkyl), —NH(heterocycloalkyl), or —N((C₁-C₃)alkyl)(heterocycloalkyl), wherein any said (C₃-C₆)alkoxy, (C₃-C₆)cycloalkyloxy-, heterocycloalkyloxy-, heterocycloalkyl, or (C₃-C₆)cycloalkyl is optionally substituted 1 or 2 times, independently, by halogen, hydroxyl, (C₁-C₃)alkoxy, amino, —NH(C₁-C₃)alkyl, —N((C₁-C₃)alkyl)₂, (C₁-C₃)alkyl, (C₁-C₃)alkoxy(C₁-C₃)alkyl-, amino(C₁-C₃)alkyl-, ((C₁-C₃)alkyl)NH(C₁-C₃)alkyl-, ((C₁-C₃)alkyl)₂N(C₁-C₃)alkyl-, (C₃-C₈)cycloalkyl, cyano, —CO₂R^{a}, —C(O)NR^{a}R^{b}, —SO₂NR^{a}R^{b}, phenyl, or heteroaryl.

6. The compound or pharmaceutically acceptable salt according to claim 1, wherein R² is (C₃-C₆)alkoxy, (C₃-C₈)cycloalkyloxy-, or heterocycloalkyloxy-, each of which is optionally substituted by hydroxyl, (C₁-C₃)alkoxy, amino, —NH(C₁-C₃)alkyl, —N((C₁-C₃)alkyl)₂, (C₁-C₃)alkyl, —CO₂R^{a}, —C(O)NR^{a}R^{b}, —SO₂NR^{a}R^{b}, phenyl, or heteroaryl.

7. The compound or pharmaceutically acceptable salt according to claim 1, wherein R² is cyclopentyloxy, cyclohexyloxy, pyrrolidinyloxy, piperidinyloxy, and tetrahydropyranyloxy, each of which is optionally substituted by hydroxyl, (C₁-C₃)alkoxy, amino, —NH(C₁-C₃)alkyl, —N((C₁-C₃)alkyl)₂, (C₁-C₃)alkyl, —CO₂R^{a}, —C(O)NR^{a}R^{b}, —SO₂NR^{a}R^{b}, phenyl, furanyl, thienyl, pyrrolyl, imidazolyl, pyrazolyl, triazolyl, tetrazolyl, oxazolyl, thiazolyl, isoxazolyl, isothiazolyl, oxadiazolyl, thiadiazolyl, pyridinyl, pyridazinyl, pyrazinyl, or pyrimidinyl, wherein R^{a} is (C₁-C₄)alkyl or phenyl(C₁-C₂)alkyl and R^{b} is hydrogen or (C₁-C₄)alkyl.

8. The compound or pharmaceutically acceptable salt according to claim 1, wherein R² is (C₁-C₄)alkoxy, cyclohexyloxy, or —NR^{a}R^{b}, wherein said cyclohexyloxy is optionally substituted by amino, —NH(C₁-C₃)alkyl, or —N((C₁-C₃)alkyl)₂.

9. The compound or pharmaceutically acceptable salt according to claim 1, wherein R² is —NR^{a}R^{b}.

10. The compound or pharmaceutically acceptable salt according to claim 9, wherein R^{a} is hydrogen, methyl, ethyl, cyclohexyl, tetrahydropyranyl, or piperidinyl, wherein said cyclohexyl is optionally substituted 1 or 2 times, independently, by fluorine, amino, dimethylamino, diethylamino, or morpholinyl, and wherein said piperidinyl is optionally substituted by methyl, ethyl, isopropyl, 2,2,2-trifluoroethyl, 3,3,3-trifluoropropyl, 2-hydroxyethyl, 1,3-dihydroxypropan-2-yl, cyclopropylmethyl, (1-methyl-1H-pyrazol-3-yl)methyl, (6-methylpyridin-2-yl)methyl, 1-ethoxy-2-methyl-1-oxopropan-2-yl, or methylsulfonyl; and R^{b} is hydrogen, methyl, or ethyl.

11. The compound or pharmaceutically acceptable salt according to claim 1, wherein R³ is halogen.

12. The compound or pharmaceutically acceptable salt according to claim 1, wherein R³ is heteroaryl which is optionally substituted 1 or 2 times, independently, by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —SR^{a}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;
each R^{c} is independently —S(O)R^{a}, —SO₂R^{a}, —NR^{a}R^{b}, —NR^{a}C(O)OR^{a}, —NR^{a}SO₂R^{b}, or —CO₂R^{a}; and
R^{a} and R^{b} are each independently hydrogen, (C₁-C₄)alkyl, (C₁-C₄)alkoxy(C₁-C₄)alkyl-, (C₃-C₆)cycloalkyl, heterocycloalkyl, phenyl, phenyl(C₁-C₂)alkyl-, heteroaryl(C₁-C₂)alkyl-, or heteroaryl, wherein any said cycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, (C₁-C₄)alkyl, halo(C₁-C₄)alkyl, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂;
or R^{a} and R^{b} taken together with the nitrogen to which they are attached represent a 5- or 6-membered saturated or unsaturated ring, optionally containing an additional heteroatom selected from oxygen, nitrogen, and sulfur, wherein said ring is optionally substituted 1, 2, or 3 times, independently, by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-, wherein said ring is optionally fused to a (C₃-C₆)cycloalkyl, heterocycloalkyl, phenyl, or heteroaryl ring;
or R^{a} and R^{b} taken together with the nitrogen to which they are attached represent a 6- to 10-membered bridged bicyclic ring system optionally fused to a (C₃-C₆)cycloalkyl, heterocycloalkyl, phenyl, or heteroaryl ring.

13. The compound or pharmaceutically acceptable salt according to claim 12, wherein R³ is pyridinyl which is optionally substituted by R^{c}—(C₁-C₆)alkyl-O—, R^{c}—(C₁-C₆)alkyl-S—, R^{c}—(C₁-C₆)alkyl-, (C₁-C₄)alkyl-heterocycloalkyl-, halogen, (C₁-C₆)alkyl, (C₃-C₆)cycloalkyl, halo(C₁-C₆)alkyl, cyano, —C(O)R^{a}, —CO₂R^{a}, —C(O)NR^{a}R^{b}, —S(O)R^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, phenyl, heteroaryl, phenyl(C₁-C₂)alkyl, or heteroaryl(C₁-C₂)alkyl;
each R^{c} is independently —S(O)R^{a}, —SO₂R^{a}, —NR^{a}R^{b}, —NR^{a}C(O)OR^{a}, —NR^{a}SO₂R^{b}, or —CO₂R^{a}; and
R^{a} and R^{b} are each independently hydrogen, (C₁-C₄)alkyl, (C₁-C₄)alkoxy(C₁-C₄)alkyl-, (C₃-C₆)cycloalkyl, heterocycloalkyl, phenyl, phenyl(C₁-C₂)alkyl-, heteroaryl(C₁-C₂)alkyl-, or heteroaryl, wherein any said cycloalkyl, heterocycloalkyl, phenyl, or heteroaryl group is optionally substituted 1, 2, or 3 times, independently, by halogen, hydroxyl, (C₁-C₄)alkoxy, amino, —NH(C₁-C₄)alkyl, —N((C₁-C₄)alkyl)₂, (C₁-C₄)alkyl, halo(C₁-C₄)alkyl, —CO₂H, —CO₂(C₁-C₄)alkyl, —CONH₂, —CONH(C₁-C₄)alkyl, —CON((C₁-C₄)alkyl)₂, —SO₂(C₁-C₄)alkyl, —SO₂NH₂, —SO₂NH(C₁-C₄)alkyl, or —SO₂N((C₁-C₄)alkyl)₂;
or R^{a} and R^{b} taken together with the nitrogen to which they are attached represent a 5- or 6-membered saturated or unsaturated ring, optionally containing an additional heteroatom selected from oxygen, nitrogen, and sulfur, wherein said ring is optionally substituted 1, 2, or 3 times, independently, by (C₁-C₄)alkyl, halo(C₁-C₄)alkyl, amino, —NH(C₁-C₄)alkyl, —N((C₁-C₄)alkyl)₂, hydroxyl, oxo, (C₁-C₄)alkoxy, or (C₁-C₄)alkoxy(C₁-C₄)alkyl-, wherein said ring is optionally fused to a (C₃-C₆)cycloalkyl, heterocycloalkyl, phenyl, or heteroaryl ring;
or R^{a} and R^{b} taken together with the nitrogen to which they are attached represent a 6- to 10-membered bridged bicyclic ring system optionally fused to a (C₃-C₆)cycloalkyl, heterocycloalkyl, phenyl, or heteroaryl ring.

14. The compound or pharmaceutically acceptable salt according to claim 13, wherein R³ is pyridinyl which is optionally substituted by heterocycloalkyl or (C₁-C₄)alkyl-heterocycloalkyl-.

15. The compound or pharmaceutically acceptable salt according to claim 1, wherein L is selected from the group consisting of:

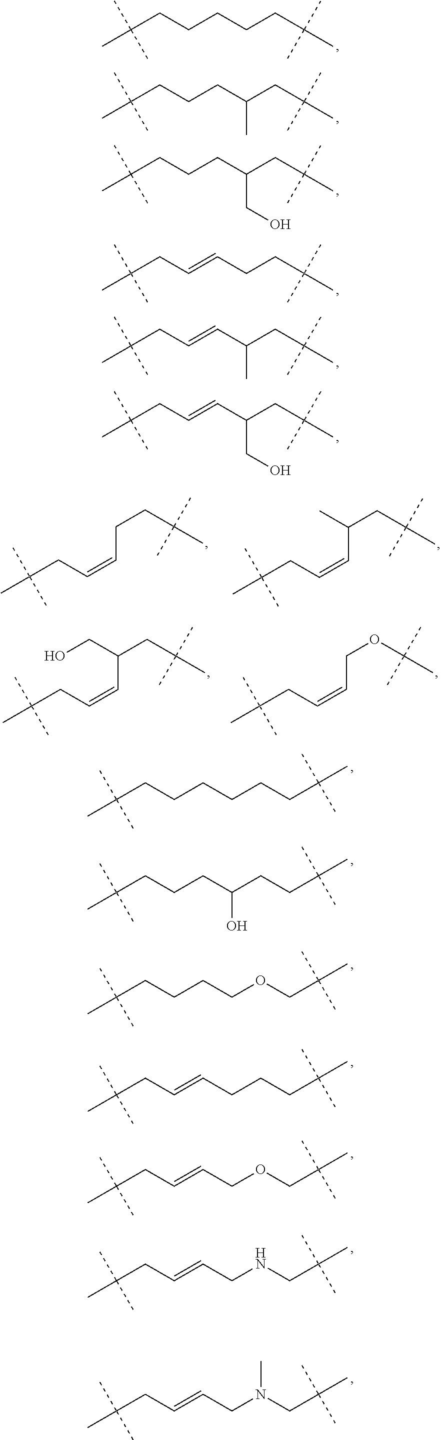

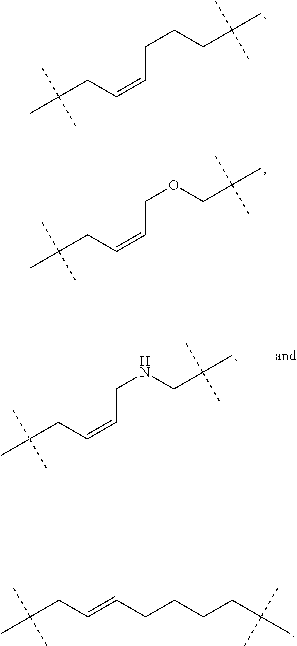

16. The compound or pharmaceutically acceptable salt according to claim 1, wherein L is (C₅-C₆)alkylenyl or (C₅-C₆)alkenylenyl.

17. The compound according to claim 1 which is:
(E)-10-((trans-4-aminocyclohexyl)oxy)-12-chloro-3-methyl-5,6,15,16-tetrahydrobenzo[c]pyrido[4,3-j][1]azacyclododecine-1,14(2H,9H)-dione;
(E)-12-chloro-10-((trans-4-(dimethylamino)cyclohexyl)oxy)-3-methyl-5,6,15,16-tetrahydrobenzo[c]pyrido[4,3-j][1]azacyclododecine-1,14(2H,9H)-dione;
12-chloro-10-((trans-4-(dimethylamino)cyclohexyl)oxy)-3-methyl-6,7,8,9,15,16-hexahydrobenzo[c]pyrido[4,3-j][1]azacyclododecine-1,14(2H,5H)-dione;
(Z)-10-((trans-4-aminocyclohexyl)oxy)-12-chloro-3-methyl-5,6,15,16-tetrahydrobenzo[c]pyrido[4,3-j][1]azacyclododecine-1,14(2H,9H)-dione;
(Z)-12-chloro-10-((trans-4-(dimethylamino)cyclohexyl)oxy)-3-methyl-5,6,15,16-tetrahydrobenzo[c]pyrido[4,3-j][1]azacyclododecine-1,14(2H,9H)-dione;
(E)-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione;
(Z)-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione;
(E)-13-chloro-11-((trans-4-(dimethylamino)cyclohexyl)oxy)-3-methyl-5,6,7,10,16,17-hexahydro-1H-benzo[c]pyrido[4,3-k][1]azacyclotridecine-1,15(2H)-dione;
(E)-10-(ethyl(tetrahydro-2H-pyran-4-yl)amino)-3,15-dimethyl-5,6,15,16-tetrahydrobenzo[c]pyrido[4,3-j][1]azacyclododecine-1,14(2H,9H)-dione;
(E)-11-(ethyl(tetrahydro-2H-pyran-4-yl)amino)-3,6-dimethyl-5,6,7,10,16,17-hexahydro-1H-benzo[h]pyrido[4,3-c][1,6]diazacyclotridecine-1,15(2H)-dione;
11-(ethyl(tetrahydro-2H-pyran-4-yl)amino)-3-methyl-7,8,9,10,16,17-hexahydrobenzo[h]pyrido[4,3-c][1,6]oxaazacyclotridecine-1,15(2H,5H)-dione;
(E)-11-(ethyl(tetrahydro-2H-pyran-4-yl)amino)-3-methyl-7,10,16,17-tetrahydrobenzo[h]pyrido[4,3-c][1,6]oxaazacyclotridecine-1,15(2H,5H)-dione;
(Z)-11-(ethyl(tetrahydro-2H-pyran-4-yl)amino)-3-methyl-7,10,16,17-tetrahydrobenzo[h]pyrido[4,3-c][1,6]oxaazacyclotridecine-1,15(2H,5H)-dione;
(Z)-10-(ethyl(tetrahydro-2H-pyran-4-yl)amino)-3-methyl-6,9,15,16-tetrahydro-1H-benzo[g]pyrido[4,3-b][1,5]oxaazacyclododecine-1,14(2H)-dione;
(E)-12-(ethyl(tetrahydro-2H-pyran-4-yl)amino)-3-methyl-5,6,7,8,17,18-hexahydrobenzo[c]pyrido[4,3-l][1]azacyclotetradecine-1,16(2H,11H)-dione;
(E)-12-chloro-10-methoxy-3-methyl-5,6,15,16-tetrahydrobenzo[c]pyrido[4,3-j][1]azacyclododecine-1,14(2H,9H)-dione;
(Z)-12-chloro-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione;
(E)-12-chloro-10-(ethyl(tetrahydro-2H-pyran-4-yl)amino)-3-methyl-5,6,15,16-tetrahydrobenzo[c]pyrido[4,3-j][1]azacyclododecine-1,14(2H,9H)-dione;
(E)-12-chloro-10-isopropoxy-3-methyl-5,6,15,16-tetrahydrobenzo[c]pyrido[4,3-j][1]azacyclododecine-1,14(2H,9H)-dione;
(E)-11-(ethyl(tetrahydro-2H-pyran-4-yl)amino)-3-methyl-5,6,7,10,16,17-hexahydro-1H-benzo[c]pyrido[4,3-k][1]azacyclotridecine-1,15(2H)-dione;
11-(ethyl(tetrahydro-2H-pyran-4-yl)amino)-3-methyl-5,6,7,8,9,10,16,17-octahydro-1H-benzo[c]pyrido[4,3-k][1]azacyclotridecine-1,15(2H)-dione;
(Z)-11-(ethyl(tetrahydro-2H-pyran-4-yl)amino)-3-methyl-5,6,7,10,16,17-hexahydro-1H-benzo[c]pyrido[4,3-k][1]azacyclotridecine-1,15(2H)-dione;
(Z)-10-(ethyl(tetrahydro-2H-pyran-4-yl)amino)-3,5-dimethyl-5,6,15,16-tetrahydrobenzo[c]pyrido[4,3-j][1]azacyclododecine-1,14(2H,9H)-di one;
(E)-10-(ethyl(tetrahydro-2H-pyran-4-yl)amino)-3,5-dimethyl-5,6,15,16-tetrahydrobenzo[c]pyrido[4,3-j][1]azacyclododecine-1,14(2H,9H)-dione;
10-(ethyl(tetrahydro-2H-pyran-4-yl)amino)-3,5-dimethyl-6,7,8,9,15,16-hexahydrobenzo[c]pyrido[4,3-j][1]azacyclododecine-1,14(2H,5H)-di one;
(E)-10-(ethyl(tetrahydro-2H-pyran-4-yl)amino)-6-(hydroxymethyl)-3-methyl-5,6,15,16-tetrahydrobenzo[c]pyrido[4,3-j][1]azacyclododecine-1,14(2H,9H)-dione;
(Z)-10-(ethyl(tetrahydro-2H-pyran-4-yl)amino)-6-(hydroxymethyl)-3-methyl-5,6,15,16-tetrahydrobenzo[c]pyrido[4,3-j][1]azacyclododecine-1,14(2H,9H)-di one;
10-(ethyl(tetrahydro-2H-pyran-4-yl)amino)-6-(hydroxymethyl)-3-methyl-6,7, 8,9,15,16-hexahydrobenzo[c]pyrido[4,3-j][1]azacyclododecine-1,14(2H,5H)-dione;
11-(ethyl(tetrahydro-2H-pyran-4-yl)amino)-7-hydroxy-3-methyl-5,6,7,8,9,10,16,17-octahydro-1H-benzo[c]pyrido[4,3-k][1]azacyclotridecine-1,15(2H)-dione;
(E)-10-(ethyl(tetrahydro-2H-pyran-4-yl)amino)-15-(2-hydroxy ethyl)-3-methyl-5,6,15,16-tetrahydrobenzo[c]pyrido[4,3-j][1]azacyclododecine-1,14(2H,9H)-dione;
(E)-10-((4,4-difluorocyclohexyl)(ethyl)amino)-3-methyl-5,6,15,16-tetrahydrobenzo[c]pyrido[4,3-j][1]azacyclododecine-1,14(2H,9H)-di one;
(E)-10-(ethyl(piperidin-4-yl)amino)-3-methyl-5,6,15,16-tetrahydrobenzo[c]pyrido[4,3-j][1]azacyclododecine-1,14(2H, 9H)-di one;
(E)-10-(ethyl(1-isopropylpiperidin-4-yl)amino)-3-methyl-5,6,15,16-tetrahydrobenzo[c]pyrido[4,3-j][1]azacyclododecine-1,14(2H,9H)-di one;
(E)-10-(ethyl(1-methylpiperidin-4-yl)amino)-3-methyl-5,6,15,16-tetrahydrobenzo[c]pyrido[4,3-j][1]azacyclododecine-1,14(2H,9H)-dione;
(E)-10-(ethyl(1-(methylsulfonyl)piperidin-4-yl)amino)-3-methyl-5,6,15,16-tetrahydrobenzo[c]pyrido[4,3-j][1]azacyclododecine-1,14(2H,9H)-di one;
(E)-10-(ethyl(1-(2-hydroxyethyl)piperidin-4-yl)amino)-3-methyl-5,6,15,16-tetrahydrobenzo[c]pyrido[4,3-j][1]azacyclododecine-1,14(2H,9H)-dione;
(E)-10-((trans-4-aminocyclohexyl)(ethyl)amino)-3-methyl-5,6,15,16-tetrahydrobenzo[c]pyrido[4,3-j][1]azacyclododecine-1,14(2H,9H)-di one;
(E)-10-((trans-4-(dimethylamino)cyclohexyl)(ethyl)amino)-3-methyl-5,6,15,16-tetrahydrobenzo[c]pyrido[4,3-j][1]azacyclododecine-1,14(2H,9H)-dione;
(E)-11-(ethyl(tetrahydro-2H-pyran-4-yl)amino)-3-methyl-5,6,7,10,16,17-hexahydro-1H-benzo[h]pyrido[4,3-c][1,6]diazacyclotridecine-1,15(2H)-dione;
(Z)-11-(ethyl(tetrahydro-2H-pyran-4-yl)amino)-3-methyl-5,6,7,10,16,17-hexahydro-1H-benzo[h]pyrido[4,3-c][1,6]diazacyclotridecine-1,15(2H)-dione;
(E)-11-(ethyl(piperidin-4-yl)amino)-3-methyl-5,6,7,10,16,17-hexahydro-1H-benzo[c]pyrido[4,3-k][1]azacyclotridecine-1,15(2H)-dione;
(E)-10-((1-(cyclopropylmethyl)piperidin-4-yl)(ethyl)amino)-3-methyl-5,6,15,16-tetrahydrobenzo[c]pyrido[4,3-j][1]azacyclododecine-1,14(2H,9H)-dione;
(E)-11-(ethyl(1-isopropylpiperidin-4-yl)amino)-3-methyl-5,6,7,10,16,17-hexahydro-1H-benzo[c]pyrido[4,3-k][1]azacyclotridecine-1,15(2H)-dione;
(E)-10-(ethyl(1-(3,3,3-trifluoropropyl)piperidin-4-yl)amino)-3-methyl-5,6,15,16-tetrahydrobenzo[c]pyrido[4,3-j][1]azacyclododecine-1,14(2H,9H)-dione;
(E)-10-(ethyl(1-ethylpiperidin-4-yl)amino)-3-methyl-5,6,15,16-tetrahydrobenzo[c]pyrido[4,3-j][1]azacyclododecine-1,14(2H, 9H)-di one;
(E)-10-(ethyl(1-((1-methyl-1H-pyrazol-3-yl)methyl)piperidin-4-yl)amino)-3-methyl-5,6,15,16-tetrahydrobenzo[c]pyrido[4,3-j][1]azacyclododecine-1,14(2H,9H)-dione;
(E)-ethyl 2-(4-(ethyl(3-methyl-1,14-dioxo-1,2,5,6,9,14,15,16-octahydrobenzo[c]pyrido[4,3-j][1]azacyclododecin-10-yl)amino)piperidin-1-yl)-2-methylpropanoate;
(E)-10-(ethyl(1-(2,2,2-trifluoroethyl)piperidin-4-yl)amino)-3-methyl-5,6,15,16-tetrahydrobenzo[c]pyrido[4,3-j][1]azacyclododecine-1,14(2H,9H)-dione;
(E)-10-(ethyl(1-((6-methylpyridin-2-yl)methyl)piperidin-4-yl)amino)-3-methyl-5,6,15,16-tetrahydrobenzo[c]pyrido[4,3-j][1]azacyclododecine-1,14(2H,9H)-di one;
(E)-10-((trans-4-(diethylamino)cyclohexyl)(ethyl)amino)-3-methyl-5,6,15,16-tetrahydrobenzo[c]pyrido[4,3-j][1]azacyclododecine-1,14(2H,9H)-dione;
(E)-10-(ethyl(trans-4-morpholinocyclohexyl)amino)-3-methyl-5,6,15,16-tetrahydrobenzo[c]pyrido[4,3-j][1]azacyclododecine-1,14(2H,9H)-di one;
(E)-10-((1-(1,3-dihydroxypropan-2-yl)piperidin-4-yl)(ethyl)amino)-3-methyl-5,6,15,16-tetrahydrobenzo[c]pyrido[4,3-j][1]azacyclododecine-1,14(2H,9H)-dione;
(Z)-10-(ethyl(piperidin-4-yl)amino)-3-methyl-5,6,15,16-tetrahydrobenzo[c]pyrido[4,3-j][1]azacyclododecine-1,14(2H,9H)-dione;
(E)-11-((trans-4-(dimethylamino)cyclohexyl)(ethyl)amino)-3-methyl-5,6,7,10,16,17-hexahydro-1H-benzo[c]pyrido[4,3-k][1]azacyclotridecine-1,15(2H)-dione;
9-(ethyl(piperidin-4-yl)amino)-3-methyl-5,8,14,15-tetrahydro-1H-benzo[c]pyrido[4,3-j][1]azacycloundecine-1,13(2H)-dione;
(E)-10-((cis-4-(dimethylamino)cyclohexyl)(ethyl)amino)-3-methyl-5,6,15,16-tetrahydrobenzo[c]pyrido[4,3-j][1]azacyclododecine-1,14(2H,9H)-dione;
(E)-10-((trans-4-((2,2-difluoroethyl)amino)cyclohexyl)(ethyl)amino)-3-methyl-5,6,15,16-tetrahydrobenzo[c]pyrido[4,3-j][1]azacyclododecine-1,14(2H,9H)-dione;
(E)-10-((trans-4-((2,2-difluoroethyl)(methyl)amino)cyclohexyl)(ethyl)amino)-3-methyl-5,6,15,16-tetrahydrobenzo[c]pyrido[4,3-j][1]azacyclododecine-1,14(2H,9H)-dione;
(E)-10-(ethyl(trans-4-((2,2,2-trifluoroethyl)amino)cyclohexyl)amino)-3-methyl-5,6,15,16-tetrahydrobenzo[c]pyrido[4,3-j][1]azacyclododecine-1,14(2H,9H)-dione;
(E)-10-(ethyl(trans-4-(methyl(2,2,2-trifluoroethyl)amino)cyclohexyl)amino)-3-methyl-5,6,15,16-tetrahydrobenzo[c]pyrido[4,3-j][1]azacyclododecine-1,14(2H,9H)-dione;
(E)-10-((trans-4-(azetidin-1-yl)cyclohexyl)(ethyl)amino)-3-methyl-5,6,15,16-tetrahydrobenzo[c]pyrido[4,3-j][1]azacyclododecine-1,14(2H,9H)-dione;
(Z)-9-((trans-4-(dimethylamino)cyclohexyl)(ethyl)amino)-3-methyl-5,8,14,15-tetrahydro-1H-benzo[c]pyrido[4,3-j][1]azacycloundecine-1,13(2H)-dione;
9-((trans-4-(dimethylamino)cyclohexyl)(ethyl)amino)-3-methyl-5,6,7,8,14,15-hexahydro-1H-benzo[c]pyrido[4,3-j][1]azacycloundecine-1,13(2H)-dione;
(E)-10-(ethyl(tetrahydro-2H-pyran-4-yl)amino)-3-methyl-5,6,15,16-tetrahydrodipyrido[3,4-c:3′,4′-j][1]azacyclododecine-1,14(2H,9H)-dione;
(E)-10-((2-hydroxyethyl)(tetrahydro-2H-pyran-4-yl)amino)-3-methyl-5,6,15,16-tetrahydrobenzo[c]pyrido[4,3-j][1]azacyclododecine-1,14(2H,9H)-dione;
(E)-10-((1-(dimethylamino)piperidin-4-yl)(ethyl)amino)-3-methyl-5,6,15,16-tetrahydrobenzo[c]pyrido[4,3-j][1]azacyclododecine-1,14(2H,9H)-dione;
(E)-10-(ethyl(2-azaspiro[3.5]nonan-7-yl)amino)-3-methyl-5,6,15,16-tetrahydrobenzo[c]pyrido[4,3-j][1]azacyclododecine-1,14(2H,9H)-dione;
(E)-10-(ethyl(2-methyl-2-azaspiro[3.5]nonan-7-yl)amino)-3-methyl-5,6,15,16-tetrahydrobenzo[c]pyrido[4,3-j][1]azacyclododecine-1,14(2H,9H)-dione;
(E)-10-(ethyl(7-azaspiro[3.5]nonan-2-yl)amino)-3-methyl-5,6,15,16-tetrahydrobenzo[c]pyrido[4,3-j][1]azacyclododecine-1,14(2H,9H)-dione;
(E)-10-(ethyl(7-methyl-7-azaspiro[3.5]nonan-2-yl)amino)-3-methyl-5,6,15,16-tetrahydrobenzo[c]pyrido[4,3-j][1]azacyclododecine-1,14(2H,9H)-dione;
(E)-10-((6-aminospiro[3.3]heptan-2-yl)(ethyl)amino)-3-methyl-5,6,15,16-tetrahydrobenzo[c]pyrido[4,3-j][1]azacyclododecine-1,14(2H,9H)-dione;
(E)-10-((6-(dimethylamino)spiro[3.3]heptan-2-yl)(ethyl)amino)-3-methyl-5,6,15,16-tetrahydrobenzo[c]pyrido[4,3-j][1]azacyclododecine-1,14(2H,9H)-dione;
(E)-10-(ethyl(2-methyl-2-azaspiro[3.3]heptan-6-yl)amino)-3-methyl-5,6,15,16-tetrahydrobenzo[c]pyrido[4,3-j][1]azacyclododecine-1,14(2H,9H)-dione;
(E)-10-(ethyl(trans-4-(m ethylamino)cyclohexyl)amino)-3-methyl-5,6,15,16-tetrahydrobenzo[c]pyrido[4,3-j][1]azacyclododecine-1,14(2H,9H)-dione;
(E)-10-(ethyl(cis-4-(methylamino)cyclohexyl)amino)-3-methyl-5,6,15,16-tetrahydrobenzo[c]pyrido[4,3-j][1]azacyclododecine-1,14(2H,9H)-dione;
(E)-10-(ethyl(cis-4-(3-fluoroazetidin-1-yl)cyclohexyl)amino)-3-methyl-5,6,15,16-tetrahydrobenzo[c]pyrido[4,3-j][1]azacyclododecine-1,14(2H,9H)-dione;
(E)-10-(ethyl(trans-4-(3-fluoroazetidin-1-yl)cyclohexyl)amino)-3-methyl-5,6,15,16-tetrahydrobenzo[c]pyrido[4,3-j][1]azacyclododecine-1,14(2H,9H)-dione;
(E)-10-(azepan-4-yl(ethyl)amino)-3-methyl-5,6,15,16-tetrahydrobenzo[c]pyrido[4,3-j][1]azacyclododecine-1,14(2H,9H)-dione;
(E)-10-(ethyl(cis-4-hydroxycyclohexyl)amino)-3-methyl-5,6,15,16-tetrahydrobenzo[c]pyrido[4,3-j][1]azacyclododecine-1,14(2H,9H)-dione;
(E)-10-(ethyl(trans-4-hydroxycyclohexyl)amino)-3-methyl-5,6,15,16-tetrahydrobenzo[c]pyrido[4,3-j][1]azacyclododecine-1,14(2H,9H)-dione;
(E)-10-((cis-4-(3,3-difluoroazetidin-1-yl)cyclohexyl)(ethyl)amino)-3-methyl-5,6,15,16-tetrahydrobenzo[c]pyrido[4,3-j][1]azacyclododecine-1,14(2H,9H)-dione;
(E)-10-((trans-4-(3,3-difluoroazetidin-1-yl)cyclohexyl)(ethyl)amino)-3-methyl-5,6,15,16-tetrahydrobenzo[c]pyrido[4,3-j][1]azacyclododecine-1,14(2H,9H)-dione;
(E)-10-(ethyl(cis-4-((3,3,3-trifluoropropyl)amino)cyclohexyl)amino)-3-methyl-5,6,15,16-tetrahydrobenzo[c]pyrido[4,3-j][1]azacyclododecine-1,14(2H,9H)-dione;
(E)-10-(ethyl(trans-4-((3,3,3-trifluoropropyl)amino)cyclohexyl)amino)-3-methyl-5,6,15,16-tetrahydrobenzo[c]pyrido[4,3-j][1]azacyclododecine-1,14(2H,9H)-di one,
(E)-10-((trans-4-(dimethyl amino)cyclohexyl)oxy)-3-methyl-5,6,15,16-tetrahydrobenzo[c]pyrido[4,3-j][1]azacyclododecine-1,14(2H,9H)-di one,
(Z)-10-((trans-4-(dimethyl amino)cyclohexyl)oxy)-3-methyl-5,6,15,16-tetrahydrobenzo[c]pyrido[4,3-j][1]azacyclododecine-1,14(2H,9H)-di one, or
(E)-10-(ethyl(cis-4-morpholinocyclohexyl)amino)-3-methyl-5,6,15,16-tetrahydrobenzo[c]pyrido[4,3-j][1]azacyclododecine-1,14(2H,9H)-di one, or a pharmaceutically acceptable salt thereof.

18-19. (canceled)

20. A pharmaceutical composition comprising the compound or pharmaceutically acceptable salt thereof according to claim 1 and a pharmaceutically acceptable excipient.

21. A method of treating cancer comprising administering to a patient with cancer a therapeutically effective amount of the compound or pharmaceutically acceptable salt thereof according to claim 1.

22. The method according to claim 21, wherein said cancer is selected from the group consisting of: brain cancer, glioblastomas, leukemias, lymphomas, Bannayan-Zonana syndrome, Cowden disease, Lhermitte-Duclos disease, breast cancer, inflammatory breast cancer, Wilm's tumor, Ewing's sarcoma, Rhabdomyosarcoma, ependymoma, medulloblastoma, colon cancer, gastric cancer, bladder cancer, head and neck cancer, kidney cancer, lung cancer, liver cancer, melanoma, renal cancer, ovarian cancer, pancreatic cancer, prostate cancer, sarcoma, osteosarcoma, giant cell tumor of bone, and thyroid cancer.

23. (canceled)
